(19) United States
(12) Patent Application Publication
(10) Pub. No.: US 2012/0010178 A1
(43) Pub. Date: Jan. 12, 2012

(54) METHODS AND COMPOUNDS FOR TREATMENT OF NEURODEGENERATIVE DISORDERS

(75) Inventors: Lee Rubin, Wellesley, MA (US); Amy Sinor, Bradfordwoods, PA (US); Nina Ruslanovna Makhortova, Brookline, MA (US); Yin Miranda Yang, Cambridge, MA (US); Monica Hayhurst Bennett, Watertown, MA (US)

(73) Assignee: PRESIDENT AND FELLOWS OF HARVARD COLLEGE, Cambridge, MA (US)

(21) Appl. No.: 13/125,376

(22) PCT Filed: Oct. 21, 2009
(86) PCT No.: PCT/US2009/061468

§ 371 Date: Sep. 28, 2011

Related U.S. Application Data
(60) Provisional application No. 61/107,280, filed on Oct. 21, 2008.
(60) Provisional application No. 61/223,366, filed on Jul. 6, 2009.

(51) Int. Cl. A61K 31/635; A61P 25/00; A61K 31/167; A61K 31/55; C12N 5/0793; A61K 31/426

(52) U.S. Cl. 514/158; 435/375; 514/371; 514/622; 514/212.06

(57) ABSTRACT

Methods, compounds and compositions for promoting motor neuron survival and the treatment of a neurodegenerative disorders such as Spinal Muscular Atrophy (SMA) are described herein.

### CROSS REFERENCE TO RELATED APPLICATIONS

This application claims the benefit under 35 U.S.C. §19(e) of U.S. Provisional Patent Application Ser. No. 61/107,280 filed on Oct. 21, 2008 and under 35 U.S.C. §19(e) of U.S. Provisional Patent Application Ser. No. 61/223,366 filed on Jul. 6, 2009, the contents of which each are incorporated herein by reference in their entirety.

### FIELD OF INVENTION

The invention relates to methods, compounds and compositions for the treatment of a neurodegenerative disorders such as Spinal Muscular Atrophy (SMA).

### BACKGROUND

Spinal Muscular Atrophy (SMA) is the leading genetic cause of death in infants. This neurodegenerative disease results from diminished levels of the protein Survival of Motor Neuron (SMN) with motor neurons being the cell type most affected, although recent evidence suggests that other cells, such as muscle, might contribute to the disease phenotype. Data derived from SMA patients and from SMA mouse models suggest that therapeutics that elevate Survival of Motor Neuron (SMN) levels will be effective in treating this disease.

Previously performed high throughput image-based screens in human patient fibroblasts were focused on identifying compounds that elevate the number of SMN-containing gems. Although the functional difference between SMN localized in the nucleus, cytoplasm or gems has not completely been clarified, it has been shown that cytoplasmic SMN apart from being phosphorylated is capable of forming functional snRNP complexes and also contains an additional isoform that is not present in the nucleus. This data indicates that compounds that increase SMN in cytoplasm could be more important as compounds that just increase the gem number.

Current treatment for SMA consists of prevention and management of the secondary effect of chronic motor unit loss. Some drugs under clinical investigation for treatment of SMA include, e.g., Butyrates, Valproic acid, Hydroxyurea and Riluzole.

Therefore, there is need in the art for compositions and methods for treatment of SMA.

### SUMMARY

The present invention is based, in part, on the characterization of the SMN regulatory pathway. The understanding of this pathway has revealed target molecules that increase SMN level or GEM level, or modulate distribution of either, which are useful in the treatment of SMA.

In one aspect, the invention provides for a method of promoting motor neuron survival, the method comprising: contacting a motor neuron with a compound that modulates a biological pathway or a target described herein.

The compounds that modulate the biological pathway or target described herein can be a small molecules, peptides, antibodies, antibody fragments, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. In some embodiments, the compound binds to at least one component in the pathway.

### BRIEF DESCRIPTION OF THE DRAWINGS

FIGS. 1a-1b depict line graphs showing the effect of cardiac glycoside ouabain on SMN levels. FIG. 1a shows the effect of ouabain on nuclear SMN. FIG. 1b shows effect of ouabain on both nuclear and cytoplasm SMN.

FIGS. 2a-2e depict line graphs showing elevation of SMN levels in fibroblasts on treatment with Ca2+ modulators ionomycin (FIG. 2a, FIG. 2b), thapsigagin (FIG. 2c, FIG. 2d) and Calcimycin (FIG. 2e).

FIG. 3 depicts line graph showing the effect of PDGF on the levels of SMN in fibroblasts.

FIGS. 4a-4b depict bar graphs showing that PDGF does not increase the levels of SMN in the presence of PDGFR inhibition, cytoplasm SMN (FIG. 4a) and nuclear SMN (FIG. 4b).

FIG. 5 depicts a line graph showing that the levels of SMN decreases in the presence of PDGFR inhibition by DMPQ.

FIG. 6 is a bar graph showing that the levels of SMN decrease when fibroblasts are treated with an anti-PDGF antibody.

FIGS. 7a-7d depict bar graphs showing that PDGF does not increase the levels of SMN in the presence of PI3 kinase inhibitors LY294002 (FIG. 7a, FIG. 7b) and PI-103 and rapamycin (FIG. 7c, FIG. 7d).

FIG. 8 is a bar graph showing that PDGF does not increase the levels of SMN in the presence of an mTOR inhibitor (rapamycin).

FIG. 9 is a line graph showing the levels of SMN decreases in the presence of PI3 kinase inhibition.

FIG. 10 is a line graph showing the effect of FGF on the levels of SMN in patient cells.

FIGS. 11a-11c depict line graphs (FIG. 11a, FIG. 11b) and bar graph (FIG. 11c) showing that serum starvation and PI3 kinase inhibition reduce the levels of SMN in patient cells, cytoplasm (FIG. 11a), nuclear (FIG. 11b) and cell (FIG. 11c).

FIGS. 12a-12b depict bar graphs showing that GSK inhibitors increase the levels of SMN the most when added to pre-starved cell synergistically with PDGF. FIG. 12a shows cytoplasm SMN and FIG. 12b shows nuclear SMN.

FIGS. 13a-13d depict line graphs showing the effect of GSK inhibitors on SMN levels in motor neurons. Alsterpaullone-2-cyanoethyl (FIG. 13a, FIG. 13c) and alsterpaullone (FIG. 13b, FIG. 13d). Hb9 motor neurons (FIG. 13a, FIG. 13b) and G93A motor neurons (FIG. 13c, FIG. 13d). Concentrations used were 0.005, 0.014, 0.04, 0.12, 0.37, 1.1, 3.3, 10 and 30 μM.

FIGS. 14a-14b depict line graphs showing the effect of GSK-3β inhibitors on motor neuron survival. (FIG. 14a) Alsterpaullone and (FIG. 14b) 2-cyanoethyl-alsterpaullone.

FIGS. 15a-15c depict bar graphs showing the dose response curves produced from numerical values output obtained from the script detection of SMN parameters. Fibroblasts were stained with anti-SMN antibody (488) and with Hoechst nuclear dye (320), cells were treated with 10 μM Proteasome inhibitor MG-132. Cytoplasm SMN (FIG. 15a), nuclear SMN (FIG. 15b) and GEM number (FIG. 15c).

FIGS. 16a-16b depict a line graph (FIG. 16a) and a bar graph (FIG. 16b) illustrating SMN increase in response to compound treatment gives better signal after the methanol/acetone fixation and also in human SMA parental carriers versus human SMA patient's fibroblasts (FIG. 16a) and SMN parameters detected by the Opera Evoteck between untreated Parent and Patient fibroblasts (FIG. 16b).

FIGS. 17a-17c depict line graphs showing dose response curves of Lactacystin (FIG. 17a), PKR inhibitor (FIG. 17b) and WIN-55, 212-2 (FIG. 17c), on increasing SMN levels in different cellular compartments.

FIGS. 18a-18 depict line graphs showing different effect of increasing concentration of SAHA (FIG. 18a) and Trichostatin A (TSA, FIG. 18b) on nuclear SMN levels and GEM number. This illustrates how different concentrations of the compound could increase Nuclear SMN and at the same time decrease Gem Number.

FIGS. 19a-19e depict the screening tree workflow and hit selection and analysis. Scheme of SMN screen (FIG. 19a), example of a 384-well plate generated heat map, cells in columns 23-24 where treated with MG-132 and cells in columns 1-2 were treated with DMSO (FIG. 19b), scatter plots show the effect of 5000 compounds screened on the level of SMN in a different cellular compartments (FIG. 19c), diagram showing the hit selection area (FIG. 19d) and the screening tree workflow (FIG. 19e).

FIGS. 20a-20d depict line graphs showing dose curve response of Ouabain—representative among abundant hit series of cardiac glycoside (FIG. 20a) and Ouabain IC50 indicating inhibition of NA/K ATPase (FIG. 20b). Ca++ ionophore Ionomycin (FIG. 20c) and RTK activator PDGF (FIG. 20d), as an example of follow on mechanism from pathways initiated by Na/K pump inhibition.

FIGS. 21a-21h depict line graph (FIG. 21a, FIG. 21b and FIG. 21c), bar graphs (FIG. 21d, FIG. 21f and FIG. 21h) and photographs (FIG. 21e) showing that PDGF modulation of SMN is receptor mediated. (FIG. 21a) Inhibitory effect of DMPQ and AG-1296 two small molecules inhibitors of PDGFR on PDGF induced SMN increase. (FIG. 21b) Modulation of SMN levels by PDGFB-BB neutralization with a-PDGF antibodies. (FIG. 21c) a-PDGF neutralization curve of PDGF in media in which fibroblasts are normally maintained. (FIG. 21d) SMN levels after treatment with 50 ng/ml of PDGF-BB. (FIG. 21e) Western blot and (FIG. 21f) Western blot quantification of PDGF treated cells vs control. (FIG. 21g) Detection of kinase activation with extracts from fibroblasts that were treated with 50 ng/ml PDGF for 30 min and subjected to http://www.rndsystems.com/product_detail_objectname_mapkarray.aspxPhospho-Kinase Proteome array. (FIG. 21h) Phosphorylation of kinases sites that become phosphorylated upon stimulation with PDGF-BB.

FIGS. 22a-22b depict lines graphs showing decreased “basal” level SMN by 20% when DMSO (FIG. 22a) or AG-1296 (FIG. 22b), is added to the media in which cells are maintained.

FIG. 23 depicts line graph showing dose response curve of PI-3 kinase inhibitor (LY294002) tested alone without the PDGF, as seen main SMN decrease occurs in Gem Intensity.

FIGS. 24a-24h depict a photograph (FIG. 24a), a bar graph (FIG. 24b) and line graphs (FIGS. 24c-24h) showing that GSK3b inhibitors increase SMN level. (FIG. 24a) GSK3b phosphorylation on Ser-9 after PDGF-BB. (FIGS. 24b and 24h) GSK3b phosphorylation on Ser-9 after PDGF-BB treatment. Patient fibroblasts treated with GSK inhibitors CHIR99021 (FIG. 24c, FIG. 24d) and Alsterpaullone (FIG. 24d, FIG. 24e and FIG. 24 g), increase SMN levels.

FIGS. 25a-25b depict line graphs showing increase in SMN after treatment with GSK inhibitors GSK XV (GSK 15) (FIG. 25a) and GSK II (GSK 2) (FIG. 25b).

FIG. 26 depicts a bar graph showing SMN-knockdown with shRNA #2 construct decreases Motor Neuron survival.

FIGS. 27a-27d depict line graphs (FIGS. 27a and 27b) and bar graphs (FIGS. 27c and 27d) showing increase in SMN levels and neuronal survival on GSK inhibition in ES-cell derived neurons. (FIG. 27a) Alsterpaullone increases Gem intensity in neurons. (FIG. 27b) Alsterpaullone increases level of SMN in neurons. (FIG. 27c) Opera Evoteck quantification of SMN decrease in different cellular compartments in ES-cell derived neurons after SMN knockdown, Gem Intensity was the most affected parameter (FIG. 27c). Time course of ES-cell derived Motor Neurons survival infected with hairpins for SMN knockdown and non-targeting control shRNA treated with DMSO, lowering of SMN level lead to Motor neuron death. (FIG. 27d) Time course of ES-cell derived Motor Neurons survival infected with hairpins for SMN knockdown and non-targeting control shRNA treated with 1.25, 2.5 or 5 uM of Alsterpaullone. Treatment with Alsterpaullone rescues the survival of SMN-KD Motor Neurons up to control level.

FIGS. 28a-28h depict line graphs showing effect of Erk inhibitors (FIG. 28a FIG. 28c) and p38 inhibitors (FIG. 28d-FIG. 28h) on SMN modulation with PDGF-BB.

FIGS. 29 and 30 depict structures of some exemplary GSK3 inhibitions.

FIGS. 31a-31b depict tables summarizing the effect on SMN protein levels and motor neuron survival of various compounds tested in motor neurons (FIG. 31a) and fibroblasts from patients (FIG. 31b).

FIGS. 32a-32b depict line graphs showing decrease of, SMN levels (FIG. 32a) and spot intensity (FIG. 32b), in ES-derived motor neurons with AKT inhibitors deguelin, roteonone and rapamycin.

FIGS. 33 and 34 Schematic representations of exemplary biological pathways and targets that elevate SMN levels.

FIGS. 35a-35e depict line graphs showing fold increase in SMN levels with cardiac glycoside Ouabin (FIG. 35a), Na⁺ channel modulator Monensin (FIG. 35b), Ca²⁺ channel modulators calcimycin (A23187) (FIG. 35c) and ionomycin (FIG. 35d), and Thapsigargin (FIG. 35e).

FIGS. 36a-36b depict line graphs showing effect of growth factors EGF (FIG. 36a) and PDGF-BB (FIG. 36b) on SMN level fold increase.

FIGS. 37a-37e depict line graphs showing the effect of PDGR inhibitors (FIGS. 37a, 37c and 37d) and PDGF neutralizing antibodies (FIGS. 37b and 37e) on SMN levels of PDGF treated (FIGS. 37a and 37b) and untreated cells (FIG. 37c-37e).

FIGS. 38a-38d depict line graphs showing effect of preincubation with ERK inhibitors PD98059 (50 μM) and U0126 (50 μM) (FIG. 38a), PI-3 kinase inhibitors LY294002 (50 μM) and PI-103 (41M) (FIG. 38b), p-38 inhibitors SB202190 and SB203583 (FIGS. 38c and 38d) on SMN levels in PDGF-BB treated cells.

FIGS. 39a-39c depict bar graphs showing knockdown of SMN in shRNA treated fibroblasts (FIGS. 39a and 39b) and in ESC-derived motor neurons (FIG. 39c). Knockdown levels were measured after 2 days (FIG. 39a) and 8 days (FIG. 39b).

FIGS. 40a-40d depict line graphs showing SMN level fold change (FIGS. 40a and 40b) and p-GSK-Ser9 phosphorylation fold change (FIGS. 40c and 40d) by treatment with GSK inhibitors CHIR98014 (FIGS. 40a and 40c) and AR014418 (FIGS. 40b and 40d). CHIR98014 increased SMN over 2.5 fold at a concentration of 7.9 μM and AR014418 increased SMN 2 fold at a concentration of 23 μM. Both inhibitors promoted phosphorylation of p-GSK-Ser9.

FIG. 41 depicts structures of various compounds used herein.

### DETAILED DESCRIPTION

As used herein, the phrase “promoting motor neuron survival” refers to an increase in survival of motor neuron cells as compared to a control. In some embodiments, contacting of a motor neuron with a compound described herein results in at least about 10%, 20%, 30%, 40%, 50% 60%, 70%, 80%, 90%, 95%, 100%, 2-fold, 3-fold, 4-fold, 5-fold or more increase in motor neuron survival relative to non treated control.

Motor neuron survival can be assessed by for example (i) increased survival time of motor neurons in culture; (ii) increased production of a neuron-associated molecule in culture or in vivo, e.g., choline acetyltransferase, acetylcholinesterase, SMN or GEMs; or (iii) decreased symptoms of motor neuron dysfunction in vivo. Such effects may be measured by any method known in the art. In one non-limiting example, increased survival of motor neurons may be measured by the method set forth in Arakawa et al. (1990, J. Neurosci. 10:3507-3515); increased production of neuron-associated molecules may be measured by bioassay, enzymatic assay, antibody binding, Northern blot assay, etc., depending on the molecule to be measured; and motor neuron dysfunction may be measured by assessing the physical manifestation of motor neuron disorder. In one embodiment, the increase in motor neuron survival can be assessed by measuring the increase in SMN protein levels and/or GEM numbers. Cell survival can also be measured by uptake of calcein AM, an analog of the viable dye, fluorescein diacetate. Calcein is taken up by viable cells and cleaved intracellularly to fluorescent salts which are retained by intact membranes of viable cells. Microscopic counts of viable neurons correlate directly with relative fluorescence values obtained with the fluorimetric viability assay. This method thus provides a reliable and quantitive measurement of cell survival in the total cell population of a given culture (Bozyczko-Coyne et al., J. Neur. Meth. 50:205-216, 1993). Other methods of assessing cell survival are described in U.S. Pat. Nos. 5,972,639; 6,077,684 and 6417,160, contents of which are incorporated herein by reference.

In vivo motor neuron survival can be assessed by an increase in motor neuron, neuromotor or neuromuscular function in a subject. In one non-limiting example, motor neuron survival in a subject can be assessed by reversion, allevation, amelioration, inhibition, slowing down or stopping of the progeression, aggravation or severity of a condition associated with motor neuron dysfunction or death in a subject, e.g., SMA or ALS.

In some embodiments, the biological pathway to be modulated is selected from the group consisting of PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof. In some embodiments, the signaling pathway is the PI-3/AKT/GSK pathway. In some embodiments, the pathway comprises GSK-3b, CDK2, CDK5, PKR or IKK-2b.

In some embodiments, the target is selected from the group consisting of Na⁺/K⁺ channel, MAPK, cannobinoid receptor, GPCR, Ca²⁺ channel, K⁺ channel, PDE5, GSK/CDK, PKR, CDK2, IKK-2, proteasome, BMP/TGFbeta receptor and dopamine receptor.

In some embodiments, the compound is selected from the group consisting of RTK activator, insulin, FGF (e.g. FGF2), EGF, NGF, TGF (e.g. TGFβ), MAPK activator, kinase inhibitor, GSK inhibitor, CDK inhibitor, PKR inhibitor, IKK inhibitor, BMP/TGFβ ligand, cannabinoid or GPCR agonists, ion channel modulator (e.g. Na⁺/K⁺ channel modulator, Ca²⁺ channel modulator, K⁺ channel modulato), PDE5 inhibitor, HDAC inhibitor, proteasome inhibitor, dopamine receptor ligand, PDGF, and combinations thereof.

Without wishing to be bound by theory, the compound functions by increasing, inhibiting, preventing, blocking, stopping and/or reducing signaling activity in a biological pathway described herein. In some embodiments, a compound described herein can alter the signaling activity by at least 5%, 10%, 20%, 30%, 40%, 50%, 60%, 70%, 80%, 90%, 95%, 100%, 2-fold, 3-fold, 4-fold, 5-fold, 10-fold or more relative to when pathway is not being modulated by the compound.

In some embodiments, the compound is a GSK inhibitor. GSK inhibitors are known widely in the art and can be grouped into different chemical classes such as pyrroloazepine, flavone, beruazepinone, bis-indole, pyrrolopyrazine, pyridyloxadiazole, pyrazolopyridine, pyrazolopyridazine, aminopyridine, pyrazoloquinoxaline, oxindole (indolinone), thiazole, bisindolylmaleimide, azainodolylmaleimide, arylindolemaleimide, aniliomaleimide, phenylaminopyridine, triazole, pyrrolopyrimidine, pyrazolopyrimidine, and chloromethyl thienyl ketone. In some embodiments, the compound is a GSK inhibitor selected from the group consisting of CHIR98014, CHIR99021, GSK1, GSK2, GSK6, GSK7, GSK8 (ARA014418), GSK 13, hymenialdisine, flavopiridol, aloisine A, aloisine B, compound 12, pyrazolopyridine 18, pyrazolopyridine 9, pyrazolopyridine 34, CT20026, compound 1, SU9516, staurosporine, compound 5a, compound 29, compound 46, GF109203x, RO318220, SB216763, SB415286, I5, CGP60474, compound 8b, and combinations thereof.

Additional GSK-3beta inhibitors amenable to the invention are described in U.S. Pat. Nos. 7,056,939, 7,045,519, 7,037,918, 6,989,382, 6,949,547, 6,872,737, 6,800,632, 6,780,625, 6,608,063, 6,489,344, 6,479,490, 6,441,053, 6,417,185, 6,323,029, 6,316,259, and 6,057,117, the contents of which each are incorporated herein by reference in their entirety.

In some embodiments, the compound is of formula (I)

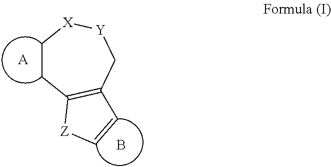

wherein:
A represents, with the adjacent ring, an optionally substituted aryl or an optionally substituted heteroaryl;
B represents, with the adjacent ring, an optionally substituted aryl or an optionally substituted heteroaryl;
X is NR^{N}, O, S, or CH₂;
Y is C(O), C(S), CH—SR^{N}CH—NHOH or S;
Z is NR^{N}, O, S or CHR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy; and
physiologically acceptable salts thereof.

In some embodiments, the compound of formula (I) has the structure shown in formula (II)

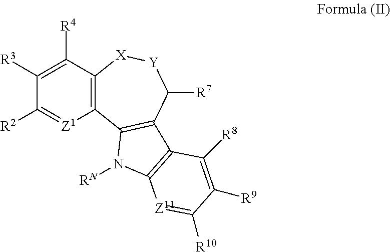

wherein:
Z¹ is N or CR¹;
Z¹¹ is N or CR¹¹;
R¹, R², R³, R⁴, R⁷, R⁸, R⁹, R¹⁰ and R¹¹ are each independently hydrogen, optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy;
R¹² is independently for each occurrence optionally substituted linear or branched alkyl, optionally substituted alkenyl, optionally substituted alkynyl, optionally substituted aryl, optionally substituted heteroaryl, optionally substituted arylalkyl, optionally substituted haloalkyl or optionally substituted alkylhydroxy;
X is NR^{N}, O, S, or CH₂;
Y is C(═O), C(═S), CH—SR^{N}CH—NHOH or S; and physiologically acceptable salts thereof.

In some embodiments, X is NH, O or CH₂. Preferably X is NH.

In some embodiments, Y is C(═O) or C(═S). Preferably Y is C(═O).

In some embodiments, Z¹ is N or CH.

In some embodiments, R², R³ and R⁴ are each H.

In some embodiments, at least one of R², R³ and R⁴ is optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N}.

In some embodiments. R² is optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N}. Preferably R² is alkyl, e.g., alkyl substituted with halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹².

In some embodiments, R² is an alkyl substituted with —CN.

In some embodiments R⁸, R⁹ and R¹⁰ are each H.

In some embodiments at least one of R⁸, R⁹ and R¹⁰ is optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃—, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N}.

In some embodiments at least one of R⁸, R⁹ and R¹⁰ is —NO₂.

In some embodiments, R⁹ is optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N}.

In some embodiments, R^{N} is H.

In some embodiments, the compound is kenpaullone (9-bromopaullone), alsterpaullone, 2-cyanoethyl-alsterpullone, 1-aza-alsterpaullon or 1-aza-kenpaullone.

In some embodiments, the compound of formula (I) has the structure shown in formula (III)

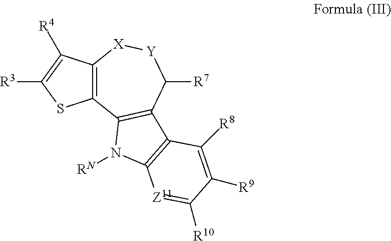

wherein:
Z¹¹ is N or CR¹¹;
R³, R⁴, R⁷, R⁸, R⁹, R¹⁰ and R¹¹ are each independently hydrogen, optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy;
R¹² is independently for each occurrence optionally substituted linear or branched alkyl, optionally substituted alkenyl, optionally substituted alkynyl, optionally substituted aryl, optionally substituted heteroaryl, optionally substituted arylalkyl, optionally substituted haloalkyl or optionally substituted alkylhydroxy;
X is NR^{N}, O, S, or CH₂;
Y is C(O), C(S), CH—SR^{N}CH—NHOH or S; and
physiologically acceptable salts thereof.

In some embodiments, the compound of formula (I) is 9-cyano-2,3-dimethoxypaullone; 2-iodopaullone; 2-bromo-9-nitropaullone; 2,3-dimethoxy-9-nitropaullone; 7-bromo-5-(4-nitrophenylhydrazono)-4,5-dihydro-1-H-[1]benzazepin2(3H)-one; 7,8-dimethoxy-5-(4-nitrophenylhydrazono)-4,5-dihydro-1H-[1]benzazepin-2-(3H)-one; 9-cyanopaullone; 9-chloropaullone; 9-trifluoromethylpaullone; 2,3-dimethoxy-9-trifluoromethylpaullone; 9-bromo-12-methyloxycarbonylmethylpaullone; 9-fluoropaullone; 9-bromo-2,3-dimethoxypaullone; 9-bromo-2,3-dimethoxypaullone; 9-methylpaullone; 10-bromopaullone; 2-bromopaullone; 11-chloropaullone; 2-(3-hydroxy-1-propinyl)-9-trifluoromethylpaullone; 9-bromo-12-(2-hydroxyethyl)-paullone; kenpaullone; Alsterpaullone; 2-cyanoetyl-alsterpaullone; 1-aza-kenpaullone; 1-aza-alsterpaullone; 9-bromo-12-methylpaullone; 9-bromo-5-(methyloxycarbonylmethyl)paullone; 11-methylpaullone; paullone; 11-ethylpaullone; 9-bromo-7,12-dihydro-6-(hydroxyamino)-indolo[2-3-d][1]benzazepine; 2,9-dibromopaullone; 11-bromopaullone; 2,3-dimethoxypaullone; 9-bromo-7,12-dihydro-6-methylthio-indolo[2-3-d][1]benzazepine; (E)-2(3-oxo-1-butenyl)-9-trifluoromethylpaullone; 9-bromo-12ethylpaullone; 9-bromo-7,12-dihydro-indolo[2-3-d][1]benzazepine-6(5H)-thione; 2-bromo-9-trifluoromethylpaullone; 2-[2-(1-hydroxycyclohexyl)ethenyl]-9-trifluoromethyl-paullone; 9-bromo-5-methylpaullone; 9-methoxypaullone; 2-iodo-9-trifluoromethylpaullone; 9-bromo-12-(tert-butyloxycarbonyl)-paullone; 9-bromo-12-(2-propenyl)paullone; 9-bromo-4-hydroxypaullone; 8,10-dichloropaullone; 5-benzyl-9-bromopaullone; 9-bromo-4-methoxypaullone; 9-bromo-5-ethylpaullone; 9-bromo-5,7bis-(tert-butyloxycarbonyl)-paullone; 4-methoxypaullone; 9-bromo-5,6,7,12-tetrahydrobenzo[6-7]cyclohept[1,2.b]indole; 2-phenyl-4-(2-thienyl)-5H-pyrido[2-3-d][1]benzazepine-6(7H)-thione; 9-bromo-5,7,12-tri-(tert-butyloxycarbonyl)-paullone; 9-bromo-5,12-bis-(tert-butyloxycarbonyl)paullone; 4-(4-chlorophenyl)-2-(2-naphthyl)-5H-pyrido[23-d][1]benzazepine-6(7H)-thione; and 5,6,7,12-tetrahydrobenzo[6-7]cyclohept[1,2-b]indole.

Other know derivatives of paullone family such as those found in Leost at al., Paullones Are Potent inhibitors of Glycogen Synthase Kinase-3B and Cyclin-dependent Kinase 5/p25, Eur. J. Biochem. (2000), 267, 5983-5994, PCT Publication Nos. WO 99/65910 and WO09/010,298, and U.S. Pat. Nos. 7,232,814 and 7,393,953 are also contemplated as suitable compounds of formula (I). The disclosures of each of the foregoing are incorporated by reference herein.

In some embodiments, the compound of formula (I) is not 9-cyano-2,3-dimethoxypaullone; 2-iodopaullone; 2-bromo-9-nitropaullone; 2,3-dimethoxy-9-nitropaullone; 7-bromo-5-(4-nitrophenylhydrazono)-4,5-dihydro-1-H-[1]benzazepin2(3H)-one; 7,8-dimethoxy-5-(4-nitrophenylhydrazono)-4,5-dihydro-1H-[1]benzazepin-2-(3H)-one; 9-cyanopaullone; 9-chloropaullone; 9-trifluoromethylpaullone; 2,3-dimethoxy-9-trifluoromethylpaullone; 9-bromo-12-methyloxycarbonylmethylpaullone; 9-fluoropaullone; 9-bromo-2,3-dimethoxypaullone; 9-bromo-2,3-dimethoxypaullone; 9-methylpaullone; 10-bromopaullone; 2-bromopaullone; 11-chloropaullone; 2-(3-hydroxy-1-propinyl)-9-trifluoromethylpaullone; 9-bromo-12-(2-hydroxyethyl)-paullone; kenpaullone; Alsterpaullone; 2-cyanoetyl-alsterpaullone; 1-aza-kenpaullone; 1-aza-alsterpaullone; 9-bromo-12-methylpaullone; 9-bromo-5-(methyloxycarbonylmethyl)paullone; 11-methylpaullone; paullone; 11-ethylpaullone; 9-bromo-7,12-dihydro-6-(hydroxyamino)-indolo[2-3-d][1]benzazepine; 2,9-dibromopaullone; 11-bromopaullone; 2,3-dimethoxypaullone; 9-bromo-7,12-dihydro-6-methylthio-indolo[2-3-d][1]benzazepine; (E)-2(3-oxo-1-butenyl)-9-trifluoromethylpaullone; 9-bromo-12-ethylpaullone; 9-bromo-7,12-dihydro-indolo[2-3-d][1]benzazepine-6(5H)-thione; 2-bromo-9-trifluoromethylpaullone; 2-[2-(1-hydroxycyclohexyl)ethenyl]-9-trifluoromethyl-paullone; 9-bromo-5-methylpaullone; 9-methoxypaullone; 2-iodo-9-trifluoromethylpaullone; 9-bromo-12-(tert-butyloxycarbonyl)-paullone; 9-bromo-12-(2-propenyl)paullone; 9-bromo-4-hydroxypaullone; 8,10-dichloropaullone; 5-benzyl-9-bromopaullone; 9-bromo-4-methoxypaullone; 9-bromo-5-ethylpaullone; 9-bromo-5,7bis-(tert-butyloxycarbonyl)-paullone; 4-methoxypaullone; 9-bromo-5,6,7,12-tetrahydrobenzo[6-7]cyclohept[1,2.b]indole; 2-phenyl-4-(2-thienyl)-5H-pyrido[2-3-d][1]benzazepine-6(7H)-thione; 9-bromo-5,7,12-tri-(tert-butyloxycarbonyl)-paullone; 9-bromo-5,12-bis-(tert-butyloxycarbonyl)paullone; 4-(4-chlorophenyl)-2-(2-naphthyl)-5H-pyrido[23-d][1]benzazepine-6(7H)-thione; and 5,6,7,12-tetrahydrobenzo[6-7]cyclohept[1,2-b]indole.

In some embodiments, the compound is of formula (IV):

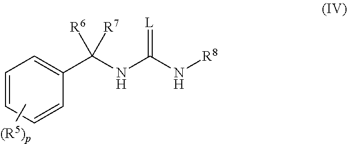

wherein:
L is O or S; and
p is 0, 1, 2, 3, 4 or 5;
each R⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁹;
each R⁶ and R⁷ is independently hydrogen, alkyl, alkenyl, alkynyl, aryl, heteroaryl, arylalkyl, haloalkyl, or alkylhydroxy;
R⁸ is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁰;
each R⁹ and R¹⁰ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, alkylhydroxy, haloalkyl or halo;
each R^{b} is independently hydrogen, alkyl, alkenyl, alkynyl, aryl, heteroaryl, arylalkyl, alkylhydroxy, or haloalkyl;
each R^{c} is independently hydrogen, alkyl, alkenyl, alkynyl, aryl, heteroaryl, arylalkyl, alkylhydroxy, or haloalkyl;
each R^{d} is independently hydrogen, alkyl, alkenyl, alkynyl, aryl, heteroaryl, arylalkyl, alkylhydroxy, or haloalkyl;
each R^{e} is independently hydrogen, alkyl, alkenyl, alkynyl, aryl, heteroaryl, arylalkyl, alkylhydroxy, or haloalkyl; and
each R^{f} is independently hydrogen, alkyl, alkenyl, alkynyl, aryl, heteroaryl, arylalkyl, alkylhydroxy, or haloalkyl.

In some embodiments, L is O or S.

In some embodiments, L is O.

In some embodiments, each R⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁹.

In some embodiments, p is 0, 1, 2, 3, 4 or 5.

In some embodiments, p is 1.

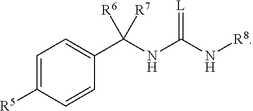

In some embodiments, the compound is

In some embodiments, R⁵ is —OR^{d}.

In some embodiments, R⁵ is —OCH₃.

In some embodiments, each R⁶ and R⁷ is independently hydrogen, alkyl, alkenyl, alkynyl, aryl, heteroaryl, arylalkyl, haloalkyl, or alkylhydroxy.

In some embodiments, R⁶ is hydrogen.

In some embodiments, R⁷ is hydrogen.

In some embodiments, R⁸ is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁰.

In some embodiments, R¹⁰ is halo, —NO₂, —SO₃⁻, —CN, —C(═O)R^{a}, —C(═O)N(R^{b})₂, —C(═O)OR^{c}, —OR^{d}, —NR^{e}₂, or —SR^{f}.

In some embodiments, R⁸ is heteroaryl substituted with 1 R¹⁰.

In some embodiments, R⁸ is a 5-membered heterocycle.

In some embodiments, R⁸ is a thiazole.

In some embodiments, the compound is

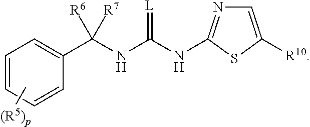

In some embodiments, R¹⁰ is —NO₂.

In some embodiments, the compound of is

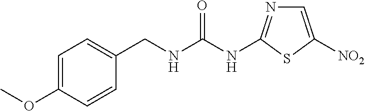

(AR-A014418, GSK8), a GSK-313 inhibitor.

In some embodiments, the compound is N-Butyl-N′-(5-nitro-1,3-thiazol-2-yl)urea; N-(5-Nitro-1,3-thiazol-2-yl)pentanamide; 1-{4-Amino-2-[(4-methoxyphenyl)amino]-1,3-thiazol-5-yl}ethanone; N-Benzyl-N′-(5-nitro-1,3-thiazol-2-yl)urea; N-(4-methoxybenzyl)-N′-(5-nitro-1,3-thiazol-2-yl)urea; 3-(4-Methoxyphenyl)-N-(5-nitro-1,3-thiazol-2-yl)propanamide; 4-(4-Methoxyphenyl)-N-(5-nitro-1,3-thiazol-2-yl)butanamide; 2-(3-Methoxyphenyl)-N-(5-nitro-1,3-thiazol-2-yl)acetamide; 2-(4-Fluorophenyl)-N-(5-nitro-1,3-thiazol-2-yl)propanamide; 2-(3-Methylphenyl)-N-(5-nitro-1,3-thiazol-2-yl)acetamide; 1-benzyl-3-naphthalen-1-yl-urea or 1-benzyl[1,3]dioxol-5-yl-3-benzyl-urea. Other compounds of formula (IV) amenable to the invention are described in U.S. Pat. No. 7,056,939 and PCT Publication Nos. WO07/017,145 and WO03/089419. The disclosures of each of the foregoing are incorporated by reference herein.

In some other embodiments, the compound is not N-Butyl-N′-(5-nitro-1,3-thiazol-2-yl)urea; N-(5-Nitro-1,3-thiazol-2-yl)pentanamide; 1-{4-Amino-2-[(4-methoxyphenyl)amino]-1,3-thiazol-5-yl}ethanone; N-Benzyl-N′-(5-nitro-1,3-thiazol-2-yl)urea; N-(4-methoxybenzyl)-N′-(5-nitro-1,3-thiazol-2-yl)urea; 3-(4-Methoxyphenyl)-N-(5-nitro-1,3-thiazol-2-yl)propanamide; 4-(4-Methoxyphenyl)-N-(5-nitro-1,3-thiazol-2-yl)butanamide; 2-(3-Methoxyphenyl)-N-(5-nitro-1,3-thiazol-2-yl)acetamide; 2-(4-Fluorophenyl)-N-(5-nitro-1,3-thiazol-2-yl)propanamide; 2-(3-Methylphenyl)-N-(5-nitro-1,3-thiazol-2-yl)acetamide; 1-benzyl-3-naphthalen-1-yl-urea or 1-benzyl[1,3]dioxol-5-yl-3-benzyl-urea.

In some embodiments, the compound is of formula (V):

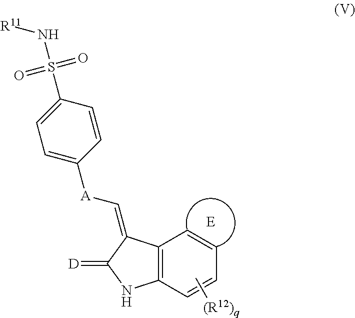

wherein:
A is NH, O, S or CH₂; and D is O or S;
E is an aryl or heteroaryl moiety;
q is 0, 1 or 2;
each R¹¹ and R¹² is independently hydrogen, alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹³;
each R¹³ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl; and
each R^{f} is independently hydrogen, alkyl, alkenyl, alkynyl, aryl, heteroaryl, arylalkyl, alkylhydroxy or haloalkyl.

In some embodiments, A is NH or 0.

In some embodiments, A is NH.

In some embodiments, D is O or S.

In some embodiments, D is O.

In some embodiments, E is an aryl or heteroaryl moiety.

In some embodiments, E is a heteroaryl moiety.

In some embodiments, E is a thiazole.

In some embodiments, the compound is

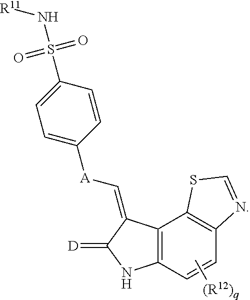

In some embodiments, R¹¹ is hydrogen, alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹³.

In some embodiments, R¹³ is halo, —NO₂, —SO₃⁻, —CN, —C(═O)R^{a}, —C(═O)N(R^{b})₂, —C(═O)OR^{c}, —OR^{d}, —NR^{e}₂, or —SR^{f}.

In some embodiments, R¹³ is heteroaryl.

In some embodiments, R¹² is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹³;

In some embodiments, q is 0, 1 or 2.

In some embodiments, q is 0.

In some embodiments, the compound is

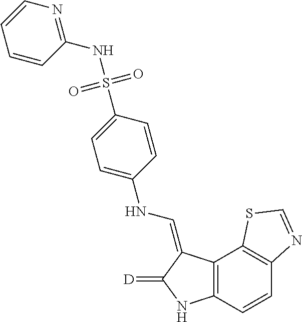

(GW8510), a GSK/CDK inhibitor.

In some other embodiments, the compound is not GW8510.

In some embodiments, the compound is of formula (VI):

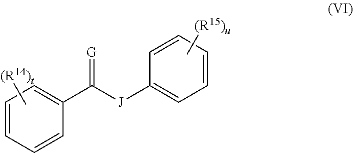

wherein:
t is 0, 1, 2, 3, 4 or 5; and
u is 0, 1, 2, 3, 4 or 5;
G is 0 or S;
J is O, S, NH or CH₂;
each R¹⁴ and R¹⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁶;
each R¹⁶ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl; and
each R^{f} is independently hydrogen, alkyl, alkenyl, alkynyl, aryl, heteroaryl, arylalkyl, alkylhydroxy or haloalkyl.

In some embodiments, G is O or S.

In some embodiments, G is O.

In some embodiments, J is O, S, NH or CH₂.

In some embodiments, J is NH.

In some embodiments, t is 0, 1, 2, 3, 4 or 5.

In some embodiments, t is 2.

In some embodiments, each R¹⁴ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁶.

In some embodiments, R¹⁶ is halo, —NO₂, —SO₃⁻, —CN, —C(═O)R^{a}, —C(═O)N(R^{b})₂, —C(═O)OR^{c}, —OR^{d}, —NR^{e}₂, or —SR^{f}.

In some embodiments, R¹⁴ is —OH.

In some embodiments, R¹⁴ is halo.

In some embodiments, R¹⁴ is —Cl.

In some embodiments, u is 0, 1, 2, 3, 4 or 5.

In some embodiments, u is 2.

In some embodiments, each R¹⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁶.

In some embodiments, R¹⁶ is halo, —NO₂, —SO₃⁻, —CN, —C(═O)R^{a}, —C(═O)N(R^{b})₂, —C(═O)OR^{c}, —OR^{d}, —NR^{e}₂, or —SR^{f}.

In some embodiments, R¹⁵ is —NO₂.

In some embodiments, R¹⁵ is halo.

In some embodiments, R¹⁵ is —Cl.

In some embodiments, R¹⁵ is alkyl substituted with 1-4 R¹⁶.

In some embodiments, R¹⁵ is substituted with 3 R¹⁶.

In some embodiments, R¹⁶ is halo.

In some embodiments, R¹⁶ is —F.

In some embodiments, the compound is

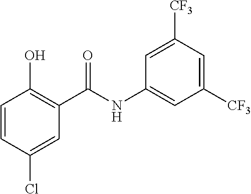

(IMD-0354), a IKK-2 inhibitor.

In some embodiments, the compound is

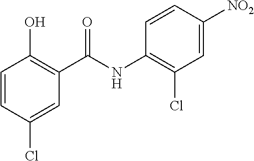

(niclosamide).

In some embodiments, compound is an inhibitor of PKR pathway and inhibits protein kinase R (PKR). PKR inhibitors include, but are not limited to, 3-[1-(3H-imidazol-4-yl)-meth-(Z)-ylidene]-5-methoxy-1,3-dihydroindol-2-one (SU9516), 2-aminopurine, 9-(4-bromo-3,5-dimethylpyridin-2-yl)-6-chloro-9H-purin-2-ylamine, 9-(4-bromo-3,5-dimethyl-pyridin-2-ylmethyl)-6-chloro-9H-purin-2-ylamine, phosphate salt, 9-(4-bromo-3,5-dimethyl-pyridin2-ylmethyl)-6-chloro-9H-purin-2-ylamine, hydrochloric acid salt, 6-bromo-9-(4-bromo-3,5-dimethyl-pyridin-2-ylmethyl)-9H-purin-2-ylamine, 6-bromo-9-(4-bromo-3,5-dimethyl-1-oxy-pyridin-2-ylmethyl)-9H-purin-2-ylamine, 2-(2-amino-6-chloro-purin-9-ylmethyl)-3,5-dimethyl-pyridin-4ol, 9-(4-allyloxy-3,5-dimethyl-pyridin-2-ylmethyl)-6-chloro-9H-purin-2-ylamine, 6-chloro-9-[4-(2-ethoxyethoxy)-3,5-dimethyl-pyridin-2-ylmethyl]-9H-purin-2ylamine, 6-chloro-9-(4-cyclopropylmethoxy-3,5-dimethylpyridin-2-ylmethyl)-9H-purin-2-ylamine, 6-chloro-9-(4 isobutoxy-3,5-dimethyl-pyridin-2-ylmethyl)-9H-purin-2ylamine, 6-chloro-9-(4-chloro-3,5-dimethyl-pyridin-2-ylmethyl)-9H-purin-2-ylamine, 6-chloro-9-(3,5-dimethylpyridin-2-ylmethyl)-9H-purin-2-ylamine, and 6-bromo-9(4-methoxy-3,5-dimethyl-pyridin-2-ylmethyl)-9H-purin-2ylamine, 3-[3,5-dimethyl-4-(4-methylpiperazine-1-carbonyl)-1H-pyrrol-2 ylmethylene)-4-pyridin-4-yl-1,3-dihydroindol-2-one, 3-[3-methyl-4-(piperidine-1-carbonyl)-! H-pyrrol-2-ylmethylene]-4-pyridin-4-yl1,3-dihydroindol-2-one, 3-(3,5-dimethyl-1H-pyrrol-2-ylmethylene)-4-pyridin-4-yl-1,3-dihydroindol-2-one, 3-[2(2-oxo-4-pyridin-4-yl-1,2-dihydroindol-3-ylidenemethyl-4,5,6,7-tetrahydro-1H-indol-3-yl]-propionic acid, 3-[5-ethyl2-(2-oxo-4-pyridin-4-yl-1,2-dihydroindol-3ylidenemethyl)-1H-pyrrol-3-yl]-propionic acid, 4-(2carboxyethyl)-2-methyl-5-(2-oxo-4-pyridin-4-yl-1,2dihydro-indol-3-ylidenemethyl)-1H-pyrrole-3-carboxylic acid ethyl ester, 3-[2,4-dimethyl-5-(2-oxo-4-pyridin-4-yl-1,2-dihydroindol-ylidenemethyl)-1H-pyrrol-3-yl]-propionic acid, 4-pyridin-4-yl-3-(4,5,6,7-tetrahydro-1H-indol-2-ylmethylene), 5-methyl-2-(2-oxo-4-pyridin-4-yl-1,2-dihydroindol-ylidenemethyl)-1H-pyrrole-3-carboxylic acid, 3-[3-(3morpholin-4-yl-propyl)-4,5,6,7-tetrahydro-1H-indol-2 ylmethylene]-4-pyridin-4-yl-1,3-dihydroindol-2-one, 3-[3-methyl-5-(4-methylpiperazine-1-carbonyl)-1H-pyrrol-ylmethylene]-4-pyridin-4-yl-1,3-dihydroindol-2-one, 3-(5-methylthiophen-2-ylmethylene)-4-pyridin-4-yl-1,3-dihydroindol-2-one, 4-[4-(2-oxo-4-pyridin-4-yl-1,2-dihydroindol3-ylidenemethyl)phenyl]-piperazine-1-carbaldehyde, 4-(2hydroxyethyl)-5-2-oxo-4-pyridin-4-yl-1,2-dihydroindol-3ylidenemethyl)-1H-pyrrole-3-carboxylic acid, [3-methyl-4(piperidine-1-carbonyl)-1H-pyrrol-2 ylmethylene]-4-piperidin-4-yl-1,3-dihydro-indol-2-one, 3-[3-methyl-4(morpholine-4-carbonyl)-1H-pyrrolylmethylene]-4piperidin-4-yl-1,3-dihydroindol-2-one, 3-(3,5-dibromo-4hydroxy-benzylidine-2-oxo-2,3-dihydro-1H-indole-5carbonitrile, 3-(3,5-dibromo-4-hydroxy-benzylidene)-5-(2-methyl-thiazol-4-yl)-1,3-dihydro-indol-2-one, 3-(3-bromo5-ethoxy-4-hydroxy-benzylidene)-5-(2-methyl-thiazol-4yl)-1,3-dihydro-indol-2-one, 3-(3,5-dichloro-4-hydroxybenzylidene-5-(2-methyl-thiazol-4-yl)-1,3-dihydro-indol-2one, (butanoyl)-1,3-dihydro-indol-2-one, 3-(3,5-dibromo-4hydroxy-benzylidene-5-(3-methyl-butanoyl-1,3-dihydroindol-2-one, 5-benzoyl-3-(3,5-dibromo-4-hydroxybenzylidene)-1,3-dihydro-indol-2-one, 5-benzoyl-3-(3,5dichloro-4-hydroxy-benzylidene)-1,3-dihydro-indol-2-one, 5-benzoyl-3-(3-bromo-5-ethoxy-4-hydroxy-benzylidene)-1,3-dihydro-indol-2-one, 3-(3,5-dichloro-4-hydroxy-benzylidene-5-(3-methyl-butanoyl)-1,3-dihydro-indol-2-one, 3-(3-bromo-5-ethoxy-4-hydroxy-benzylidene)-5-(3-methylbutanoyl)-1,3-dihydro-indol-2-one, 3-(3,5-dibromo-4-hydroxy-benzylidene-5-(pyridine-3-carbonyl)-1,3-dihydro-indol-2-one, 3-(3,5-dichloro-4-hydroxy-benzylidene)-5(pyridine-3-carbonyl)-1,3-dihydro-indol-2-one, 3-(3,5dibromo-4-hydroxy-benzylidene)-5-(pyridine-4-carbonyl)1,3-dihydro-indol-2-one, 3-(3-bromo-5-ethoxy-4-hydroxybenzylidene)-5-(pyridine-4-carbonyl)-1,3-dihydro-indol-2one, 3-(3,5-dichloro-4-hydroxy-benzylidene)-5-(pyridine-4carbonyl)-1,3-dihydro-indol-2-one, 3-(3-bromo-5-ethoxy-4hydroxy-benzylidene)-5-(pyridine-3-carbonyl)-1,3-dihydro-indol-2-one, 3-(3,5-dichloro-4-hydroxybenzylidene-5-(oxazol-5-yl)-1,3-dihydro-indol-2-one, 3-(3,4-dibromo-4-hydroxy-benzylidene)-5-(oxazol-5-yl)-1,3dihydro-indol-2-one, 3-(3,5-dibromo-4-hydroxybenzylidene)-5-(2-ethyl-thiazol-4-yl)-1,3-dihydro-indol-2one, 3-(3,5-dibromo-4-hydroxy-benzylidene)-2-oxo-2,3-dihydro-1H-indol-5-carboxylic acid methyl ester, 3-(3,5dibromo-4-hydroxy-benzylidene-5-(furan-2-carbonyl)-1,3dihydro-indol-2-one, 3-(3,5-dichloro-4-hydroxybenzylidene-5-(furan-2-carbonyl)-!,3-dihydro-indol-2-one, 3-(3-bromo-5-ethoxy-4-hydroxy-benzylidene-5-(furan-2-carbonyl)-1,3-dihydro-indol-2-one, 5-cyclopropanecarbonyl-3-(3,5-dibromo-4-hydroxy-benzylidene)-1,3-dihydroindol-2-one, 5-aminomethyl-3-(3,5-dibromo-4-hydroxybenzylidene)-1,3-dihydro-indol-2-one, 5-cyclopentanecarbonyl-3-(3,5-dibromo-4-hydroxy-benzylidene)-1,3-dihydro-indol-2-one, 3-(3,5-dichloro-4-hydroxy-benzylidene)-2-oxo-2,3-dihydro-1H-indole-5-carboxylic acid methyl ester, 3-(3,5-dibromo-4-hydroxybenzylidene)-1,3-dihydro-indol-2-one, 3-(3,5-dibromo-4hydroxy-benzylidene)-5-(thiophene-2-carbonyl)-1,3-dihydro-indol-2-one, 5-(2-amino-thiazol-4-yl)-3-(3,5-dibromo-4-hydroxy-benzylidene)-1,3-dihydro-indol-2-one, 3-(3,5-dibromo-4-hydroxy-benzylidene)-5-(imidazo[1,2a]pyridin-2-yl)-1,3-dihydro-indol-2-one, 3-(3,5-dibromo-4-hydroxy-benzylidene)-5-propionyl-1,3-dihydro-indol-2one, 3-(3,5-dibromo-4-hydroxy-benzylidene)-2-oxo-2,3-dihydro-1H-indole-5-sulfonic acid amide, 3-(3,5-dibromo-4-hydroxy-benzylidene)-2-oxo-2,3dihydro-1H-indole-5sulfonic acid N,N-diethylamide, 3-(3,5-dibromo-4-hydroxybenzylidene)-5-(pyrrolidine-1-sulfonyl)-1,3-dihydro-indol2-one, 3-(3,5-dibromo-4-hydroxy-benzylidene)-2-oxo-2,3-dihydro-1H-indol-5-sulfonic acid (N-2-dimethylaminoethyl)-N-methyl-amide, 3-(3,5-dibromo-4-hydroxybenzylidene)-5-(isoxazole-5-carbonyl)-1,3-dihydro-indol2-one, 5-chloro-3-(3,5-dibromo-4-hydroxy-benzylidene)-1,3-dihydro-indol-2-one, 5-chloro-3-(3,5-dichloro-4-hydroxy-benzylidene)-1,3-dihydro-indol-2-one, 3-(3,5dibromo-4-hydroxy-benzylidene)-5-trifluoromethoxy-1,3dihydro-indol-2-one, 5-bromo-3-(3,5-dichloro-4-hydroxybenzylidene)-1,3-dihydro-indol-2-one, 3-(3-bromo-5-ethoxy-4-hydroxy-benzylidene)-5-iodo-1,3-dihydro-indol2-one, 3-(3-bromo-4-hydroxy-5-methoxy-benzylidene)-5 iodo-1,3-dihydro-indol-2-one, 5-bromo-3-(3,5-diiodo-4-hydroxy-benzylidene)-1,3-dihydro-indol-2-one, 3-(3,5diiodo-4-hydroxy-benzylidene)-5-trifluoromethoxy-1,3dihydro-indol-2-one, 3-(3-bromo-4-hydroxy-5-methoxybenzylidene)-1,3-dihydro-indol-2-one, 3-(3,5-dinitro-4-hydroxy-benzylidene)-1,3-dihydro-indol-2-one, 3-(3,5dichloro-4-hydroxy-benzylidene)-!,3-dihydro-indol-2-one, 3-(3-chloro-4-hydroxy-5-methoxy-benzylidene)-!,3-dihydro-indol-2-one, 3-(3,5-diiodo-4-hydroxy-benzylidene)-1,3dihydro-indol-2-one, 3-(3-bromo-4-hydroxy-5-methoxybenzylidene)-5-chloro-1,3-dihydro-indol-2-one, 5-chloro-3-(3,5-dinitro-4-hydroxy-benzylidene)-1,3-dihydro-indol-2one, 5-chloro-3-(4-hydroxy-3-methoxy-5-nitrobenzylidene)-1,3-dihydro-indol-2-one, 5-chloro-3-(3-chloro-4-hydroxy-5-methoxy-benzylidene)-1,3-dihydroindol-2-one, 5-chloro-3-(3,5-diiodo-4-hydroxybenzylidene)-1,3-dihydro-indol-2-one, 5-bromo-3-(3-bromo-5-ethoxy-4-hydroxy-benzylidene)-1,3-dihydroindol-2-one, 5-bromo-3-(3-bromo-4-hydroxy-5-methoxybenzylidene)-1,3-dihydro-indol-2-one, 3-(3-bromo-4-hydroxy-5-methoxy-benzylidene)-5,6-difluoro-1,3-dihydroindol-2-one, 3-(3-bromo-5-ethoxy-4-hydroxy-benzylidene)-5-trifluoromethoxy-1,3-dihydro-indol-2-one, 3-(3,5-dichloro-4-hydroxy-benzylidene)-5-trifluoromethoxy-1,3dihydro-indol-2-one, 3-(3-bromo-4-hydroxy-5-methoxybenzylidene)-5-trifluoromethoxy-1,3-dihydro-indol-2-one, 3-(3,5-dibromo-4-hydroxy-benzylidene)-5,7-dinitro-1,3-dihydro-indol-2-one, 3-(3,5-dibromo-4-hydroxybenzylidene)-5-nitro-1,3-dihydro-indol-2-one, 3-(3,5-dibromo-4-hydroxy-benzylidene)-7-iodo-1,3-dihydro-indol-2-one, 3-(3,5-dichloro-4-hydroxy-benzylidene)-5-nitro-1,3-dihydro-indol-2-one, 3-(3,5-dibromo-4-hydroxy-benzylidene)7-iodo-1,3-dihydro-indol-2-one, 7-bromo-3-(3,5-dichloro-4-hydroxy-benzylidene)-1,3-dihydro-indol-2-one, 3-(3bromo-5-ethoxy-4-hydroxy-benzylidene)-5-nitro-1,3-dihydro-indol-2-one, and 2-(N-{3-[3-(3,5-dibromo-4hydroxy-benzylidene)-2-oxo-2,3-dihydro-1H-indol-5-yl]-2oxo-ethyl}-N-methyl-amino)-acetamide. Analogs of SU9516 and PKR inhibitor are described in Yu et al., Biochem. Pharmacol. 64:10911100, 2002, Lane et al., Cancer Res. 61:6170-6177, 2001, Jammi et al., Biochem. Biophys. Res. Commun. 308:50-57, 2003, Shimazawa et al, Neurosci. Lett. 409:192-195, 2006, Peel, J. Neuropathol. Exp. Neurol. 63:97-105, 2004, Bando et al, Neurochem. Int. 46:11-18, 2005, Peel et al. Hum. Mol. Genet. 10:1531-1538, 2001, and Chang et al, J. Neurochem. 83:1215-1225, 2002, the contents of which each are incorporated herein by reference in their entirety.

In some embodiments, PKR inhibitor is not 3-[1-(3Himidazol-4-yl)-meth-(Z)-ylidene]-5-methoxy-1,3-dihydroindol-2-one (SU9516), 2-aminopurine, 9-(4-bromo-3,5-dimethylpyridin-2-yl)-6-chloro-9H-purin-2-ylamine, 9-(4-bromo-3,5-dimethyl-pyridin-2-ylmethyl)-6-chloro-9H-purin-2-ylamine, phosphate salt, 9-(4-bromo-3,5-dimethyl-pyridin2-ylmethyl)-6-chloro-9H-purin-2-ylamine, hydrochloric acid salt, 6-bromo-9-(4-bromo-3,5-dimethyl-pyridin-2-ylmethyl)-9H-purin-2-ylamine, 6-bromo-9-(4-bromo-3,5-dimethyl-1-oxy-pyridin-2-ylmethyl)-9H-purin-2-ylamine, 2-(2amino-6-chloro-purin-9-ylmethyl)-3,5-dimethyl-pyridin-4ol,9-(4-allyloxy-3,5-dimethyl-pyridin-2-ylmethyl)-6-chloro-9H-purin-2-ylamine, 6-chloro-9-[4-(2-ethoxyethoxy)-3,5-dimethyl-pyridin-2-ylmethyl]-9H-purin-2ylamine, 6-chloro-9-(4-cyclopropylmethoxy-3,5-dimethylpyridin-2-ylmethyl)-9H-purin-2-ylamine, 6-chloro-9-(4 isobutoxy-3,5-dimethyl-pyridin-2-ylmethyl)-9H-purin-2ylamine, 6-chloro-9-(4-chloro-3,5-dimethyl-pyridin-2-ylmethyl)-9H-purin-2-ylamine, 6-chloro-9-(3,5-dimethylpyridin-2-ylmethyl)-9H-purin-2-ylamine, and 6-bromo-9(4-methoxy-3,5-dimethyl-pyridin-2-ylmethyl)-9H-purin-2ylamine, 3-[3,5-dimethyl-4-(4-methylpiperazine-1-carbonyl)-1H-pyrrol-2 ylmethylene)-4-pyridin-4-yl-1,3-dihydroindol-2-one, 3-[3-methyl-4-(piperidine-1-carbonyl)-! H-pyrrol-2-ylmethylene]-4-pyridin-4-yl1,3-dihydroindol-2-one, 3-(3,5-dimethyl-1H-pyrrol-2-ylmethylene)-4-pyridin-4-yl-1,3-dihydroindol-2-one, 3-[2(2-oxo-4-pyridin-4-yl-1,2-dihydroindol-3-ylidenemethyl-4,5,6,7-tetrahydro-1H-indol-3-yl]-propionic acid, 3-[5-ethyl2-(2-oxo-4-pyridin-4-yl-1,2-dihydroindol-3-ylidenemethyl)-1H-pyrrol-3-yl]-propionic acid, 4-(2-carboxyethyl)-2-methyl-5-(2-oxo-4-pyridin-4-yl-1,2dihydro-indol-3-ylidenemethyl)-1H-pyrrole-3-carboxylic acid ethyl ester, 3-[2,4-dimethyl-5-(2-oxo-4-pyridin-4-yl-1,2-dihydroindol-ylidenemethyl)-1H-pyrrol-3-yl]-propionic acid, 4-pyridin-4-yl-3-(4,5,6,7-tetrahydro-1H-indol-2-ylmethylene), 5-methyl-2-(2-oxo-4-pyridin-4-yl-1,2-dihydroindol-ylidenemethyl)-1H-pyrrole-3-carboxylic acid, 3-[3-(3morpholin-4-yl-propyl)-4,5,6,7-tetrahydro-1H-indol-2 ylmethylene]-4-pyridin-4-yl-1,3-dihydroindol-2-one, 3-[3-methyl-5-(4-methylpiperazine-1-carbonyl)-1H-pyrrol-ylmethylene]-4-pyridin-4-yl-1,3-dihydroindol-2-one, 3-(5-methylthiophen-2-ylmethylene)-4-pyridin-4-yl-1,3-dihydroindol-2-one, 4-[4-(2-oxo-4-pyridin-4-yl-1,2-dihydroindol3-ylidenemethyl)phenyl]-piperazine-1-carbaldehyde, 4-(2hydroxyethyl)-5-2-oxo-4-pyridin-4-yl-1,2-dihydroindol-3ylidenemethyl)-1H-pyrrole-3-carboxylic acid, [3-methyl-4(piperidine-1-carbonyl)-1H-pyrrol-2 ylmethylene]-4-piperidin-4-yl-1,3-dihydro-indol-2-one, 3-[3-methyl-4(morpholine-4-carbonyl)-1H-pyrrolylmethylene]-4piperidin-4-yl-1,3-dihydroindol-2-one, 3-(3,5-dibromo-4hydroxy-benzylidine-2-oxo-2,3-dihydro-1H-indole-5carbonitrile, 3-(3,5-dibromo-4-hydroxy-benzylidene)-5-(2-methyl-thiazol-4-yl)-1,3-dihydro-indol-2-one, 3-(3-bromo5-ethoxy-4-hydroxy-benzylidene)-5-(2-methyl-thiazol-4-yl)-1,3-dihydro-indol-2-one, 3-(3,5-dichloro-4-hydroxybenzylidene-5-(2-methyl-thiazol-4-yl)-1,3-dihydro-indol-2one, (butanoyl)-1,3-dihydro-indol-2-one, 3-(3,5-dibromo-4-hydroxy-benzylidene-5-(3-methyl-butanoyl-1,3-dihydroindol-2-one, 5-benzoyl-3-(3,5-dibromo-4-hydroxybenzylidene)-1,3-dihydro-indol-2-one, 5-benzoyl-3-(3,5dichloro-4-hydroxy-benzylidene)-1,3-dihydro-indol-2-one, 5-benzoyl-3-(3-bromo-5-ethoxy-4-hydroxy-benzylidene)-1,3-dihydro-indol-2-one, 3-(3,5-dichloro-4-hydroxy-benzylidene-5-(3-methyl-butanoyl)-1,3-dihydro-indol-2-one, 3-(3-bromo-5-ethoxy-4-hydroxy-benzylidene)-5-(3-methylbutanoyl)-1,3-dihydro-indol-2-one, 3-(3,5-dibromo-4-hydroxy-benzylidene-5-(pyridine-3-carbonyl)-1,3-dihydro-indol-2-one, 3-(3,5-dichloro-4-hydroxy-benzylidiene)-5(pyridine-3-carbonyl)-1,3-dihydro-indol-2-one, 3-(3,5dibromo-4-hydroxy-benzylidene)-5-(pyridine-4-carbonyl)1,3-dihydro-indol-2-one, 3-(3-bromo-5-ethoxy-4-hydroxybenzylidene)-5-(pyridine-4-carbonyl)-1,3-dihydro-indol-2one, 3-(3,5-dichloro-4-hydroxy-benzylidene)-5-(pyridine-4carbonyl)-1,3-dihydro-indol-2-one, 3-(3-bromo-5-ethoxy-4-hydroxy-benzylidene)-5-(pyridine-3-carbonyl)-1,3-dihydro-indol-2-one, 3-(3,5-dichloro-4-hydroxybenzylidene-5-(oxazol-5-yl)-1,3-dihydro-indol-2-one, 3-(3,4-dibromo-4-hydroxy-benzylidene)-5-(oxazol-5-yl)-1,3-dihydro-indol-2-one, 3-(3,5-dibromo-4-hydroxybenzylidene)-5-(2-ethyl-thiazol-4-yl)-1,3-dihydro-indol-2one, 3-(3,5-dibromo-4-hydroxy-benzylidene)-2-oxo-2,3dihydro-1H-indol-5-carboxylic acid methyl ester, 3-(3,5dibromo-4-hydroxy-benzylidene-5-(furan-2-carbonyl)-1,3dihydro-indol-2-one, 3-(3,5-dichloro-4-hydroxybenzylidene-5-(furan-2-carbonyl)-!,3-dihydro-indol-2-one, 3-(3-bromo-5-ethoxy-4-hydroxy-benzylidene-5-(furan-2carbonyl)-1,3-dihydro-indol-2-one, 5-cyclopropanecarbonyl-3-(3,5-dibromo-4-hydroxy-benzylidene)-1,3-dihydroindol-2-one, 5-aminomethyl-3-(3,5-dibromo-4-hydroxybenzylidene)-1,3-dihydro-indol-2-one, 5-cyclopentanecarbonyl-3-(3,5-dibromo-4-hydroxy-benzylidene)-1,3-dihydro-indol-2-one, 3-(3,5-dichloro-4-hydroxy-benzylidene)-2-oxo-2,3-dihydro-1H-indole-5-carboxylic acid methyl ester, 3-(3,5-dibromo-4-hydroxybenzylidene)-1,3-dihydro-indol-2-one, 3-(3,5-dibromo-4-hydroxy-benzylidene)-5-(thiophene-2-carbonyl)-1,3dihydro-indol-2-one, 5-(2-amino-thiazol-4-yl)-3-(3,5-dibromo-4-hydroxy-benzylidene)-1,3-dihydro-indol-2-one, 3-(3,5-dibromo-4-hydroxy-benzylidene)-5-(imidazo[1,2a]pyridin-2-yl)-1,3-dihydro-indol-2-one, 3-(3,5-dibromo-4-hydroxy-benzylidene)-5-propionyl-1,3-dihydro-indol-2one, 3-(3,5-dibromo-4-hydroxy-benzylidene)-2-oxo-2,3dihydro-1H-indole-5-sulfonic acid amide, 3-(3,5-dibromo-4-hydroxy-benzylidene)-2-oxo-2,3dihydro-1H-indole-5sulfonic acid N,N-diethylamide, 3-(3,5-dibromo-4-hydroxybenzylidene)-5-(pyrrolidine-1-sulfonyl)-1,3-dihydro-indol2-one, 3-(3,5-dibromo-4-hydroxy-benzylidene)-2-oxo-2,3dihydro-1H-indol-5-sulfonic acid (N-2-dimethylaminoethyl)-N-methyl-amide, 3-(3,5-dibromo-4-hydroxybenzylidene)-5-(isoxazole-5-carbonyl)-1,3-dihydro-indol2-one, 5-chloro-3-(3,5-dibromo-4-hydroxy-benzylidene)-1,3-dihydro-indol-2-one, 5-chloro-3-(3,5-dichloro-4-hydroxy-benzylidene)-1,3-dihydro-indol-2-one, 3-(3,5dibromo-4-hydroxy-benzylidene)-5-trifluoromethoxy-1,3dihydro-indol-2-one, 5-bromo-3-(3,5-dichloro-4-hydroxybenzylidene)-1,3-dihydro-indol-2-one, 3-(3-bromo-5-ethoxy-4-hydroxy-benzylidene)-5-iodo-1,3-dihydro-indol2-one, 3-(3-bromo-4-hydroxy-5-methoxy-benzylidene)-5 iodo-1,3-dihydro-indol-2-one, 5-bromo-3-(3,5-diiodo-4-hydroxy-benzylidene)-1,3-dihydro-indol-2-one, 3-(3,5diiodo-4-hydroxy-benzylidene)-5-trifluoromethoxy-1,3dihydro-indol-2-one, 3-(3-bromo-4-hydroxy-5-methoxybenzylidene)-1,3-dihydro-indol-2-one, 3-(3,5-dinitro-4hydroxy-benzylidene)-1,3-dihydro-indol-2-one, 3-(3,5dichloro-4-hydroxy-benzylidene)-!,3-dihydro-indol-2-one, 3-(3-chloro-4-hydroxy-5-methoxy-benzylidene)-!,3-dihydro-indol-2-one, 3-(3,5-diiodo-4-hydroxy-benzylidene)-1,3dihydro-indol-2-one, 3-(3-bromo-4-hydroxy-5-methoxybenzylidene)-5-chloro-1,3-dihydro-indol-2-one, 5-chloro-3-(3,5-dinitro-4-hydroxy-benzylidene)-1,3-dihydro-indol-2one, 5-chloro-3-(4-hydroxy-3-methoxy-5-nitrobenzylidene)-1,3-dihydro-indol-2-one, 5-chloro-3-(3-chloro-4-hydroxy-5-methoxy-benzylidene)-1,3-dihydroindol-2-one, 5-chloro-3-(3,5-diiodo-4-hydroxybenzylidene)-1,3-dihydro-indol-2-one, 5-bromo-3-(3-bromo-5-ethoxy-4-hydroxy-benzylidene)-1,3-dihydroindol-2-one, 5-bromo-3-(3-bromo-4-hydroxy-5-methoxybenzylidene)-1,3-dihydro-indol-2-one, 3-(3-bromo-4-hydroxy-5-methoxy-benzylidene)-5,6-difluoro-1,3-dihydroindol-2-one, 3-(3-bromo-5-ethoxy-4-hydroxy-benzylidene)-5-trifluoromethoxy-1,3-dihydro-indol-2-one, 3-(3,5-dichloro-4-hydroxy-benzylidene)-5-trifluoromethoxy-1,3dihydro-indol-2-one, 3-(3-bromo-4-hydroxy-5-methoxybenzylidene)-5-trifluoromethoxy-1,3-dihydro-indol-2-one, 3-(3,5-dibromo-4-hydroxy-benzylidene)-5,7-dinitro-1,3-dihydro-indol-2-one, 3-(3,5-dibromo-4-hydroxybenzylidene)-5-nitro-1,3-dihydro-indol-2-one, 3-(3,5-dibromo-4-hydroxy-benzylidene)-7-iodo-1,3-dihydro-indol-2-one, 3-(3,5-dichloro-4-hydroxy-benzylidene)-5-nitro-1,3-dihydro-indol-2-one, 3-(3,5-dibromo-4-hydroxy-benzylidene)-7-iodo-1,3-dihydro-indol-2-one, 7-bromo-3-(3,5-dichloro-4-hydroxy-benzylidene)-1,3-dihydro-indol-2-one, 3-(3bromo-5-ethoxy-4-hydroxy-benzylidene)-5-nitro-1,3dihydro-indol-2-one, and 2-(N-{3-[3-(3,5-dibromo-4-hydroxy-benzylidene)-2-oxo-2,3-dihydro-1H-indol-5-yl]-2oxo-ethyl}-N-methyl-amino)-acetamide.

In some embodiments, the compound is a CDK inhibitor. Exemplary CDK inhibitors include, but are not limited to, 2-(3-Hydroxypropylamino)-6(β-hydroxybenzylamino)-9-isopropylpurine, 2-bromo-12,13-dihydro-5H-indolo[2,3-a]pyrrolo[3,4-c]carbazole-5, 7(6H)-dione, 3-(2-Chloro-3-indolylmethylene)-1,3-dihydroindol-2-one, 2(bis-(Hydroxyethyl)amino)-6-(4-methoxybenzylamino)-9-isopropyl-purine, 3-Amino-1Hpyrazolo[3,4-b]quinoxaline, 5-amino-3-((4-(aminosulfonyl)phenyl)amino)-N-(2,6-difluorophenyl)-1H1,2,4-triazole-1-carbothioamide, aloisine A, aloisine RP106, alsterpaullone 2-cyanoethyl, alvocidib, aminopurvalanol A, bohemine, CGP74514A, ethyl-(6-hydroxy-4-phenylbenzo[4,5]furo[2,3-b])pyridine-3-carboxylate, fisetin, N4-(6-Aminopyrimidin-4-yl)-sulfanilamide, flavopiridol, kenpaullone, NSC 625987, NU6102, NU6140, olomoucine, olomoucine II, roscovitine, SU9516, WR 216174, p21Cip1 (CDKN1A), p27Kip1, (CDKN1B), and analogs thereof. Additional CDK inhibitors are described in U.S. Pat. Nos. 7,084,271, 7,078,591, 7,078,525, 7,074,924, 7,067,661, 6,992,080, 6,939,872, 6,919,341, 6,710,227, 6,683,095, 6,677,345, 6,610,684, 6,593,356, 6,569,878, 6,559,152, 6,531,477, 6,500,846, 6,448,264, and 6,107,305, the contents of which each are incorporated herein by reference in their entirety.

In some embodiments, the compound is a HDAC inhibitor. Inhibitors of HDAC include small molecular weight carboxylates (e.g., less than about 250 amu), hydroxamic acids, benzamides, epoxyketones, cyclic peptides, and hybrid molecules. (See, for example, Drummond D. C., et al. Annu. Rev. Pharmacol. Toxicol. (2005) 45: 495-528, (including specific examples therein) which is hereby incorporated by reference in its entirety). Non-limiting examples HDAC inhibitors include, but are not limited to, Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (i.e., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (i.e., 6-(3-chlorophenylureido)caproic hydroxamic acid), AOE (2-amino-8-oxo-9,10-epoxydecanoic acid), CHAP31 and CHAP 50. Other inhibitors include, for example, dominant negative forms of the HDACs (e.g., catalytically inactive forms) siRNA inhibitors of the HDACs, and antibodies that specifically bind to the HDACs. HDAC inhibitors are commercially available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich. Further HDAC inhibitors amenable to the invention include, but are not limited to, those that are described in U.S. Pat. Nos. 7,183,298; 6,512,123; 6,541,661; 6,531,472; 6,960,685; 6,897,220; 6,905,669; 6,888,207; 6,800,638 and 7,169,801, and U.S. patent application Ser. Nos. 10/811,332; 12/286,769; 11/365,268; 11/581,570; 10/509,732; 10/546,153; 10/381,791 and 11/516,620, the contents of which each are incorporated herein by reference in their entirety.

In some embodiments, the HDAC inhibitor is not trichostatin, butyrates (e.g., sodium butyrate, arginine butyrate, and butyric acid), or trapoxin.

In some embodiments, the compound is a proteasome inhibitor. Exemplary proteasome inhibitors amenable to the invention include, but are not limited to those that are described in U.S. Pat. Nos. 5,693,617; 5,780,454; 5,83,4487; 6,465,433; 6,794,516; 6,747,150; 6,117,887; 6,133,308; 6,6617,317; 6,294,560; 6,849,743; 6,310.057; 6,566,553; 6,07,5150; 6,083,903; 6,066,730; 6,297,217 and 6,462,019, the contents of which each are incorporated herein by reference in their entirety. In one embodiment, the proteasome inhibitor is not lacacystin or those described in U.S. Pat. Publication No. 2007/0207144.

In some embodiments, the compound is a dopamine receptor ligand. Exemplary dopamine receptor ligands amenable to the invention include, but are not limited to those that are described in U.S. Pat. Nos. 6,469,141; 5,998,414; 6,107,313; 5,849,765; 5,861,407; 5,798,350; 6,103,715; 5,576,314; 5,538,965; 5,968,478; 5,700,445; 5,407,823 and 5,602,121, the contents of which each are incorporated herein by reference in their entirety.

In some embodiments, the compound is a cannabinoid (CB) receptor agonist. In one embodiment, the cannabinoid receptor agonist is WIN55, 212-2 or anandamide. Other exemplary cannabinoid receptor agonists amenable to the invention include, but are not limited to those that are described in U.S. Pat. Nos. 5,607,933; 5,324,737; 5,013,837; 5,081,122; 6,825,209; 5,817,651; 7,057,076; 5,068,234; 5,605,906; 6,995,187; 6,166,066; 6,509,367; 6,100,259; 7,119,108; 4,973,587; 5,112,820; 7,037,910; 5,948,777; 5,925,768; 6,013,648; 6,864,291; 6,903,137; 6,943,266 and 5,596,106, the contents of which each are incorporated herein by reference in their entirety.

In some embodiments, the compound is FGF, EGF, NGF, TGF, PDGF, PDGF-BB or insulin, which compound activates PI-3K signaling pathway.

In some embodiments, the compound is an activator of PI-3K pathway, which compound activates PI3K, PDK or PKB.

In some embodiments, the compound inhibits IκB kinase 2 (IKK-2). Exemplary IKK-2 inhibitors include, but are not limited to SC-514, SPC-839, IKK-2 inhibitor IV (CAS: 507475-17-4) and IKK-2 inhibitor VI. Other IKK-2 inhibitors amenable to the present invention include those described in U.S. Pat. Nos. 7,122,544; 6,462,036; and 7,125,896, and U.S. patent application Ser. Nos. 11/271,598; 11/211,383; 10/542,044; 11/272,401; 11/430,215; 11/346,986; and 10/542,326, the contents of which each are incorporated herein by reference in their entirety. Further IKK-2 inhibitors are described in Bingham, A. H., et al., Bioorg. Med. Chem. (2003), 14, 409-412 and Liddle, J., et al., Bioorg. Med. Chem. Lett. (2009), 19, 2504-2508, the contents of which each are incorporated herein by reference in their entirety.

In some embodiments, the compound is a modulator of TGF-β signaling. Exemplary modulators of TGF-β signaling include, but are not limited to, AP-12009 (TGF-β Receptor type II antisense oligonucleotide), Lerdelimumab (CAT 152, antibody a TGF-β Receptor type II) GC-1008 (antibody to all isoforms of human TGF-β), ID11 (antibody to all isoforms of murine TGF-β), soluble TGF-β, soluble TGF-β Receptor type II, dihydropyrroloimidazole analogs (e.g., SKF-104365), triarylimidazole analogs (e.g., SB-202620 (4-(4-(4-fluorophenyl)-5-(pyridin-4-yl)-1H-imidazol-2-yl)benzoic acid) and SB-203580 (4-(4-Fluorophenyl)-2-(4-methylsulfinyl phenyl)-5-(4-pyridyl)-1H-imidazole)), RL-0061425, 1,5-naphthyridine aminothiazole and pyrazole derivatives (e.g., 4-(6-methyl-pyridin-2-yl)-5-(1,5-naphthyridin-2-yl)-1,3-thiazole-2-amine and 2-[3-(6-methyl-pyridin-2-yl)-1H-pyrazole-4-yl]-1,5-naphthyridine), SB-431542 (4-(5-Benzo[1,3]dioxol-5-yl-4-pyridin-2-yl-1H-imidazol-2-yl)-benzamide), GW788388 (4-(4-(3-(pyridin-2-yl)-1H-pyrazol-4-yl)pyridin-2-yl)-N-(tetrahydro-2H-pyran-4-yl)benzamide), A-83-01 (3-(6-Methyl-2-pyridinyl)-N-phenyl-4-(4-quinolinyl)-1H-pyrazole-1-carbothioamide), Decorin, Lefty 1, Lefty 2, Follistatin, Noggin, Chordin, Cerberus, Gremlin, Inhibin, BIO (6-bromo-indirubin-3′-oxime), Smad proteins (e.g., Smad6, Smad7), Cystatin C, soluble TGF-β Receptor type I, AP-11014 (TGF-β Receptor type I antisense oligonucleotide), Metelimumab (CAT 152, TGF-β Receptor type I antibody), LY550410, LY580276 (3-(4-fluorophenyl)-5,6-dihydro-2-(6-methylpyridin-2-yl)-4H-pyrrolo[1,2-b]pyrazole), LY364947 (4-[3-(2-Pyridinyl)-1H-pyrazol-4-yl]quinoline), LY2109761, LY573636 (N4(5-bromo-2-thienyl)sulfonyl)-2,4-dichlorobenzamide), SB-505124 (2-(5-Benzo[1,3]dioxol-5-yl-2-tert-butyl-3H-imidazol-4-yl)-6-methylpyridine), SD-208 (2-(5-Chloro-2-fluorophenyl)-4-[(4-pyridyl)amino]pteridine), SD-093, KI2689, SM16, FKBP12 protein, 3-(4-(2-(6-methylpyridin-2-yl)H-imidazo[1,2-a]pyridin-3-yl)quinolin-7-yloxy)-N,N-dimethylpropan-1-amine, and analogs thereof. Other modulators of TGF-β signaling amenable to the invention are described in Callahan, J. F. et al., J. Med. Chem. 45, 999-1001 (2002); Sawyer, J. S. et al., J. Med. Chem. 46, 3953-3956 (2003; Sawyer J. S. et al., Bioorg. Med. Chem. Lett. 14, 3581-3584 (2004); Gellibert, F. et al., J. Med. Chem. 47, 4494-4506 (2004); Yingling, J. M. et al., Nature Rev. Drug Disc. 3, 1011-1022 (2004); Tojo, M. et al., Cancer Sci. 96: 791-800 (2005); Valdimarsdottir, G. et al., APMIS 113, 773-389 (2005); Petersen et al. Kidney International 73, 705-715 (2008); Yingling, J. M. et al., Nature Rev. Drug Disc. 3, 1011-1022 (2004); Byfield, S. D. et al., Mol. Pharmacol., 65, 744-752 (2004); Byfield, S. D., and Roberts, A. B., Trends Cell Biol. 14, 107-111 (2004); Dumont, N, et al., Cancer Cell 3, 531-536 (2003); WO Publication No. 2002/094833; WO Publication No. 2004/026865; WO Publication No. 2004/067530; WO Publication No. 209/032667; WO Publication No. 2004/013135; WO Publication No. 2003/097639; WO Publication No. 2007/048857; WO Publication No. 2007/018818; WO Publication No. 2006/018967; WO Publication No. 2005/039570; WO Publication No. 2000/031135; WO Publication No. 1999/058128; WO Publication No. 2004/026871; WO Publication No. 2004/021989; WO Publication No. 2004/026307; WO Publication No. 2000/012497; U.S. Pat. No. 6,509,318; U.S. Pat. No. 6,090,383; U.S. Pat. No. 6,419,928; U.S. Pat. No. 9,927,738; U.S. Pat. No. 7,223,766; U.S. Pat. No. 6,476,031; U.S. Pat. No. 6,419,928; U.S. Pat. No. 7,030125; U.S. Pat. No. 6,943,191; U.S. Pat. No. 5,731,144; U.S. Pat. No. 7,151,169; U.S. Publication No. 2005/0245520; U.S. Publication No. 2004/0147574; U.S. Publication No. 2007/0066632; U.S. Publication No. 2003/0028905; U.S. Publication No. 2005/0032835; U.S. Publication No. 2008/0108656; U.S. Publication No. 2004/015781; U.S. Publication No. 2004/0204431; U.S. Publication No. 2006/0003929; U.S. Publication No. 2007/0155722; U.S. Publication No. 2004/0038856; U.S. Publication No. 2005/0245508; U.S. Publication No. 2004/0138188 and U.S. Publication No. 2009/0036382, contents of all of which are herein incorporated in their entireties. Oligonucleotide based modulators of TGF-β signaling, such as siRNAs and antisense oligonucleotides, are described in U.S. Pat. No. 5,731,424; U.S. Pat. No. 6,124,449; U.S. Publication Nos. 2008/0015161; 2006/0229266; 2004/0006030; 2005/0227936 and 2005/0287128, contents of all of which are herein incorporated in their entireties. Other antisense nucleic acids and siRNAs can be obtained by methods known to one of ordinary skill in the art.

In some embodiments, BMP/TGFβ ligand is BMP4 (bone morphogenetic protein 4). Other exemplary BMP/TGFβ modulators are described in U.S. Pat. Nos. 7,223,766 and 7,354,722, the contents of which each are incorporated herein by reference in their entirety.

In some embodiments, compound is a Na⁺, K⁺ and/or Ca²⁺, ion channel modulator. Exemplary ion channel modulators are described in U.S. Pat. Nos. 6,184,231; 6,479,498; 6,646,012; 5,565,483; 5,871940; 6,172,085; 5,242,947; 7,132,422; 6,756,400; 7,183,323; 7,226,950 and 6,872,741, and U.S. patent application Ser. Nos. 11/434,920; 11/546,669; 10/514,150; 11/450,695; 11/216,376; 10/743,280; 11/434,627; 10/977,609; 11/811,909; 11/418,163; 11/556,354; 11/432,997; 11/216,899; 11/517,754; 11/418,278; 10/792,688; 11/574,751; 11/643,622; 11/266,142; 10/935,008; 12/158,491; 10/450,215; 10/427,847 and 10/838,087, the contents of which each are incorporated herein by reference in their entirety. In some embodiments, Na⁺/K⁺ channel modulator is a cardiac glycoside selected from the group consisting of Ouabain, Digoxin, Dititoxin, Lanatoside C, and combinations thereof. In some embodiments, Ca²⁺ channel modulator is Thapsigargin, ionomycin or Calcimycin. In some embodiments, K⁺ channel modulator is Veratridine, Monensin NA or Valinomycin.

In some embodiments, MAPK activator is Anysomycin or Coumermycin.

Exemplary inhibitors of PDE5 are described in U.S. Pat. Nos. 6,869,974; 6,680,047; 6,635,274; 6,555,663 and 6,472,425, the contents of which each are incorporated herein by reference in their entirety. In some embodiments, PDE5 inhibitor is selected from the group consisting of MBCQ, Dipyridamole, spironolactone, bucladesine, and combinations thereof.

In some embodiments, the compound is an activator of RTK signaling. Exemplary modulators of RTK signaling are described in U.S. Pat. Nos. 5,196,446; 5,374,652; 6,316,635; 7,214,700; 6,569,868; 5,302,606; and 6,849,641, the contents of which each are incorporated herein by reference in their entirety. In some embodiments, RTK activator is PDGF-BB.

In some embodiments, the compound is a growth factor.

In one embodiment, the compound is not that described in U.S. Pat. Publication No. 2007/0207144.

Nucleic acid modulators of biological pathways and targets include, but are not limited to, antisense oligonucleotide, siRNA, shRNA, ribozyme, aptamers, decoy oligonucleotides. Methods of preparing such nucleic acids are known in the art and easily available to those skilled in the art.

In some embodiments, amino acid based molecule, such a peptides, oligopeptides and proteins, can be used to modulate the biological pathways or targets described herein.

In some embodiments, antibodies can be used to modulate the biological pathways or targets described herein. As used herein, the term “antibody” includes complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)₂, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred. Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites. Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific. Thus, antibodies useful in the methods described herein include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabodies, triabodies, and the like that bind specifically with an antigen. While both polyclonal and monoclonal antibodies can be used in the methods described herein, it is preferred that a monoclonal antibody is used where conditions require increased specificity for a particular protein. Antibodies can be raised against a biological pathway component or target by methods known to those skilled in the art. Such methods are described in detail, for example, in Harlow et al., 1988 in: Antibodies, A Laboratory Manual, Cold Spring Harbor, N.Y.

Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

In some embodiments, a compound described herein promotes motor neuron survival by increasing SMN protein levels in motor neuron. In some embodiments, the SMN protein levels are increased by about at least 5%, 10%, 20%, 30%, 40%, 50%, 60%, 70%, 80%, 90%, 95%, 100%, 1.1-fold, 1.25-fold, 1.5-fold, 1.75-fold, 2-fold, 3-fold, 4-fold, 5-fold, 10-fold or more relative to when cell is not contacted with a compound described herein.

Without wishing to be bound by theory, some of the compounds described herein can promote motor neuron survival without increasing SMN protein levels and/or GEMs in the motor neurons. In some embodiments, a compound described herein promotes motor neuron survival without increasing SMN protein levels in motor neuron. For a non-limiting example, kenpaullone and compounds GSK1 and GSK13 promote motor neuron survival without increasing SMN protein levels in the motor neuron.

In another aspect, the invention provides for a method of increasing SMN protein levels in a motor neuron, the method comprising: contacting a motor neuron with a compound described herein, wherein the signaling pathway is selected from the group consisting of PI3K signaling pathway, Akt signaling pathway, MAPK signaling pathway, PDGF pathway, RAS pathway, eIF2 pathway, GSK pathway, PKR pathway, Insulin Receptor Pathway, mTOR pathway, EGF pathway, NGF pathway, FGF pathway, BMP/TGFβ pathway, receptor tyrosine kinase (RTK) pathway, and combinations thereof. In some embodiments of this and other aspects described herein, the signaling pathway is the PI-3/AKT/GSK pathway.

In some embodiments of this aspect, the compound is a GSK inhibitor selected from the group consisting of compounds of formula (I)-(VI), CHIR99021, CHIR98014, GSK2, GSK6, GSK7, GSK8, GSK9, GSK15, hymenialdisine, flavopiridol, aloisine A, aloisine B, compound 12, pyrazolopyridine 18, pyrazolopyridine 9, pyrazolopyridine 34, CT20026, compound I, SU9516, staurosporine, compound 5a, compound 29, compound 46, GF109203x, RO318220, SB216763, SB415286, 15, CGP60474, compound 8b, and combinations thereof.

In some embodiments of this aspect, the compound is not kenpaullone, GSK1, GSK10, GSK11, GSK12, GSK13 or GSK 17.

In some embodiments of this aspect, the compound is not an Akt inhibitor.

Increase in SMN protein levels can be accomplished without an increase in SMN RNA levels. For example, compounds described herein can enhance the translation of SMN RNAs, can stabilize the SMN protein against degradation or both. In some embodiments, the compound increases SMN protein level by activation of protein synthesis e.g., translation.

In some embodiments, the SMN protein levels are increased by about at least 5%, 10%, 20%, 30%, 40%, 50%, 60%, 70%, 80%, 90%, 95%, 100%, 1.1-fold, 1.25-fold, 1.5-fold, 1.75-fold, 2-fold, 3-fold, 4-fold, 5-fold, 10-fold or more relative to when cell is not contacted with a compound described herein.

### Motor Neurons

In some embodiments, the motor neurons comprise a mutation in a gene associated with a neurodegenerative disorder. One non limiting example of a gene associated with a neurodegenerative disorder is SMN1. Another non limiting example of a gene associated with a neurodegenerative disorder is SOD1. A variety of SOD1 mutant alleles are known to be associated with SMA and/or ALS, including without limitation, SOD1G93A.

In some embodiments, methods of the invention employ cells that are not motor neurons, wherein the cells can comprise a mutation in a gene associated with a neurodegenerative disease. In one non-limiting example, some methods the present invention employ fibroblasts comprising a mutation in a gene associated with a neurodegenerative disease. In some embodiments, methods of the invention employ fibroblasts comprising a mutation in a SOD1 gene, such as, without limitation, SOD1G93A.

As used herein, the term “SOD1” refers to either the gene encoding superoxide dismutase 1 or the enzyme encoded by this gene. The SOD1 gene or gene product is known by other names in the art including, but not limited to, ALS1, Cu/Zn superoxide dismutase, indophenoloxidase A, IPOA, and SODC_HUMAN. Those of ordinary skill in the art will be aware of other synonymous names that refer to the SOD1 gene or gene product. The SOD1 enzyme neutralizes supercharged oxygen molecules (called superoxide radicals), which can damage cells if their levels are not controlled. The human SOD1 gene maps to cytogenetic location 21q22.1. Certain mutations in SOD1 are associated with ALS in humans including, but not limited to, Ala4Val, Gly37Arg and Gly93Ala, and more than one hundred others. Those of ordinary skill in the art will be aware of these and other human mutations associated with ALS. Certain compositions and methods of the present invention comprise or employ cells comprising a SOD1 mutation.

“SOD 1 mutations” refer to mutations in the SOD1 gene (NC_{—}000021.8; NT_{—}011512.11; AC_{—}000064.1; NW_{—}927384.1; AC_{—}000153.1; NW_{—}001838706.1 NM_{—}000454.4; NP_{—}000445.1 and NCBI Entrez GeneID: 6647) including but are not limited to Ala4Val, Cys6Gly, Val7Glu, Leu8Val, Gly10Val, Gly12Arg, Val14Met, Gly16Ala, Asn19Ser, Phe20Cys, Glu21Lys, Gln22Leu, Gly37Arg, Leu38Arg, Gly41Ser, His43Arg, Phe45Cys, His46Arg, Val47Phe, His48Gln, Glu49Lys, Thr54Arg, Ser59Ile, Asn65Ser, Leu67Arg, Gly72Ser, Asp76 Val, His80Arg, Leu84Phe, Gly85Arg, Asn86Asp, Val87Ala, Ala89Val, Asp90Ala, Gly93Ala, Ala95Thr, Asp96Asn, Val97Met, Glu100Gly, Asp101Asn, Ile104Phe, Ser105Leu, Leu106Val, Gly108Val, Ile112Thr, Ile113Phe, Gly114Ala, Arg115Gly, Val118Leu, Ala140Gly, Ala145Gly, Asp124Val, Asp124Gly, Asp125His, Leu126Ser, Ser134Asn, Asn139His, Asn139Lys, Gly141Glu, Leu144Phe, Leu144Ser, Cys146Arg, Ala145Thr, Gly147Arg, Val148Gly, Val148Ile, Ile149Thr, Ile151Thr, and Ile151Ser. SOD1 is also known as ALS, SOD, ALS1, IPOA, homodimer SOD1. “SOD 1 mutation” databases can be found at Dr. Andrew C. R. Martin website at the University College of London (www.bioinfo.org.uk), the ALS/SOD1 consortium website (www.alsod.org) and the human gene mutation database (HGMD®) at the Institute of Medical Genetics at Cardiff, United Kingdom.

Contacting of Motor Neurons with Compounds

Motor neurons can be contacted with the compounds described herein in a cell culture e.g., in vitro or ex vivo, or administrated to a subject, e.g., in vivo. In some embodiments of the invention, a compound described herein can be administrated to a subject to treat, prevent, and/or diagnose neurodegenerative disorders, including those described herein.

The term “contacting” or “contact” as used herein in connection with contacting a motor neuror cell includes subjecting the cell to an appropriate culture media which comprises the indicated compound or agent. Where the motor neuron is in vivo, “contacting” or “contact” includes administering the compound or agent in a pharmaceutical composition to a subject via an appropriate administration route such that the compound or agent contacts the motor neuron in vivo. Measurement of cell survival can be based on the number of viable cells after period of time has elapsed after contacting of cells with a compound or agent. For example, number of viable cells can be counted after about at least 5 minutes, 10 minutes, 20 minutes, 30 minutes, 40 minute, 40 minutes, 590 minutes, 1 hour, hours, 2 hours, 4 hours, 6 hours, 12 hours, 24 hours, 2 days, 3 days or more and compared to number of viable cells in a non treated control.

For in vitro methods, motor neurons can be obtained from different sources. For example, motor neurons can be obtained from a subject, or derived from non motor neuron cells from a subject. In some embodiments, motor neuron is a whole cell. In some embodiments, the subject is suffering from a neurodegenerative disorder. In some embodiments, the subject is suffering from SMA or ALS. In some embodiments, the subject is a carrier e.g., a symptom-free carrier. In some embodiments, motor neuron cells are derived from a subject's embryonic stem cells (ESCs). In some embodiments, the subject is human. In some embodiments, the subject is mouse. In some embodiments, mouse is a transgenic mouse. Methods of inducing motor neuron differentiation from embryonic stem cells are known in the art, for example as described in Di Giorgio et al., Nature Neuroscience (2007), published online 15 Apr. 2007; doi:10.1038/nn1885 and Wichterle et al., Cell (2002) 110:385-397. In some instances induced pluripotent stem cells can be generated from a subject and then differentiated into motor neurosn. One exemplary method of deriving motor neurons from a subject is described in Dimos, J. T., et. al. Science (2008) 321, 1218-122 (Epub Jul. 31, 2008).

For in vivo methods, a therapeutically effective amount of a compound described herein can be administered to a subject. Methods of administering compounds to a subject are known in the art and easily available to one of skill in the art.

As one of skill in the art is aware, promoting survival of motor neuron cells in a subject can lead to treatment, prevention or amelioration of a number of neurodegenerative disorders. By “neurodegenerative disorder” is meant any disease or disorder caused by or associated with the deterioration of cells or tissues of the nervous system. Exemplary neurodegenerative disorders are polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy (SMA), SteeleRichardson-Olszewski disease, and Tabes dorsalis.

The motor neuron diseases (MND) are a group of neurodegenerative disorders that selectively affect motor neurons, the nerve cells that control voluntary muscle activity including speaking, walking, breathing, swallowing and general movement of the body. Skeletal muscles are innervated by a group of neurons (lower motor neurons) located in the ventral horns of the spinal cord which project out the ventral roots to the muscle cells. These nerve cells are themselves innervated by the corticospinal tract or upper motor neurons that project from the motor cortex of the brain. On macroscopic pathology, there is a degeneration of the ventral horns of the spinal cord, as well as atrophy of the ventral roots. In the brain, atrophy may be present in the frontal and temporal lobes. On microscopic examination, neurons may show spongiosis, the presence of astrocytes, and a number of inclusions including characteristic “skein-like” inclusions, bunina bodies, and vacuolisation. Motor neuron diseases are varied and destructive in their effect. They commonly have distinctive differences in their origin and causation, but a similar result in their outcome for the patient: severe muscle weakness. Amyotrophic lateral sclerosis (ALS), primary lateral sclerosis (PLS), progressive muscular atrophy (PMA), pseudobulbar palsy, progressive bulbar palsy, spinal muscular atrophy (SMA) and post-polio syndrome are all examples of MND. The major site of motor neuron degeneration classifies the disorders. As used herein, the phrase “motor neuron degeneration” or “degeneration of motor neuron” means a condition of deterioration of motor neurons, wherein the neurons die or change to a lower or less functionally-active form.

Common MNDs include amyotrophic lateral sclerosis, which affects both upper and lower motor neurons. Progressive bulbar palsy affects the lower motor neurons of the brain stem, causing slurred speech and difficulty chewing and swallowing. Individuals with these disorders almost always have abnormal signs in the arms and legs. Primary lateral sclerosis is a disease of the upper motor neurons, while progressive muscular atrophy affects only lower motor neurons in the spinal cord. Means for diagnosing MND are well known to those skilled in the art. Non limiting examples of symptoms are described below.

### Spinal Muscular Atrophy (SMA)

Spinal Muscular Atrophy (SMA) refers to a number of different disorders, all having in common a genetic cause and the manifestation of weakness due to loss of the motor neurons of the spinal cord and brainstem. Weakness and wasting of the skeletal muscles is caused by progressive degeneration of the anterior horn cells of the spinal cord. This weakness is often more severe in the legs than in the arms. SMA has various forms, with different ages of onset, patterns of inheritance, and severity and progression of symptoms. Some of the more common SMAs are described below.

Defect in SMN gene products are considered as the major cause of SMA and SMN protein levels correlate with survival of subject suffering from SMA. The most common form of SMA is caused by mutation of the SMN gene. The region of chromosome 5 that contains the SMN (survival motor neuron) gene has a large duplication. A large sequence that contains several genes occurs twice in adjacent segments. There are thus two copies of the gene, SMN1 and SMN2. The SMN2 gene has an additional mutation that makes it less efficient at making protein, though it does so in a low level. SMA is caused by loss of the SMN1 gene from both chromosomes. The severity of SMA, ranging from SMA 1 to SMA 3, is partly related to how well the remaining SMN 2 genes can make up for the loss of SMN 1.

SMA type I, also called Werdnig-Hoffmann disease, is evident by the time a child is 6 months old. Symptoms may include hypotonia (severely reduced muscle tone), diminished limb movements, lack of tendon reflexes, fasciculations, tremors, swallowing and feeding difficulties, and impaired breathing. Some children also develop scoliosis (curvature of the spine) or other skeletal abnormalities. Affected children never sit or stand and the vast majority usually die of respiratory failure before the age of 2.

Symptoms of SMA type II usually begin after the child is 6 months of age. Features may include inability to stand or walk, respiratory problems, hypotonia, decreased or absent tendon reflexes, and fasciculations. These children may learn to sit but do not stand. Life expectancy varies, and some individuals live into adolescence or later.

Symptoms of SMA type III (Kugelberg-Welander disease) appear between 2 and 17 years of age and include abnormal gait; difficulty running, climbing steps, or rising from a chair; and a fine tremor of the fingers. The lower extremities are most often affected. Complications include scoliosis and joint contractures—chronic shortening of muscles or tendons around joints, caused by abnormal muscle tone and weakness, which prevents the joints from moving freely.

Other forms of SMA include e.g., Hereditary Bulbo-Spinal SMA Kennedy's disease (X linked, Androgen receptor), SMA with Respiratory Distress (SMARD 1) (chromsome 11, IGHMBP2 gene), Distal SMA with upper limb predominance (chromosome 7, glycyl tRNA synthase), and X-Linked infantile SMA (gene UBE1)

Current treatment for SMA consists of prevention and management of the secondary effect of chronic motor unit loss. Some drugs under clinical investigation for the treatment of SMA include butyrates, Valproic acids, hydroxyurea and Riluzole.

Symptoms of Fazio-Londe disease appear between 1 and 12 years of age and may include facial weakness, dysphagia (difficulty swallowing), stridor (a high-pitched respiratory sound often associated with acute blockage of the larynx), difficulty speaking (dysarthria), and paralysis of the eye muscles. Most individuals with SMA type III die from breathing complications.

Kennedy disease, also known as progressive spinobulbar muscular atrophy, is an X-linked recessive disease. Daughters of individuals with Kennedy disease are carriers and have a 50 percent chance of having a son affected with the disease. Onset occurs between 15 and 60 years of age. Symptoms include weakness of the facial and tongue muscles, hand tremor, muscle cramps, dysphagia, dysarthria, and excessive development of male breasts and mammary glands. Weakness usually begins in the pelvis before spreading to the limbs. Some individuals develop noninsulin-dependent diabetes mellitus.

The course of the disorder varies but is generally slowly progressive. Individuals tend to remain ambulatory until late in the disease. The life expectancy for individuals with Kennedy disease is usually normal.

Congenital SMA with arthrogryposis (persistent contracture of joints with fixed abnormal posture of the limb) is a rare disorder. Manifestations include severe contractures, scoliosis, chest deformity, respiratory problems, unusually small jaws, and drooping of the upper eyelids.

Amyotrophic lateral sclerosis (ALS), also called Lou Gehrig's disease or classical motor neuron disease, is a progressive, ultimately fatal disorder that eventually disrupts signals to all voluntary muscles. In the United States, doctors use the terms motor neuron disease and ALS interchangeably. Both upper and lower motor neurons are affected. Approximately 75 percent of people with classic ALS will also develop weakness and wasting of the bulbar muscles (muscles that control speech, swallowing, and chewing). Symptoms are usually noticed first in the arms and hands, legs, or swallowing muscles. Muscle weakness and atrophy occur disproportionately on both sides of the body. Affected individuals lose strength and the ability to move their arms, legs, and body. Other symptoms include spasticity, exaggerated reflexes, muscle cramps, fasciculations, and increased problems with swallowing and forming words. Speech can become slurred or nasal. When muscles of the diaphragm and chest wall fail to function properly, individuals lose the ability to breathe without mechanical support. Although the disease does not usually impair a person's mind or personality, several recent studies suggest that some people with ALS may have alterations in cognitive functions such as problems with decision-making and memory. ALS most commonly strikes people between 40 and 60 years of age, but younger and older people also can develop the disease. Men are affected more often than women. Most cases of ALS occur sporadically, and family members of those individuals are not considered to be at increased risk for developing the disease. However, there is a familial form of ALS in adults, which often results from mutation of the superoxide dismutase gene, or SOD1, located on chromosome 21. In addition, a rare juvenile-onset form of ALS is genetic. Most individuals with ALS die from respiratory failure, usually within 3 to 5 years from the onset of symptoms. However, about 10 percent of affected individuals survive for 10 or more years.

Progressive bulbar palsy, also called progressive bulbar atrophy, involves the bulb-shaped brain stem—the region that controls lower motor neurons needed for swallowing, speaking, chewing, and other functions. Symptoms include pharyngeal muscle weakness (involved with swallowing), weak jaw and facial muscles, progressive loss of speech, and tongue muscle atrophy. Limb weakness with both lower and upper motor neuron signs is almost always evident but less prominent. Affected persons have outbursts of laughing or crying (called emotional lability). Individuals eventually become unable to eat or speak and are at increased risk of choking and aspiration pneumonia, which is caused by the passage of liquids and food through the vocal folds and into the lower airways and lungs. Stroke and myasthenia gravis each have certain symptoms that are similar to those of progressive bulbar palsy and must be ruled out prior to diagnosing this disorder. In about 25 percent of ALS cases early symptoms begin with bulbar involvement. Some 75 percent of individuals with classic ALS eventually show some bulbar involvement. Many clinicians believe that progressive bulbar palsy by itself, without evidence of abnormalities in the arms or legs, is extremely rare.

Pseudobulbar palsy, which shares many symptoms of progressive bulbar palsy, is characterized by upper motor neuron degeneration and progressive loss of the ability to speak, chew, and swallow. Progressive weakness in facial muscles leads to an expressionless face. Individuals may develop a gravelly voice and an increased gag reflex. The tongue may become immobile and unable to protrude from the mouth. Individuals may also experience emotional lability.

Primary lateral sclerosis (PLS) affects only upper motor neurons and is nearly twice as common in men as in women. Onset generally occurs after age 50. The cause of PLS is unknown. It occurs when specific nerve cells in the cerebral cortex (the thin layer of cells covering the brain which is responsible for most higher level mental functions) that control voluntary movement gradually degenerate, causing the muscles under their control to weaken. The syndrome—which scientists believe is only rarely hereditary—progresses gradually over years or decades, leading to stiffness and clumsiness of the affected muscles. The disorder usually affects the legs first, followed by the body trunk, arms and hands, and, finally, the bulbar muscles. Symptoms may include difficulty with balance, weakness and stiffness in the legs, clumsiness, spasticity in the legs which produces slowness and stiffness of movement, dragging of the feet (leading to an inability to walk), and facial involvement resulting in dysarthria (poorly articulated speech). Major differences between ALS and PLS (considered a variant of ALS) are the motor neurons involved and the rate of disease progression. PLS may be mistaken for spastic paraplegia, a hereditary disorder of the upper motor neurons that causes spasticity in the legs and usually starts in adolescence. Most neurologists follow the affected individual's clinical course for at least 3 years before making a diagnosis of PLS. The disorder is not fatal but may affect quality of life. PLS often develops into ALS.

Progressive muscular atrophy (PMA) is marked by slow but progressive degeneration of only the lower motor neurons. It largely affects men, with onset earlier than in other MNDs. Weakness is typically seen first in the hands and then spreads into the lower body, where it can be severe. Other symptoms may include muscle wasting, clumsy hand movements, fasciculations, and muscle cramps. The trunk muscles and respiration may become affected. Exposure to cold can worsen symptoms. The disease develops into ALS in many instances.

Post-polio syndrome (PPS) is a condition that can strike polio survivors decades after their recovery from poliomyelitis. PPS is believed to occur when injury, illness (such as degenerative joint disease), weight gain, or the aging process damages or kills spinal cord motor neurons that remained functional after the initial polio attack. Many scientists believe PPS is latent weakness among muscles previously affected by poliomyelitis and not a new MND. Symptoms include fatigue, slowly progressive muscle weakness, muscle atrophy, fasciculations, cold intolerance, and muscle and joint pain. These symptoms appear most often among muscle groups affected by the initial disease. Other symptoms include skeletal deformities such as scoliosis and difficulty breathing, swallowing, or sleeping. Symptoms are more frequent among older people and those individuals most severely affected by the earlier disease. Some individuals experience only minor symptoms, while others develop SMA and, rarely, what appears to be, but is not, a form of ALS. PPS is not usually life threatening. Doctors estimate the incidence of PPS at about 25 to 50 percent of survivors of paralytic poliomyelitis.

In some preferred embodiments, neurodegenerative disease is SMA or ALS.

By “treatment, prevention or amelioration of neurodegenerative disorder” is meant delaying or preventing the onset of such a disorder (e.g. death of motor neurons), at reversing, alleviating, ameliorating, inhibiting, slowing down or stopping the progression, aggravation or deterioration the progression or severity of such a condition. In one embodiment, the symptom of a neurodegenerative disorder is alleviated by at least 20%, at least 30%, at least 40%, or at least 50%. In one embodiment, the symptom of a neurodegenerative disease is alleviated by more that 50%. In one embodiment, the symptom of a neurodegenerative disorder is alleviated by 80%, 90%, or greater. Treatment also includes improvements in neuromuscular function. In some embodiments, neuromuscular function improves by at least about 10%, 20%, 30%, 40%, 50% or more.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. In some embodiments of the invention, subject suffers from a neurodegenerative disease.

In some embodiments of the methods described herein further comprise selecting a subject diagnosed with a neurodegenerative disease. A subject suffering from a neurodegenerative disease can be selected based on the symptoms presented. For example a subject suffering from SMA may show symptoms of hypotonia, diminished limb movements, lack of tendon reflexes, fasciculations, tremors, swallowing, feeding difficulties, imparired breating, scoliosis or other skeletal abnoramlities, inability to stand or walk, abnormal gait, difficulty running, difficulty climbing steps, difficulty rising from a chair, and/or fine tremor of the fingers.

In another aspect of the invention, the compounds described herein inhibit the activity of at least one kinase. In some embodiments of this and other aspects described herein, the kinase is selected from the group consisting of phosphoinositide 3-kinases (PI-3 kinases), phosphoinositide dependant kinase 1 (PDK1), SGK, glycogen synthase kinase 3 (GSK-3), inhibitor of IκB kinase 2 (IKK2), cyclin dependant kinase 2 (CDK2), and RNA dependant protein kinase.

In some embodiments of this and other aspects described herein, the compounds described herein inhibit the activity of at least two different kinases. For example, a compound can inhibit the activity of GSK-3 and a second kinase. In some embodiments, the second kinase is CDK.

In one aspect, the invention features a method for identifying a compound that modulates, e.g., increases the SMN level in a cell, modulates, e.g., increases the level of GEM in a cell, or modulates distribution of SMN or GEM in a cell. In one embodiment the method comprises one or both of two steps: a first step in which a candidate compound is contacted with a cell and is evaluated for the ability to modulate SMN or GEM level or distribution; and a second step in which a compound is contacted with a cell, in which cell SMN expression is reduced, and evaluated for the ability to rescue the cell having reduced SMN.

In an embodiment, the level of SMN is not lowered in the first step, or if lowered, is not lowered as much as in the second step, or is lowered by a different mechanism than used in the second step.

In an embodiment the method further comprises comparing the ability of the compound to modulate SMN or GEM level or distribution in the first cell with a reference and if the reference is met, proceeding to the second step.

In an embodiment, the first step is performed prior to the second step.

In an embodiment, the first and second cell are from the same taxon, e.g., species, e.g., they are both mammalian, e.g., primate, e.g., human, or rodent, e.g., mouse or rat.

In an embodiment, the first and second cell are from different taxa, e.g., species, e.g., one is rodent, e.g., mouse or rat, and the other is primate, e.g., human.

In an embodiment, the first cell is from a human and the second cell is a mouse cell, e.g., a mouse ES cell-derived motor neuron.

In another aspect, the invention described herein features a method for identifying a compound that modulates the SMN level in a cell, the method comprising treating a cell with a compound, and evaluating the SMN level in the cell and/or different cellular compartments, thereby determining whether the compound regulates the SMN level.

In some embodiments, the cell is a human cell or a mouse cell. In some embodiments, the cell is a whole cell. In some embodiments, the cell is from a subject, e.g., a patient. In some embodiments, the subject, e.g., a patient, is suffering from a neurodegenerative disorder. In some embodiments, the neurodegenerative disorder is SMA. In an embodiment the cell is from a carrier, e.g., a symptom-free carrier. In some embodiments, the cell is a fibroblast. In some embodiments, the cell is a neuron. In some embodiments, the neuron is a motor neuron. In some embodiments, the motor neuron is SMN-deficient. In some embodiments, the SMN level is evaluated by an image-based method. In some embodiments, the compound increases the SMN level by at least about 1.1, 1.25, 1.5, 1.75, 2, 3, 4, 5, 10 or greater fold. In some embodiments, the cellular compartment is cytoplasm, nucleus or gem. In some embodiments, the compound decrease the SMN level by at least about 5%, 10%, 15%, 20%, 25%, 30%, 35%, 40%, 45%, 50%, 55%, 60%, 65%, 70%, 75%, 80%, 85%, 90%, 95%, 98%, or 99%.

In an embodiment the method further comprises a second step in which a compound tested in the first step is tested in a second step, e.g., a second step in which the compound is evaluated for the ability to rescue a cell in which SMN expression is reduced. In an embodiment the cell in the second step is a neuron, e.g., a motor neuron, e.g., an ES-derived motor neuron. In an embodiment the expression of SMN is reduced in the cell, e.g., by gene silencing, e.g., by siRNA, e.g., with a shRNA, that targets SMN. In an embodiment, the cell having reduced SMN expression is contacted with the compound and the ability of the compound to promote survival, or rescue, the cell having reduced SMN expression is evaluated. E.g., the cell is cultured under conditions which minimize survival of cells having reduced SMN expression, contacted with the compound, and survival of the cell evaluated.

In one aspect, the invention described herein features a method for identifying a biological pathway that regulates the SMN level in a cell, the method comprising: identifying a compound that regulates the SMN level in a cell using a method described herein, and establishing the cellular target of the compound, thereby determining whether the biological pathway comprising the cellular target regulates the SMN level.

In some embodiments, the compound has known biological activity and/or cellular target(s). In some embodiments, the compound has known to modulate a biological pathway. In some embodiments, the cell is a human cell or a mouse cell. In some embodiments, the cell is a whole cell. In some embodiments, the cell is from a subject, e.g., a patient. In some embodiments, the subject, e.g., a patient is suffering from a neurodegenerative disorder. In some embodiments, the neurodegenerative disorder is SMA. In one embodiment, the cell is from a carrier, e.g., a symptom-free carrier. In some embodiments, the cell is a fibroblast. In some embodiments, the cell is a neuron. In some embodiments, the neuron is a motor neuron. In some embodiments, the motor neuron is SMN-deficient. In some embodiments, the SMN level is evaluated by an image-based method. In some embodiments, the compound increases the SMN level by at least about 1.1, 1.25, 1.5, 1.75, 2, 3, 4, 5, 10 or greater fold. In some embodiments, the cellular compartment is cytoplasm, nucleus or gem. In some embodiments, the compound decrease the SMN level by at least about 5%, 10%, 15%, 20%, 25%, 30%, 35%, 40%, 45%, 50%, 55%, 60%, 65%, 70%, 75%, 80%, 85%, 90%, 95%, 98%, or 99%.

In one aspect, the invention described herein features a method for treating a neurodegenerative disorder in a subject, the method comprising: administering a therapeutically effective amount of a compound to the subject, wherein the compound elevates SMN level relative to a standard.

In some embodiments, the method further comprises identifying the compound that elevates the SMN level in a cell using a method described herein.

In some embodiments, the subject is a human. In some embodiments, the neurodegenerative disorder is SMA.

In some embodiments, the compound is a compound described herein. In some embodiments, the compound is of formula (I), (II), (III), (IV), (V), or (VI) as described herein. In some embodiments, the compound is a Na+/K+ channel modulator e.g., cardiac glocosides (e.g., Ouabain, Digoxin, Dititoxin or Lanatoside C). In some embodiments, the compound is an activator of MAPK (e.g., Anysomycin or Coumermycin). In some embodiments, the compound is a cannabinoid receptor or GPCR agonist (e.g., WIN 55,212-2 or Anandamide). In some embodiments, the compound is a Ca2+ channel modulator (e.g., Thapsigargin, Ionomycin or Calcimycin). In some embodiments, the compound is a K⁺ channel modulator (e.g., Veratridine, Monensin Na or Valinomycin). In some embodiments, the compound is a PDE5 inhibitor (e.g., MBCQ or Dipyridamole). In some embodiments, the compound is a kinase inhibitor. In some embodiments, the kinase inhibitor is an inhibitor of GSK/CDK e.g., Alsterpaullone or its structural analogs (e.g., 1-aza-alsterpaullone or 2-cyanoethyl-alsterpaullone). In some embodiments, the kinase inhibitor is an inhibitor of GSK e.g., AR-A014418, CHIR98014 or CHIR99021. In some embodiments, the GSK inhibitor increases motor neuron survival.

In some embodiments, the kinase inhibitor is an inhibitor of PKR. In some embodiments, the kinase inhibitor is an inhibitor of CDK2 e.g., GW8510. In some embodiments, the kinase inhibitor is an inhibitor of IKK-2 e.g., IMD-0354 or its structural analog Niclosamide. In some embodiments, the compound is an HDAC inhibitor e.g., trichostatin. In some embodiments, the compound is a proteasome inhibitor. In some embodiments, the compound is a BMP/TGFβ ligand e.g., BMP4. In some embodiments, the compound is a Dopamine receptor ligand.

In one aspect, the invention described herein features a method for treating a neurodegenerative disorder in a subject, the method comprising: administering a modulator of a biological pathway or target, wherein the modulator elevates the SMN level relative to a standard.

In some embodiments, the biological pathway or target is selected on the basis of modulation of a biological pathway or target described herein.

In some embodiments, the method further comprises identifying a biological pathway that regulates the SMN level in a cell using a method described herein.

In some embodiments, the neurodegenerative disorder is SMA. In some embodiments, the biological pathway is PI-3/AKT/GSK pathway. In some embodiments, the biological pathway is PI-3K signaling pathway. In some embodiments, the biological pathway is Akt signaling pathway. In some embodiments, the biological pathway is PDGF pathway. In some embodiments, the biological pathway is PKR pathway. In some embodiments, the biological pathway is Insulin Receptor pathway. In some embodiments, the biological pathway is MAPK signaling pathway. In some embodiments, the biological pathway is Ras pathway. In some embodiments, the biological pathway is eIF2 pathway. In some embodiments, the biological pathway is mTOR pathway. In some embodiments, the biological pathway is NGF signaling pathway. In some embodiments, the biological pathway is EGF pathway. In some embodiments, the biological pathway is FGF pathway. In some embodiments, the biological pathway is TGF pathway. In some embodiments, the biological pathway is GSK signaling pathway. In some embodiments, the biological pathway is BMP pathway.

In some embodiments, the modulator is a compound described herein. In some embodiments, the compound is of formula (I), (II), (III), (IV), (V), or (VI) as described herein. In some embodiments, the modulator of the biological pathway is a small molecule, an antibody, or a nuclear acid. In some embodiments, modulator of the biological pathway binds to at least one component in the pathway. In some embodiments, the modulator of the PI-3K signaling pathway is PDGF, PDGF-BB or insulin. In some embodiments, the modulator of the PI-3K signaling pathway is FGF, EGF, NGF or TGF. In some embodiments, the modulator of the PI-3K signaling pathway activates PI3K, PDK or PKB. In some embodiments, the modulator of the PI-3K signaling or GSK3 signaling pathway is a GSK inhibitor. In some embodiments, the GSK inhibitor is Alsterpaullone or its structural analogs (e.g., 1-aza-alsterpaullone or 2-cyanoethyl-alsterpaullone), AR-A014418, CHIR98104 or CHIR99021. In some embodiments, the GSK inhibitor increases motor neuron survival. In some embodiments, the modulator of PKR pathway inhibits PKR. In some embodiments, the modulator elevates the SMN level by activation of protein synthesis e.g., translation. In some embodiments, the pathway comprises GSK-3b, CDK2, CDK5, PKR or IKK-2b.

In some embodiments, the modulator of a biological pathway is a compound that inhibits cyclin-dependent kinase (CDK) or glycogen synthase kinase (GSK).

In some embodiments, the modulator is used in combination with another therapeutic agent e.g., Butyrates, Valproic acid, Hydroxyurea or Riluzole.

In one aspect, the invention described herein features a method of treating SMA in a subject, the method comprising: identifying a subject in need of modulation of the level of SMN on the basis of modulation of a biological pathway or target described herein, and treating the subject with a compound that modulates the level of SMN.

In some embodiments, the subject is treated on the basis of identifying that the subject is in need of modulation of the level of SMN.

In some embodiments, the method further comprises identifying a compound that elevates the SMN level in a cell using a method described herein.

In some embodiments, the subject is a human. In some embodiments, the subject is suffering from a neurodegenerative disorder. In some embodiments, the neurodegenerative disorder is SMA.

In some embodiments, the compound is a compound described herein. In some embodiments, the compound is of formula (I), (II), (III), (IV), (V), or (VI) as described herein. In some embodiments, the compound is a Na⁺/K⁺ channel modulator e.g., cardiac glycosides (e.g., Ouabain, Digoxin, Dititoxin or Lanatoside C). In some embodiments, the compound is an activator of MAPK (e.g., Anysomycin or Coumermycin). In some embodiments, the compound is a cannabinoid receptor or GPCR agonist (e.g., WIN 55, 212-2 or Anandamide). In some embodiments, the compound is a Ca2+ channel modulator (e.g., Thapsigargin, Ionomycin or Calcimycin). In some embodiments, the compound is a K⁺ channel modulator (e.g., Veratridine, Monensin Na or Valinomycin). In some embodiments, the compound is a PDE5 inhibitor (e.g., MBCQ or Dipyridamole). In some embodiments, the compound is a kinase inhibitor. In some embodiments, the kinase inhibitor is an inhibitor of GSK/CDK e.g., Alsterpaullone or its structural analogs (e.g., 1-aza-alsterpaullone or 2-cyanoethyl-alsterpaullone). In some embodiments, the kinase inhibitor is an inhibitor of GSK e.g., AR-A014418, CHIR98014 or CHIR99021. In some embodiments, the GSK inhibitor increases motor neuron survival.

In some embodiments, the kinase inhibitor is an inhibitor of PKR. In some embodiments, the kinase inhibitor is an inhibitor of CDK2 e.g., GW8510. In some embodiments, the kinase inhibitor is an inhibitor of IKK-2 e.g., IMD-0354 or its structural analog Niclosamide. In some embodiments, the compound is an HDAC inhibitor e.g., trichostatin. In some embodiments, the compound is a proteasome inhibitor. In some embodiments, the compound is a BMP/TGFβ ligand e.g., BMP4. In some embodiments, the compound is a Dopamine receptor ligand.

In some embodiments, the compound is used in combination with another therapeutic agent e.g., Butyrates, Valproic acid, Hydroxyurea or Riluzole.

In some embodiments, the method further comprises identifying a biological pathway that regulates the SMN level in a cell using a method described herein.

In some embodiments, the neurodegenerative disorder is SMA. In an embodiment, the biological pathway is PI-3/AKT/GSK pathway. In some embodiments, the biological pathway is PI-3K signaling pathway. In some embodiments, the biological pathway is Akt signaling pathway. In some embodiments, the biological pathway is PDGF pathway. In some embodiments, the biological pathway is PKR pathway. In some embodiments, the biological pathway is Insulin Receptor pathway. In some embodiments, the biological pathway is MAPK signaling pathway. In some embodiments, the biological pathway is Ras pathway. In some embodiments, the biological pathway is eIF2 pathway. In some embodiments, the biological pathway is mTOR pathway. In some embodiments, the biological pathway is NGF signaling pathway. In some embodiments, the biological pathway is EGF pathway. In some embodiments, the biological pathway is FGF pathway. In some embodiments, the biological pathway is TGF pathway. In some embodiments, the biological pathway is GSK signaling pathway. In some embodiments, the biological pathway is BMP pathway. In some embodiments, the agonist or antagonist of the biological pathway is a small molecule, an antibody, or a nuclear acid. In some embodiments, the agonist or antagonist of the biological pathway binds to at least one component in the pathway. In some embodiments, the agonist of the PI-3K signaling pathway is PDGF, PDGF-BB or insulin.

In some embodiments, the agonist of the PI-3K signaling pathway is FGF, EGF, NGF or TGF. In some embodiments, the agonist of the PI-3K signaling pathway activates PI3K, PDK or PKB. In some embodiments, the agonist of the PI-3K signaling or GSK3 signaling pathway is a GSK inhibitor. In some embodiments, the GSK inhibitor is Alsterpaullone or its structural analogs (e.g., 1-aza-alsterpaullone or 2-cyanoethyl-alsterpaullone), AR-A014418, CHIR98014 or CHIR99021. In some embodiments, the GSK inhibitor increases motor neuron survival. In some embodiments, the antagonist of PKR pathway inhibits PKR. In some embodiments, the agonist or antagonist elevates the SMN level by activation of protein synthesis e.g., translation. In some embodiments, the pathway comprises GSK-3b, CDK2, CDK5, PKR or IKK-2b.

In some embodiments, the antagonist of a biological pathway is a compound that inhibits cyclin-dependent kinase (CDK) or glycogen synthase kinase (GSK).

In some embodiments, the antagonist of a biological pathway is a compound that inhibits protein kinase R.

In some embodiments, the compound is used in combination with another therapeutic agent e.g., Butyrates, Valproic acid, Hydroxyurea or Riluzole.

In one aspect, the invention described herein features a method of treating SMA in a subject, the method comprising: selecting a compound on the basis that the compound modulates the level of SMN by modulating of a biological pathway or target described herein, and administering to the subject the selected compound.

In some embodiments, the method further comprises identifying a compound that elevates the SMN level in a cell using a method described herein.

In some embodiments, the subject is a human. In some embodiments, the subject is suffering from a neurodegenerative disorder. In some embodiments, the neurodegenerative disorder is SMA.

In some embodiments, the compound is a compound described herein. In some embodiments, the compound is of formula (I), (II), (III), (IV), (V), or (VI) as described herein. In some embodiments, the compound is a Na⁺/K⁺ channel modulator e.g., cardiac glocosides (e.g., Ouabain, Digoxin, Dititoxin or Lanatoside C). In some embodiments, the compound is an activator of MAPK (e.g., Anysomycin or Coumermycin). In some embodiments, the compound is a cannabinoid receptor or GPCR agonist (e.g., WIN 55, 212-2 or Anandamide). In some embodiments, the compound is Ca2+ channel modulator (e.g., Thapsigargin, Ionomycin or Calcimycin). In some embodiments, the compound is a K⁺ channel modulator (e.g., Veratridine, Monensin Na or Valinomycin). In some embodiments, the compound is a PDE5 inhibitor (e.g., MBCQ or Dipyridamole). In some embodiments, the compound is a kinase inhibitor. In some embodiments, the kinase inhibitor is an inhibitor of GSK/CDK e.g., Alsterpaullone or its structural analogs (e.g., 1-aza-alsterpaullone or 2-cyanoethyl-alsterpaullone). In some embodiments, the kinase inhibitor is an inhibitor of GSK e.g., AR-A014418, CHIR98014 or CHIR99021. In some embodiments, the GSK inhibitor increases motor neuron survival.

In some embodiments, the kinase inhibitor is an inhibitor of PKR. In some embodiments, the kinase inhibitor is an inhibitor of CDK2 e.g., GW8510. In some embodiments, the kinase inhibitor is an inhibitor of IKK-2 e.g., IMD-0354 or its structural analog Niclosamide. In some embodiments, the compound is an HDAC inhibitor. In some embodiments, the compound is a proteasome inhibitor. In some embodiments, the compound is a BMP/TGFβ ligand. In some embodiments, the compound is a Dopamine receptor ligand.

In some embodiments, the compound is used in combination with another therapeutic agent e.g., Butyrates, Valproic acid, Hydroxyurea or Riluzole.

In some embodiments, the method further comprises identifying a biological pathway that regulates the SMN level in a cell using a method described herein

In some embodiments, the neurodegenerative disorder is SMA. In an embodiment, the biological pathway is PI-3/AKT/GSK pathway. In some embodiments, the biological pathway is PI-3K signaling pathway. In some embodiments, the biological pathway is Akt signaling pathway. In some embodiments, the biological pathway is PDGF pathway. In some embodiments, the biological pathway is PKR pathway. In some embodiments, the biological pathway is Insulin Receptor pathway. In some embodiments, the biological pathway is MAPK signaling pathway. In some embodiments, the biological pathway is Ras pathway. In some embodiments, the biological pathway is eIF2 pathway. In some embodiments, the biological pathway is mTOR pathway. In some embodiments, the biological pathway is NGF pathway. In some embodiments, the biological pathway is EGF pathway. In some embodiments, the biological pathway is FGF pathway. In some embodiments, the biological pathway is TGF pathway. In some embodiments, the biological pathway is GSK pathway. In some embodiments, the biological pathway is BMP pathway. In some embodiments, the agonist or antagonist of the biological pathway is a small molecule, an antibody, or a nuclear acid. In some embodiments, the agonist or antagonist of the biological pathway binds to at least one component in the pathway. In some embodiments, the agonist of the PI-3K signaling pathway is PDGF, PDGF-BB or insulin. In some embodiments, the agonist of the PI-3K signaling pathway is FGF, EGF, NGF or TGF. In some embodiments, the agonist of the PI-3K signaling pathway activates PI3K, PDK or PKB.

In some embodiments, the agonist of the PI-3K signaling or GSK3 signaling pathway is a GSK inhibitor. In some embodiments, the GSK inhibitor is Alsterpaullone or its structural analogs (e.g., 1-aza-alsterpaullone or 2-cyanoethyl-alsterpaullone) or AR-A014418. In some embodiments, the GSK inhibitor increases motor neuron survival. In some embodiments, the antagonist of PKR pathway inhibits PKR. In some embodiments, the agonist or antagonist elevates the SMN level by activation of protein synthesis e.g., translation. In some embodiments, the pathway comprises GSK-3b, CDK2, CDK5, PKR or IKK-2b.

In some embodiments, the antagonist of a biological pathway is a compound that inhibits cyclin-dependent kinase (CDK) or glycogen synthase kinase (GSK).

In one aspect, the invention teaches a method for treating a neurological disorder such as SMA in a subject using a compound that increases motor neuron survival and/or improves neuromuscular function e.g., by modulating the level of SMN e.g., by modulating a biological pathway or target described herein, and administering to the subject the selected compound.

In one aspect, the invention features a kit comprising: a compound identified by the method described herein, and instructions to treat a neurodegerative disorder e.g. SMA using the method described herein.

### Formulations and Administration

For administration to a subject, the compounds can be administered orally, parenterally, for example, subcutaneously, intravenously, intramuscularly, intraperitoneally, by intranasal instillation, or by application to mucous membranes, such as, that of the nose, throat, and bronchial tubes. One method for targeting the nervous system, such as spinal cord glia, is by intrathecal delivery. The targeted compound is released into the surrounding CSF and/or tissues and the released compound can penetrate into the spinal cord parenchyma, just after acute intrathecal injections. For a comprehensive review on drug delivery strategies including CNS delivery, see Ho et al., Curr. Opin. Mol. Ther. (1999), 1:336-3443, Groothuis et al., J. Neuro Virol. (1997), 3:387-400. and Jan, Drug Delivery SYstmes: Technologies and Commercial Opportunities, Decision Resources, 1998 and

They can be administered alone or with suitable pharmaceutical carriers, and can be in solid or liquid form such as, tablets, capsules, powders, solutions, suspensions, or emulsions.

As used herein, the term “administered” refers to the placement of a compound described herein, into a subject by a method or route which results in at least partial localization of the compound at a desired site. A compound described herein can be administered by any appropriate route which results in effective treatment in the subject, i.e. administration results in delivery to a desired location in the subject where at least a portion of the composition delivered. Exemplary modes of administration include, but are not limited to, injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The compounds can be formulated in pharmaceutically acceptable compositions which comprise a therapeutically-effective amount of the compound, formulated together with one or more pharmaceutically acceptable carriers (additives) and/or diluents. The compounds can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

“PEG” means an ethylene glycol polymer that contains about 20 to about 2000000 linked monomers, typically about 50-1000 linked monomers, usually about 100-300. Polyethylene glycols include PEGs containing various numbers of linked monomers, e.g., PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof.

The compounds can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting an ActRIIB compound with one or more excipients or carriers. In general, the formulations are prepared by uniformly and intimately bringing into association one or more compounds with liquid excipients or finely divided solid excipients or both, and then, if appropriate, shaping the product.

The preparative procedure may include the sterilization of the pharmaceutical preparations. The compounds may be mixed with auxiliary agents such as lubricants, preservatives, stabilizers, salts for influencing osmotic pressure, etc., which do not react deleteriously with the compounds.

Examples of injectable form include solutions, suspensions and emulsions. Injectable forms also include sterile powders for extemporaneous preparation of injectible solutions, suspensions or emulsions. The compounds of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatinA suitable carrier is 5% dextrose in saline. Frequently, it is desirable to include additives in the carrier such as buffers and preservatives or other substances to enhance isotonicity and chemical stability.

In some embodiments, compounds can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

In one embodiment, the compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like.

The compounds can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds may be incorporated with excipients and used in the form of tablets, capsules, elixirs, suspensions, syrups, and the like. Such compositions and preparations should contain at least 0.1% of compound. The percentage of the agent in these compositions may, of course, be varied and may conveniently be between about 2% to about 60% of the weight of the unit. The amount of compound in such therapeutically useful compositions is such that a suitable dosage will be obtained. Preferred compositions according to the present invention are prepared so that an oral dosage unit contains between about 100 and 2000 mg of compound.

Examples of bases useful for the formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives.

The compositions can be given as a bolus dose, to maximize the circulating levels for the greatest length of time after the dose. Continuous infusion may also be used after the bolus dose.

The compounds can also be administrated directly to the airways in the form of an aerosol. For administration by inhalation, the compounds in solution or suspension can be delivered in the form of an aerosol spray from pressured container or dispenser which contains a suitable propellant, e.g., a gas such as carbon dioxide, or hydrocarbon propellant like propane, butane or isobutene. The compounds can also be administrated in a no-pressurized form such as in an atomizer or nebulizer.

The compounds can also be administered parenterally. Solutions or suspensions of these compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of compound calculated to produce the desired therapeutic effect in association with the required pharmaceutical carrier.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

The compounds can be administrated to a subject in combination with a pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference. In some embodiments, the pharmaceutically active agent is selected from the group consisting of butyrates, valproic acid, hydroxyuirae and Riluzole.

The compound and the pharmaceutically active agent may be administrated to the subject in the same pharmaceutical composition or in different pharmaceutical compositions (at the same time or at different times). In some embodiments, the pharmaceutically active compound is a

The amount of compound which can be combined with a carrier material to produce a single dosage form will generally be that amount of the compound which produces a therapeutic effect. Generally out of one hundred percent, this amount will range from about 0.1% to 99% of compound, preferably from about 5% to about 70%, most preferably from 10% to about 30%.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

The pharmaceutical compositions can be included in a container, pack, or dispenser together with instructions for administration.

As used herein, the term “therapeutically effective amount” means an amount of the compound which is effective to promote the survival of motor neuron cells or to prevent or slow the death of such cells. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in neurodegenerative disorders.

Guidance regarding the efficacy and dosage which will deliver a therapeutically effective amount of a compound to treat SMA can be obtained from animal models of SMA, see e.g., those described in Hsieh-Li et al. Nature Genetics. 2000; 24:66-70 and references cited therein.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, GDF-8 binding assays, and immunological assays.

The dosage may be determined by a physician and adjusted, as necessary, to suit observed effects of the treatment. Generally, the compositions are administered so that compound is given at a dose from 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. For antibody compounds, one preferred dosage is 0.1 mg/kg of body weight (generally 10 mg/kg to 20 mg/kg). If the antibody is to act in the brain, a dosage of 50 mg/kg to 100 mg/kg is usually appropriate.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dos can be administered at one time or divided into subdoses, e.g., 2-4 subdoses and administered over a period of time, e.g., at appropriate intervals through the day or other appropriate schedule. Such sub-doses can be administered as unit dosage forms. Examples of dosing schedules are administration once a week, twice a week, three times a week, daily, twice daily, three times daily or four or more times daily.

### Survival of Motor Neuron (SMN)

Survival of Motor Neuron (SMN) is also known as Component of gems 1 or Gemin-1. In human, there are two genes associated with this protein: SMN1 (also known as SMN or SMNT) and SMN2 (also known as SMNC).

The nucleotide and amino acid sequences of human SMN are disclosed in the art e.g., Lefebvre S. et al., Cell 80:155-165 (1995); Buerglen L. et al., Genomics 32:479-482 (1996); Chen Q. et al., Genomics 48:121-127 (1998); Gennarelli M. et al., Biochem. Biophys. Res. Commun. 213:342-348 (1995); Schmutz J. et al., Nature 431:268-274 (2004); The MGC Project Team, Genome Res. 14:2121-2127 (2004); and van der Steege G. et al., Eur. J. Hum. Genet. 3:87-95 (1995). The nucleotide and amino acid sequences of mouse SMN are disclosed in the art e.g., Viollet L. et al., Genomics 40:185-188 (1997); Didonato C. J. et al., Genome Res. 7:339-352 (1997); Schrank B. et al., Proc. Natl. Acad. Sci. U.S.A. 94:9920-9925 (1997); and The MGC Project Team; Genome Res. 14:2121-2127 (2004).

The SMN complex plays an essential role in spliceosomal snRNP assembly in the cytoplasm and is required for pre-mRNA splicing in the nucleus. It may also play a role in the metabolism of snoRNPs.

The SMN protein is localized in the cytoplasm, nucleus and gem. ((subnuclear structures next to coiled bodies, called Gemini of Cajal bodies (Gems)).

The SMN protein is expressed in a wide variety of tissues. E.g., it is expressed at high levels in brain, kidney and liver, moderate levels in skeletal and cardiac muscle, and low levels in fibroblasts and lymphocytes. It is also seen at high levels in spinal cord and is present in osteoclasts and mononuclear cells (at protein level).

The unprocessed human SMN protein is 294 amino acid in length with a molecular weight of about 32 kDa. The unprocessed mouse SMN protein is 288 amino acid in length with a molecular weight of about 31 kDa.

SMN is part of a stable complex that contains at least six other proteins, named Gemins 2-7, and is found in all metazoan cells. SMN protein is localized in the cytoplasm and in the nuclear structures called Gems that appear to be similar to and possibly interact with coiled bodies. The full spectrum of SMN functions in nucleus and cytoplasm has not been determined. In the cytoplasm, the SMN complex known to be a core mediator of assembly and trafficking of spliceosomal snRNP, and Gems are thought to be the centers of pre-mRNA splicing orchestrated by the SMN complex. It has been shown that cytosolic components phosphorylate SMN and transform the complex in to the active state. Interestingly, in both cases of overexpression of SMNΔexon7 and SMN+exon7, the number of nuclear gems was increased, however only the later construct promoted SMN increase in the cytoplasm. Although SMN protein is ubiquitously expressed, the main questions about why only motor neurons die during SMA, and which pool of SMN is essential for these cells are still remain. It has been presumed that disturbance in snRNP formation and splicing of motor neuron specific genes are attributers of such specificity. And indeed, it has been shown that ability of SMN complex to perform snRNP assembly determines the survival of animal with SMA (Workman E, HMG, 2009). In particular, there was noted a reduction of minor snRNP's that are responsible for splicing of approximately 700 genes (Gabanella F, PLoS, 2007). Second hypothesis points at the loss of function that SMN possibly has in motor neuronal axons. For example, reduced levels of β-actin mRNA transport to the growth cones alter the number of certain Ca2+ channels (Rossoll W, JCB, 2003). Recently, cytoplasmic LSm4 protein known as part of the RNP complex associated with axonal RNA transport was shown to bind to SMN (Beattie, 2008 review). Apart from splicing, SMN protein has also been reported to influence several other cellular activities such as transcription, ribosomal assembly, and apoptosis. Therefore SMN localization may reflect its multiple roles and their diversity is still a matter of further study.

SMA has recently attracted a great deal of attention from researchers because of its monogenic nature and seemingly straightforward path to the clinic. Data obtained from experiments on fibroblasts derived from SMA patients and from SMA mouse models suggest that therapeutics that elevate Survival of Motor Neuron (SMN) levels will be effective in treating this disease (citation). Previously, large scale screens have been performed using libraries of diverse chemical structures with unknown biological activities in attempt to identify a scaffold that would be a clinical candidate for treating SMA disease. There are studies that describe screens based on the enzyme-reporter assays designed to identify compounds that stimulate Smn2 transcriptionor facilitate the splicing of Exon 7. It is well understood that reduced SMN levels is what ultimately causes SMA and these reporter assays only identify compounds that modulate the level of mRNA and are not designed to find compounds that elevate SMN protein, alter SMN localization or stability. One of the promising high-throughput assays was established to study the mechanism of snRNP assembly and identified compounds that decrease the efficiency of this process; however no compounds were detected that promoted it (Dreyfuss, 2008). Several other screens were directed toward finding compounds that increase the number of nuclear SMN-containing gems. However, since it has already been mentioned that the functional pools of SMN in motor neurons have not been identified conclusively, Gem assays may fail to discover the most effective compounds. Another problem with these assays is that the cells used have no obvious disease related phenotype. Therefore, this presents an additional challenge when trying to determine if compounds found in a primary screen can be effective in modifying the physiological defects that underlie SMA.

Results described herein show that there are several different classes of compounds that appear able to increase SMN. One of these pathways—PI-3 kinase, activated by different receptor tyrosine (RTK) ligands, is particularly effective. A downstream target of this pathway, GSK3 kinase, seems to be especially important. Inhibitors of this kinase elevate SMN levels in both patient fibroblasts and in motor neurons, derived from mouse embryonic stem cells. Importantly, they also decrease motor neuron death that follows reduction of SMN levels.

Biological Pathways and Targets that Modulate Levels of SMN

Non-limiting examples of the biological pathways that modulate levels of SMN include e.g. PI3K signaling pathway, Akt signaling pathway, MAPK signaling pathway, PDGF pathway, RAS pathway, eIF2 pathway, GSK pathway, PKR pathway, Insulin Receptor Pathway, mTOR pathway, EGF pathway, NGF pathway, FGF pathway and BMP/TGFβ pathway. Non-limiting examples of the targets that modulate levels of SMN include e.g. components of the biological pathways described herein. In some preferred embodiments, the targets that modulate levels of SMN include e.g., Na+/K+ channel, MAPK, cannobinoid receptor, GPCR, Ca2+ channel, K+ channel, PDE5, GSK/CDK, GSK, PKR, CDK2, IKK-2, HDAC, proteasome, BMP/TGFβ receptor and Dopamine receptor.

### PI3K Signaling Pathway

The definition and details of the PI3K signaling pathway are disclosed in the art e.g., Abell K. and Watson, C. J. Cell Cycle. 4, 897-900 (2005); Brachmann, S. M. et al., Mol. Cell. Biol. 25, 2593-2606 (2005); Katso R. et al., Annu. Rev. Cell Dev. Biol. 17, 615-675 (2001); and Vanhaesebroeck B. and Waterfield M. D. Exp. Cell Res. 253, 239-254 (1999).

### Akt Signaling Pathway

The definition and details of the PI3K signaling pathway are disclosed in the art e.g., Downward, J. Curr. Opin. Cell Biol. 10, 262-267 (1988); Jimenez, C. et al., J. Biol. Chem. 277(44):41556-41562 (2002); Kitamura, T. et al., Mol. Cell. Biol. 19, 6286-6296 (1999); Ruggero D. and Sonenberg N. Oncogene. 24, 7426-34 (2005); Testa J. R. and Tsichlis P. N. Oncogene. 7391-7393 (2005); and Zhou X. M. et al., J. Biol. Chem. 275, 25046-25051 (2000).

### MAPK Signaling Pathway

The definition and details of the MAPK signaling pathway are disclosed in the art e.g., Ichijo H. et al., Science 275, 90-94 (1997); Qiu M. S. and Green S. H. Neuron 7, 937-946 (1991); Rubinfeld H. and Seger R. Mol. Biotechnol. 31, 151-174 (2005); and Yoon S, and Seger R. Growth Factors. 24, 21-44 (2006).

### PDGF Pathway

The definition and details of the PDGF pathway are disclosed in the art e.g., Fredriksson L. et al., J. Biol. Chem. 280, 26856-26862 (2005); Hauck C. R. et al., J. Biol. Chem. 275, 41092-41099 (2000); Hoch R. V. and Soriano P. Development 130, 4769-4784 (2003); Jiang B. et al., Surgery. 120, 427-431 (1996); and Reigstad L. J. et al., FEBS J. 272, 5723-5741 (2005).

### RAS Pathway

The definition and details of the RAS pathway are disclosed in the art e.g., Ada-Nguema A. S. et. al. J. Cell Sci. 119, 1307-1319 (2006); Hofer F. et al., Pro. Natl. Acad. Sci. 91, 11089-11093 (1994); Kikuchi A. et. al. Mol. Cell. Biol. 14, 7483-7491 (1994); Rodriguez-Viciana, P. et al., Nature 370, 527-532; and Rubio, I. et al., Biochem. J. 326, 891-895 (1997).

eIF2 Pathway

The definition and details of the eIF2 pathway are disclosed in the art e.g., Clemens M. J. Prog. Mol. Subcell. Biol. 27, 57-89 (2001); Proud C. G. Semin. Cell Dev. Biol. 16, 3-12 (2005); and Wek R. C. et al., Biochem. Soc. Trans. 34, 7-11 (2006).

### GSK3 Pathway

The definition and details of the GSK3 pathway are disclosed in the art e.g., Biondi R. M. and Nebreda A. R. Biochem J. 372, 1-13 (2003); Jope R. S. and Johnson G. V. Trends Biochem Sci. 29, 95-102 (2004); and Polakis P. Curr. Biol. 12, R499—R501 (2002).

### PKR Pathway

The definition and details of the PKR pathway are disclosed in the art e.g., Bennett R. L. et al., Blood. 108, 821-829 (2006); Donze O. et al., EMBO J. 23, 564-571 (2004); Guerra S. et al., J. Biol. Chem. 281, 18734-18745 (2006); and Li S. et al., Proc. Natl. Acad. Sci. USA. 103, 10005-10010 (2006).

### Insulin Receptor Pathway

The definition and details of the Insulin Receptor pathway are disclosed in the art e.g., Dudek H. et al., Science. 275, 661-665 (1997); Pandini G. et al., J. Biol. Chem. 277, 39684-39695 (2002); and White M. F. and Myers M. G. In Endocrinology (DeGroot, L. J., and Jameson, J. L., eds), W. B. Saunders Co., Philadelphia (2001).

mTOR Pathway

The definition and details of the mTOR pathway are disclosed in the art e.g., Gingras A. C. et al., Genes Dev. 15, 807-826 (2001); Hannan K. M. et al., Mol. Cell. Biol. 23, 8862-8877 (2003); Kim D. H. et al., Cell 110, 163-175 (2002); Kumar V. et al., J. Biol. Chem. 275, 10779-10787 (2000); and Raught B. et al., Proc. Natl. Acad. Sci. USA 98, 7037-7044 (2001).

### EGF Pathway

The definition and details of the EGF pathway are disclosed in the art e.g., Carpenter G. and Ji Q. Exp. Cell Res. 253, 15-24 (1999); Garcia R. et al., Oncogene 20, 2499-2513 (2001); Henson E. S. and Gibson S. B. Cell Signal. (2006); Olayioye M. A. et al., J. Biol. Chem. 274, 17209-17218 (1999); Guren T. K. et al. J. Cell Physiol. 196, 113-123 (2003); and Sato K. et al., J. Biol. Chem. 277, 29568-29576 (2002).

### NGF Pathway

The definition and details of the EGF pathway are disclosed in the art e.g., Coulson E. J. Prog. Brain Res. 146, 41-62 (2004); Huang E. J. and Reichardt L. F. Annu. Rev. Biochem. 72, 609-642 (2003); Miller F. D. and Kaplan D. R. Cell Mol. Life. Sci. 58, 1045-1053 (2001); and Rabizadeh S. and Bredesen D. E. Cytokine Growth Factor Rev. 14, 225-239 (2003).

### FGF Pathway

The definition and details of the FGF pathway are disclosed in the art e.g., Lee P. L. et al., Science. 245, 57-60 (1989); Mignatti P. et al., J. Cell Physiol. 151, 81-93 (1992); Miki T. et al., Proc. Natl. Acad. Sci. USA. 89, 246-250 (1992); Gringel S. et al., J. Biol. Chem. 385, 1203-1208 (2004); and Ornitz D. M. and Itoh, N. Genome Biol. 2, 1-12 (2001); Sorensen V. et al., Bioessays. 28, 504-514 (2006).

### BMP/TGFβ Pathway

The definition and details of the BMP/TGFβ pathway are disclosed in the art e.g., Kawabata M. and Miyazono K., J. Biochem. (Tokyo), 125, 9-16 (1999); Wrana J. L., Miner. Electrolyte Metab., 24, 120-130 (1998); and Markowitz S. D., and Roberts A. B., Cytokine Growth Factor Rev., 7, 93-102 (1996).

### Phosphoinositide 3-Kinases

Phosphoinositide 3-kinases (PI 3-kinases or PI3Ks) are a family of related enzymes that are capable of phosphorylating the 3 position hydroxyl group of the inositol ring of phosphatidylinositol (PtdIns).http://en.wikipedia.org/wiki/Phosphoinositide_{—}3-kinase-cite_note-0. They are also known as phosphatidylinositol-3-kinases.

PI3Ks interact with the IRS (Insulin receptor substrate) in order to regulate glucose uptake through a series of phosphorylation events. The phosphoinositol-3-kinase family is composed of Class I, II and Class III, with Class I the only ones able to convert PI(4,5)P2 to PI(3,4,5)P3 on the inner leaflet of the plasma membrane.

Class I PI3K are heterodimeric molecules composed of a regulatory and a catalytic subunit; they are further divided between IA and IB subsets on sequence similarity. Class IA PI3K are composed of one of five regulatory p85a, p55α, p50α, p85β or p55γ subunit attached to a p110α, β or δ catalytic subunit. The first three regulatory subunits are all splice variants of the same gene (Pik3r1), the other two being expressed by other genes (Pik3r2 and Pik3r3, p85β and p55γ, respectively). The most highly expressed regulatory subunit is p85α, all three catalytic subunits are expressed by separate genes (Pik3ca, Pik3cb and Pik3cd for p110α, p110β and p110δ, respectively). The first two p110 isoforms (α and β) are expressed in all cells, but p110δ is primarily expressed in leukocytes and it has been suggested it evolved in parallel with the adaptive immune system. The regulatory p101 and catalytic p110γ subunits comprise the type IB PI3K and are encoded by a single gene each.

Class II comprises three catalytic isoforms (C2α, C2β, and C2γ), but unlike Classes I and III, no regulatory proteins. These enzymes catalyse the production of PI(3)P from PI (may also produce PI(3,4)P2 from PI(4)P). C2α and C2β are expressed throughout the body, however expression of C2γ is limited to hepatocytes. The distinct feature of Class II PI3Ks is the C-terminal C2 domain. This domain lacks critical Asp residues to coordinate binding of Ca²⁺, which suggests class II PI3Ks bind lipids in a Ca²⁺ independent manner.

Class III are similar to II in that they bias the production of PI(3)P from PI, but are more similar to Class I in structure, as they exist as a heterodimers of a catalytic (Vps34) and a regulatory (p150) subunits. Class III seems to be primarily involved in the trafficking of proteins and vesicles.

All PI 3-kinases are inhibited by the drugs wortmannin and LY294002, although certain member of the class II PI 3-kinase family show decreased sensitivity.

PI 3-kinases have been linked to an extraordinarily diverse group of cellular functions, including cell growth, proliferation, differentiation, motility, survival and intracellular trafficking. Many of these functions relate to the ability of class I PI 3-kinases to activate protein kinase B (PKB, aka Akt). The class IA PI 3-kinase p110a is mutated in many cancers. The PtdIns(3,4,5)P₃ phosphatase PTEN which antagonises PI 3-kinase signalling is absent from many tumors. Hence, PI 3-kinase activity contributes significantly to cellular transformation and the development of cancer. The p110δ and p110γ isoforms regulate different aspects of immune responses. PI 3-kinases are also a key component of the insulin signaling pathway.

AKT is activated as a result of PI3-kinase activity, because AKT requires the formation of the PtdIns(3,4,5)P3 (or “PIP3”) molecule in order to be translocated to the cell membrane. At PIP3, AKT is then phosphorylated by phosphoinositide dependent kinase 1 (PDK1), and is thereby activated. The “PI3-k/AKT” signaling pathway has been shown to be required for an extremely diverse array of cellular activities such as cellular proliferation and survival.

In addition to AKT and PDK1, one other related serine threonine kinase is bound at the PIP3 molecule created as a result of PI3-kinase activity, SGK.

PI3K has also been implicated in Long term potentiation (LTP).

The PI3K pathway also recruits many other proteins downstream, including mTOR, GSK3β, and PSD-95. The PI3K-mTOR pathway leads to the phosphorylation of p70S6K, a kinase which facilitates translational activity.

### Glycogen Synthase Kinase 3 (GSK-3)

Glycogen synthase kinase 3 (GSK-3) is a serine/threonine protein kinase. In mammals GSK-3 is encoded by two known genes GSK-3α, http://en.wikipedia.org/wiki/GSK3A and β.

The nucleotide and amino acid sequences of human GSK-3α are disclosed in the art e.g., Hoshino T. et al., “Isolation of cDNA clones for human glycogen synthase kinase 3alpha.”, Submitted (NOV-1997) to the EMBL/GenBank/DDBJ databases; Grimwood J., et al., Nature 428:529-535 (2004); and The MGC Project Team, Genome Res. 14:2121-2127 (2004). The nucleotide and amino acid sequences of human GSK-3β are disclosed in the art e.g., Stambolic V. and Woodgett J. R. Biochem. J. 303:701-704 (1994); The MGC Project Team, Genome Res. 14:2121-2127 (2004); Rhoads A. R. et al., Mol. Psychiatry. 4:437-442 (1999); and Lau K. F. et al., Genomics 60:121-128 (1999).

GSK-3α is implicated in the hormonal control of several regulatory proteins including glycogen synthase, MYB and the transcription factor JUN. GSK-3β participates in the Wnt signaling pathway. It is implicated in the hormonal control of several regulatory proteins including glycogen synthase, MYB and the transcription factor JUN. It also phosphorylates JUN at sites proximal to its DNA-binding domain, thereby reducing its affinity for DNA. It phosphorylates MUC1 in breast cancer cells, and decreases the interaction of MUC1 with CTNNB1/beta-catenin. GSK-3β is inhibited when phosphorylated by AKT1.

GSK-3β is expressed in testis, thymus, prostate and ovary and weakly expressed in lung, brain and kidney.

The unprocessed human GSK-3α protein is 483 amino acid in length with a molecular weight of about 51 kDa. The unprocessed human GSK-3β protein is 420 amino acid in length with a molecular weight of about 47 kDa.

### Ca²⁺ Channel

A Calcium channel is an ion channel which displays selective permeability to calcium ions. It is also called as voltage-dependent calcium channel, although there are also ligand-gated calcium channels.

Calcium channel blockers are a class of drugs and natural substances with effects on many excitable cells of the body such as the muscle of the heart, smooth muscles of the vessels or neuron cells. Classes of calcium channel blockers include e.g., Dihydropyridine, Phenylalkylamine, Benzothiazepine.

cGMP-Specific 3′,5′-cyclic Phosphodiesterase (PDE5)

PDE5 refers to a cGMP-binding, cGMP-specific phosphodiesterase, a member of the cyclic nucleotide phosphodiesterase family. This phosphodiesterase specifically hydrolyzes cGMP to 5′-GMP. It is involved in the regulation of intracellular concentrations of cyclic nucleotides and is important for smooth muscle relaxation in the cardiovascular system. Human PDE5 is expressed in aortic smooth muscle cells, heart, placenta, skeletal muscle and pancreas and, to a much lesser extent, in brain, liver and lung.

A PDE5 inhibitor, is a drug used to block the degradative action of PDE5 on cyclic GMP in the smooth muscle cells lining the blood vessels supplying the corpus cavernosum of the penis. These drugs are used in the treatment of erectile dysfunction, Because PDE5 is also present in the arterial wall smooth muscle within the lungs, PDE5 inhibitors have also been explored for the treatment of pulmonary hypertension, a disease in which blood vessels in the lungs become abnormally narrow.

### Cannabinoid Receptors

The cannabinoid receptors refer to members of the family of guanine-nucleotide-binding protein (G-protein) coupled receptors which inhibit adenylate cyclase activity in a dose-dependent, stereoselective and pertussis toxin-sensitive manner. The cannabinoid receptors have been found to be involved in the cannabinoid-induced CNS effects (including alterations in mood and cognition) experienced by users of marijuana. Their ligands are known as cannabinoids or endocannabinoids.

### Histone Deacetylase (HDAC)

Histone deacetylases (HDAC) are a class of enzymes that remove acetyl groups from an ε-N-acetyl lysine amino acid on a histone. Exemplary HDACs include those Class I HDAC: HDAC1, HDAC2, HDAC3, HDAC8; and Class II HDACs: HDAC4, HDAC5, HDAC6, HDAC7A, HDAC9, HDAC10. Type I mammalian HDACs include: HDAC1, HDAC2, HDAC3, HDAC8, and HDAC11. Type II mammalian HDACs include: HDAC4, HDAC5, HDAC6, HDAC7, HDAC9, and HDAC1.

### Cardiac Glycosides

Cardiac glycosides are drugs used in the treatment of congestive heart failure and cardiac arrhythmia. Cardiac glycosides work by inhibiting the Na⁺/K⁺ pump. This causes an increase in the level of sodium ions in the myocytes, which then leads to a rise in the level of calcium ions. This inhibition increases the amount of Ca²⁺ ions available for contraction of the heart muscle, improves cardiac output and reduces distention of the heart.

### Inhibitor of IκB Kinase 2 (IKK2)

IKK2 is a protein which is a component of a cytokine-activated intracellular pathway involved in triggering immune responses. Activation of IKK2 leads to phosphorylation of the inhibitor of Nuclear Transcription factor kappa-B (IkB). Phosphorylation of IkB causes the degradation of the inhibitor IkB via the ubiquination pathway, thereby allowing the transcription factor NFkB to enter the cell's nucleus and activate various genes involved in inflammation and other immune responses.

IKK2 plays a significant factor in the state of brain cells after a stroke. Experimental mice that had an overactive form of IKK2 experienced the loss of many more neurons than controls did after a stroke-simulating event.

### Cyclin-Dependent Kinase 2 (CDK2)

The protein encoded by this gene is a member of the cyclin-dependent kinase family of Ser/Thr protein kinases. This protein kinase is highly similar to the gene products of S. cerevisiae cdc28, and S. pombe cdc2. It is a catalytic subunit of the cyclin-dependent kinase complex, whose activity is restricted to the G1-S phase of the cell cycle, and is essential for the G1/S transition. This protein associates with and regulated by the regulatory subunits of the complex including cyclin E or A. Cyclin E binds G1 phase Cdk2, which is required for the transition from G1 to S phase while binding with Cyclin A is required to progress through the S phase. Its activity is also regulated by phosphorylation. Two alternatively spliced variants and multiple transcription initiation sites of this gene have been reported. The role of this protein in G1-S transition has been recently questioned as cells lacking Cdk2 are reported to have no problem during this transition.

Known CDK inhibitors are p21Cip1 (CDKN1A) and p27Kip1 (CDKN1B). Drugs which inhibit Cdk2 and arrest the cell cycle may reduce the sensitivity of the epithelium to many cell cycle-active antitumor agents and therefore represent a strategy for prevention of chemotherapy-induced alopecia.

Compounds that Elevate Levels of SMN

Non-limiting examples of compounds that elevate, levels of SMN include e.g., activators and inhibitors that modulate the biological pathways and targets described herein. In some embodiments, the compound is a compound described herein. In some embodiments, the compound is of formula (I), (II), (III), (IV), (V), or (VI) as described herein. In some preferred embodiments, the compounds include e.g., PDGF (e.g., PDGF-BB), Insulin, FGF (e.g., FGF2), EGF, NGF, TGF (e.g., TGFβ), Na+/K+ channel modulators e.g., cardiac glycosides (e.g., Ouabain, Digoxin, Dititoxin and Lanatoside C), activators of MAPK (e.g., Anysomycin or Coumermycin), cannabinoid receptor or GPCR agonists (e.g., WIN 55, 212-2 or Anandamide), Ca2+ channel modulators (e.g., Thapsigargin, Ionomycin or Calcimycin), K+ channel modulators (e.g., Veratridine, Monensin Na or Valinomycin), PDE5 inhibitors (e.g., MBCQ or Dipyridamole), kinase inhibitors, inhibitors of GSK/CDK e.g., Alsterpaullone or its structural analogs (e.g., 1-aza-alsterpaullone or 2-cyanoethyl-alsterpaullone) and other inhibitors of GSK/CDK described herein (the use of paullones for making compounds is disclosed in U.S. Pat. No. 7,232,814), inhibitors of GSK e.g., AR-A014418, CHIR98014, CHIR99021 and other inhibitors of GSK described herein, inhibitors of PKR, inhibitor of CDK2 e.g., GW8510 and other inhibitors of CDK2 described herein, inhibitors of IKK-2 e.g., IMD-0354 or its structural analog Niclosamide and other inhibitor of IKK02 described herein, HDAC inhibitor e.g., trichostatin, proteasome inhibitors, BMP/TGFβ ligands e.g., BMP4, and Dopamine receptor ligands.

Compounds that can be used to elevate the levels of SMN in a subject are described herein. For example, a compound that modulates a target or pathway described herein can be used to modulate (increase) the levels of SMN in a subject. Representative compounds that can be used to elevate the levels of SMN include the compounds of formulas (I), (II), (III), (IV), (V), and (VI), and other compounds described herein.

### Kits

A compound described herein can be provided in a kit. The kit includes (a) the compound, e.g., a composition that includes the compound, and (b) informational material. The informational material can be descriptive, instructional, marketing or other material that relates to the methods described herein and/or the use of the compound for the methods described herein. For example, the informational material describes methods for administering the compound to alter lifespan regulation or at least one symptom of aging or an age related disease.

In one embodiment, the informational material can include instructions to administer the compound in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., an adult human. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the modulator and/or its use in the methods described herein. Of course, the informational material can also be provided in any combination of formats.

In addition to the compound, the composition of the kit can include other ingredients, such as a solvent or buffer, a stabilizer or a preservative, and/or a second agent for treating a condition or disorder described herein, e.g. increased pancreatic islet mass. Alternatively, the other ingredients can be included in the kit, but in different compositions or containers than the compound. In such embodiments, the kit can include instructions for admixing the compound and the other ingredients, or for using the modulator together with the other ingredients.

The compound can be provided in any form, e.g., liquid, dried or lyophilized form. It is preferred that the compound be substantially pure and/or sterile. When the compound is provided in a liquid solution, the liquid solution preferably is an aqueous solution, with a sterile aqueous solution being preferred. When the compound is provided as a dried form, reconstitution generally is by the addition of a suitable solvent. The solvent, e.g., sterile water or buffer, can optionally be provided in the kit.

The kit can include one or more containers for the composition containing the compound. In some embodiments, the kit contains separate containers, dividers or compartments for the compound (e.g., in a composition) and informational material. For example, the compound (e.g., in a composition) can be contained in a bottle, vial, or syringe, and the informational material can be contained in a plastic sleeve or packet. In other embodiments, the separate elements of the kit are contained within a single, undivided container. For example, the compound (e.g., in a composition) is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the compound (e.g., in a composition). For example, the kit includes a plurality of syringes, ampules, foil packets, or blister packs, each containing a single unit dose of the compound. The containers of the kits can be air tight and/or waterproof.

The compound (e.g., in a composition) can be administered to a subject, e.g., an adult subject, e.g., a subject in need of preserved pancreatic islet mass. The method can include evaluating a subject, e.g., to evaluate pancreatic islet mass, and thereby identifying a subject as having increased islet mass or being pre-disposed it.

### Summary

In this work, the first high-content screen for bioactive small molecules that increase the SMN protein level in different cellular compartments in fibroblasts derived from SMA patients was conducted. AS shown in the examples herein, detection of SMN protein increase only based on the gem number does not necessarily reveal the most potent compounds. Since it is not known increasing which form of SMN in the cell is the most important, we think that our high content approach of measuring SMN increase in the cytoplasm and the nucleus is providing the most accurate information.

In patients with SMA, higher level of SMN protein appears to be associated with less severe disease phenotype, and these general findings have been confirmed in mouse studies (citation). This suggests a clear therapeutic strategy of identifying a drug that would increase SMN protein levels in patients with SMA. In this study several compound classes were found that actively increased SMN protein in patient fibroblasts. However, it can be more important to identify biological pathways that play a role in modulating the level of SMN. Understanding what pathways are involved can help in finding better ways of rescuing motor neurons, the cells that are mainly affected in SMA disease.

Screening of utilized in this study revealed multiple hits from a series of cardiac glycosidase that are known to increase intracellular concentrations of Na⁺ and Ca²⁺ and activate kinase signaling cascade downstream of RTK (citation). Pathway involved in the SMN regulation were further investigated using additional chemical tools targeting different components of those pathways. Results confirmed that ion modulators, mainly Ca²⁺ and Na⁺ agonists, so called ionophore compounds that increase concentration of Na⁺ and Ca²⁺ in the cell were able to increase SMN.

According to several other reports, cardiac glycosides are also known to activate EGFR signaling cascade, MAPk and PI-3K signaling, and some reports indicate that there due to Ca2+ oscillations, NF-kb transcription factor shuttling in to the nucleus can occur. Whether manipulation with some of the described pathways could have any influence on SMN level was investigated next Kinase signaling cascades were of particular interest, since previously it has been reported in the literature that modest increase in intracellular Ca2+ also triggers signaling cascade in which CaM-KK can directly activates AKT kinase {Yano, 1998 #9}. It also had been recently demonstrated that GSK inhibitors Alsterpaullone and SB216763 are able to protect PC12 cells from EGTA induced death, showing that Ca2⁺ and GSK kinase signaling indeed could be linked (Takadera T, 2009). Hypothesis of RTK activation was tested by using biological tools such as RTK receptor ligands in order to prove observations seen with compounds. Testing of various growth factors that could be able to also increase SMN led to the identification of PDGF, one of the most potent SMN protein up-regulators. Pre-incubation of fibroblasts with both inhibitors of PDGFR, small molecules DMPQ and AG-1296 was sufficient to prevent PDGF effect on SMN, observation that was also confirmed with the α-PDGF neutralizing antibodies. It was also observed that We found inhibitors of the PI-3 kinase pathway LY294002 and PI-103, but not ERK inhibitors PD58059 and U0-126, affected SMN level and were able to prevent PDGF induced SMN increase in all cellular compartments. Accordingly to the previous observation that p38 kinase activation also able to increase SMN (McKenzie), it was shown that both p38 inhibitors SB202190 and SB203580 were able to prevent SMN increase induced by PDGF, but only in the cytoplasm and not in the nucleus. Downstream of PI-3 kinase pathway there were several GSK inhibitors that were able to increase SMN level in patient derived fibroblasts and ESC-derived motor neurons.

The most important advancement in the screening approach was the utilization of an SMA cellular model employing mESC-derived MN in which SMN has been knocked down. It was observed that reduction of the SMN level more than 70% leads to motor neuronal loss. shRNA targeted to the mouse SMN gene were used to reduce SMN, and the ability of a compound to rescue the survival defect seen when SMN is knocked down in these mouse ESC derived MNs was investigated. Interestingly, the majority of the fibroblast-active compound classes were not able to prevent neuronal death or increase SMN level. However, it was observed that compounds targeting the PI-3/AKT/GSK pathway are potent modulators of SMN levels in fibroblasts and Alsterpaullone was able to rescue the survival of ESC derived motor neurons lacking SMN. This indicates that PI-3K/AKT/GSK pathway may play an important role in regulating SMN in different cell types. Without wishing to be bound by theory, in motor neurons, regulation of SMN can be achieved by modulating this pathway through cell-specific receptors. It is known that PDGF receptors are not expressed on mature motor neurons, explaining why PDGF had no effect on elevating SMN and prolonging MN survival (data not shown).

Again, without wishing to be bound by theory, upstream receptors/factors can also be utilized that physiologically regulate SMN level specifically in the motor neurons. For example, these upstream receptors can include neurotrphins, neuromediators, glia-secreted factors etc. A high-content screening assay can be established using ES-cell derived Motor Neurons as described herein. This screen then can be used to look for bioactive small molecules and receptor ligands that are present in the CNS (and their analogs) that would be able to rescue the survival of ES-cell derived motor neurons with SMN knockdown. Active compounds then can be assayed to see if they can elevate the SMN exclusively in motor neurons, by the mechanisms that are specific only for these disease-related cells.

Although motor neuron death is a significant feature of SMA disease, there is recent information that suggests that muscle dysfunction or malformation may also occur (citation). While SMN protein appears to have multiple cellular roles, and it is not yet clear which may support neuromuscular development and function. Since we developed SMN-deficient motor neuronal model assay, it will be important to functionally characterize behavior of these cells in the presence or absence of muscle cells that are normal or deficient in SMN to determine if a motor neuron or neuromuscular phenotype can be observed in vitro. Then, the ability of SMN-elevating compounds, many of which have already been identified in this work, to correct phenotypic differences established in motor neurons or motor neuron-muscle cultures in vitro can be examined.

### DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, the term “modulate” means to cause or facilitate a qualitative or quantitative change, alteration, or modification in a molecule, a process, pathway, or phenomenon of interest. Without limitation, such change may be an increase, decrease, a change in binding characteristics, or change in relative strength or activity of different components or branches of the process, pathway, or phenomenon. The term “modulator” refers to any molecule or compound that causes or facilitates a qualitative or quantitative change, alteration, or modification in a process, pathway, or phenomenon of interest.

As used herein, the phrase “modulation of a biological pathway” refers to modulation of activity of at least one component of the biological pathway. It is contemplated herein that modulator of the signaling pathway can be, for example, a receptor ligand (e.g., a small molecule, an antibody, an siRNA), a ligand sequestrant (e.g., an antibody, a binding protein), a modulator of phosphorylation of a pathway component or a combination of such modulators.

One of skill in the art can easily test a compound to determine if it modulates a signaling pathway by assessing, for example, phosphorylation status of the receptor or expression of downstream proteins controlled by the pathway in cultured cells and comparing the results to cells not treated with a modulator. A modulator is determined to be a signaling pathway modulator if the level of phosphorylation of the receptor or expression of downstream proteins in a culture of cells is reduced by at least 20% compared to the level of phosphorylation of the receptor or expression of downstream proteins in cells that are cultured in the absence of the modulator; preferably the level of phosphorylation is altered by at least 30%, at least 40%, at least 50%, at least 60%, at least 70%, at least 80%, at least 90%, at least 95%, or at least 99% in the presence of a pathway modulator.

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, an “RNA interference molecule” refers to a compound which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), microRNA (miRNA) and small molecules which interfere with or inhibit expression of a target gene by RNA interference (RNAi).

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (e.g. The succession of letters used as abbreviations for bases) that biochemically characterizes a specific nucleic acid. A polynucleotide sequence presented herein is presented in a 5′ to 3′ direction unless otherwise indicated.

The nucleic acid molecules that modulate the biological pathways or targets described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. Proc. Natl. Acad. Sci. USA 91:3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. . . . A polypeptide that has a nonpolypeptide moiety covalently or noncovalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms “alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “halo” refers to any radical of fluorine, chlorine, bromine or iodine.

The term “acyl” refers to an alkylcarbonyl, cycloalkylcarbonyl, arylcarbonyl, heterocyclylcarbonyl, or heteroarylcarbonyl substituent, any of which may be further substituted by substituents. Exemplary acyl groups include, but are not limited to, (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions.

The term “alkyl” refers to saturated non-aromatic hydrocarbon chains that may be a straight chain or branched chain, containing the indicated number of carbon atoms (these include without limitation methyl, ethyl, propyl, allyl, or propargyl), which may be optionally inserted with N, O, S, SS, SO₂, C(O), C(O)O, OC(O), C(O)N or NC(O). For example, C₁-C₆ indicates that the group may have from 1 to 6 (inclusive) carbon atoms in it.

The term “alkenyl” refers to an alkyl that comprises at least one double bond. Exemplary alkenyl groups include, but are not limited to, for example, ethenyl, propenyl, butenyl, 1-methyl-2-buten-1-yl and the like.

The term “alkynyl” refers to an alkyl that comprises at least one triple bond.

The term “alkoxy” refers to an —O-alkyl radical.

The term “aminoalkyl” refers to an alkyl substituted with an amino.

The term “mercapto” refers to an —SH radical.

The term “thioalkoxy” refers to an —S-alkyl radical.

The term “aryl” refers to monocyclic, bicyclic, or tricyclic aromatic ring system wherein 0, 1, 2, 3, or 4 atoms of each ring may be substituted by a substituent. Examplary aryl groups include, but are not limited to, phenyl, naphthyl, anthracenyl, azulenyl, fluorenyl, indanyl, indenyl, naphthyl, phenyl, tetrahydronaphthyl, and the like.

The term “arylalkyl” refers to alkyl substituted with an aryl.

The term “cyclyl” or “cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Exemplary cycloalkyl groups include, but are not limited to, cyclopropyl, cyclobutyl, cyclopentyl, cyclopentenyl, cyclohexyl, cyclohexenyl, cycloheptyl, cyclooctyl, and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, and the like.

The term “heteroarylalkyl” refers to an alkyl substituted with a heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examplary heterocyclyl groups include, but are not limited to piperazinyl, pyrrolidinyl, dioxanyl, morpholinyl, tetrahydrofuranyl, and the like.

The term “haloalkyl” refers to an alkyl group having one, two, three or more halogen atoms attached thereto. Exemplary haloalkyl groups include, but are not limited to chloromethyl, bromoethyl, trifluoromethyl, and the like.

The term “optionally substituted” means that the specified group or moiety, such as an aryl group, heteroaryl group and the like, is unsubstituted or is substituted with one or more (typically 1-4 substituents) independently selected from the group of substituents listed below in the definition for “substituents” or otherwise specified.

The term “substituents” refers to a group “substituted” on an alkyl, alkenyl, alkynyl, cycloalkyl, aryl, heterocyclyl, heteroaryl, acyl, amino group at any atom of that group. Suitable substituents include, without limitation, halo, hydroxy, oxo, nitro, haloalkyl, alkyl, alkenyl, alkynyl, alkaryl, aryl, aralkyl, alkoxy, aryloxy, amino, acylamino, alkylcarbanoyl, arylcarbamoyl, aminoalkyl, alkoxycarbonyl, carboxy, hydroxyalkyl, alkylthio, CF₃, N-morpholino, phenylthio, alkanesulfonyl, arenesulfonyl, alkanesulfonamido, arenesulfonamido, aralkylsulfonamido, alkylcarbonyl, acyloxy, cyano or ureido. In some embodiments, substituent can itself be optionally substituted. In some cases, two substituents, together with the carbons to which they are attached to can form a ring.

The present invention may be as defined in anyone of the following numbered paragraphs.

1. A method for identifying a compound that modulates the SMN level in a cell, the method comprising: treating a cell with a compound, and evaluating the SMN level in the cell and/or different cellular compartments, thereby determining whether the compound regulates the SMN level.
2. The method of paragraph 1, wherein the cell is a human cell or a mouse cell.
3. The method of paragraph 1, wherein the cell is a whole cell.
4. The method of paragraph 1, wherein the cell is from a subject, e.g., a patient.
5. The method of paragraph 4, wherein the patient is suffering from a neurodegenerative disorder.
6. The method of paragraph 5, wherein the neurodegenerative disorder is SMA.
7. The method of paragraph 1, wherein the cell is a fibroblast.
8. The method of paragraph 1, wherein the cell is a neuron.
9. The method of paragraph 8, wherein neuron is a motor neuron.
10. The method of paragraph 9, wherein the motor neuron is SMN-deficient.
11. The method of paragraph 1, wherein the SMN level is evaluated by an image-based method.
12. The method of paragraph 1, wherein the compound increases the SMN level by at least about 1.1, 1.25, 1.5, 1.75, 2, 3, 4, 5, 10 or greater fold.
13. The method of paragraph 1, wherein the cellular compartment is cytoplasm, nucleus or gem.
14. The method of paragraph 1, wherein the compound decrease the SMN level by at least about 5%, 10%, 15%, 20%, 25%, 30%, 35%, 40%, 45%, 50%, 55%, 60%, 65%, 70%, 75%, 80%, 85%, 90%, 95%, 98%, or 99%.
15. The method of paragraph 1, wherein the compound is a Na⁺/K⁺ channel modulator e.g., cardiac glocosides (e.g., Ouabain, Digoxin, Dititoxin or Lanatoside C).
16. The method of paragraph 1, wherein the compound is an activator of MAPK (e.g., Anysomycin or Coumermycin).
17. The method of paragraph 1, wherein the compound is a cannabinoid receptor or GPCR agonist (e.g., WIN 55, 212-2 or Anandamide).
18. The method of paragraph 1, wherein the compound is a Ca2+ channel modulator (e.g., Thapsigargin, Ionomycin or Calcimycin).
19. The method of paragraph 1, wherein the compound is a K⁺ channel modulator (e.g., Veratridine, Monensin Na or Valinomycin).
20. The method of paragraph 1, wherein the compound is a PDE5 inhibitor (e.g., MBCQ or Dipyridamole).
21. The method of paragraph 1, wherein the compound is a kinase inhibitor.
22. The method of paragraph 21, wherein the kinase inhibitor is an inhibitor of GSK/CDK e.g., Alsterpaullone or its structural analogs (e.g., 1-aza-alsterpaullone or 2-cyanoethyl-alsterpaullone).
23. The method of paragraph 21, wherein the kinase inhibitor is an inhibitor of GSK e.g., AR-A014418 or CHIR99021.
24. The method of paragraph 23, wherein the GSK inhibitor increases motor neuron survival.
25. The method of paragraph 21, wherein the kinase inhibitor is an inhibitor of PKR.
26. The method of paragraph 21, wherein the kinase inhibitor is an inhibitor of CDK2 e.g., GW8510.
27. The method of paragraph 21, wherein the kinase inhibitor is an inhibitor of IKK-2 e.g., IMD-0354 or its structural analog Niclosamide.
28. The method of paragraph 1, wherein the compound is an HDAC inhibitor e.g., trichostatin.
29. The method of paragraph 1, wherein the compound is a proteasome inhibitor.
30. The method of paragraph 1, wherein the compound is a BMP/TGFβ ligand e.g., BMP4.
31. The method of paragraph 1, wherein the compound is a Dopamine receptor ligand.

The method of paragraph 1, wherein the compound is a modulator of the PI-3/AKT/GSK pathway.

32. The method of paragraph 1, wherein the compound is a modulator of the PI-3K signaling pathway.
33. The method of paragraph 1, wherein the compound is a modulator of the Akt signaling pathway.
34. The method of paragraph 1, wherein the compound is a modulator of the PDGF pathway.
35. The method of paragraph 1, wherein the compound is a modulator of the PKR pathway.
36. The method of paragraph 1, wherein the compound is a modulator of the Insulin Receptor pathway.
37. The method of paragraph 1, wherein the compound is a modulator of the MAPK signaling pathway.
38. The method of paragraph 1, wherein the compound is a modulator of the Ras pathway.
39. The method of paragraph 1, wherein the compound is a modulator of the eIF2 pathway.
40. The method of paragraph 1, wherein the compound is a modulator of the mTOR pathway.
41. The method of paragraph 1, wherein the compound is a modulator of the NGF pathway.
42. The method of paragraph 1, wherein the compound is a modulator of the EGF pathway.
43. The method of paragraph 1, wherein the compound is a modulator of the FGF pathway.
44. The method of paragraph 1, wherein the compound is a modulator of the TGF pathway.
45. The method of paragraph 1, wherein the compound is a modulator of the GSK3 signaling pathway.
46. The method of paragraph 1, wherein the compound is a modulator of the BMP pathway.
47. The method of paragraph 1, wherein the compound is:

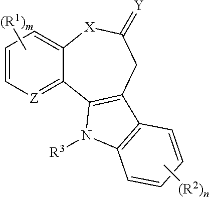

wherein:
X is NH, O, S or CH₂; and
Y is O or S;
Z is N or CH;
m is 0, 1, 2, 3 or 4;
n is 0, 1, 2, 3 or 4;
each R¹ and R² is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁴;
R³ is hydrogen, alkyl, alkenyl, alkynyl, aryl, heteroaryl, arylalkyl, haloalkyl or alkylhydroxy;
each R⁴ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, alkylhydroxy, haloalkyl or halo;
each R^{b} is independently hydrogen, alkyl, alkenyl, alkynyl, aryl, heteroaryl, arylalkyl, alkylhydroxy, or haloalkyl;
each R^{c} is independently hydrogen, alkyl, alkenyl, alkynyl, aryl, heteroaryl, arylalkyl, alkylhydroxy, or haloalkyl;
each R^{d} is independently hydrogen, alkyl, alkenyl, alkynyl, aryl, heteroaryl, arylalkyl, alkylhydroxy, or haloalkyl;
each R^{e} is independently hydrogen, alkyl, alkenyl, alkynyl, aryl, heteroaryl, arylalkyl, alkylhydroxy, or haloalkyl;
each R^{f} is independently hydrogen, alkyl, alkenyl, alkynyl, aryl, heteroaryl, arylalkyl, alkylhydroxy, or haloalkyl.
   48. The method of paragraph 47, wherein X is NH, O, S or CH₂.
   49. The method of paragraph 47, wherein X is NH.
   50. The method of paragraph 47, wherein Y is O or S.
   51. The method of paragraph 47, wherein Y is O.
   52. The method of paragraph 47, wherein Z is N or CH.
   53. The method of paragraph 47, wherein Z is N.
   54. The method of paragraph 47, wherein Z is CH.
   55. The method of paragraph 47, wherein m is 0, 1, 2, 3, or 4.
   56. The method of paragraph 47, wherein m is 0.
   57. The method of paragraph 47, wherein m is 1.
   58. The method of paragraph 47, wherein R¹ is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁴.
   59. The method of paragraph 47, wherein R¹ is alkyl.
   60. The method of paragraph 47, wherein R¹ is alkyl substituted with 1 R⁴.
   61. The method of paragraph 47, wherein R⁴ is halo, —NO₂, —SO₃⁻, —CN, —C(═O)R^{a}, —C(═O)N(R^{b})₂, —C(═O)OR^{c}, —OR^{d}, —NR^{e}₂, or —SR^{f}_{.}
   62. The method of paragraph 47, wherein R⁴ is —CN.
   63. The method of paragraph 1, wherein the compound is:

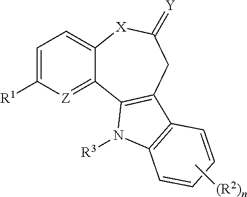

64. The method of paragraph 63, wherein n is 0, 1, 2, 3, or 4.
65. The method of paragraph 63, wherein n is 1.
66. The method of paragraph 63, wherein R² is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁴.
67. The method of paragraph 63, wherein R⁴ is halo, —NO₂, —SO₃⁻, —CN, —C(═O)R^{a}, —C(═O)N(R^{b})₂, —C(═O)OR^{c}, —OR^{d}, —NR^{e}₂, or —SR^{f}.
68. The method of paragraph 63, wherein R² is —NO₂.
69. The method of paragraph 1, wherein the compound is:

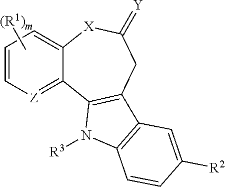

70. The method of paragraph 69, wherein R³ is hydrogen, alkyl, alkenyl, alkynyl, aryl, heteroaryl, arylalkyl, haloalkyl, or alkylhydroxy.
71. The method of paragraph 69, wherein R³ is hydrogen.
72. The method of paragraph 69, wherein the compound is selected from the following:

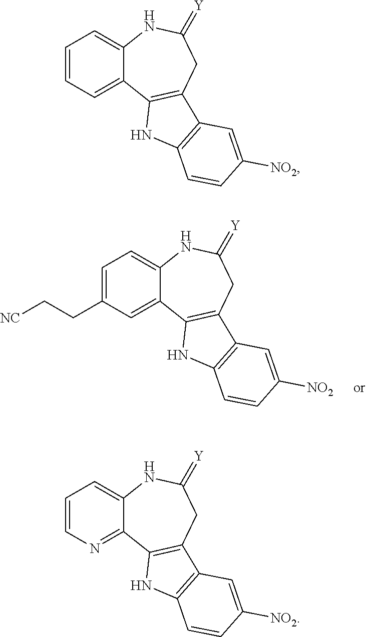

73. The method of paragraph 1, wherein the compound is:

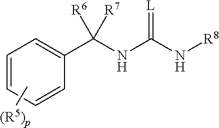

wherein:
L is O or S; and
p is 0, 1, 2, 3, 4 or 5;
each R⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁹;
each R⁶ and R⁷ is independently hydrogen, alkyl, alkenyl, alkynyl, aryl, heteroaryl, arylalkyl, haloalkyl, or alkylhydroxy;

R⁸ is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁰;
each R⁹ and R¹⁰ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, alkylhydroxy, haloalkyl or halo;
each R^{b} is independently hydrogen, alkyl, alkenyl, alkynyl, aryl, heteroaryl, arylalkyl, alkylhydroxy, or haloalkyl;
each R^{c} is independently hydrogen, alkyl, alkenyl, alkynyl, aryl, heteroaryl, arylalkyl, alkylhydroxy, or haloalkyl;
each R^{d} is independently hydrogen, alkyl, alkenyl, alkynyl, aryl, heteroaryl, arylalkyl, alkylhydroxy, or haloalkyl;
each R^{e} is independently hydrogen, alkyl, alkenyl, alkynyl, aryl, heteroaryl, arylalkyl, alkylhydroxy, or haloalkyl;
each R^{f} is independently hydrogen, alkyl, alkenyl, alkynyl, aryl, heteroaryl, arylalkyl, alkylhydroxy, or haloalkyl.
   74. The method of paragraph 73, wherein L is O or S.
   75. The method of paragraph 73, wherein L is O.
   76. The method of paragraph 73, wherein each R⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁹.
   77. The method of paragraph 73, wherein p is 0, 1, 2, 3, 4 or 5.
   78. The method of paragraph 73, wherein p is 1.
   79. The method of paragraph 1, wherein the compound is:

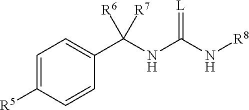

80. The method of paragraph 79, wherein R⁵ is —OR^{d}.
81. The method of paragraph 79, wherein R⁵ is —OCH₃.
82. The method of paragraph 79, wherein each R⁶ and R⁷ is independently hydrogen, alkyl, alkenyl, alkynyl, aryl, heteroaryl, arylalkyl, haloalkyl, or alkylhydroxy.
83. The method of paragraph 79, wherein R⁶ is hydrogen.
84. The method of paragraph 79, wherein R⁷ is hydrogen.
85. The method of paragraph 79, wherein R⁸ is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁰.
86. The method of paragraph 79, wherein R¹⁰ is halo, —NO₂, —SO₃⁻, —CN, —C(═O)R^{a}, —C(═O)N(R^{b})₂, —C(═O)OR^{c}, —OR^{d}, —NR^{e}₂, or —SR^{f}.
87. The method of paragraph 79, wherein R⁸ is heteroaryl substituted with 1 R¹⁰.
88. The method of paragraph 79, wherein R⁸ is a 5-membered heterocycle.
89. The method of paragraph 79, wherein R⁸ is a thiazole.
90. The method of paragraph 1, wherein the compound is:

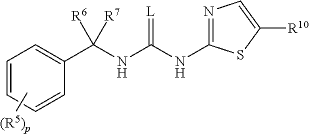

91. The method of paragraph 90, wherein R¹⁰ is —NO₂.
92. The method of paragraph 90, wherein the compound is:

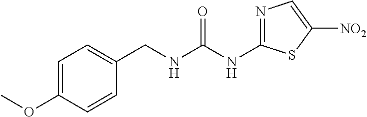

93. The method of paragraph 1, wherein the compound is:

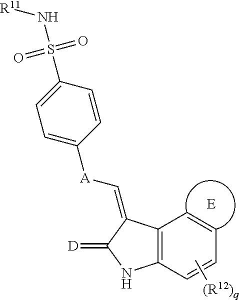

wherein:
A is NH, O, S or CH₂; and
D is O or S;
E is an aryl or heteroaryl moiety;
q is 0, 1 or 2;
each R¹¹ and R¹² is independently hydrogen, alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹³;
each R¹³ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl;
each R^{f} is independently hydrogen, alkyl, alkenyl, alkynyl, aryl, heteroaryl, arylalkyl, alkylhydroxy or haloalkyl.
   94. The method of paragraph 93, wherein A is NH or O.
   95. The method of paragraph 93, wherein A is NH.
   96. The method of paragraph 93, wherein D is O or S.
   97. The method of paragraph 93, wherein D is O.
   98. The method of paragraph 93, wherein E is an aryl or heteroaryl moiety.
   99. The method of paragraph 93, wherein E is a heteroaryl moiety.
   100. The method of paragraph 93, wherein E is a thiazole.
   101. The method of paragraph 93, wherein the compound is:

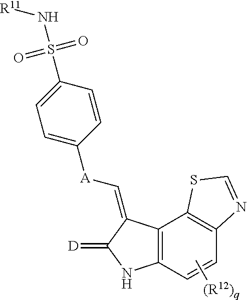

102. The method of paragraph 93, wherein R¹¹ is hydrogen, alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹³.
103. The method of paragraph 93, wherein R¹³ is halo, —NO₂, —SO₃⁻, —CN, —C(═O)R^{a}, —C(═O)N(R^{b})₂, —C(═O)OR^{c}, —OR^{d}, —NR^{e}₂, or —SR^{f}.
104. The method of paragraph 93, wherein R¹¹ is heteroaryl.
105. The method of paragraph 93, wherein R¹² is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹³;
106. The method of paragraph 93, wherein q is 0, 1 or 2.
107. The method of paragraph 93, wherein q is 0.
108. The method of paragraph 93, wherein the compound is:

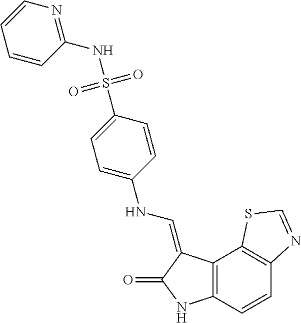

109. The method of paragraph 1, wherein the compound is:

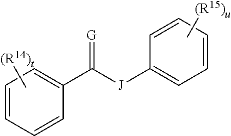

wherein:
t is 0, 1, 2, 3, 4 or 5; and
u is 0, 1, 2, 3, 4 or 5;
G is 0 or S;
J is O, S, NH or CH₂;
each R¹⁴ and R¹⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁶;
each R¹⁶ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl;
each R^{f} is independently hydrogen, alkyl, alkenyl, alkynyl, aryl, heteroaryl, arylalkyl, alkylhydroxy or haloalkyl.
   110. The method of paragraph 109, wherein G is O or S.
   111. The method of paragraph 109, wherein G is O.
   112. The method of paragraph 109, wherein J is O, S, NH or CH₂.
   113. The method of paragraph 109, wherein J is NH.
   114. The method of paragraph 109, wherein t is 0, 1, 2, 3, 4 or 5.
   115. The method of paragraph 109, wherein t is 2.
   116. The method of paragraph 109, wherein each R¹⁴ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁶.
   117. The method of paragraph 109, wherein R¹⁶ is halo, —NO₂, —SO₃⁻, —CN, —C(═O)R^{a}, —C(═O)N(R^{b})₂, —C(═O)OR^{c}, —OR^{d}, —NR^{e}₂, or —SR^{f}.
   118. The method of paragraph 109, wherein R¹⁴ is —OH.
   119. The method of paragraph 109, wherein R¹⁴ is halo.
   120. The method of paragraph 109, wherein R¹⁴ is —Cl.
   121. The method of paragraph 109, wherein u is 0, 1, 2, 3, 4 or 5.
   122. The method of paragraph 109, wherein u is 2.
   123. The method of paragraph 109, wherein each R¹⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁶.
   124. The method of paragraph 109, wherein leis halo, —NO₂, —SO₃⁻, —CN, —C(═O)R^{a}, —C(═O)N(R^{b})₂, —C(═O)OR^{c}, —OR^{d}, —NR^{e}₂, or —SR^{f}.
   125. The method of paragraph 109, wherein R¹⁵ is —NO₂.
   126. The method of paragraph 109, wherein R¹⁵ is halo.
   127. The method of paragraph 109, wherein R¹⁵ is —Cl.
   128. The method of paragraph 109, wherein R¹⁵ is alkyl substituted with 1-4 R¹⁶.
   129. The method of paragraph 109, wherein R¹⁵ is substituted with 3 R¹⁶.
   130. The method of paragraph 109, wherein R¹⁶ is halo.
   131. The method of paragraph 109, wherein R¹⁶ is —F.
   132. The method of paragraph 109, wherein the compound is selected from the following:

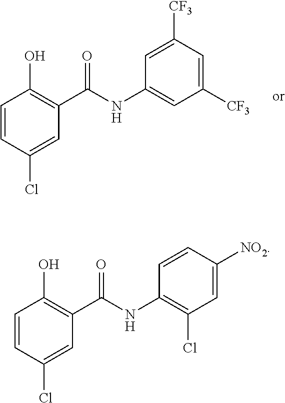

133. The method of paragraph 1, wherein the compound is a growth factor.
134. A method for identifying a compound that modulates the SMN level, the GEM level, or the distribution of either, in a cell comprising: a first step in which a candidate compound is contacted with a cell and is evaluated for the ability to modulate SMN or GEM level or distribution; and a second step in which a compound is contacted with a cell in which SMN expression is reduced and evaluated for the ability to rescue the cell having reduced SMN.
135. The method of paragraph 134, wherein the cell in the first step is from a subject suffering from a neurodegenerative disorder.
136. The method of paragraph 134, wherein the cell in the second step is a neuron, e.g., a motor neuron, e.g., an ES-derived motor neuron.
137. The method of paragraph 134, wherein the cell is cultured under conditions which minimize survival of cells having reduced SMN expression, contacted with the compound, and survival of the cell evaluated.
138. The method of paragraph 134, further comprising evaluating the compound for the ability to modulate the PI-3/AKT/GSK pathway.
139. The method of paragraph 134, wherein the expression of SMN is reduced in the second cell by gene silencing.
140. A method of promoting motor neuron cell survival, the method comprising: contacting a motor neuron cell with a compound of formula (VI)

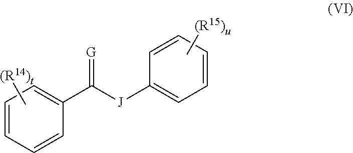

wherein:
t is 0, 1, 2, 3, 4 or 5; and
u is 0, 1, 2, 3, 4 or 5;
G is O or S;
J is O, S, NH or CH₂;
each R¹⁴ and R¹⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁶;
each R¹⁶ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl; and
each R^{f} is independently hydrogen, alkyl, alkenyl, alkynyl, aryl, heteroaryl, arylalkyl, alkylhydroxy or haloalkyl.
   141. A method of promoting motor neuron cell survival, the method comprising: contacting a motor neuron cell with a compound of formula (I)

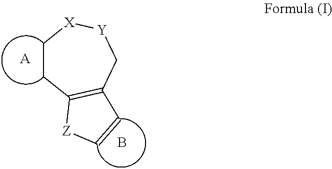

wherein:
A represents, with the adjacent ring, an optionally substituted aryl or an optionally substituted heteroaryl;
B represents, with the adjacent ring, an optionally substituted aryl or an optionally substituted heteroaryl;
X is NR^{N}, O, S, or CH₂;
Y is C(O), C(S), CH—SR^{N}CH—NHOH or S;
Z is NR^{N}, O, S or CHR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy; and
physiologically acceptable salts thereof.
   142. The method of paragraph 141, wherein the compound is:

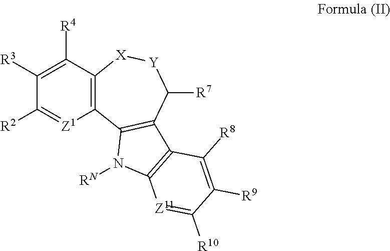

wherein:
Z¹ is N or CR¹;
Z¹¹ is N or CR¹¹;
R¹, R², R³, R⁴, R⁷, R⁸, R⁹, R¹⁰ and R¹¹ are each independently hydrogen, optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy;
R¹² is independently for each occurrence optionally substituted linear or branched alkyl, optionally substituted alkenyl, optionally substituted alkynyl, optionally substituted aryl, optionally substituted heteroaryl, optionally substituted arylalkyl, optionally substituted haloalkyl or optionally substituted alkylhydroxy;
X is NR^{N}, O, S, or CH₂;
Y is C(O), C(S), CH—SR^{N}CH—NHOH or S; and
physiologically acceptable salts thereof.
   143. The method of paragraph 141, wherein the compound is:

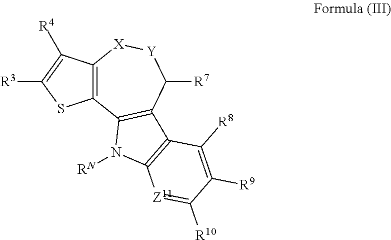

wherein:
Z¹¹ is N or CR¹¹;
R³, R⁴, R⁷, R⁸, R⁹, R¹⁰ and R¹¹ are each independently hydrogen, optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy;
R¹² is independently for each occurrence optionally substituted linear or branched alkyl, optionally substituted alkenyl, optionally substituted alkynyl, optionally substituted aryl, optionally substituted heteroaryl, optionally substituted arylalkyl, optionally substituted haloalkyl or optionally substituted alkylhydroxy;
X is NR^{N}, O, S, or CH₂;
Y is C(O), C(S), CH—SR^{N}CH—NHOH or S; and
physiologically acceptable salts thereof.
   144. A method of promoting motor neuron cell survival, the method comprising:
contacting a motor neuron cell with a compound of formula (IV)

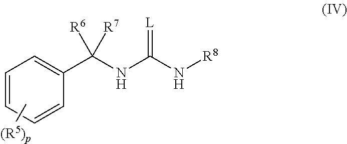

wherein:
L is O or S; and
p is 0, 1, 2, 3, 4 or 5;
each R⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁹;
each R⁶ and R⁷ is independently hydrogen, alkyl, alkenyl, alkynyl, aryl, heteroaryl, arylalkyl, haloalkyl, or alkylhydroxy;
R⁸ is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁰;
each R⁹ and R¹⁰ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, alkylhydroxy, haloalkyl or halo;
each R^{b} is independently hydrogen, alkyl, alkenyl, alkynyl, aryl, heteroaryl, arylalkyl, alkylhydroxy, or haloalkyl;
each R^{c} is independently hydrogen, alkyl, alkenyl, alkynyl, aryl, heteroaryl, arylalkyl, alkylhydroxy, or haloalkyl;
each R^{d} is independently hydrogen, alkyl, alkenyl, alkynyl, aryl, heteroaryl, arylalkyl, alkylhydroxy, or haloalkyl;
each R^{e} is independently hydrogen, alkyl, alkenyl, alkynyl, aryl, heteroaryl, arylalkyl, alkylhydroxy, or haloalkyl; and
each R^{f} is independently hydrogen, alkyl, alkenyl, alkynyl, aryl, heteroaryl, arylalkyl, alkylhydroxy, or haloalkyl.
   145. A method of promoting motor neuron cell survival, the method comprising:
contacting a motor neuron cell with a compound of formula (V)

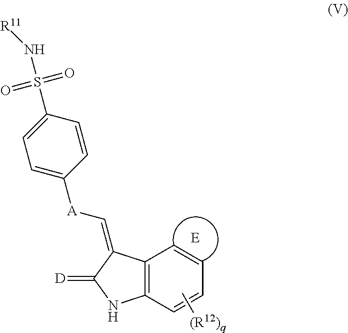

wherein:
A is NH, O, S or CH₂; and
D is O or S;
E is an aryl or heteroaryl moiety;
q is 0, 1 or 2;
each R¹¹ and R¹² is independently hydrogen, alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹³;
each R¹³ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl; and
each R^{f} is independently hydrogen, alkyl, alkenyl, alkynyl, aryl, heteroaryl, arylalkyl, alkylhydroxy or haloalkyl.
   146. The method of any of paragraphs 140-145, wherein the motor neuron comprises a mutation in gene encoding SMN1.
   147. The method of any of paragraphs 140-146, wherein the motor neuron comprises a mutation in gene encoding superoxide mutase 1 (SOD1).
   148. The method of any of paragraphs 140-147, wherein the motor neuron comprises a G->A mutation at position 93 of gene encoding SOD1.
   149. The method of any of paragraphs 140-148, wherein the compound increases survival of motor neuron (SMN) protein levels.
   150. The method of any of paragraphs 140-149, wherein the compound does not increase SMN protein levels.
   151. The method of any of paragraphs 140-149, wherein the compound modulates a biological pathway that increases SMN protein levels.
   152. The method of any of paragraphs 140-151, wherein the biological pathway is selected from the group consisting of PI-3 signaling pathway, AKT signaling pathway and glycogen synthase kinase (GSK) pathway.
   153. The method of any of paragraphs 140-152, wherein the biological pathway is GSK signaling pathway.
   154. The method of any of paragraphs 140-153, wherein said compound inhibits the activity of at least one kinase.
   155. The method of any of paragraphs 140-154, wherein the kinase is cyclin-dependent kinase (CDK).
   156. The method of any of paragraphs 140-154, wherein the kinase is GSK-3.
   157. The method of any of paragraphs 140-154, wherein the kinase is IKK-2.
   158. The method of any of paragraphs 140-154, wherein the compound inhibits the activity of two or more kinases.
   159. The method of paragraph 158, wherein the compound inhibits GSK-3 and at least one other kinase.
   160. The method of paragraph 159, wherein the other kinase is CDK.
   161. The method of any of paragraphs 140-160, wherein the contact is in vitro.
   162. The method of any of paragraphs 140-160, wherein the contact is in vivo.
   163. The method of any of paragraphs 140-160, wherein in vivo contact is in a mouse.
   164. The method of any of paragraphs 140-160, wherein in vivo contact is in a subject, where the subject is selected for treatment of a neurodegenerative disorder characterized by degeneration of motor neurons.
   165. The method of any of paragraphs 140-164, wherein the subject is human.
   166. The method of any of paragraphs 140-165, wherein the neurodegenerative disorder is characterized by a mutation in the SMN gene.
   167. The method of any of paragraphs 140-166, wherein the neurodegenerative disorder is characterized by diminished levels of SMN protein.
   168. The method of any of paragraphs 140-167, wherein the neurodegenerative disorder is spinal muscular atrophy (SMA).
   169. The method of any of paragraphs 140-167, wherein the neurodegenerative disorder is amyotrophic lateral sclerosis (ALS).
   170. A method of promoting motor neuron survival, the method comprising: contacting a motor neuron with a compound that modulates a biological pathway or a target.
   171. The method of paragraph 170, wherein the biological pathway is selected from the group consisting of PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof.
   172. The method of any of paragraphs 140-171, wherein the biological pathway is selected from the group consisting of PI-3 signaling pathway, AKT signaling pathway and glycogen synthase kinase (GSK) pathway.
   173. The method of any of paragraphs 140-172, wherein the biological pathway is GSK signaling pathway.
   174. The method of any of paragraphs 140-173, wherein the target is selected from the group consisting of Na⁺/K⁺ channel, MAPK, cannobinoid receptor, GPCR, Ca²⁺ channel, K⁺ channel, PDE5, GSK/CDK, PKR, CDK2, IKK-2, proteasome, BMP/TGFbeta receptor and dopamine receptor.
   175. The method of any of paragraphs 140-173, wherein the target is GSK-3b, CDK2, CDK5, PKR or IKK-2b.
   176. The method of any of paragraphs 140-175, wherein the compound is selected from the group consisting of formula (I), (II), (III), (IV), (V), (VI), cardiac glocoside, activator of MAPK, cannabinoid receptor or GPCR agonist, Ca²⁺ channel modulator, K⁺ channel modulator, PDE5 inhibitor, kinase inhibitor, HDAC inhibitor, proteasome inhibitor, BMP/TGFβ ligand, Dopamine receptor ligand, modulator of PI-3K signaling pathway, modulator of Akt signaling pathway, modulator of PDGF pathway, modulator of PKR pathway, modulator of Insulin Receptor pathway, modulator of MAPK signaling pathway, modulator of Ras pathway, modulator of eIF2 pathway, modulator of mTOR pathway, modulator of NGF pathway, modulator of EGF pathway, modulator of FGF pathway, modulator of TGF pathway, modulator of GSK pathway, modulator of BMP pathway, and combinations thereof.
   177. The method of any of paragraphs 140-176, wherein the motor neuron comprises a mutation in gene encoding SMN1.
   178. The method of any of paragraphs 140-177 wherein the motor neuron comprises a mutation in gene encoding superoxide mutase 1 (SOD1).
   179. The method of any of paragraphs 140-1789, wherein the motor neuron comprises a G->A mutation at position 93 of gene encoding SOD1.
   180. The method of any of paragraphs 140-179, wherein the compound increases survival of motor neuron (SMN) protein levels.
   181. The method of any of paragraphs 140-179, wherein the compound does not increase SMN protein levels.
   182. The method of any of paragraphs 140-180, wherein the compound modulates a biological pathway that increases SMN protein levels.
   183. The method of any of paragraphs 140-182, wherein said compound inhibits the activity of at least one kinase.
   184. The method of any of paragraphs 140-183, wherein the kinase is cyclin-dependent kinase (CDK).
   185. The method of any of paragraphs 140-184, wherein the kinase is GSK-3.
   186. The method of any of paragraphs 140-184, wherein the kinase is IKK-2.
   187. The method of any of paragraphs 140-186, wherein the compound inhibits the activity of two or more kinases.
   188. The method of paragraph 187, wherein the compound inhibits GSK-3 and at least one other kinase.
   189. The method of paragraph 188, wherein the other kinase is CDK.
   190. The method of any of paragraphs 140-189, wherein the contact is in vitro.
   191. The method of any of paragraphs 140-189, wherein the contact is in vivo.
   192. The method of any of paragraphs 140-189, wherein in vivo contact is in a mouse.
   193. The method of any of paragraphs 140-189, wherein in vivo contact is in a subject, where the subject is selected for treatment of a neurodegenerative disorder characterized by degeneration of motor neurons.
   194. The method of any of paragraphs 140-193, wherein the subject is human.
   195. The method of any of paragraphs 140-194, wherein the neurodegenerative disorder is characterized by a mutation in the SMN gene.
   196. The method of any of paragraphs 140-195, wherein the neurodegenerative disorder is characterized by diminished levels of SMN protein.
   197. The method of any of paragraphs 140-196, wherein the neurodegenerative disorder is spinal muscular atrophy (SMA).
   198. The method of any of paragraphs 140-196, wherein the neurodegenerative disorder is amyotrophic lateral sclerosis (ALS).
   199. The use of a compound of formula (VI) to promote motor neuron cell survival by contacting the compound with the motor neuron, wherein the compound comprises:

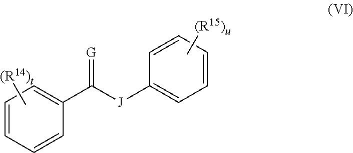

wherein:
t is 0, 1, 2, 3, 4 or 5; and
u is 0, 1, 2, 3, 4 or 5;
G is 0 or S;
J is O, S, NH or CH₂;
each R¹⁴ and R¹⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁶;
each R¹⁶ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl; and
each R^{f} is independently hydrogen, alkyl, alkenyl, alkynyl, aryl, heteroaryl, arylalkyl, alkylhydroxy or haloalkyl.
   200. The use of a compound of formula (I) to promote motor neuron cell survival by contacting the compound with the motor neuron, wherein the compound comprises:

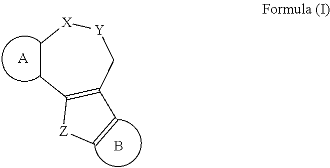

wherein:
A represents, with the adjacent ring, an optionally substituted aryl or an optionally substituted heteroaryl;
B represents, with the adjacent ring, an optionally substituted aryl or an optionally substituted heteroaryl;
X is NR^{N}, O, S, or CH₂;
Y is C(O), C(S), CH—SR^{N}CH—NHOH or S;
Z is NR^{N}, O, S or CHR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy; and
physiologically acceptable salts thereof.
   201. The use of the compound of paragraph 200, wherein the compound is:

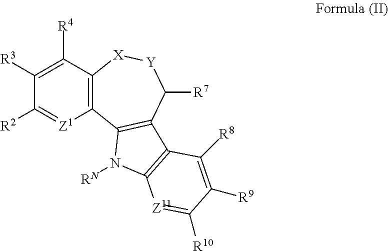

wherein:
Z¹ is N or CR¹;
Z¹¹ is N or CR¹¹;

R¹, R², R³, R⁴, R⁷, R⁸, R⁹, R¹⁰ and R¹¹ are each independently hydrogen, optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy;
R¹² is independently for each occurrence optionally substituted linear or branched alkyl, optionally substituted alkenyl, optionally substituted alkynyl, optionally substituted aryl, optionally substituted heteroaryl, optionally substituted arylalkyl, optionally substituted haloalkyl or optionally substituted alkylhydroxy;
X is NR^{N}, O, S, or CH₂;
Y is C(O), C(S), CH—SR^{N}CH—NHOH or S; and
physiologically acceptable salts thereof.
   202. The use of the compound of paragraph 200, wherein the compound is:

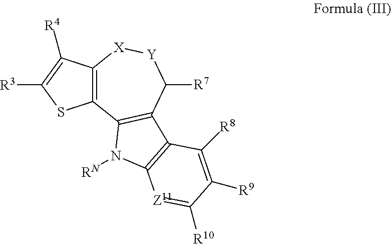

wherein:
Z¹¹ is N or CR¹¹;
R³, R⁴, R⁷, R⁸, R⁹, R¹⁰ and R¹¹ are each independently hydrogen, optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂, —SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy;
R¹² is independently for each occurrence optionally substituted linear or branched alkyl, optionally substituted alkenyl, optionally substituted alkynyl, optionally substituted aryl, optionally substituted heteroaryl, optionally substituted arylalkyl, optionally substituted haloalkyl or optionally substituted alkylhydroxy;
X is NR^{N}, O, S, or CH₂;
Y is C(O), C(S), CH—SR^{N}CH—NHOH or S; and
physiologically acceptable salts thereof.
   203. The use of a compound of formula (IV) to promote motor neuron cell survival by contacting the compound with the motor neuron, wherein the compound comprises:

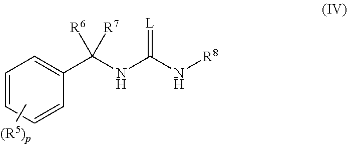

wherein:
L is O or S; and
p is 0, 1, 2, 3, 4 or 5;
each R⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁹;
each R⁶ and R⁷ is independently hydrogen, alkyl, alkenyl, alkynyl, aryl, heteroaryl, arylalkyl, haloalkyl, or alkylhydroxy;
R⁸ is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁰;
each R⁹ and R¹⁰ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, alkylhydroxy, haloalkyl or halo;
each R^{b} is independently hydrogen, alkyl, alkenyl, alkynyl, aryl, heteroaryl, arylalkyl, alkylhydroxy, or haloalkyl;
each R^{c} is independently hydrogen, alkyl, alkenyl, alkynyl, aryl, heteroaryl, arylalkyl, alkylhydroxy, or haloalkyl;
each R^{d} is independently hydrogen, alkyl, alkenyl, alkynyl, aryl, heteroaryl, arylalkyl, alkylhydroxy, or haloalkyl;
each R^{e} is independently hydrogen, alkyl, alkenyl, alkynyl, aryl, heteroaryl, arylalkyl, alkylhydroxy, or haloalkyl; and
each R^{f} is independently hydrogen, alkyl, alkenyl, alkynyl, aryl, heteroaryl, arylalkyl, alkylhydroxy, or haloalkyl.
   204. The use of a compound of formula (V) to promote motor neuron cell survival by contacting the compound with the motor neuron, wherein the compound comprises:

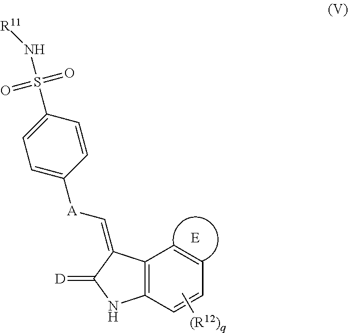

wherein:
A is NH, O, S or CH₂; and
D is O or S;
E is an aryl or heteroaryl moiety;
q is 0, 1 or 2;
each R¹¹ and R¹² is independently hydrogen, alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹³;
each R¹³ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl; and
each R^{f} is independently hydrogen, alkyl, alkenyl, alkynyl, aryl, heteroaryl, arylalkyl, alkylhydroxy or haloalkyl.
   205. The use of the compound of any of paragraphs 199-204, wherein the motor neuron comprises a mutation in gene encoding SMN1.
   206. The use of the compound of any of paragraphs 199-205, wherein the motor neuron comprises a mutation in gene encoding superoxide mutase 1 (SOD1).
   207. The use of the compound of any of paragraphs 199-206, wherein the motor neuron comprises a G->A mutation at position 93 of gene encoding SOD1.
   208. The use of the compound of any of paragraphs 199-207, wherein the compound increases survival of motor neuron (SMN) protein levels.
   209. The use of the compound of any of paragraphs 199-207, wherein the compound does not increase SMN protein levels.
   210. The use of the compound of any of paragraphs 199-208, wherein the compound modulates a biological pathway that increases SMN protein levels.
   211. The use of the compound of any of paragraphs 199-210, wherein the biological pathway is selected from the group consisting of PI-3 signaling pathway, AKT signaling pathway and glycogen synthase kinase (GSK) pathway.
   212. The use of the compound of any of paragraphs 199-211, wherein the biological pathway is GSK signaling pathway.
   213. The use of the compound of any of paragraphs 199-212, wherein said compound inhibits the activity of at least one kinase.
   214. The use of the compound of any of paragraphs 199-213, wherein the kinase is cyclin-dependent kinase (CDK).
   215. The use of the compound of any of paragraphs 199-213, wherein the kinase is GSK-3.
   216. The use of the compound of any of paragraphs 199-213, wherein the kinase is IKK-2.
   217. The use of the compound of any of paragraphs 199-213, wherein the compound inhibits the activity of two or more kinases.
   218. The use of the compound of paragraph 217, wherein the compound inhibits GSK-3 and at least one other kinase.
   219. The use of the compound of paragraph 218, wherein the other kinase is CDK.
   220. The use of the compound of any of paragraphs 199-219, wherein the contact is in vitro.
   221. The use of the compound of any of paragraphs 199-219, wherein the contact is in vivo.
   222. The use of the compound of any of paragraphs 199-218, wherein in vivo contact is in a mouse.
   223. The use of the compound of any of paragraphs 199-218, wherein in vivo contact is in a subject, where the subject is selected for treatment of a neurodegenerative disorder characterized by degeneration of motor neurons.
   224. The use of the compound of any of paragraphs 199-223, wherein the subject is human.
   225. The use of the compound of any of paragraphs 199-224 wherein the neurodegenerative disorder is characterized by a mutation in the SMN gene.
   226. The use of the compound of any of paragraphs 199-225, wherein the neurodegenerative disorder is characterized by diminished levels of SMN protein.
   227. The use of the compound of any of paragraphs 199-226, wherein the neurodegenerative disorder is spinal muscular atrophy (SMA).
   228. The use of the compound of any of paragraphs 199-226, wherein the neurodegenerative disorder is amyotrophic lateral sclerosis (ALS).
   229. A use of the compound of promoting motor neuron survival, the method comprising: contacting a motor neuron with a compound that modulates a biological pathway or a target.
   230. The use of the compound of paragraph 229, wherein the biological pathway is selected from the group consisting of PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof.
   231. The use of the compound of any of paragraphs 119-230, wherein the biological pathway is selected from the group consisting of PI-3 signaling pathway, AKT signaling pathway and glycogen synthase kinase (GSK) pathway.
   232. The use of the compound of any of paragraphs 119-231, wherein the biological pathway is GSK signaling pathway.
   233. The use of the compound of any of paragraphs 119-232, wherein the target is selected from the group consisting of Na⁺/K⁺ channel, MAPK, cannobinoid receptor, GPCR, Ca²⁺ channel, K⁺ channel, PDE5, GSK/CDK, PKR, CDK2, IKK-2, proteasome, BMP/TGFbeta receptor and dopamine receptor.
   234. The use of the compound of any of paragraphs 199-233, wherein the target is GSK-3b, CDK2, CDK5, PKR or IKK-2b.
   235. The use of the compound of any of paragraphs 199-234, wherein the compound is selected from the group consisting of formula (I), (II), (III), (IV), (V), (VI), cardiac glocoside, activator of MAPK, cannabinoid receptor or GPCR agonist, Ca²⁺ channel modulator, K⁺ channel modulator, PDE5 inhibitor, kinase inhibitor, HDAC inhibitor, proteasome inhibitor, BMP/TGFβ ligand, Dopamine receptor ligand, modulator of PI-3K signaling pathway, modulator of Akt signaling pathway, modulator of PDGF pathway, modulator of PKR pathway, modulator of Insulin Receptor pathway, modulator of MAPK signaling pathway, modulator of Ras pathway, modulator of eIF2 pathway, modulator of mTOR pathway, modulator of NGF pathway, modulator of EGF pathway, modulator of FGF pathway, modulator of TGF pathway, modulator of GSK pathway, modulator of BMP pathway, and combinations thereof.
   236. The use of the compound of any of paragraphs 199-235, wherein the motor neuron comprises a mutation in gene encoding SMN1.
   237. The use of the compound of any of paragraphs 199-236 wherein the motor neuron comprises a mutation in gene encoding superoxide mutase 1 (SOD1).
   238. The use of the compound of any of paragraphs 199-237, wherein the motor neuron comprises a G->A mutation at position 93 of gene encoding SOD1.
   239. The use of the compound of any of paragraphs 199-238, wherein the compound increases survival of motor neuron (SMN) protein levels.
   240. The use of the compound of any of paragraphs 199-238, wherein the compound does not increase SMN protein levels.
   241. The use of the compound of any of paragraphs 199-238, wherein the compound modulates a biological pathway that increases SMN protein levels.
   242. The use of the compound of any of paragraphs 199-241, wherein said compound inhibits the activity of at least one kinase.
   243. The use of the compound of any of paragraphs 199-242, wherein the kinase is cyclin-dependent kinase (CDK).
   244. The use of the compound of any of paragraphs 199-242, wherein the kinase is GSK-3.
   245. The use of the compound of any of paragraphs 199-242, wherein the kinase is IKK-2.
   246. The use of the compound of any of paragraphs 199-242, wherein the compound inhibits the activity of two or more kinases.
   247. The use of the compound of paragraphs 246, wherein the compound inhibits GSK-3 and at least one other kinase.
   248. The use of the compound of paragraph 247, wherein the other kinase is CDK.
   249. The use of the compound of any of paragraphs 199-248, wherein the contact is in vitro.
   250. The use of the compound of any of paragraphs 199-248, wherein the contact is in vivo.
   251. The use of the compound of any of paragraphs 199-248, wherein in vivo contact is in a mouse.
   252. The use of the compound of any of paragraphs 199-248, wherein in vivo contact is in a subject, where the subject is selected for treatment of a neurodegenerative disorder characterized by degeneration of motor neurons.
   253. The use of the compound of any of paragraphs 199-252, wherein the subject is human.
   254. The use of the compound of any of paragraphs 199-253, wherein the neurodegenerative disorder is characterized by a mutation in the SMN gene.
   255. The use of the compound of any of paragraphs 199-254, wherein the neurodegenerative disorder is characterized by diminished levels of SMN protein.
   256. The use of the compound of any of paragraphs 199-255, wherein the neurodegenerative disorder is spinal muscular atrophy (SMA).
   257. The use of the compound of any of paragraphs 119-255, wherein the neurodegenerative disorder is amyotrophic lateral sclerosis (ALS).
   258. A method of promoting motor neuron cell survival, the method comprising: contacting a motor neuron cell with a compound selected from the group consisting of Kenpaullone, Alsterpaullone, 2-cyanoethyl-alsterpaullone, CHIR98014, CHIR99021, GSK1, GSK2, GSK6, GSK7, GSK8, GSK 13, and combinations thereof.
   259. A method of increasing SMN protein levels in a cell, the method comprising: contacting a cell with a compound selected from the group consisting of Alsterpaullone, 2-cyanoethyl-alsterpaullone, CHIR98014, CHIR99021, GSK2, GSK6, GSK7, GSK8, GSK9, GSK15, Oubain, Digoxin, Dititoxin, Lanatoside, PDGF, platelet derived PDGF, PDGF-BB, PDGF-AB, PDGF-DD, PDGF-CC, PDGF-AA, FGF, Trichostatin, Lactacystin, MG-132, BMP4, SAHA, PKR inhibitor, WIN 55, 212-2, Ionomycin, Thapsigargin, Calcimycin, Anysomycin, Coumermycin A₁, ceramide, Veratridine, Monensin Na, Valinomycin IMD-0354, Nicolsamide, MBCQ, Dipyridamole, and combinations thereof.
   260. The method of paragraph 258 or 259, wherein the motor neuron comprises a mutation in gene encoding SMN1.
   261. The method of any of paragraphs 258-260, wherein the motor neuron comprises a mutation in gene encoding superoxide mutase 1 (SOD1).
   262. The method of any of paragraphs 258-261, wherein the motor neuron comprises a G->A mutation at position 93 of gene encoding SOD1.
   263. The method of any of paragraphs 258-262, wherein the compound modulates a biological pathway or a target, wherein the biological pathway or target is selected from the group consisting of PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, Na⁺/K⁺ channel, MAPK, cannobinoid receptor or GPCR, Ca²⁺channel, K⁺ channel, PDE5, GSK/CDK, PKR, CDK2, IKK-2, proteasome, BMP/TGFbeta receptor and dopamine receptor.
   264. The method of any of paragraphs 258-263, wherein the contact is in vitro.
   265. The method of any of paragraphs 258-263, wherein the contact is in vivo.
   266. The method of any of paragraphs 258-263, wherein in vivo contact is in a mouse.
   267. The method of any of paragraphs 258-263, wherein in vivo contact is in a subject, where the subject is selected for treatment of a neurodegenerative disorder characterized by degeneration of motor neurons.
   268. The method of any of paragraphs 258-267, wherein the subject is human.
   269. The method of any of paragraphs 258-268, wherein the neurodegenerative disorder is characterized by a mutation in the SMN gene.
   270. The method of any of paragraphs 258-269, wherein the neurodegenerative disorder is characterized by diminished levels of SMN protein.
   271. The method of any of paragraphs 258-270, wherein the neurodegenerative disorder is spinal muscular atrophy (SMA).
   272. The method of any of paragraphs 258-270, wherein the neurodegenerative disorder is amyotrophic lateral sclerosis (ALS).

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### High Content Drug Screens in Fibroblasts and Motor Neurons Reveal New Modulators of SMN Protein

The inventors have adopted an unbiased, image-based approach to identify compounds and pathways that elevate SMN levels. It is also a first high-content drug screen that evaluated SMN increase in a whole cell and different cellular compartments.

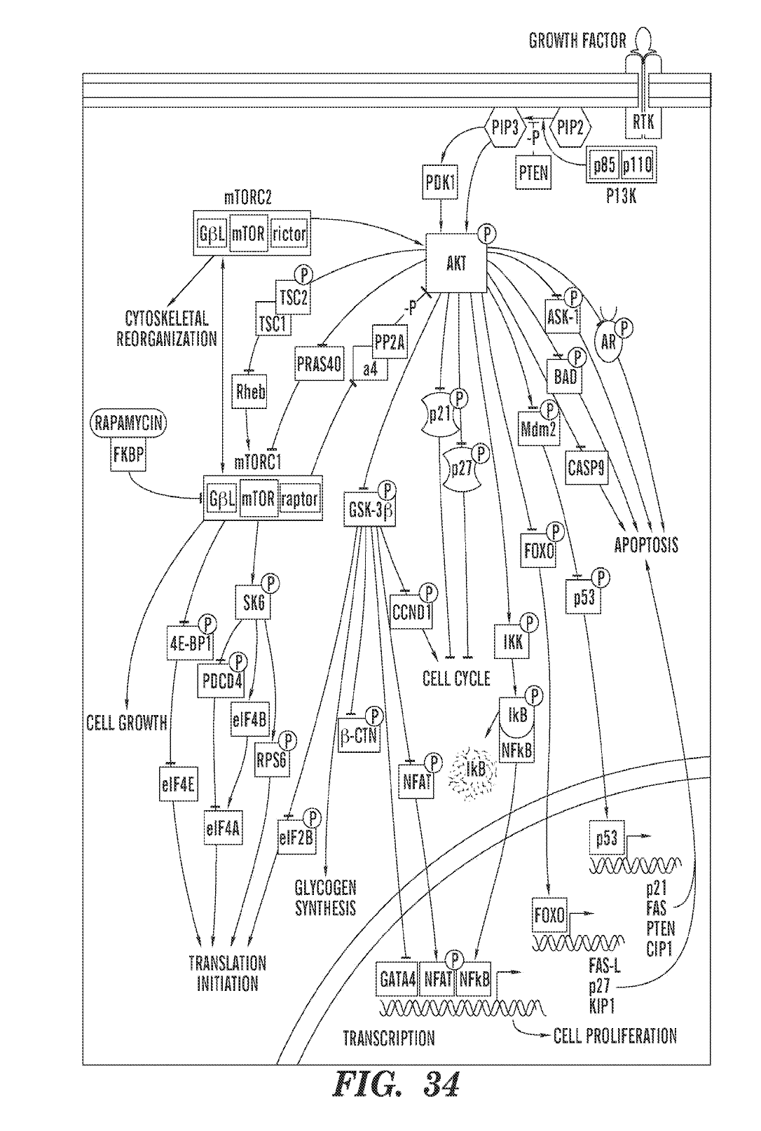

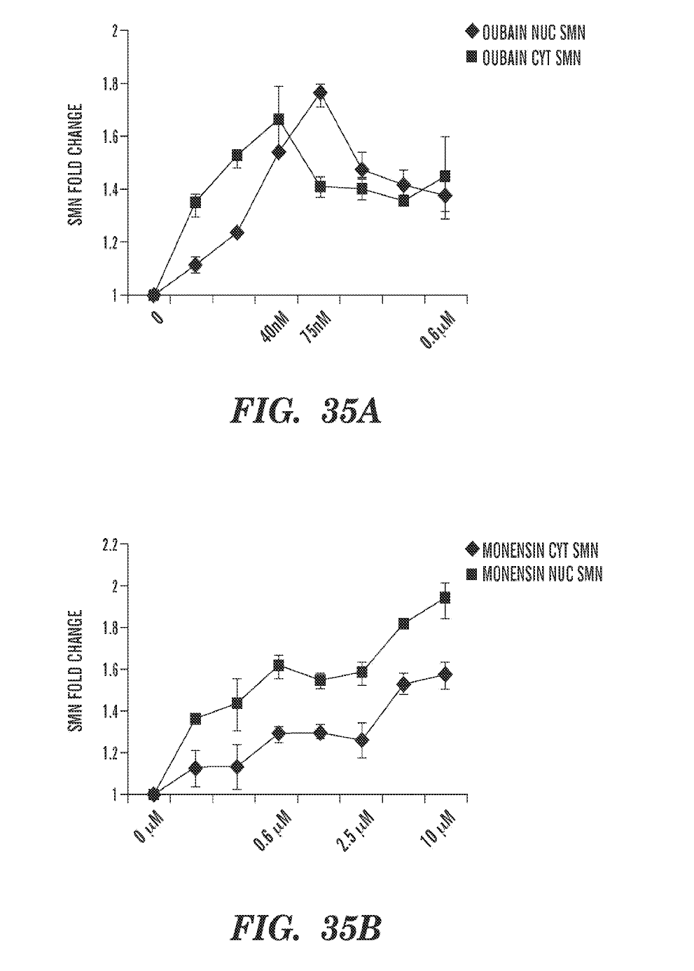

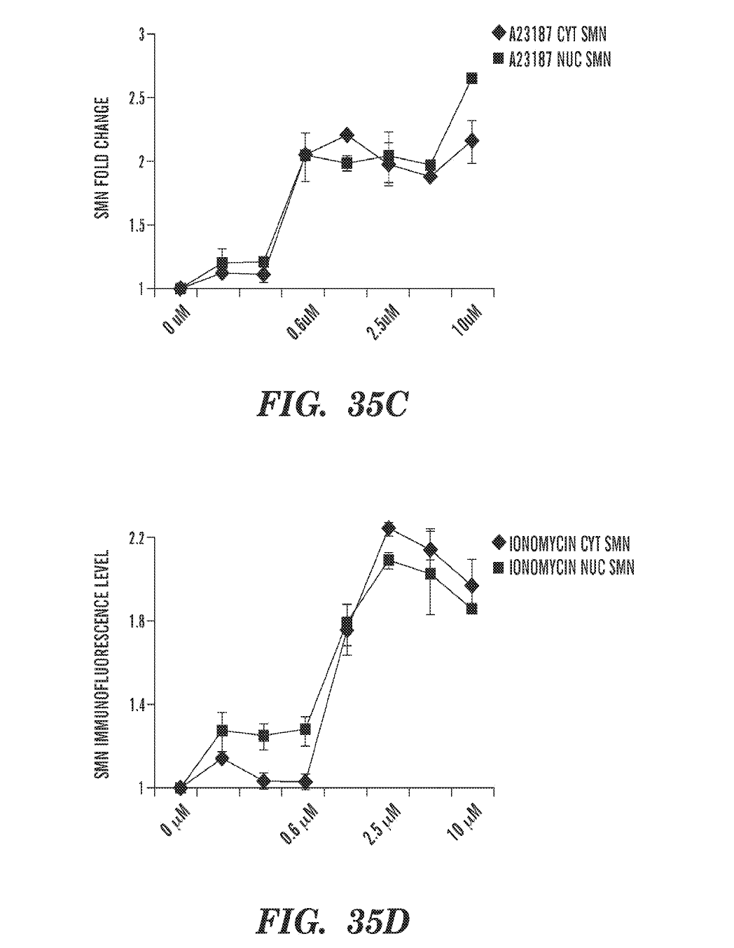

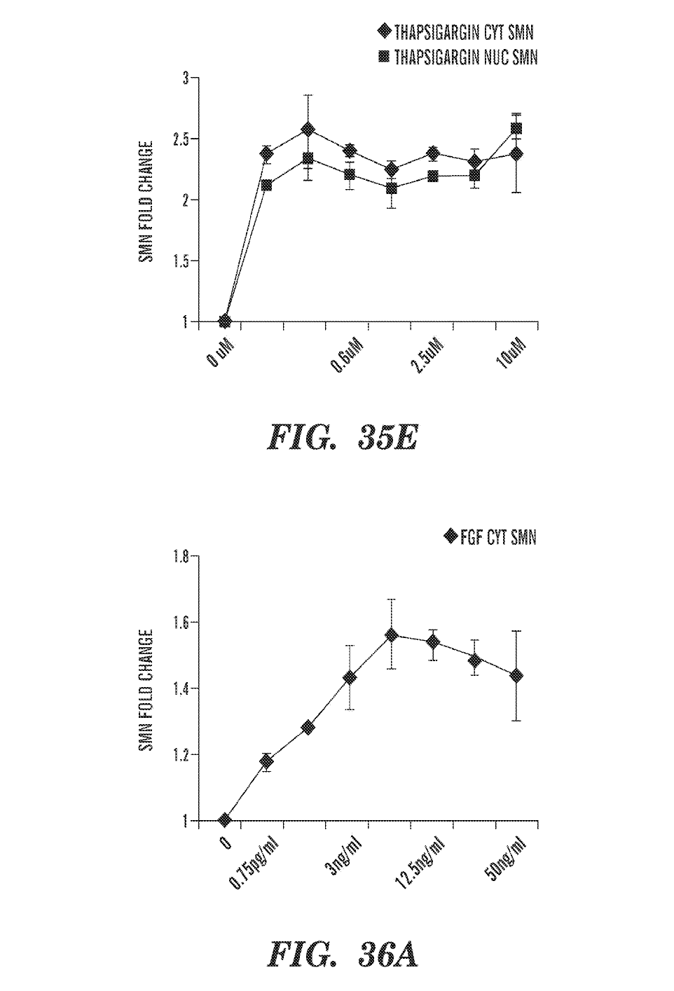

The inventors have screened both normal and SMN-deficient human fibroblasts and mouse motor neurons using compound diversity libraries and annotated compound collections. The inventors are particularly interested in identifying pathways that regulate SMN levels physiologically. Examples of the biological pathways and targets that elevates the SMN levels are shown in FIGS. 34 and 35.

The new technology described herein that combines automation, high-resolution confocal visualization and precise biological targeting sets a new standard for drug development efforts toward finding the cure from SMA.

### Fibroblast Screen

The inventors screened 5000 pharmacologically active compounds with well-characterized activities and known targets in the cell at a range of concentrations to reveal their true activities that would point out the biological pathway involved in SMN regulation. Human fibroblasts were incubated for 2-3 days with compounds from annotated collections. Cells were fixed and stained with an anti-SMN antibody. Confocal imaging was used to quantify SMN fluorescence in nucleus and cytoplasm and count Gems. Data were collected and subsequently analyzed by clustering compounds by suspected mechanism of action and chemical structure.

Through their screening efforts they identified a number of new highly fibroblasts-active compounds, which were able to increase SMN in several cellular compartments, such as cardiac glycosides (e.g., Ouabain, Digoxin, Dititoxin and Lanatoside C), activators of MAPK (e.g., Anysomycin (activates p38 and Erk1/2), Coumermycin A₁ (stimulates Raf-1 and MAP kinase), ceramide (activates MAPK, PKC and PI-3 kinase)) cannabinoid receptor or GPCR agonists (e.g., WIN 55, 212-2 (activates p42 and p44 MAP kinase), Anandamide) that are known to activate intracellular signaling pathways, compounds that increased concentration of Ca²⁺ (e.g., Thapsigargin, Ionomycin, Calcimycin) and K⁺ (e.g., Veratridine, Monensin Na, Valinomycin), PDE5 inhibitors (e.g., MBCQ, Dipyridamole), HDAC inhibitors (e.g., SAHA), and proteasome inhibitors (e.g., Lactacystin, MG-132). Apart from it, we focused our attention on compounds that obviously decreased SMN, and to this group belongs compounds that inhibit PI-3 kinase (e.g., LY294002, PI-103, Rapamycin, PI-828, PI 3-Kg Inhibitor (e.g., EMD #528106)) and PDGF receptor inhibitors (e.g., DMPQ and AG-1296). Through the follow on study of intracellular mechanisms that modulate SMN levels, the inventors found that SMN is up-regulated upon activation of PI-3 kinase pathways, and the strongest activator is platelet derived PDGF or human recombinant PDGF-BB.

Since targeting kinases with inhibitors is the most promising strategy for finding compounds for clinical application, the inventors also focused on identification of known kinase inhibitors that are able to increase SMN. The inventors found that SMN is increase after treatment with inhibitors of GSK/CDK-Alsterpaullone and it structural analogs (e.g., 1-aza-Alsterpaullone, 2-cyan-Alsterpaullone) and GSK inhibitor AR-A014418, PKR inhibitor (6,8-Dihydro-8-(1H-imidazol-5-ylmethylene)-7H-pyrrolo[2,3-g]benzothiazol-7-one, also known as SU9516), and it's structural analog CDK2 inhibitor GW8510, IKK-2 inhibitor IMD-0354 and it's structural analog Niclosamide.

Activation of PI-3 kinase and inhibition of its downstream GSK kinase makes a connected pathway, since it is known that PI-3/AKT inactivates GSK. Apart from it, cycling D is also being regulated by PI-3/AKT, and inhibitors of CDK could exert their action downstream of the same pathway. As for kinase inhibitors like Rapamycin (inhibitor of mTOR) that decreases SMN and PKR inhibitors that activate translation and increase SMN, they also fit within the PI-3/AKT pathway that is known to be a strong inducer of protein synthesis, if it is the case that SMN is regulated translationally. The inventors focused on narrowing down inhibition of which kinase is involved in direct SMA increase using lentiviral shRNA knockdown of 5 specific genes: GSK-3b, CDK2, CDK5, PKR, IKK-2b.

Specifically, fibroblasts from patients suffering from SMA were treated with Ionomycin (a channel modulator), Ouabain (a cardiac glycoside and channel modulator), PDGF, SAHA (an HDAC inhibitor), Lactacystin (a proteasome inhibitor) or WIN 55,212-2 (a cannabinoid receptor inhibitor and GPCR ligand) for 72 hours. The cells were fixed with methanol/acetone and stained with Pharmingen BD antibodies, followed by 488 AlexaFluor staining. Images were obtained with 60× lens on Zeiss LSM510 META on an inverted confocal microscope. The levels of both cytoplasmic and nuclear SMN were measured (data not shown). An increase in SMN levels was seen with these compounds. Table 1 summarizes distribution of hit compound based on their biological activity.

TABLE 1
Biological activity distribution of hit compounds.

| | |
|---|---|
| Ion channel | 17% |
| Kinase | 6% |
| Lox | 1% |
| MDR | 1% |
| NMDR | 1% |
| Opioid | 2% |
| Other | 19% |
| Protein phosphatase | 1% |
| Protein synthesis | 2% |
| Protein transport | 1% |
| Serotonin | 6% |
| Tubulin | 14% |
| Tubuline/kinase | 1% |
| Adenosine | 1% |
| Adrenoceptor | 1% |
| Chalcone | 4% |
| Cyclic peptide | 1% |
| DNA | 7% |
| Dopamine | 4% |
| GABA | 1% |
| Histamine | 1% |
| Hormone | 7% |

### SMA Motor Neuron Screen

The inventors screened 5000 pharmacologically active compounds with well-characterized activities and known targets in the cell at a range of concentrations to reveal their true activities that would point out the biological pathway involved in SMN regulation. Mouse HB9-GFP derived SMN deficient motor neuron cells were incubated for 2 days with compounds from libraries and collections. Cells were fixed and stained with an anti-SMN antibody. Confocal imaging was used to quantify SMN fluorescence in nucleus and cytoplasm and count Gems. Data were collected and subsequently analyzed by clustering compounds by suspected mechanism of action and chemical structure.

Through their screening efforts they identified a number of new highly motor neuron-active compounds, which were able to increase SMN in several cellular compartments, such as HDAC inhibitors (e.g., Trichostatin), proteasome inhibitors, BMP/TGFβ ligands (e.g., BMP4), Dopamine Receptor ligands, and GSK-3β inhibitors.

### Example 2

### The Effect of Cardiac Glycosides on the Levels of SMN in Patient Fibroblasts

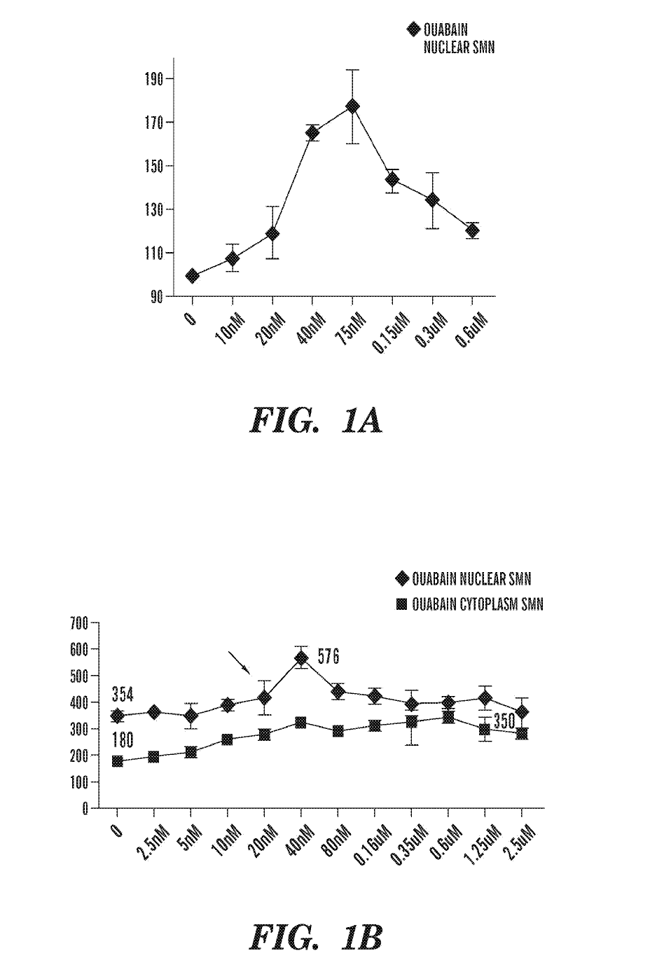

Many cardiac glycosides scored in the fibroblast screen. For example, fibroblasts from patients suffering from SMA were treated with Ouabain, Digoxin or Lanatoside at a concentration of 0, 2.5 nM, 5 nM, 10 nM, 20 nM, 40 nM, 80 nM, 0.16 nM, 0.35 nM, 0.6 nM, 1.25 nM or 2.5 nM for 72 hours. The cells were fixed with methanol/acetone and stained with Pharmingen BD antibodies, followed by 488 AlexaFluor staining. Images were obtained with 60× lens on Zeiss LSM510 META on an inverted confocal microscope. The levels of both cytoplasmic and nuclear SMN were quantified and are shown in FIG. 1.

### Example 3

### The Effect of Ca2+Modulators on the Levels of SMN in Patient Fibroblasts

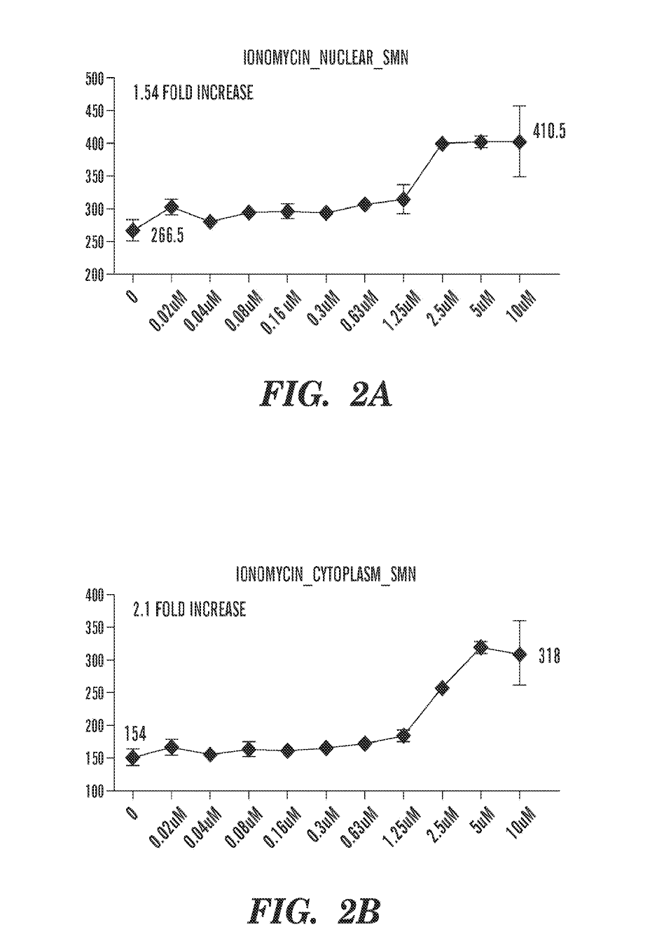

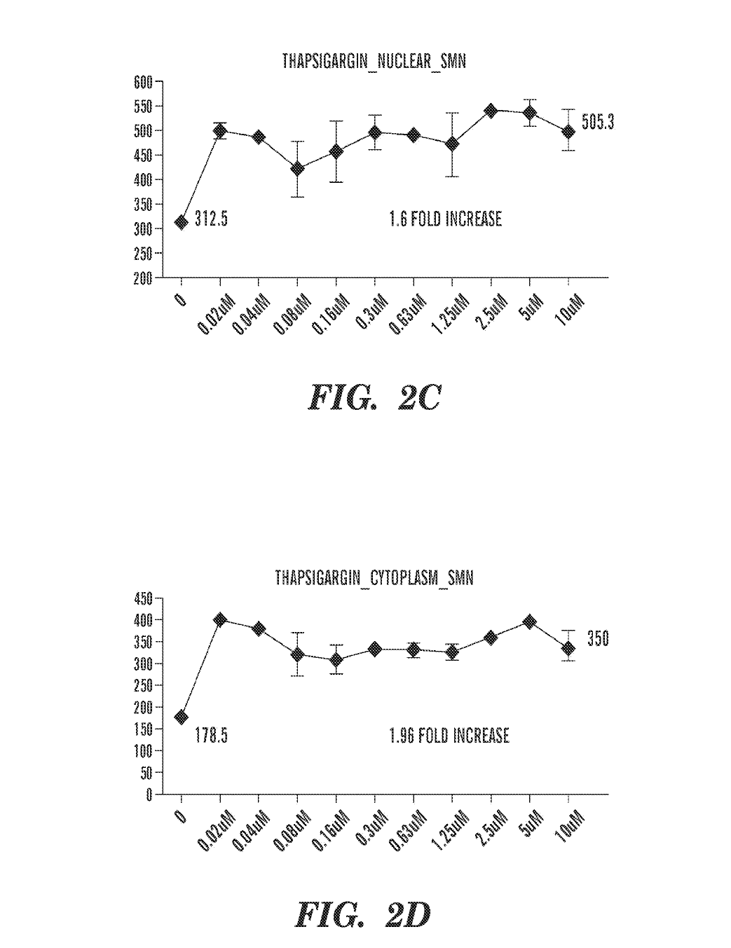

Fibroblasts from patients suffering from SMA were treated with Ionomycin, Calcimycin, Thapsigargin, Ryanodine, Cyclopiazonic acid or Bay K8644 for 72 hours. The levels of both cytoplasmic and nuclear SMN were detected and quantified using the method described in Example 2. The results shown in FIG. 2 indicate that Ionomycin, Calcimycin and Thapsigargin are active in elevating the levels of SMN. Ryanodine has a weak effect, while Cyclopiazonic acid and Bay K8644 are not active in activation of SMN.

### Example 4

### The Effect of FGF on the Levels of SMN in Patient Fibroblasts

Fibroblasts from patients suffering from SMA were treated with FGF2 at a concentration of 0, 20 pg/ml, 40 pg/ml, 75 pg/ml, 0.15 ng/ml, 0.3 ng/ml, 0.63 ng/ml, 1.25 ng/ml, 2.5 ng/ml, 5 ng/ml or 10 ng/ml for 72 hours. The levels of both cytoplasmic and nuclear SMN were detected and quantified using the method described in Example 2. The results shown in FIG. 10 indicate that FGF increases SMN levels.

### Example 5

### The Effect of PDGF on the Levels of SMN in Patient Fibroblasts

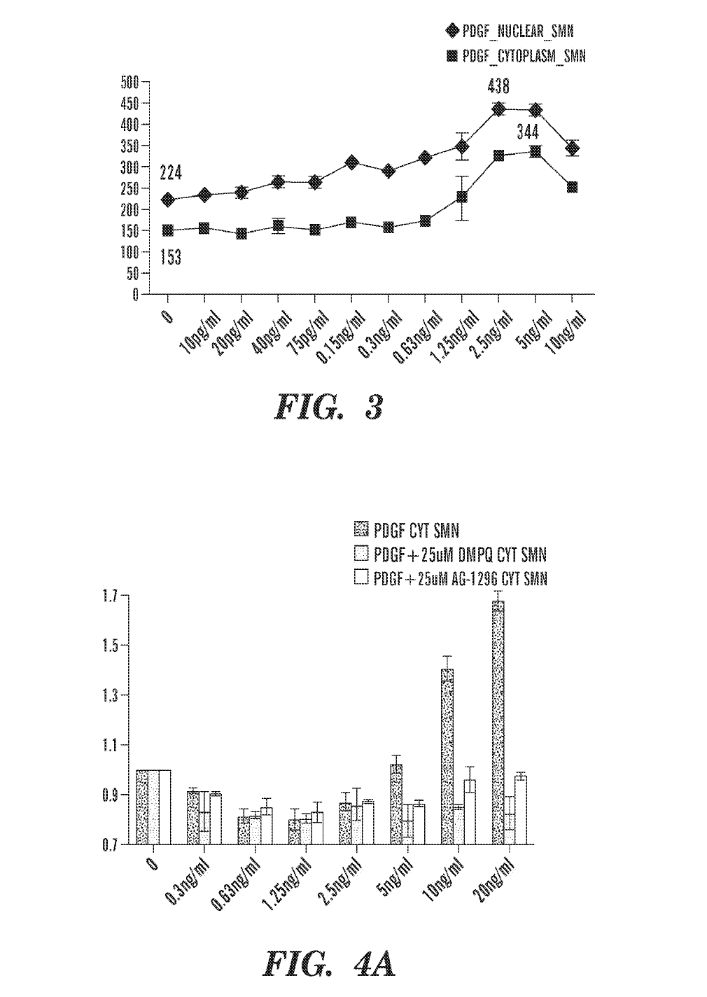

Fibroblasts from patients suffering from SMA were treated with PDGF at a concentration of 0, 10 pg/ml, 20 pg/ml, 40 pg/ml, 75 pg/ml, 0.15 ng/ml, 0.3 ng/ml, 0.63 ng/ml, 1.25 ng/ml, 2.5 ng/ml, 5 ng/ml or 10 ng/ml for 72 hours. The levels of both cytoplasmic and nuclear SMN were detected and quantified using the method described in Example 2. 2.3 fold increase in cytoplasmic SMN and 1.95 fold increase in nuclear SMN were observed as shown in FIG. 3.

### Example 6

### The Effect of PDGF Isoforms on Activation of SMN

The effect of various PDGF isoforms (e.g., platelet derived PDGF, recombinant PDGF-BB, recombinant PDGF-AB, recombinant PDGF-DD, recombinant PDGF-CC and recombinant PDGF-AA) on activating SMN were assessed using the method described in Example 2. The results shown in Table 2 indicate that PDGF-BB is the strongest activator of SMN.

TABLE 2
Effect of various PDGF isoforms on increasing SMN levels.

| Human PDGF | Avg. SMN fold increase |
|---|---|
| PDGF, platelet derived | 2-2.3 |
| PDGF-BB rec. | 2-2.3 |
| PDGF-AB rec. | 1.7-1.9 |
| PDGF-DD rec. | 1.6-1.8 |
| PDGF-CC rec. | 1.4-1.6 |
| PDGF-AA rec. | 1.3-1.4 |

*rec. = recombinant

### Example 7

### The Stimulatory Effect of PDGF on Activation of SMN is Abrogated by PDGFR inhibitors

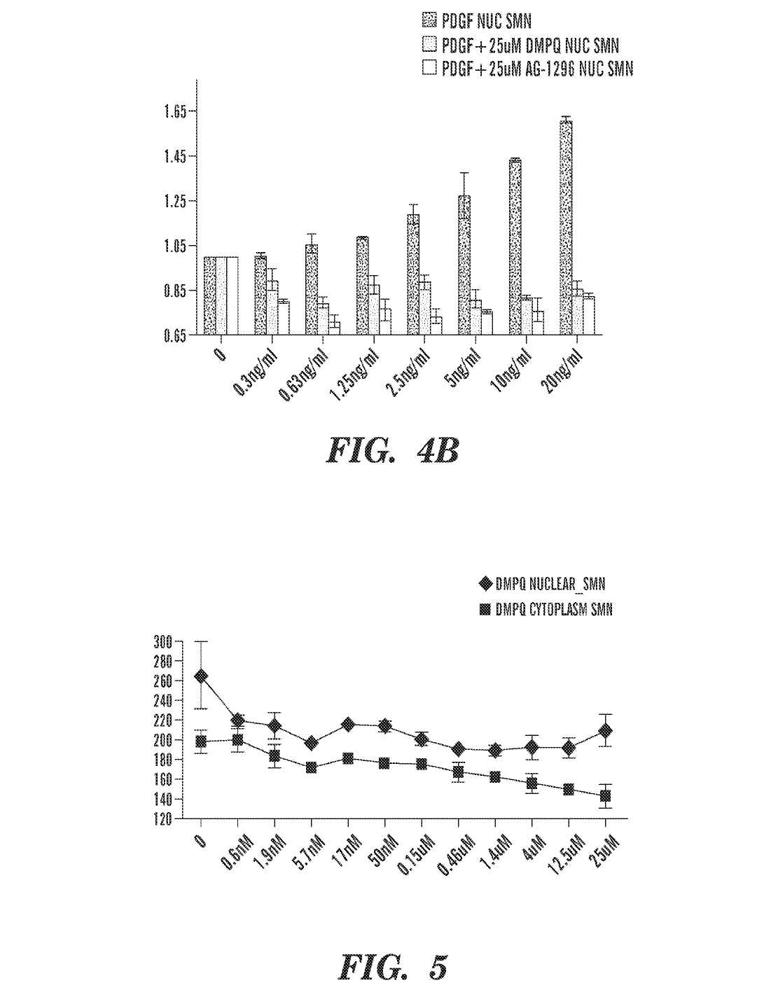

Fibroblasts from patients suffering from SMA were treated with PDGF at a concentration of 0, 0.3 ng/ml, 0.63 ng/ml, 1.25 ng/ml, 2.5 ng/ml, 5 ng/ml, 10 ng/ml or 20 ng/ml, in the absence or in the presence of 25 uM DMPQ or 25 uM AG-1296 respectively for 72 hours. The levels of both cytoplasmic and nuclear SMN were detected and quantified using the method described in Example 2. The results shown in FIG. 4 indicate that the stimulatory effect of PDGF on activation of both cytoplasmic and nuclear SMN is abrogated by PDGFR inhibitors.

### Example 8

### The Effect of PDGFR Inhibitor on the Levels of SMN

Fibroblasts from patients suffering from SMA were treated with DMPQ dihydrochloride at a concentration of 0, 0.6 nM, 1.9 nM, 5.7 nM, 17 nM, 50 nM, 0.15 uM, 0.46 uM, 1.4 uM, 4 uM, 12.5 uM or 25 uM for 72 hours. The levels of both cytoplasmic and nuclear SMN were detected and quantified using the method described in Example 2. The results shown in FIG. 5 indicates that the levels of both cytoplasmic and nuclear SMN decrease upon PDGFR inhibition.

### Example 9

### The Effect of Anti-PDGF Antibody on the Levels of SMN

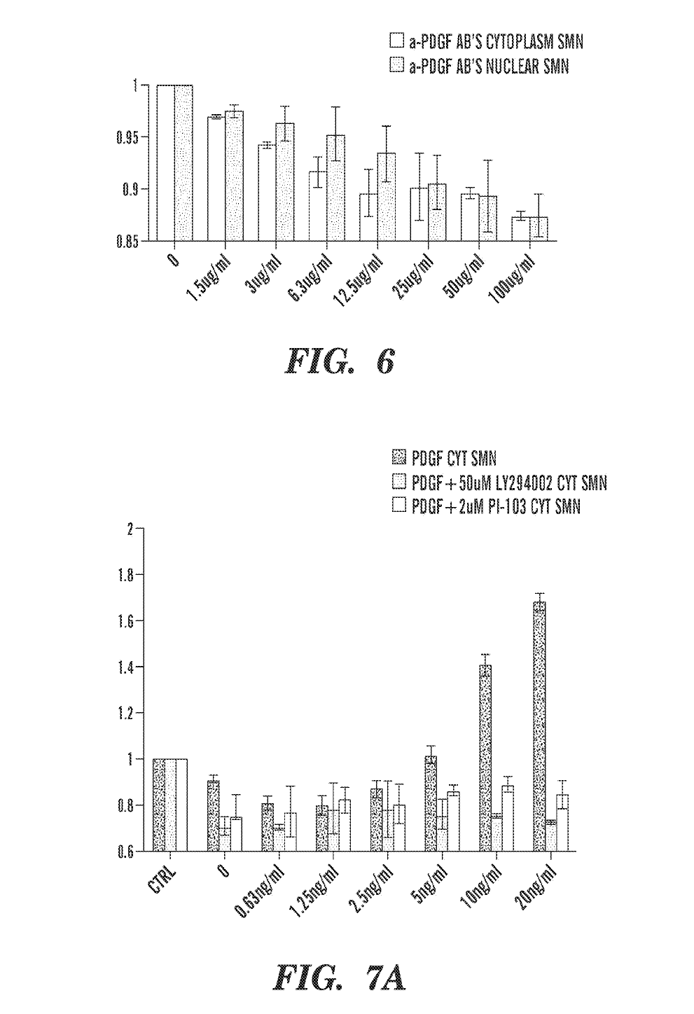

Fibroblasts from patients suffering from SMA were treated with an anti-PDGF antibody at a concentration of 0, 1.5 ug/ml, 3 ug/ml, 6.3 ug/ml, 12.5 ug/ml, 25 ug/ml, 50 ug/ml or 100 ug/ml for 72 hours. The levels of both cytoplasmic and nuclear SMN were detected and quantified using the method described in Example 2. The results shown in FIG. 6 indicate that the levels of both cytoplasmic and nuclear SMN decrease upon anti-PDGF antibody neutralization.

### Example 10

### The Stimulatory Effect of PDGF on Activation of SMN is Abrogated by PI3 Kinase Inhibitors

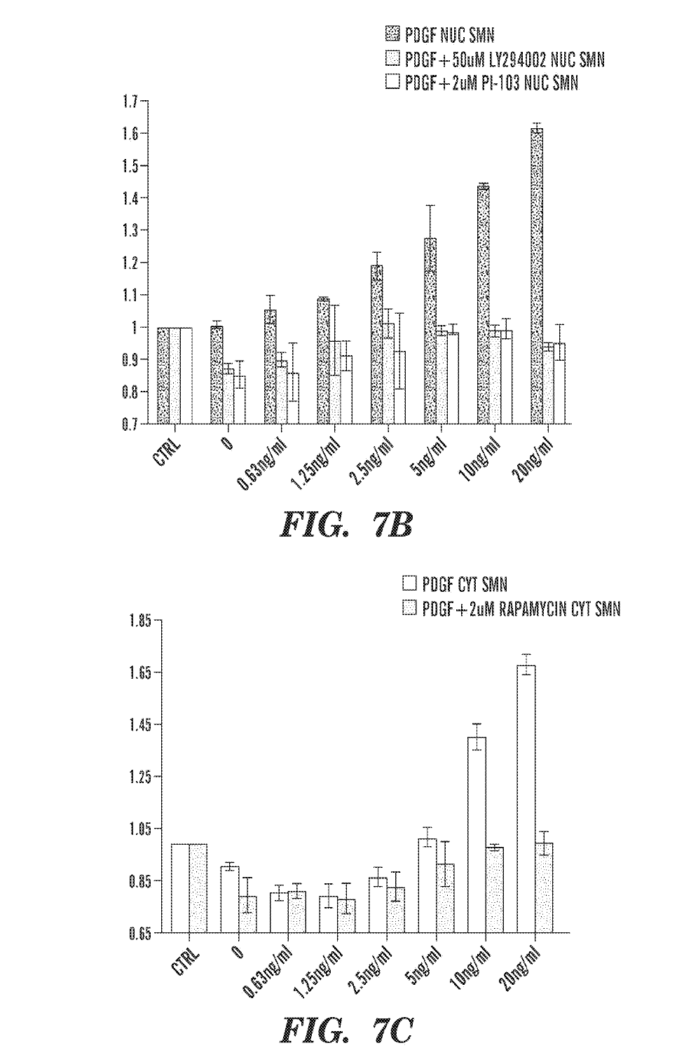

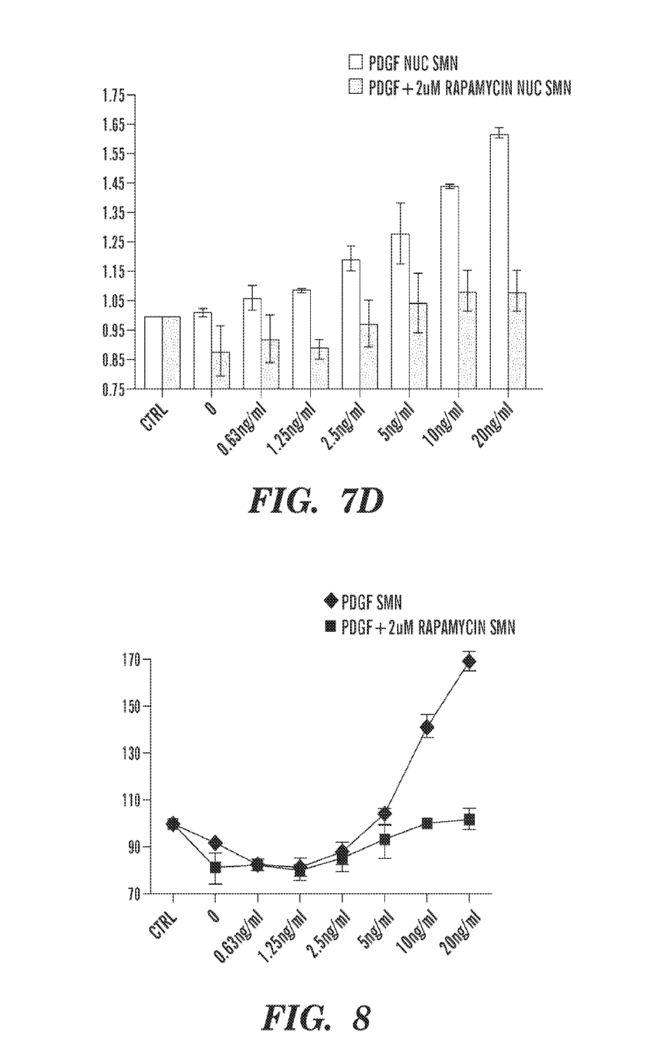

Fibroblasts from patients suffering from SMA were either control treated or treated with PDGF at a concentration of 0, 0.63 ng/ml, 1.25 ng/ml, 2.5 ng/ml, 5 ng/ml, 10 ng/ml or 20 ng/ml, in the absence or in the presence of 50 uM LY294002 or 2 uM P1-103 respectively for 72 hours. The levels of both cytoplasmic and nuclear SMN were detected and quantified using the method described in Example 2. The results shown in FIG. 7 indicate that the stimulatory effect of PDGF on activation of both cytoplasmic and nuclear SMN is abrogated by PI3 kinase inhibitors.

### Example 11

### The Stimulatory Effect of PDGF on Activation of SMN is Abrogated by an mTOR Inhibitor

Fibroblasts from patients suffering from SMA were either control treated or treated with PDGF at a concentration of 0, 0.63 ng/ml, 1.25 ng/ml, 2.5 ng/ml, 5 ng/ml, 10 ng/ml or 20 ng/ml, in the absence or in the presence of 2 uM Papamycin for 72 hours. The levels of both cytoplasmic and nuclear SMN were detected and quantified using the method described in Example x. The results shown in FIG. 8 indicate that the stimulatory effect of PDGF on activation of both cytoplasmic and nuclear SMN is abrogated by an mTOR inhibitor.

### Example 12

### The Effect of PI3 kinase Inhibitor on the Levels of SMN

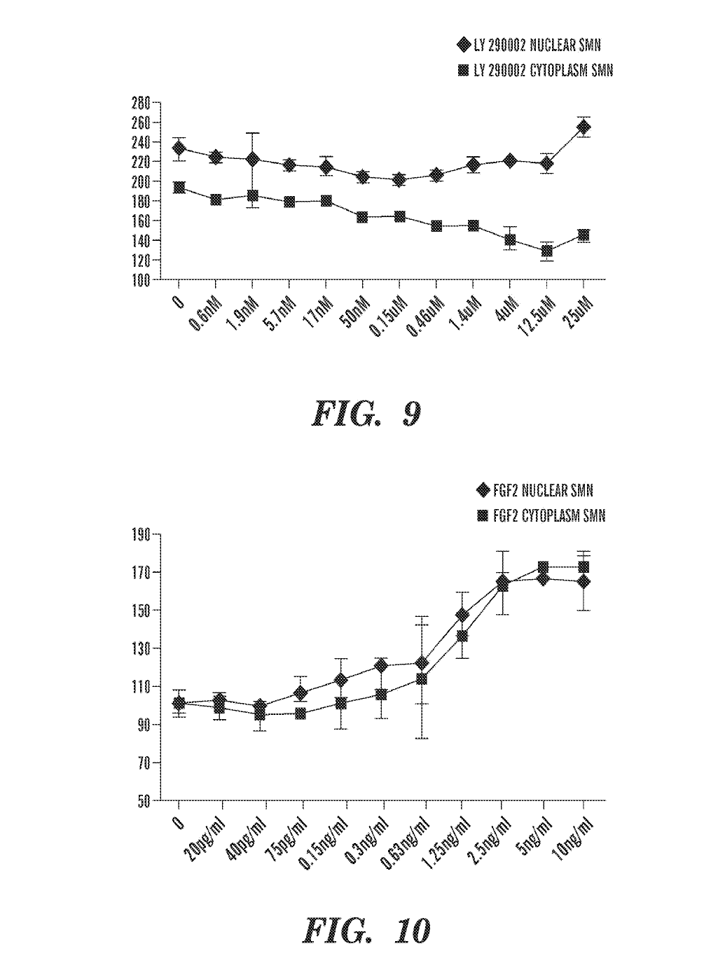

Fibroblasts from patients suffering from SMA were treated with LY290002 at a concentration of 0, 20 pg/ml, 40 pg/ml, 75 pg/ml, 0.15 ng/ml, 0.3 ng/ml, 0.63 ng/ml, 1.25 ng/ml, 2.5 ng/ml, 5 ng/ml or 10 ng/ml for 72 hours. The levels of both cytoplasmic and nuclear SMN were detected and quantified using the method described in Example 2. The results shown in FIG. 9 indicate that the levels of both cytoplasmic and nuclear SMN decrease upon FGF2 treatment.

### Example 13

### The Effect of FGF on the Levels of SMN

Fibroblasts from patients suffering from SMA were treated with FGF2 at a concentration of 0, 0.6 nM, 1.9 nM, 5.7 nM, 17 nM, 50 nM, 0.15 uM, 0.46 uM, 1.4 uM, 4 uM, 12.5 uM or 25 uM for 72 hours. The levels of both cytoplasmic and nuclear SMN were detected and quantified using the method described in Example 2 (data not shown). The results indicated that the levels of both cytoplasmic and nuclear SMN decrease upon PI-3 kinase inhibition.

### Example 14

### The Effect of Serum Starvation on the Levels of SMN

Fibroblasts from patients suffering from SMA were serum starved for 72 hours. The cells were then cultured in medium with either 10% serum or 0.5% serum and fixed three days later. The levels of both cytoplasmic and nuclear SMN were detected and quantified using the method described in Example 2 (data not shown). The results showed that serum starvation reduces SMN level by 15% in patient cells.

### Example 15

### The Effect of Serum Starvation Along with PI3 Kinase Inhibition on the levels of SMN

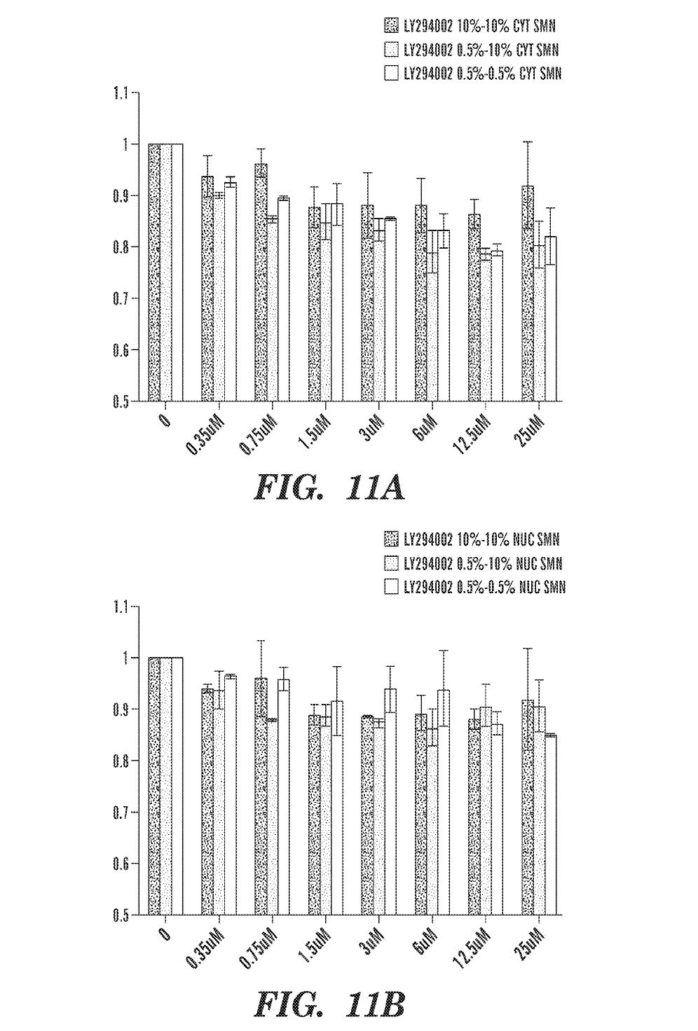

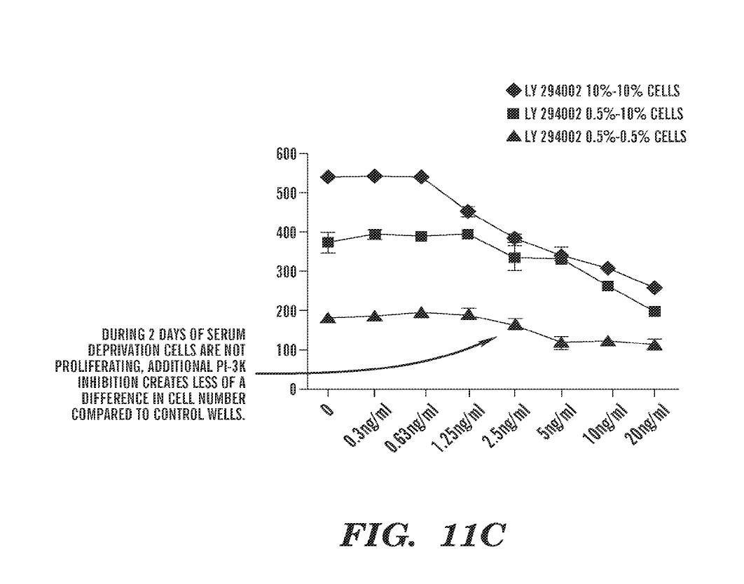

Fibroblasts from patients suffering from SMA were serum starved for 48 hours (control cells were cultured in the medium with 10% serum). The cells were then cultured in medium with either 10% or 0.5% serum and treated with LY294002 at a concentration of 0, 0.35 uM, 0.75 uM, 1.5 uM, 3 uM, 6 uM, 12.5 uM or 25 uM for another 3 days. The levels of both cytoplasmic and nuclear SMN were detected and quantified using the method described in Example 2. The results shown in FIG. 11 indicate that a combinatory treatment of 0.5% serum and a PI-3 kinase inhibitor reduces SMN by 20% in patient cells.

### Example 16

### GSK Inhibitors Increase the SMN Level when Added to Pre-Starved Cell Synergistically with PDGF

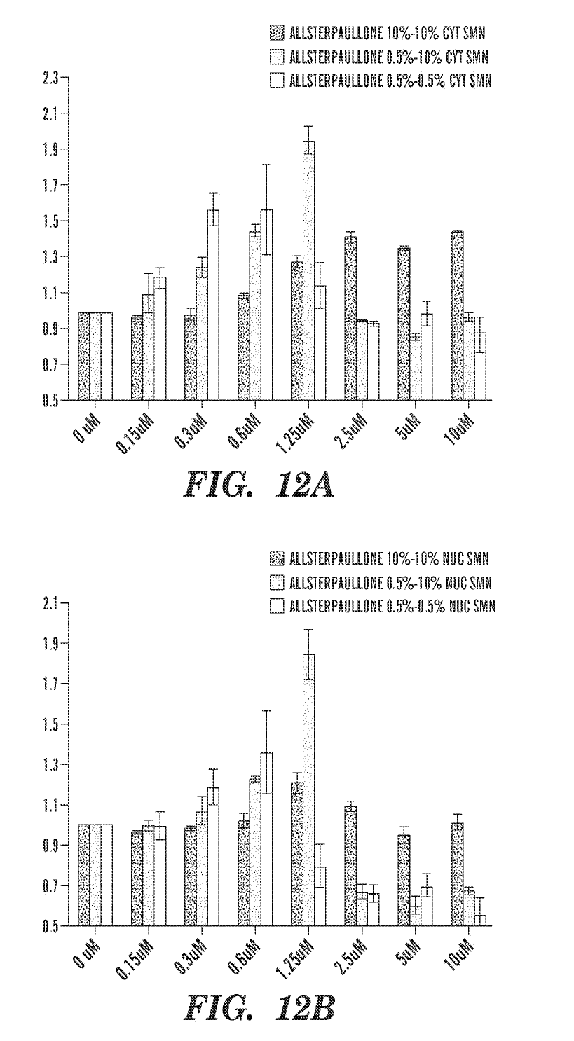

Fibroblasts from patients suffering from SMA were serum starved for 48 hours (control cells were cultured in the medium with 10% serum). The cells were then cultured in medium with either 10% or 0.5% serum and treated with Alsterpaullone at a concentration of 0, 0.15 uM, 0.3 uM, 0.6 uM, 1.25 uM, 2.5 uM, 5 uM or 10 uM for another 3 days. The levels of both cytoplasmic and nuclear SMN were detected and quantified using the method described in Example 2. The results shown in FIG. 12 indicate that Alsterpaullone has a weak effect on fed cells, a moderate effect (1.6 fold) on pre/post-starved cells, but a strong effect (1.9 fold) on pre-starved cell that received fresh media with 10% serum along with compound since PDGF inhibits GSK synergistically with Alsterpaullone. These results suggest that serum starvation inhibits the PI-3/Akt pathway, and keeps the GSK activity high. When Alsterpaullone is added—there is “active GSK target to inhibit”—leading to bigger fold effect on SMN. Presence of PDGF in 10% serum synergistically works to suppress GSK, by PI-3/Akt activation, and therefore best effect on SMN were observed. In case when cells were not starved—presence of PDGF in media containing 10% serum is keeping GSK suppressed, therefore GSK inhibitors have less of an effect.

### Example 17

### The Effect of HDAC Inhibitors on the Levels of SMN in Motor Neurons

Mouse motor neurons were treated with Trichastatin. The levels of both cytoplasmic and nuclear SMN were detected and quantified using the method described in Example 2 (data not shown). The results indicated that HDAC inhibitors increase SMN levels in motor neurons.

### Example 18

### The Effect of Proteasome Inhibitors on the Levels of SMN in Motor Neurons

Mouse motor neurons were treated with proteasome inhibitors. The levels of both cytoplasmic and nuclear SMN were detected and quantified using the method described in Example 2 (data not shown). The results indicated that proteasome inhibitors increase SMN levels in motor neurons.

### Example 19

### The Effect of BMP/TGFβ Ligands on the Levels of SMN in Motor Neurons

Mouse motor neurons were treated with BMP4. The levels of both cytoplasmic and nuclear SMN were detected and quantified using the method described in Example 2 (data not shown). The results indicated that BMP/TGFβ increase SMN levels in motor neurons.

### Example 20

### The Effect of Dopamine Receptor Ligands on the Levels of SMN in Motor Neurons

Mouse motor neurons were treated with Dopamine receptor ligands. The levels of both cytoplasmic and nuclear SMN were detected and quantified using the method described in Example 2 (data not shown). The results indicate that Dopamine receptor ligands increase SMN levels in motor neurons.

### Example 21

### The Effect of GSK-313 Inhibitors on the Levels of SMN in Motor Neurons

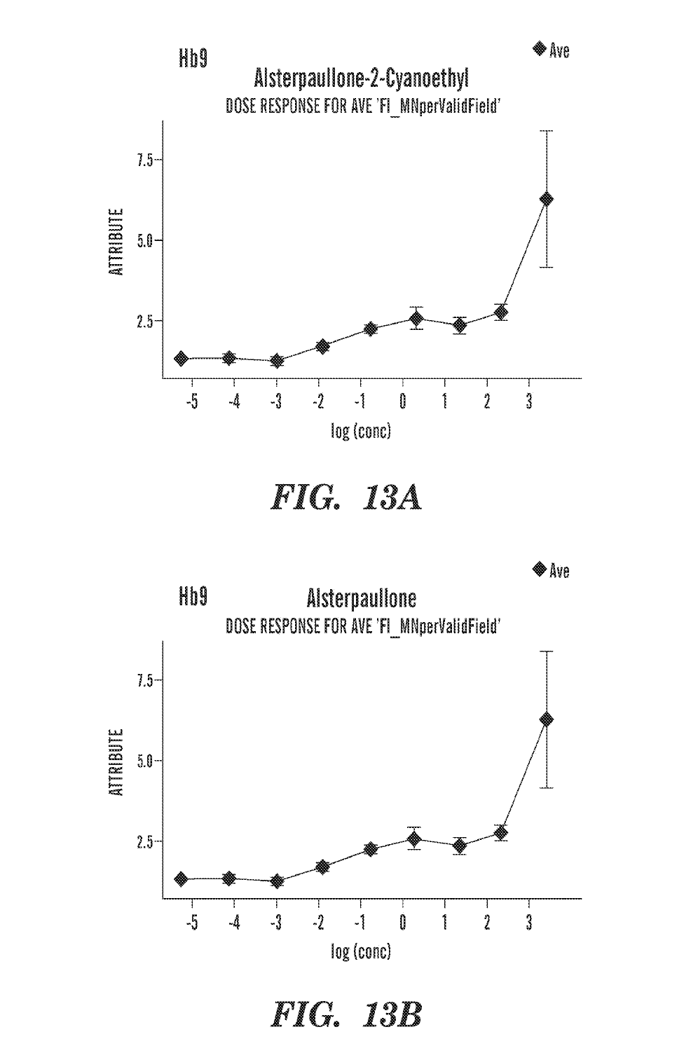

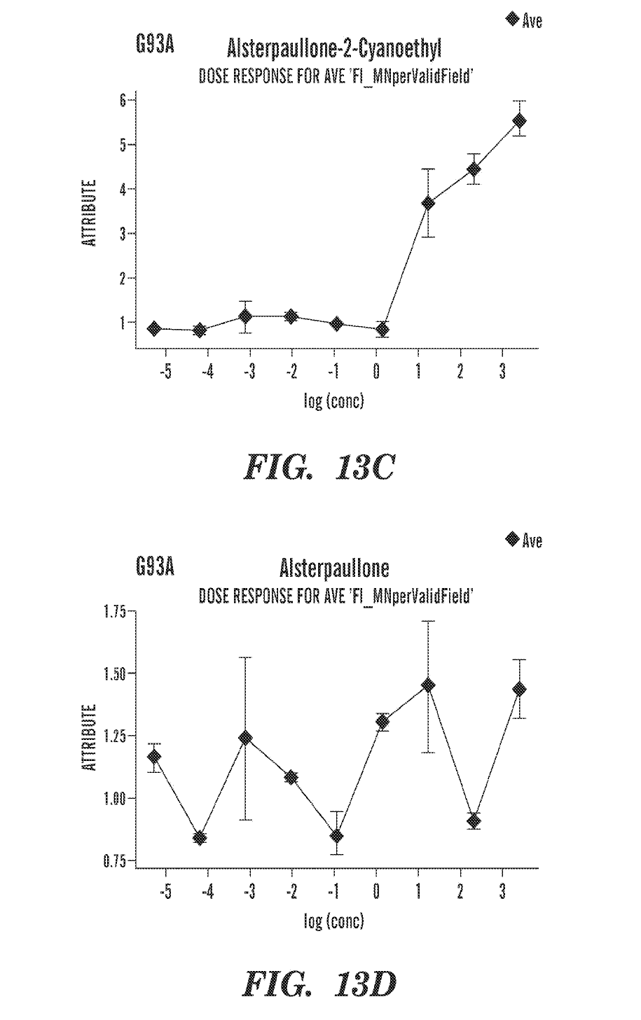

Mouse motor neurons were treated with Dopamine receptor ligands. The levels of both cytoplasmic and nuclear SMN were detected and quantified using the method described in Example 2. The results shown in FIG. 13 indicate that GSK-313 inhibitors increase SMN levels in motor neurons.

### Example 22

### The Effect of GSK-313 Inhibitors on Motor Neuron Survival

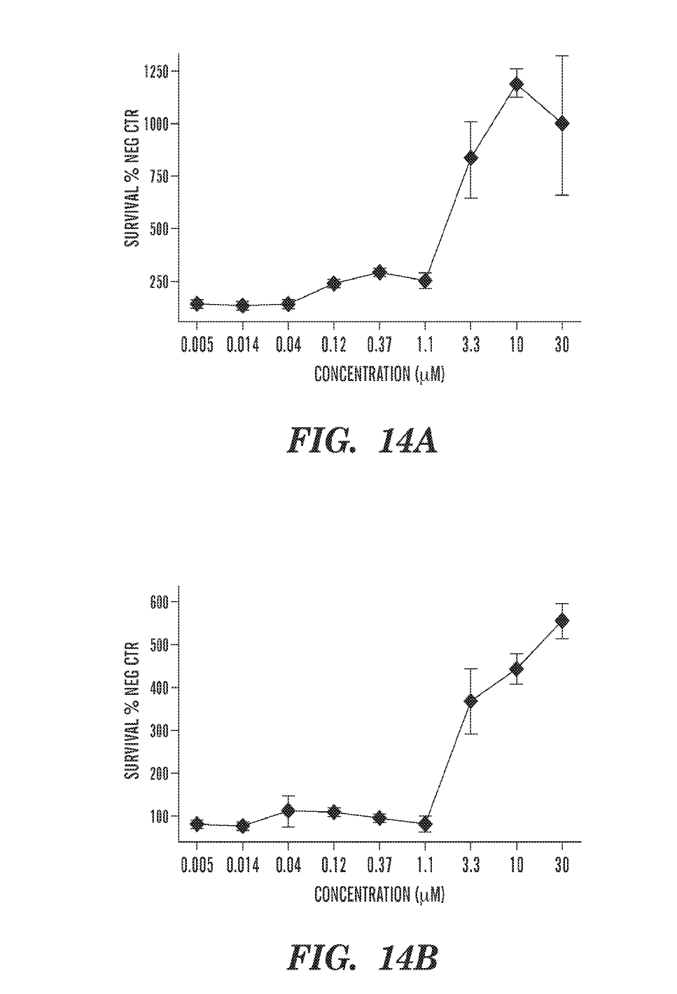

Mouse motor neurons were treated with GSK-313 inhibitors. The levels of both cytoplasmic and nuclear SMN were detected and quantified using the method described in Example 2. The results shown in FIG. 14b indicate that GSK-313 inhibitors increase motor neuron survival.

### Example 23

### Principle of SMN High-Content Detection and Small Molecules that Increase SMN in Different Cellular Compartments

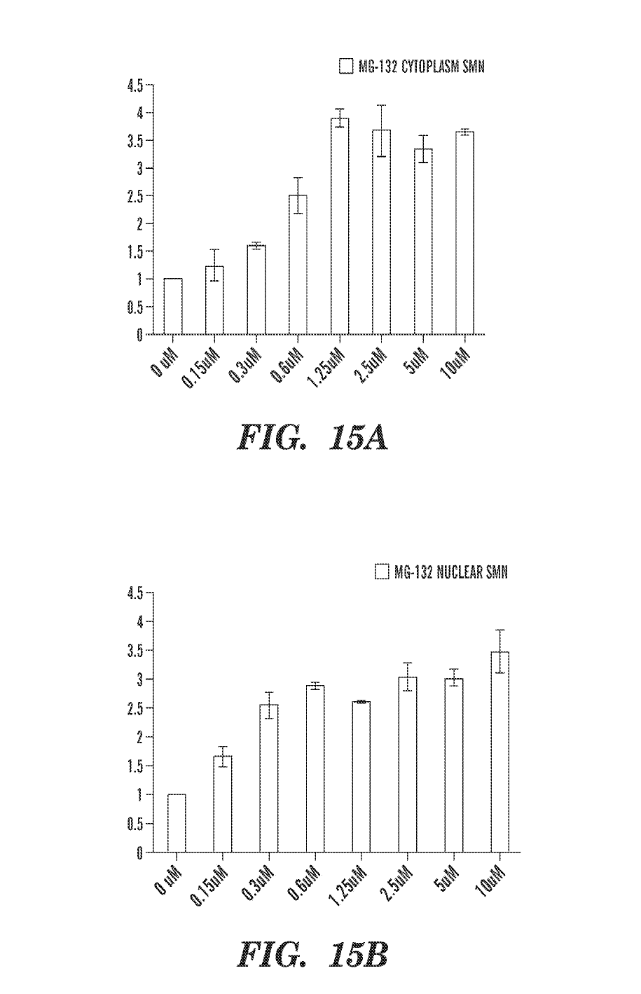

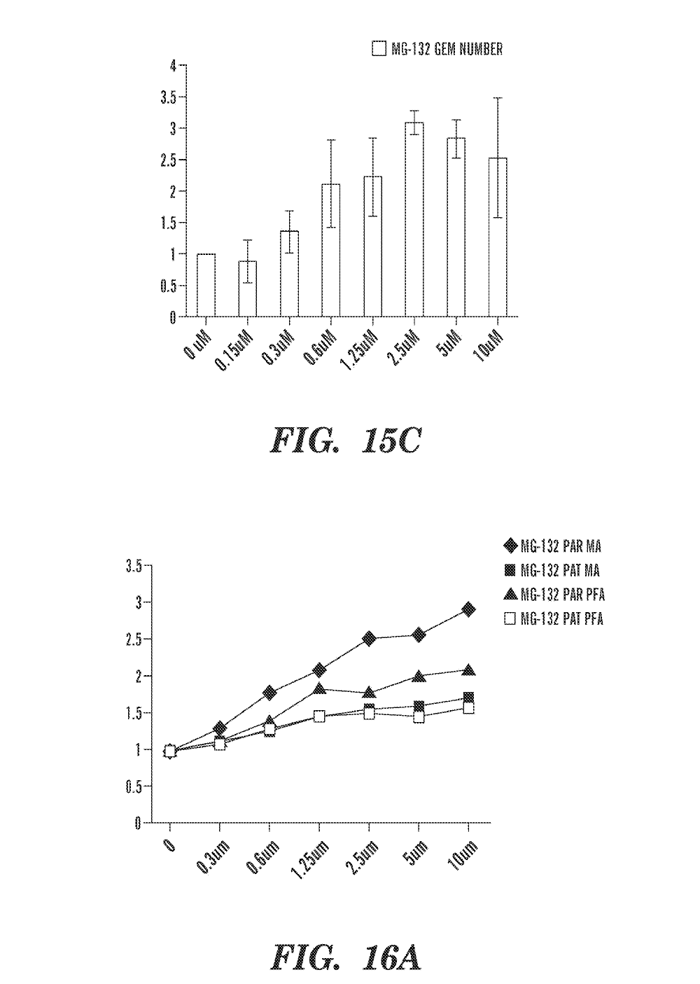

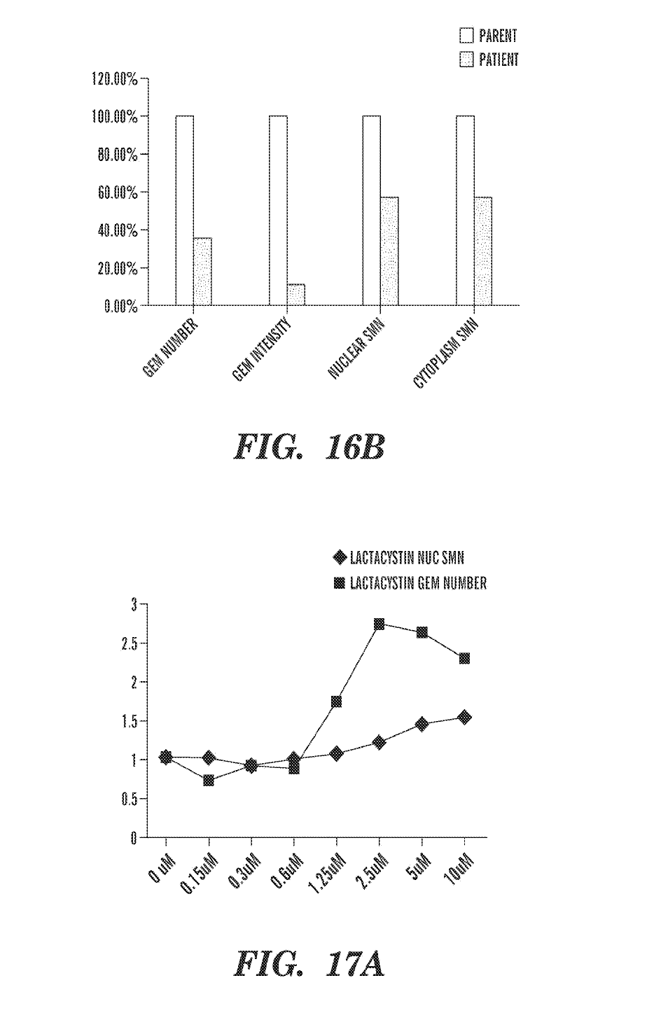

An assay was established for high-content evaluation of SMN protein level in different cellular compartments after treatment with small molecules. SMN protein was visualized by immunofluorescent staining and multiple fields from each plate well were scanned by an automated confocal Opera microscope. During assay development we customized image analysis script written with the PerkinElmer Acapella language to detect the SMN differences in nuclei, cytoplasm as well as the number and intensity of GEMs in control cells and cells treated and with the proteasome inhibitor MG-132 previously shown to increase SMN level human fibroblasts isolated from patients with SMA. For each well, the algorithm generated quantitative parameters representing number of cells, as well as SMN fluorescent intensity per cell in cytoplasm, nucleus, number and intensity of GEMs (FIG. 15). During the optimization phase we found that fixation with ice cold methanol/acetone yielded the best SMN signal to noise ratio between MG-132 treated and control cells (FIG. 16a). In addition human fibroblasts isolated from asymptomatic parental carriers of SMA despite the higher SMN baseline level (FIG. 16b) had a greater response to drug treatment compared to the SMA patient fibroblasts (FIG. 16a). During the assay transfer to the robotic platform fixation with the ice cold methanol was technically limited, therefore to preserve the maximum possible window we chose to perform the screen on parental fibroblast with PFA fixation.

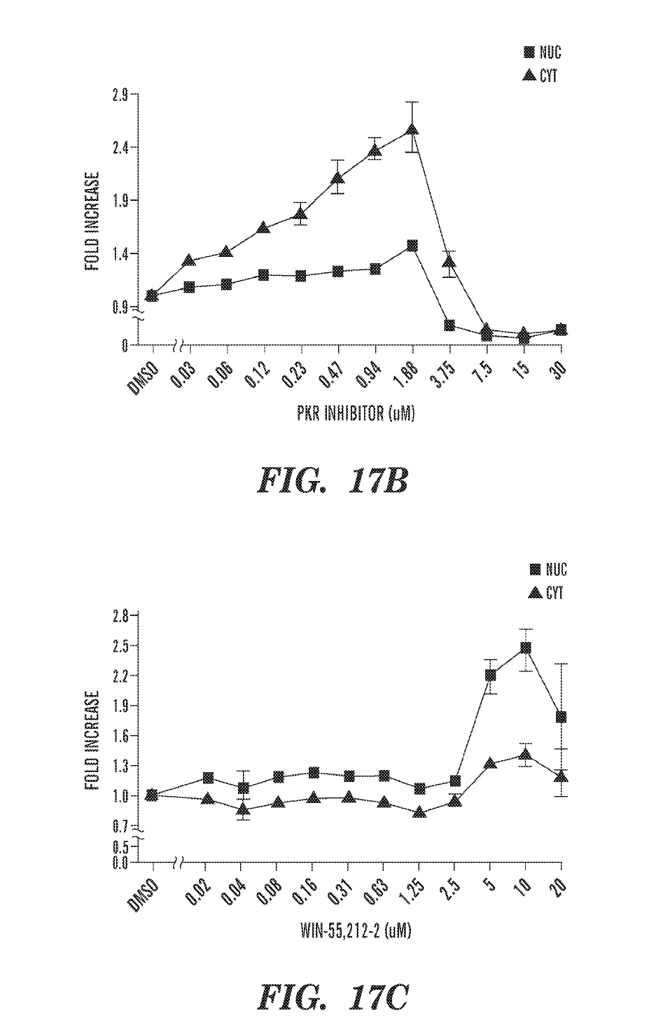

Prior to assay development a small number of compounds were pre-tested, and interestingly, SMN increase following treatment with different compounds was not homogeneous, it was observed that four SMN parameters described above were increased to a different degree depending on compound used. Certain compounds produced better SMN fold increase in cytoplasm (PKR inhibitor, 2-fold in cytoplasm vs 1.5 fold in nucleus), some in nucleus (WIN 55,212-2.2-fold in nucleus vs 1.5-fold in cytoplasm), and some mostly affected GEMs (Lactacystein), these observations were confirmed by the numerical output of the described parameters (FIG. 17). In general, compounds that increased SMN in the cytoplasm also increased SMN in the nucleus.

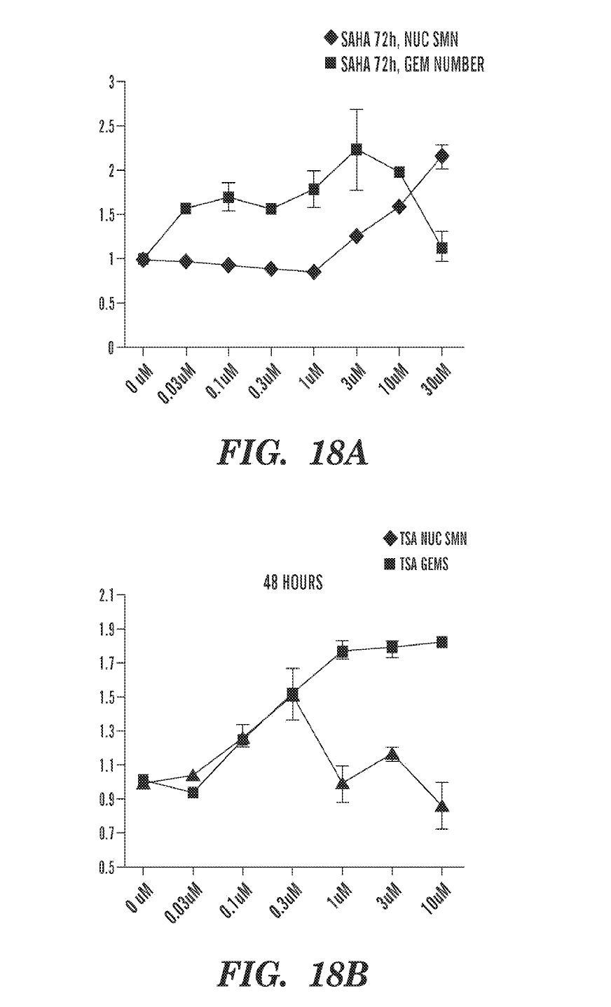

It was also noticed that concentration of compound and incubation length also had an impact on ratio between GEM number and total nuclear SMN. Compounds at their lower concentrations/shorter incubation time induced distinctive GEM formation while total nuclear SMN remained low. The same compounds at the higher dosage/longer incubation produced higher nuclear SMN values, whereas number of GEM decreased. Such dynamic effect can be due to the nuclear relative saturation with SMN that created low contrast for microscopic GEM detection (FIG. 18) These example shows, that classical approach of simple GEMs counting will not necessarily reveal the most potent compounds that increase SMN. However, some compounds that produced particularly impressive increases in nuclear appeared to decrease gem counts. A potential explanation for this is that the gems become harder to detect as the overall nuclear “background” increases. For example HDAC inhibitors, such as SAHA and Trichostatin, which scored in the screen, increased GEM counts at the lower concentrations, but at the higher dosage they appeared to increased nuclear SMN predominantly (FIGS. 18a and 18b).

For fibroblast assays, human fibroblasts lines GM09677 (derived from 2-year old patient with SMA type 1 disease) and GM03814 (derived from unaffected parental SMA carriers) were used (Coriell Cell Repositories). It was observed that cells from GM03814 line gave better signal to noise ratio during the screening (FIG. 16b). For secondary assays cells from GM09677 line were used.

Fibroblasts were grown in MEM media (Invitrogen) supplemented with 5% Fetal Bovine Serum (FBS, Invitrogen), 2 mM glutamine and 1% penicillin-streptomycin (100 U/ml) in a presence of 5% CO₂ at 37° C. For drug treatments, cells were plated and treated 24 h later with 10 μM MG132 (Biomol International), 5 mM Calpeptin (Biomol International), 10 mM ammonium chloride, 1 μM myristoylated PKI, or vehicle for the designated times.

### Example 24

### Screening Tree Workflow and Bioactive Small Molecule Hit Selection and Analysis

The composition of the diverse 5,000 compound small molecule library used for screening included the LOPAC¹²⁸⁰ (Library of Pharmacologically Active compounds from Sigma-Aldrich) Collection, The Spectrum Collection (Microsource) and the Prestwick Chemical Library, and a custom validation plate of 289 chemicals affecting kinases, ion channels and neuroactive compounds.

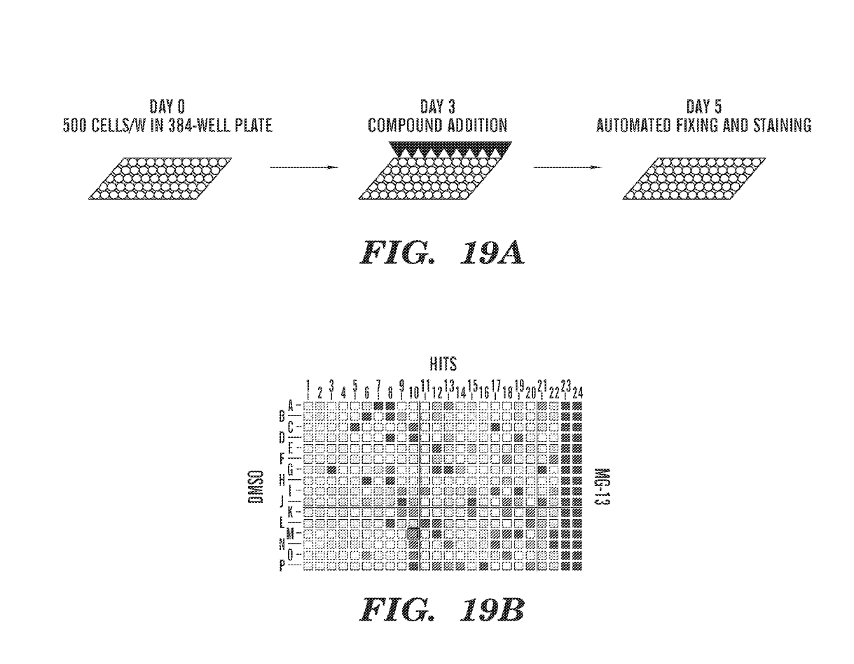

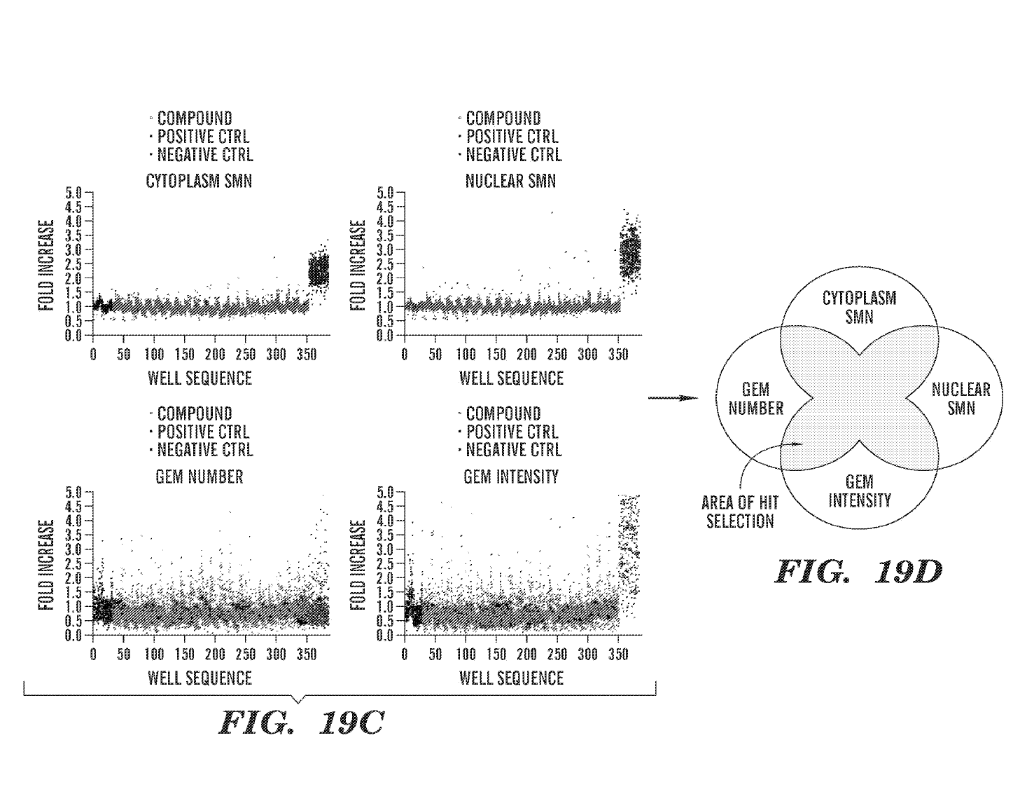

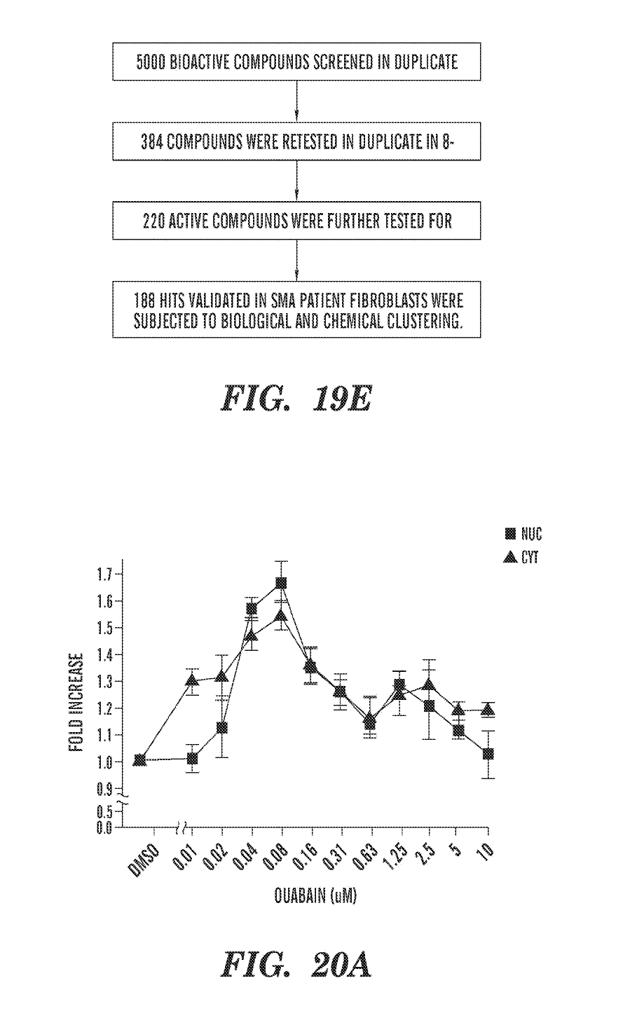

Following optimization of assay conditions on day 0, total of 500 human fibroblasts (GM09677 line) per well were seeded in 384-well plates, on day 3 they were treated with compounds (robot CyBio) from the annotated collections and on the 5 day plates were fixed and stained (robot MAPD) with an anti-SMN antibody and secondary antibody conjugated with 448 fluorophore (FIG. 19a). Compounds from the libraries were added in duplicate at three final concentrations of 10, 1 and 0.1 uM. Along with the library compounds, MG-132 was added as a positive control to all screening plates at concentration of 10 uM and as a negative control, cells were treated with the solvent used for library preparation—DMSO accordingly to the concentration of compounds on that plate.

SMN detection was done on the automated confocal microscope Opera PerkinElmer with separate fluorescent exposures to 488 nM laser for and a UV light source to detect nuclei stained with Hoechst 33258 with a 20× objective. Human fibroblasts isolated from asymptomatic parental carriers of SMA despite the higher SMN baseline level had a greater response to drug treatment compared to the SMA patient fibroblasts (FIG. 16a), therefore, screening was performed using parental fibroblasts.

Example of the heat map generated by the software is shown here (FIG. 19b). After scanning on the Opera, an XML file extraction workflow, designed with Inforsense KDE, was used to assemble the data from multiple replicate plates of each HCS experiment including the quantitative parameters described above and chemical identities, chemical concentrations and indications of which wells were used for positive and negative controls. Scatter plots were generated representing results of high-content screening for small molecules that modulate SMN level in cytoplasm, nucleus, the number of gems, and total (integrated) SMN intensity in gems (FIG. 19c). The fold-induction of SMN intensity values for each well treated with a library chemical were normalized to the SMN average intensity in multiple negative control wells per plate.

The high content screening data were further coupled with compound structural data, then stored and analyzed using the IDBS Activity Base (version 7.1) software package. Molecular clustering was performed using the maximum dissimilarity method in the Scitegic Pipeline Pilot software. Chemical fingerprint descriptors are used by the algorithm to identify clusters using comparative Tanimoto similarity coefficients. The pharmacological data has been collected from various sources, such as Leadscope Marketed Drugs Database, DrugBank, Pharmaprojects and the chemical vendor.

Since it is not known which form of SMN increase is the most essential, hits that scored in a duplicate at least in two different parameters greater than 1.3 fold at any of the three concentrations were selected (FIG. 19d). Total of 384 primary hits that increased SMN levels were re-tested in a secondary assay in 8-point dose-response curves done in a duplicate. Further, 220 compounds were screened in the absence of anti-SMN primary antibody with the same assay conditions, to filter out the fluorescent compounds. There were 32 fluorescent compounds, which emitted at 488 wavelength. In the end, as outlined in a hit screening tree (FIG. 19e), 188 compounds with confirmed hits with activity validated in SMA patient fibroblasts.

### Compound Hit Classes, Following on the Leading Biological Pathways.

Final 188 hit compounds were analyzed and clustered based on their chemical structure and based on annotated mechanism of action and cellular targets. Molecular clustering was performed using the maximum dissimilarity method in the Scitegic Pipeline Pilot software. Chemical fingerprint descriptors are used by the algorithm to identify clusters using comparative Tanimoto similarity coefficients. Representative active chemical scaffold of the series that were identified (data not shown), however compounds were not followed unless their mechanism of action was annotated.

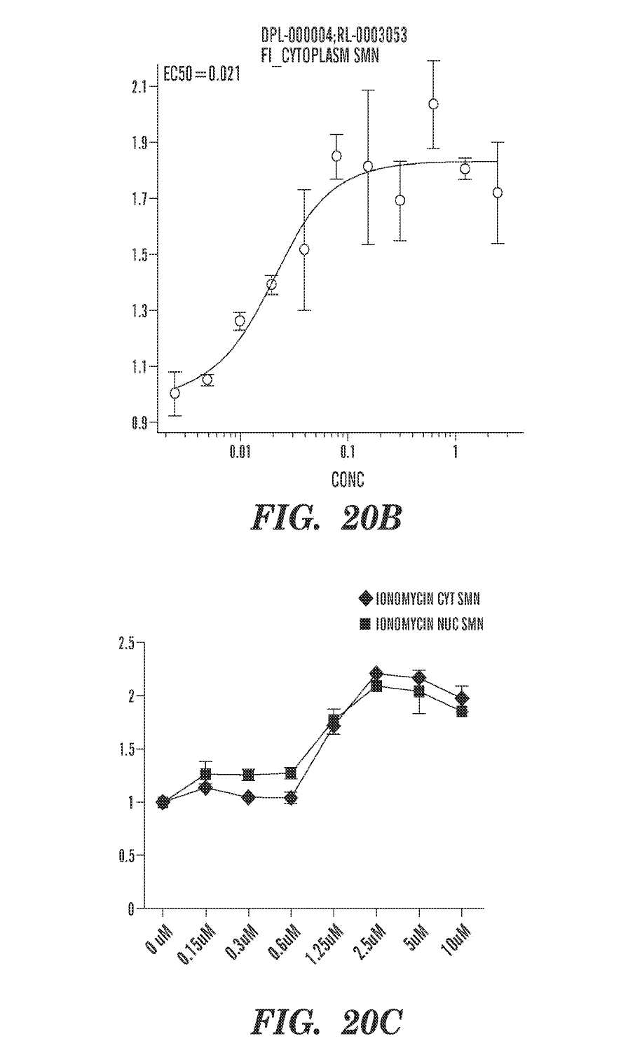

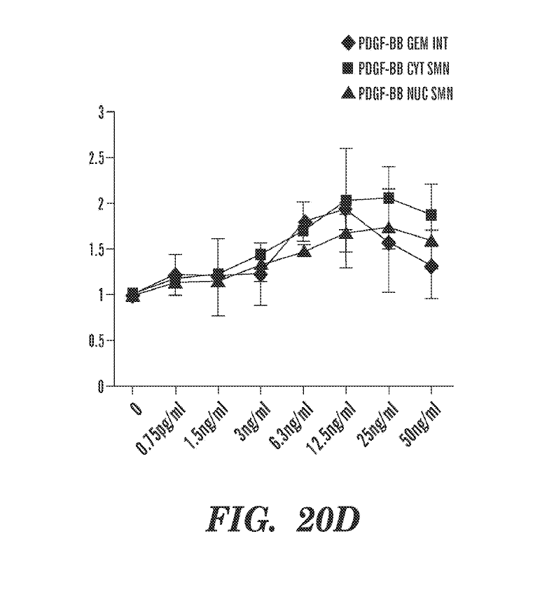

Confirmed hits were chosen for further work based again on attempts to identify pathways that could be physiologically regulating SMN levels. The pharmacological data has been collected from various sources, such as Leadscope Marketed Drugs Database, DrugBank, Pharmaprojects and the chemical vendor. Compounds that had putative druggable targets were focused on. One hit class that consistently emerged were Na, K-ATPase inhibitors such as cardiac glycoside-ouabain. Ouabain was recently identified in blood, adrenal glands, and hypothalamus of mammals and was proposed to act as endogenous cardiotonic steroid. Follow on compounds were tested in fibroblasts derived from SMA patients using optimized Methanol/Acetone fixation protocol without the use of the robotics, however data were obtained by Opera Evoteck system. Multiple cardiac glycosides scored as hits, with their ability to increase SMN levels correlating well with their IC50s towards this transporter (FIG. 20 a, b). Two different intracellular signaling pathways that may mediate the effects of Na/K ATPase inhibition were probed; the first being increased intracellular Ca²⁺. Ca²⁺ ionophore Ionomycin was tested and it was able to increase SMN level (FIGS. 2 and 20c). In addition, other types of compounds that increase intracellular Ca²⁺ like Thapsigargin and Calcimycin were also tested and were generally active (FIG. 2).

### Example 25

### PDGF Physiological Modulation of SMN is Receptor Mediated

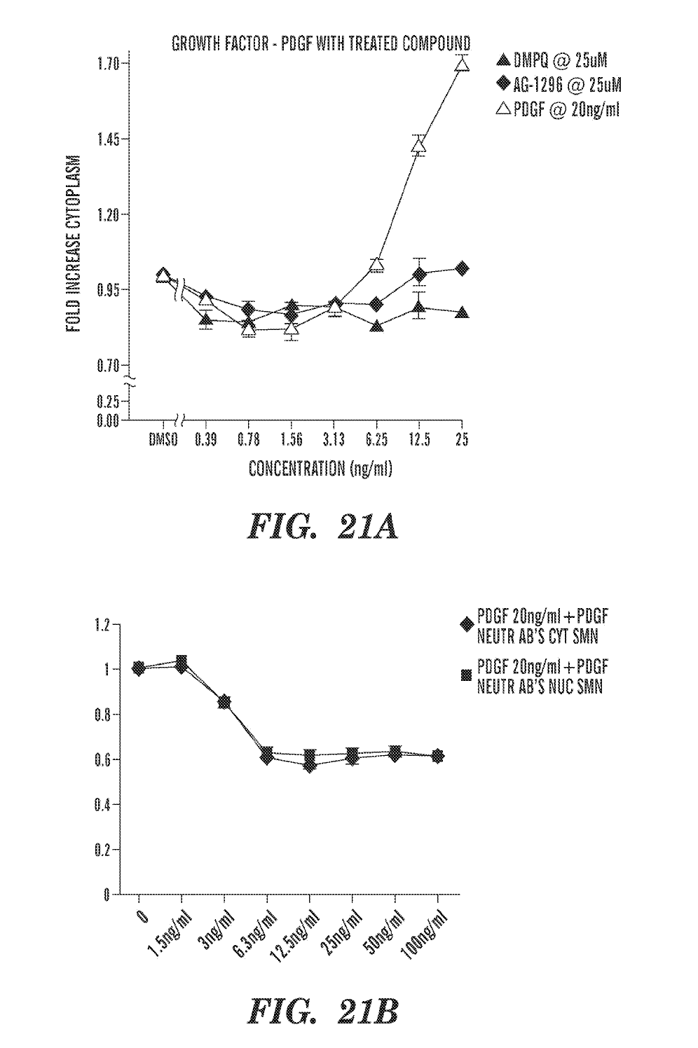

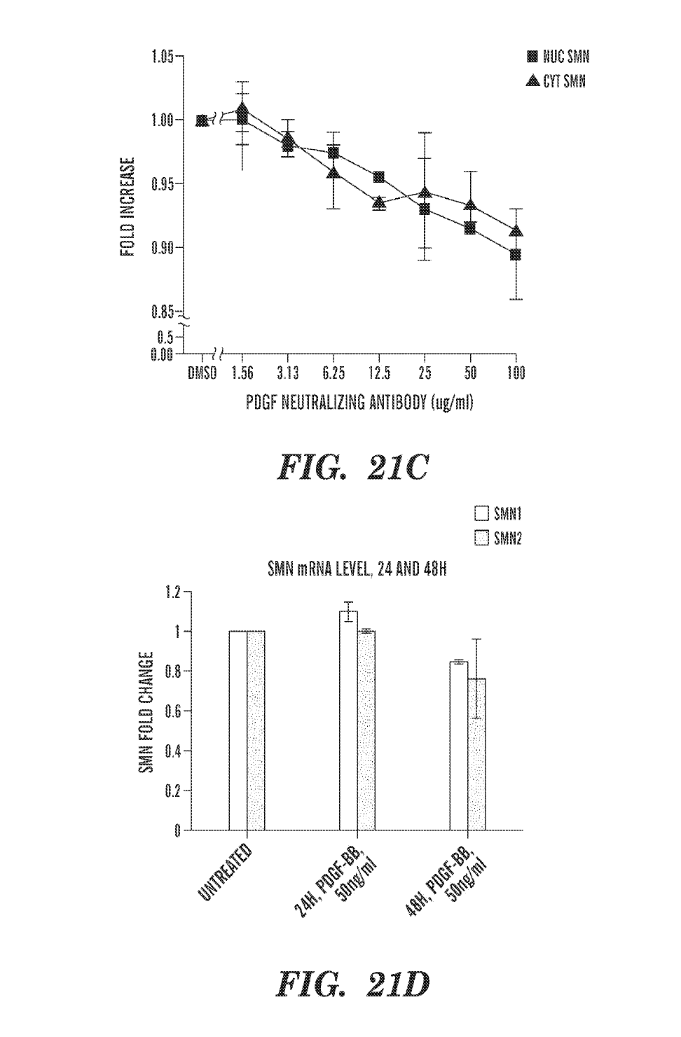

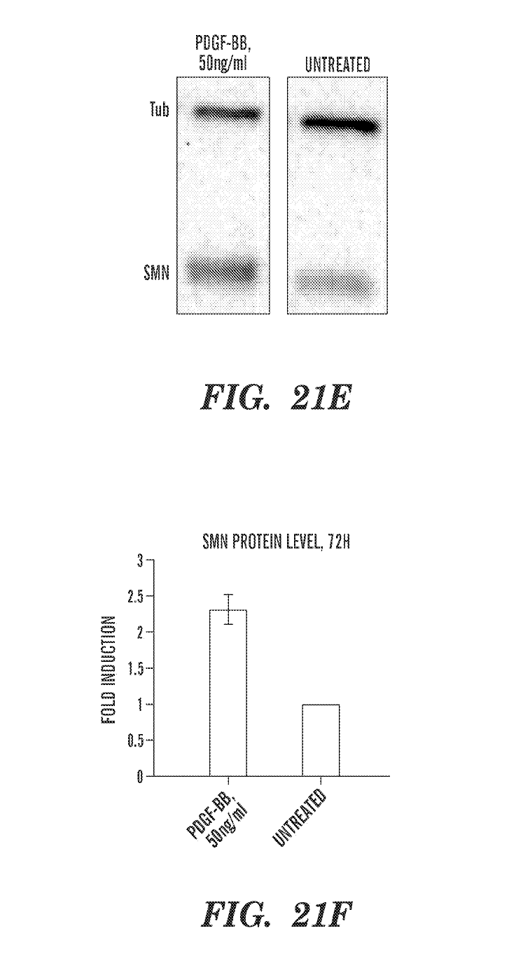

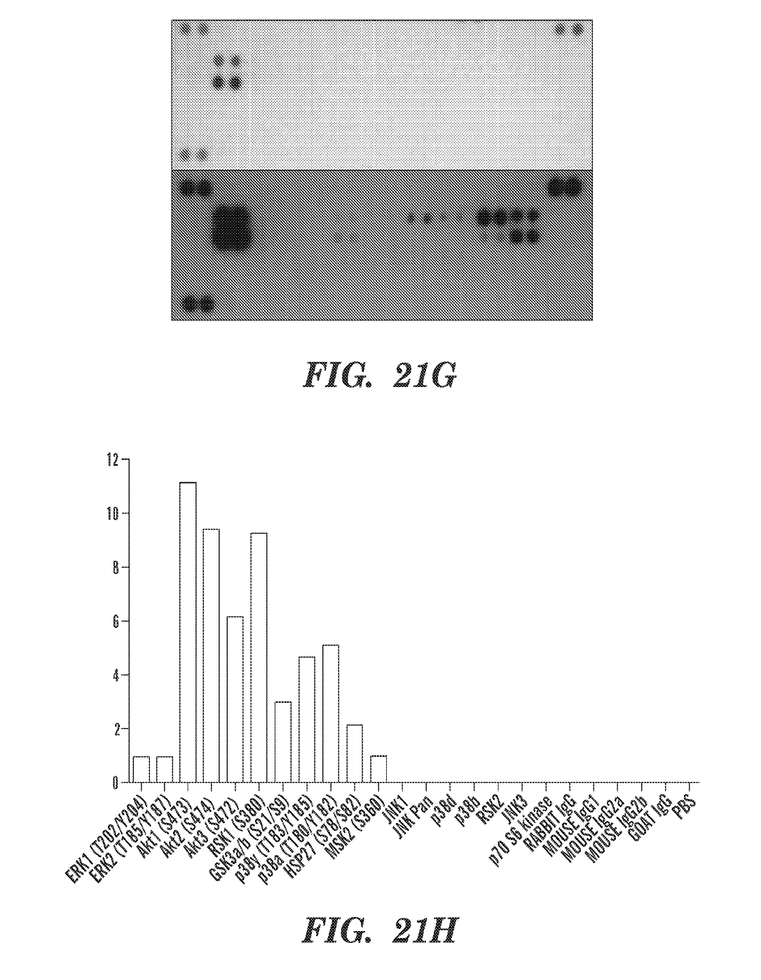

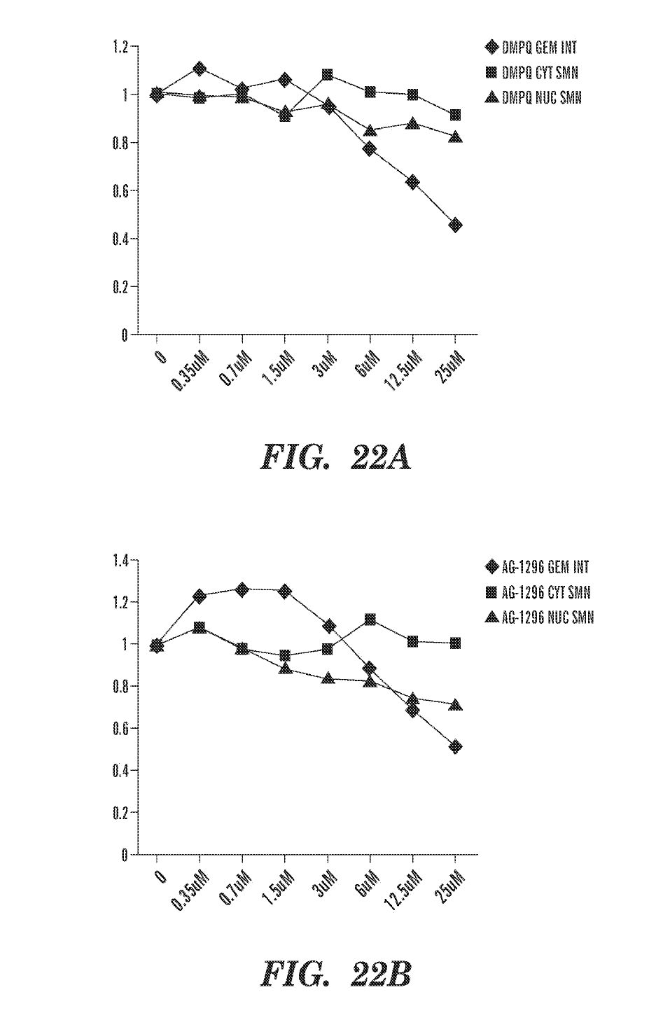

Further interest in Ouabain was also supported by its recently emerging role as activator of the receptor tyrosine kinase (RTK) signaling pathways, suggesting another possible mechanism involved in regulating SMN protein levels. In support of the second hypothesis, during screening anisomycin and coumermycin—compounds that activate kinases downstream of RTK—were also detected, both were able to increase SMN level at 0.1 uM and at 10 uM accordingly (data not shown). So next the question of whether growth factors that specifically stimulate pathways downstream of RTK like PI-3 and MAPK would be able to increase SMN level was addressed. Several different ligands for RTKs were tested and identified that PDGF-BB was the most potent growth factor that elevated SMN protein levels (FIG. 20d). Although there is much more known about growth factor specificity and downstream pathways that they activate, whether PDGF effect on SMN was mediated through the binding to the PDGFR was tested next. The SMN-increasing effects of PDGF were blocked by DMPQ and AG-1236, two small molecules inhibitors of the PDGF receptor (FIG. 21a). In addition PDGF neutralizing antibody titration were performed in the cells incubated with 20 ng/ml platelet-derived PDGF. Depending on the antibody concentration, gradient of SMN level decrease reaching 40% was observed (FIG. 21b). Moreover, as might be predicted, since there are appreciable levels of PDGF in the media serum in which the fibroblasts are normally maintained, simply adding PDGFR inhibitors DMPQ and AG-1296 to the media decreases the “basal” level of SMN in these cells down to 15-20%, (FIG. 22). Same effect was reproduced by adding PDGF neutralizing antibody to the media, confirming that PDGF can be a physiologically relevant regulator of SMN levels (FIG. 21c). In order to understand, at what step of SMN protein production such regulation occurs, a qRT-PCR was performed to assess the effect of the PDGF-BB on the level of SMN1 and SMN2 transcripts over the course of three days at the concentration of 50 ng/ml, that was efficiently increasing SMN protein level as seeing by the immunofluorescence on the Opera and in addition confirmed by western blot (FIG. 21e, f). Surprisingly, there was little or no increase in SMN transcripts, indicating that PDGF regulates SMN post-transcriptionally (FIG. 21d). Phospho-Mitogen-activated Protein Kinase antibody array was used and relative phosphorylation of 19 kinases was compared between the PDGF treated sample and the MOCK treated sample (FIG. 21g). It was observed that in these cells PDGF stimulation primarily lead to phosphorylation of Akt, RSK1, p38 and GSKa/b, having little effect on Erk1 and 2 kinases (FIG. 21h).

### Example 26

### Inhibition of PI-3/Akt Kinase Pathway Blocks PDGF Stimulated SMN Increase in all Cellular Compartments

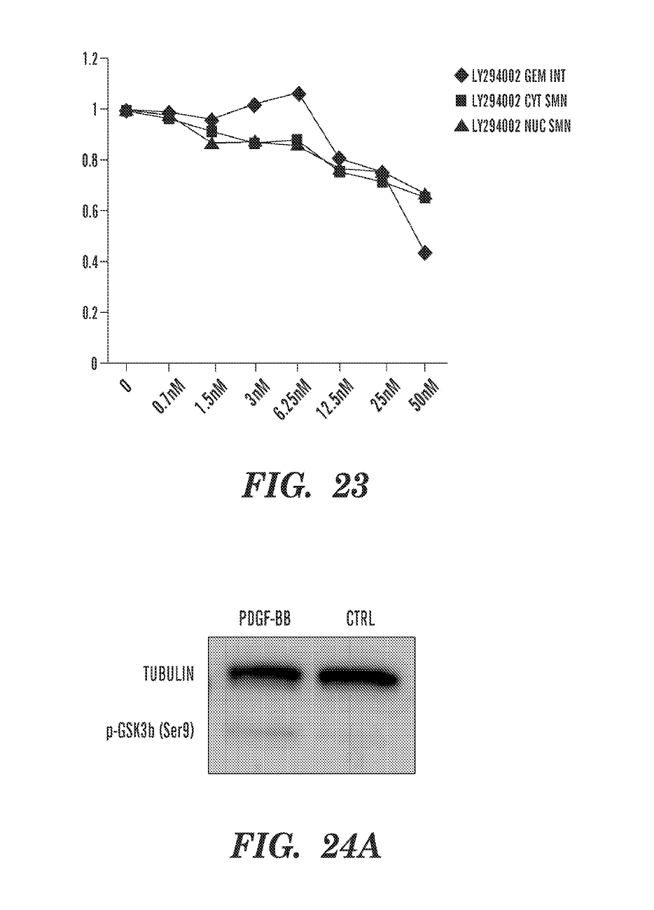

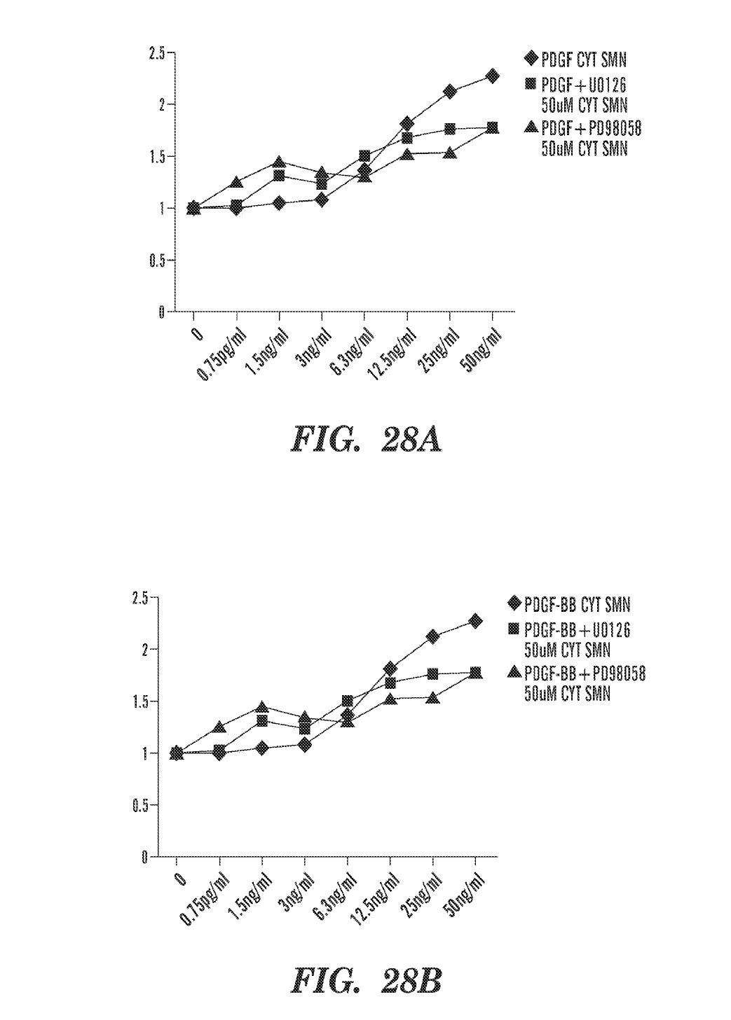

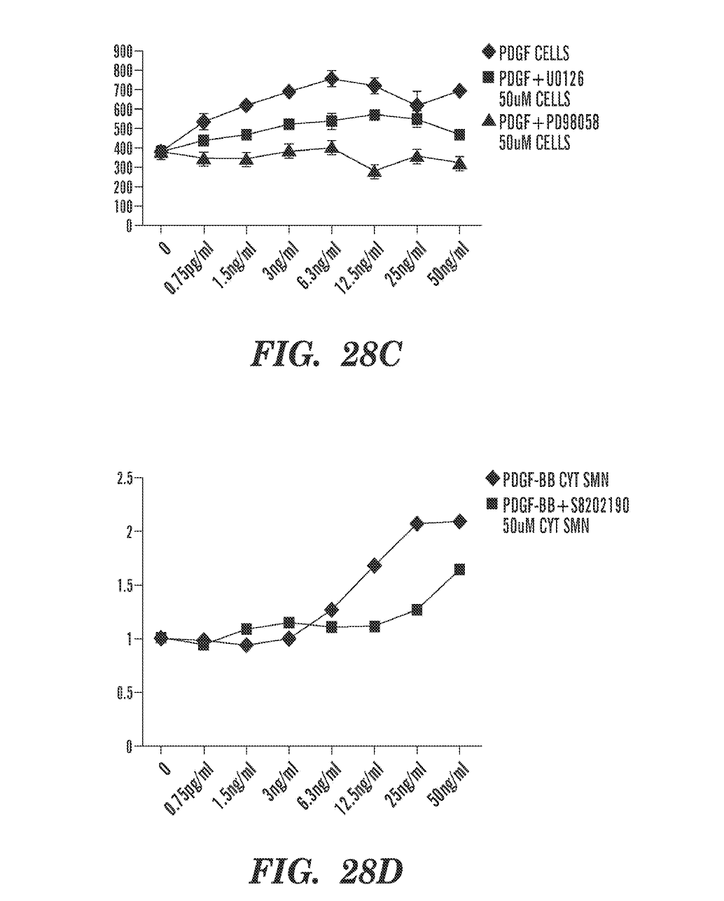

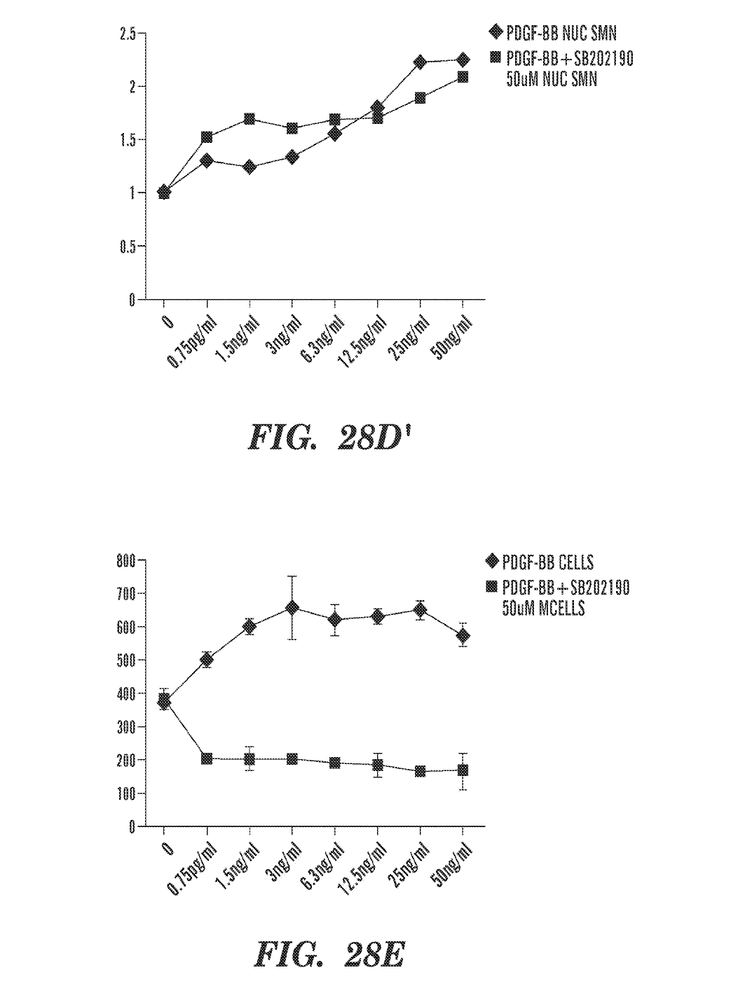

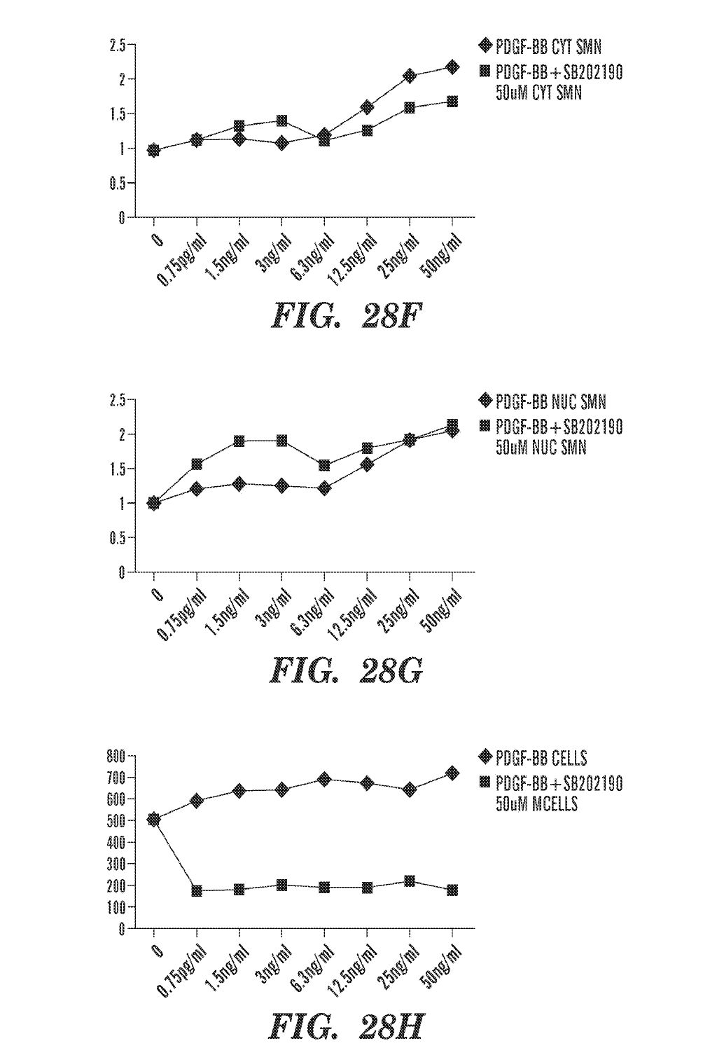

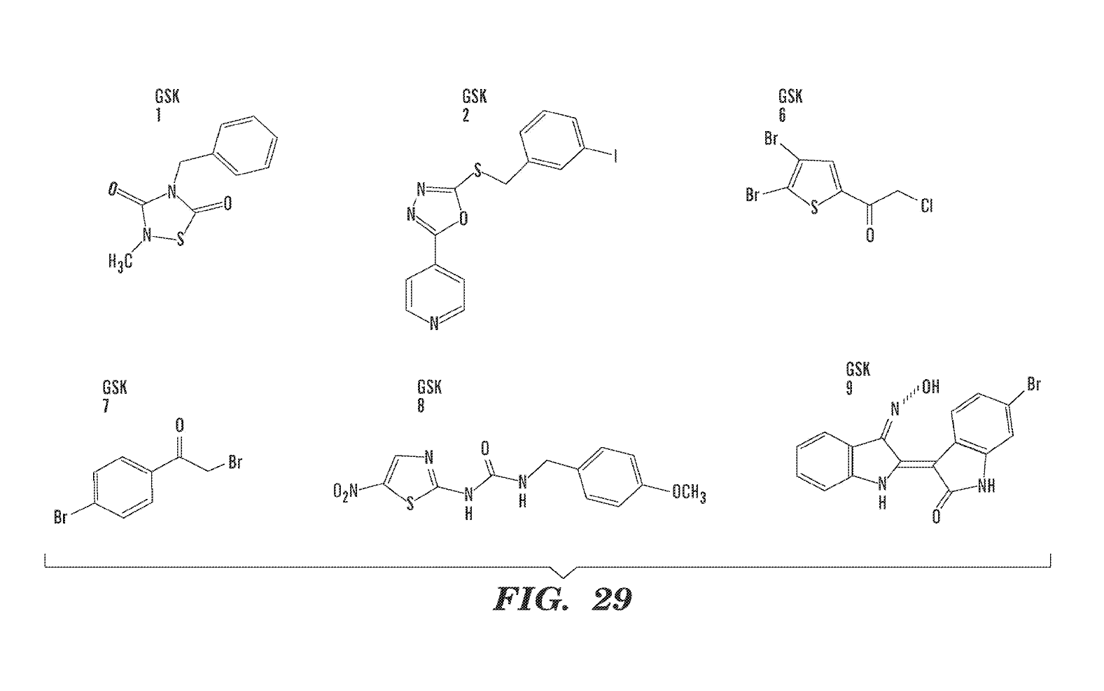

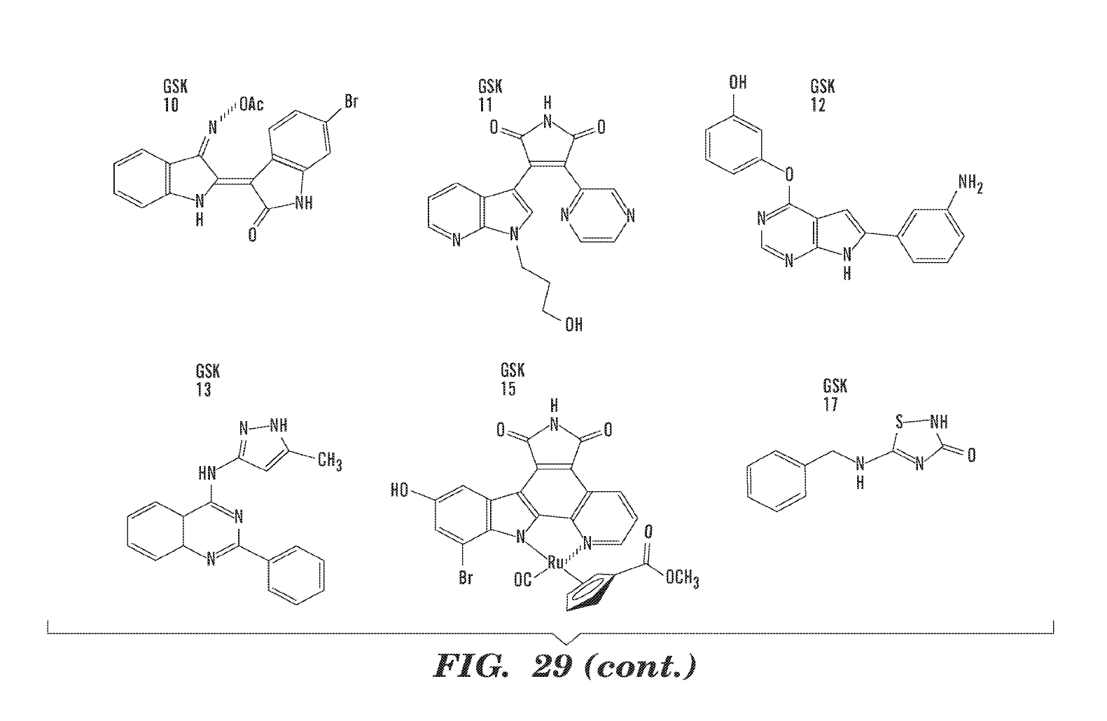

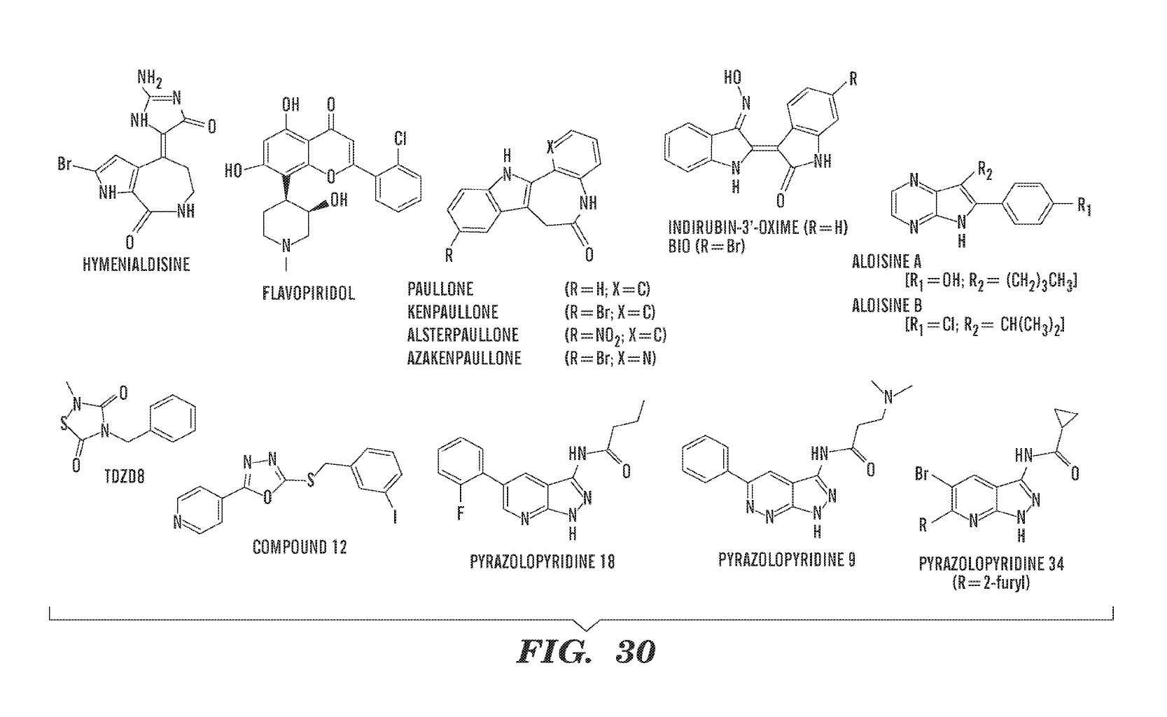

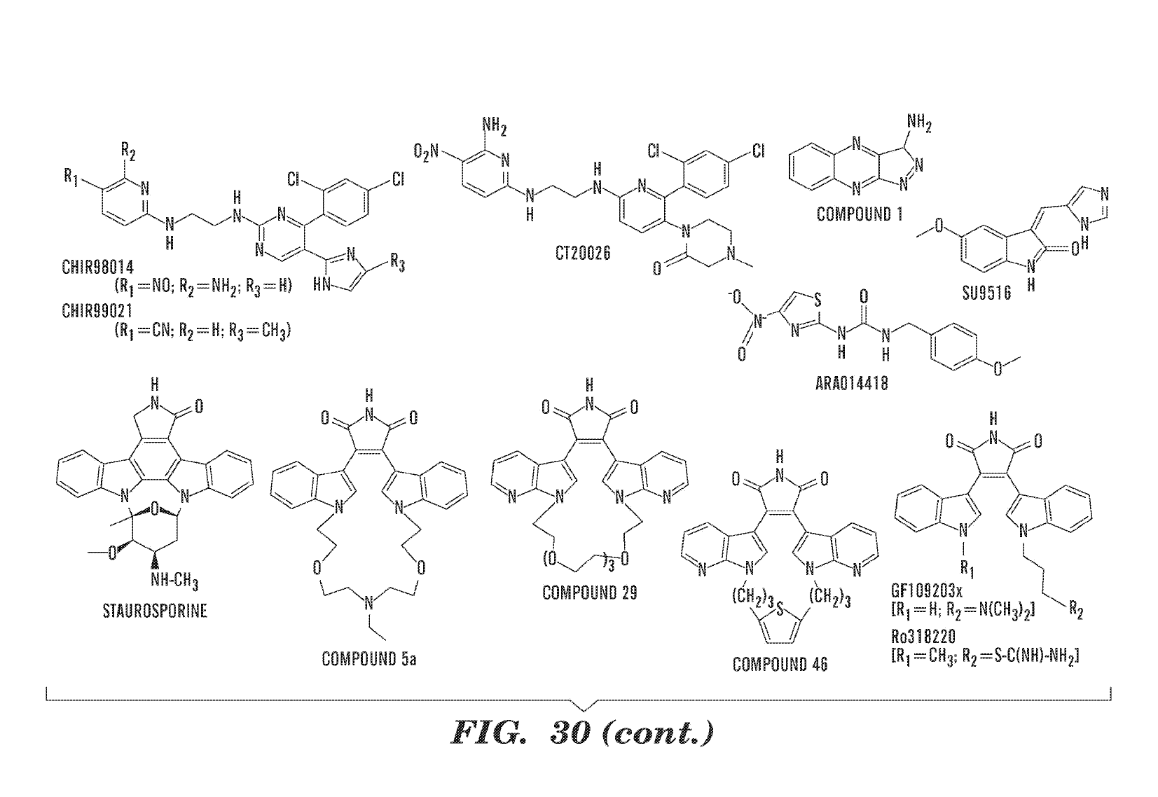

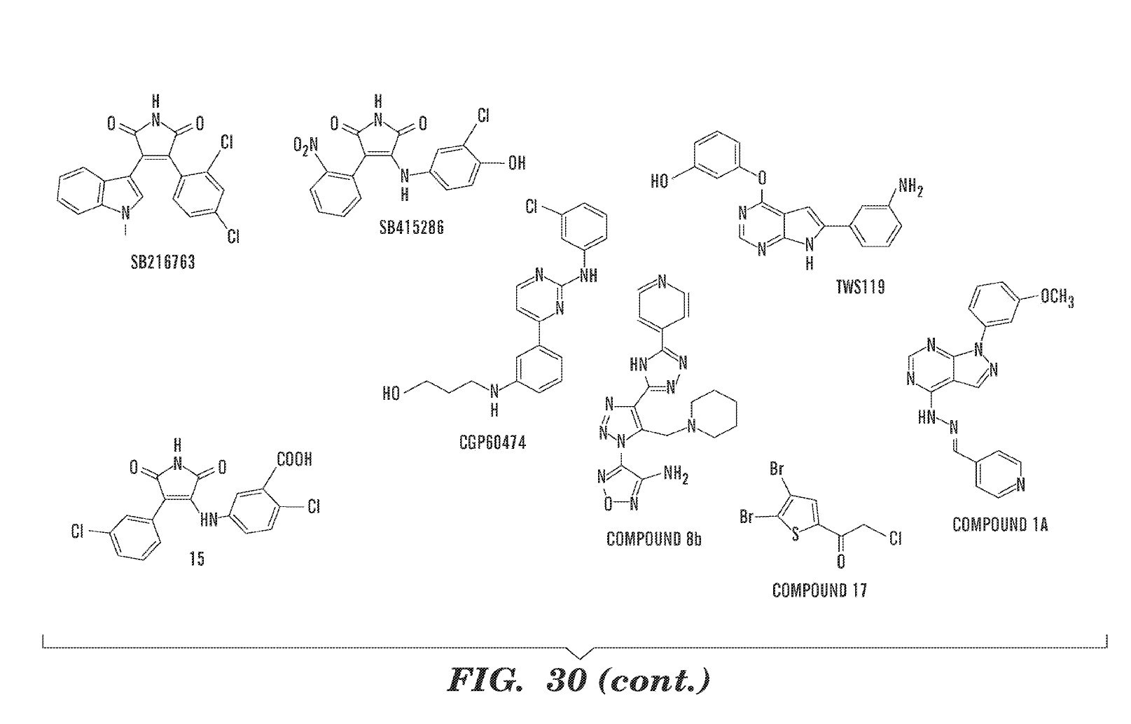

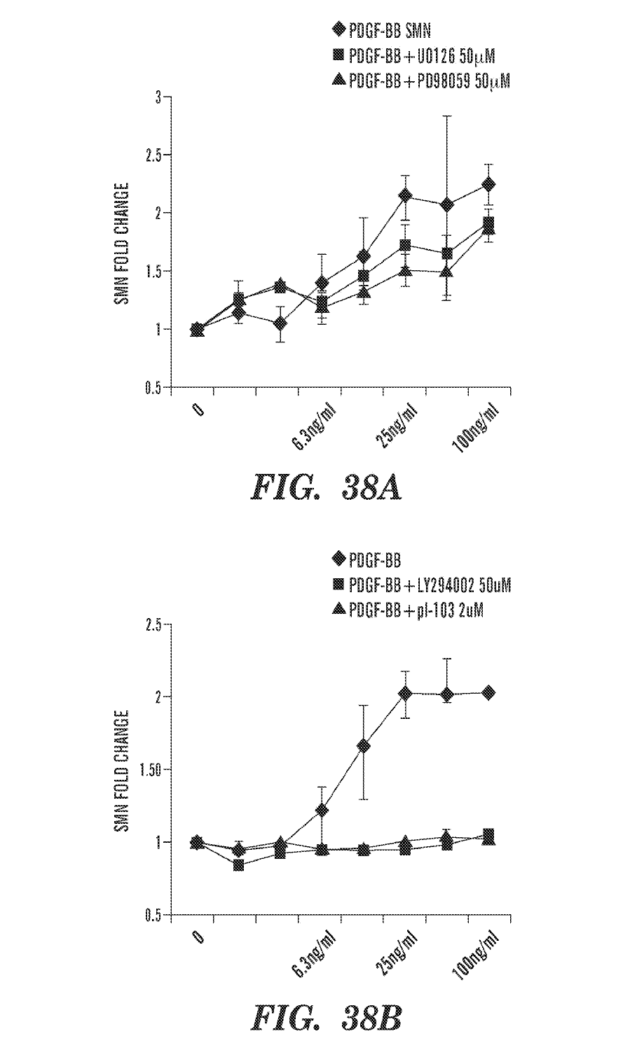

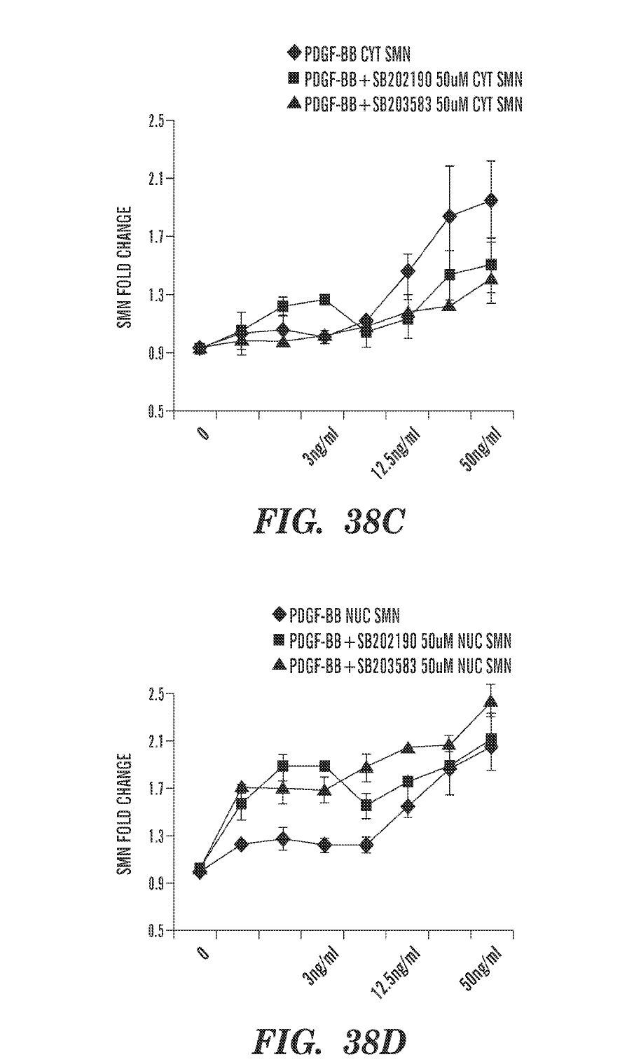

In order to find what pathway downstream of PDGFR is responsible for SMN regulation, druggable kinases downstream of PDGFR prior to the PDGF stimulation were individually inhibited, to see which inhibitor will be able to block the PDGF effect on SMN increase. A range of concentrations were tested in order not to miss the effects. SMN level were evaluated by Opera Evoteck in each cellular compartment and to assess the cell numbers that are summarized in the Table 3. It was observed that the PDGF-mediated increase of Smn levels could be completely abolished by the presence of downstream PI-3 kinase inhibitor LY294002, PI 3-K inhibitor and dual inhibitor of PI-3 kinases and mTOR, compound PI-103 (FIG. 38b). To a lesser degree SMN increase by PDGF was prevented by the Rapamycin. Since RSK1 kinase was phosphorylated in the kinase array, the only available specific RSK1 inhibitor SL-0101 was tested, and it didn't have an inhibitory effect. Pre-treatment with p38 inhibitors SB202190 and SB203580 lead to compartment-specific partial inhibition of SMN in the cytoplasm (FIGS. 28 and 38c) while leaving nuclear SMN unaffected (FIGS. 28 and 38d), and Erk1/2 kinase inhibitors, PD98059 and U0126, had only partially inhibited SMN increase caused by PDGF (FIG. 28, 38a). It is important to note, that p38 and Erk inhibitors also decreased the cell proliferation, similarly to PI-3 K inhibitors, but SMN inhibition was specific to PI3-Kinase inhibitors. When PI-3 kinase inhibitor was added to a regular media in which cells were maintained, it had a profound inhibitory effect on SMN levels, decreasing its values additional 40% from the baseline level (FIG. 23).

TABLE 3
Summary of analysis of downstream kinases that become
activated upon fibroblast stimulation with PDGF.

| | | Concentration | SMN | SMN | SMN GEM | Cell |
|---|---|---|---|---|---|---|
| Target | Inhibitor | nM | Cytoplam | Nuclear | Intensity | Number |
| PI-3 | LY294002 | 50 | 100% | 100% | 100% | Yes |
| Kinase | PI-3Ky | 30 | 100% | 100% | 100% | Yes |
| mTOR | Rapamycin | 0.05 | 80% | 80-100 | 80-100 | |
| Erk | U0126 | 50 | 20-30% | 10 | 10 | No |
| | PD98059 | 50 | 20% | 20 | 20 | Yes |
| P38 | SB203580 | 25, 50 | 20 | 0% | some | Yes |
| | SB202190 | 25, 50 | 20% | 0% | some | Yes |
| RSK1 | SL-0101 | 50 | 20 | 20 | 20 | NO |
| (p90) | | | | | | |

### Example 27

### GSK Inhibitors Increase SMN level in SMA Patient Fibroblasts

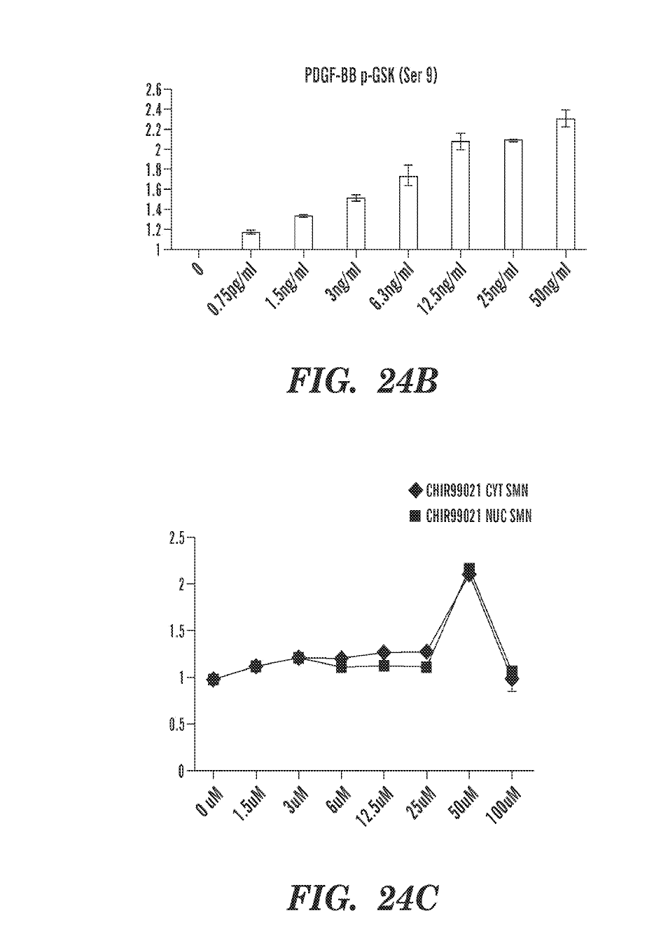

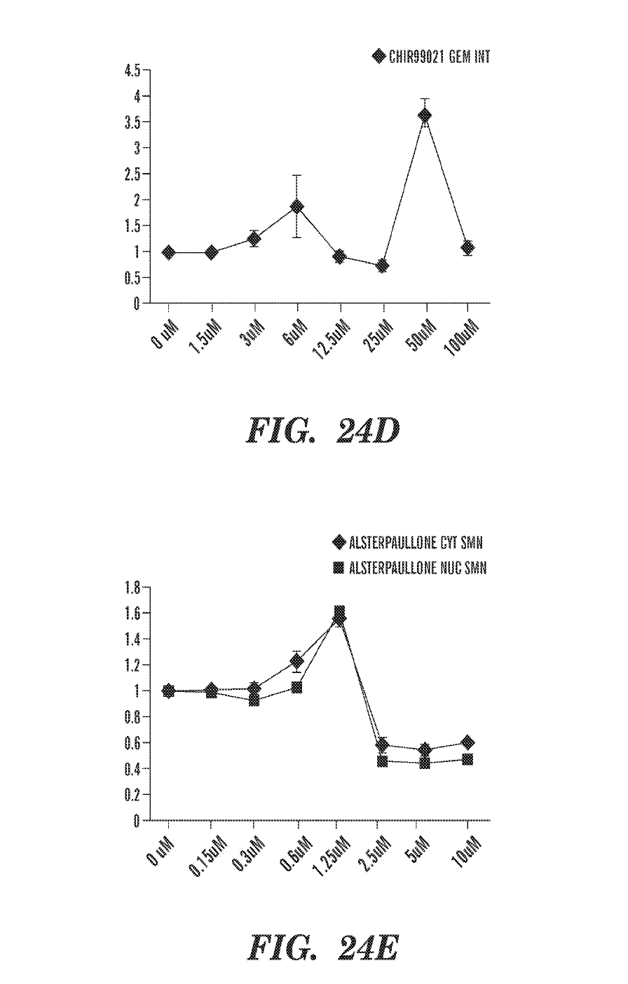

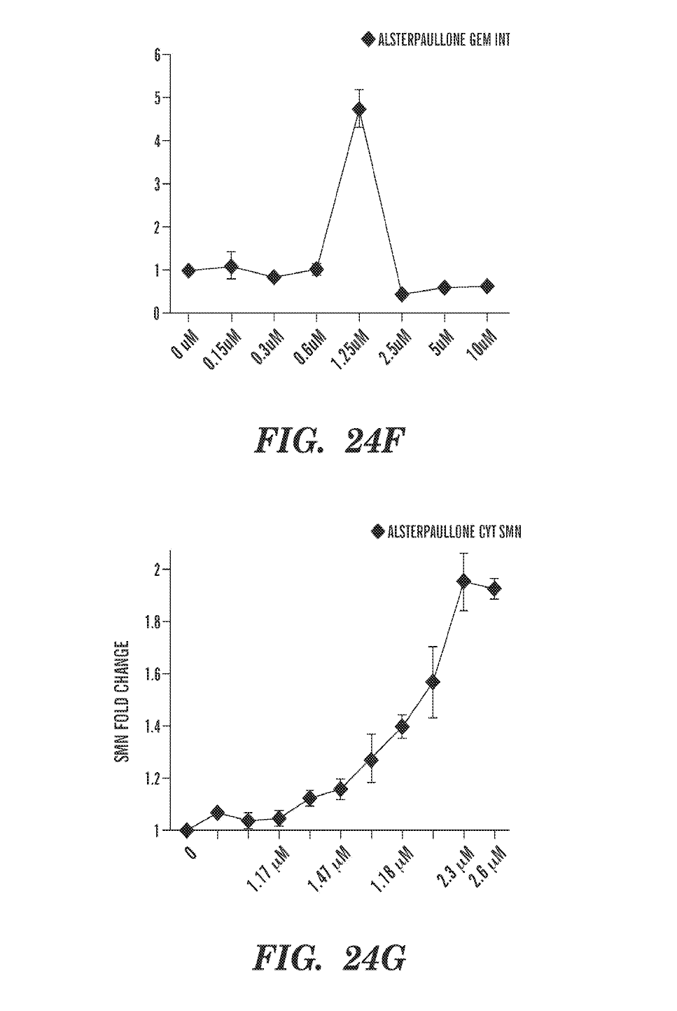

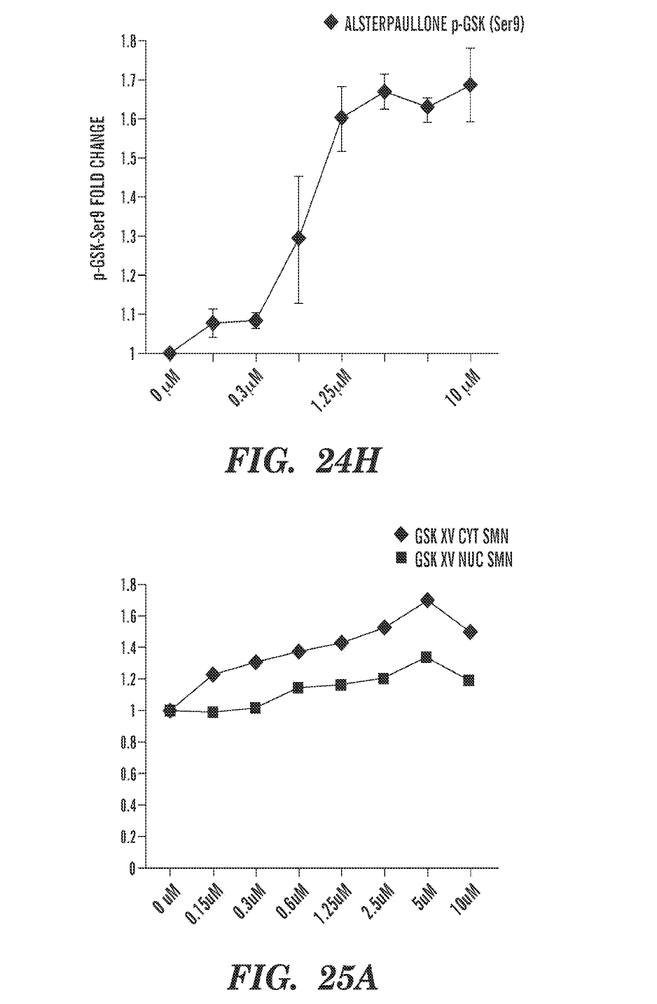

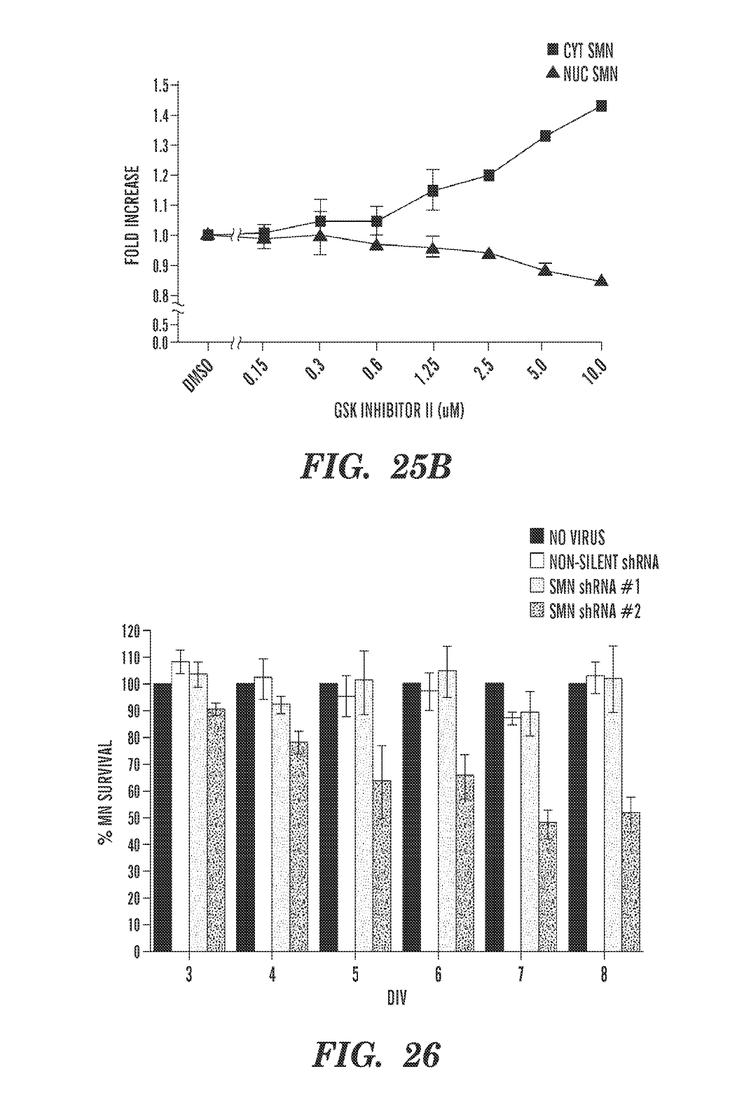

Another kinase downstream of different RTKs, including PDGFR, and of PI3-K/AKT signaling is GSK313 that previously has been reported to be involved in ALS, neurodegenerative disorders associated with motor neuron death. Usually kinase phosphorylation acts as an activation switch, in contrast to this regulatory pattern, phosphorylation of GSK on Ser-9 leads to the inhibition of its activity, thereby releasing the block from multiple downstream targets that are being controlled by GSK kinase. Activation of PI3-K/Akt kinase pathway is known to have a strong inhibitory effect on GSK. Whether such an event takes place in the system outlined herein, cellular lysates obtained from patients fibroblasts were stimulated with 50 ng/ml of PDGF-BB for 1 hour by a western blot (FIG. 24a). It was also observed that PDGF phosphorylates GSK313 in a dose-dependent manner, inhibiting its activity, potentially explaining how it stimulates SMN levels (FIG. 24b). Interestingly, similar to SMN, p-GSK kinase phosphorylated on the Ser9 is also distributed throughout the cell, but was seen to concentrates in the nuclear Gem-like structures (data not shown). However the patterns of protein localization of SMN and p-GSK do not strictly overlap as merge of confocal image of Ser9 p-GSK in a 546 channel and SMN 488 showed no exclusive co-localization (data not shown). Consistent with the screening data, there were several inhibitors of GSK that increase SMN levels in human fibroblasts (FIG. 25a,b). Most active compounds were CHIR99021 (FIG. 24b,c) and Alsterpaullone (FIG. 24d,e).

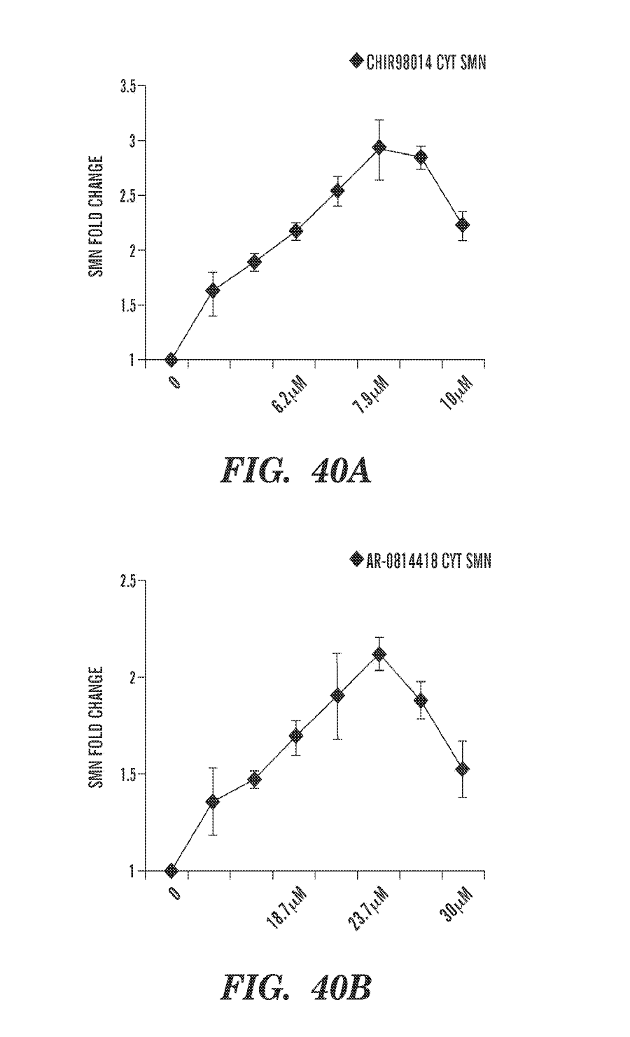

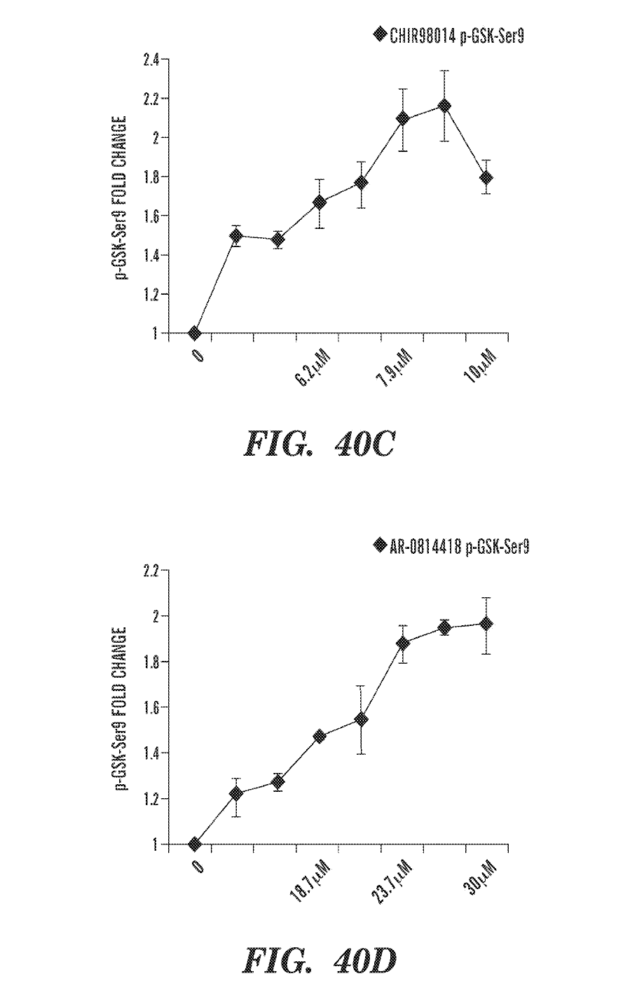

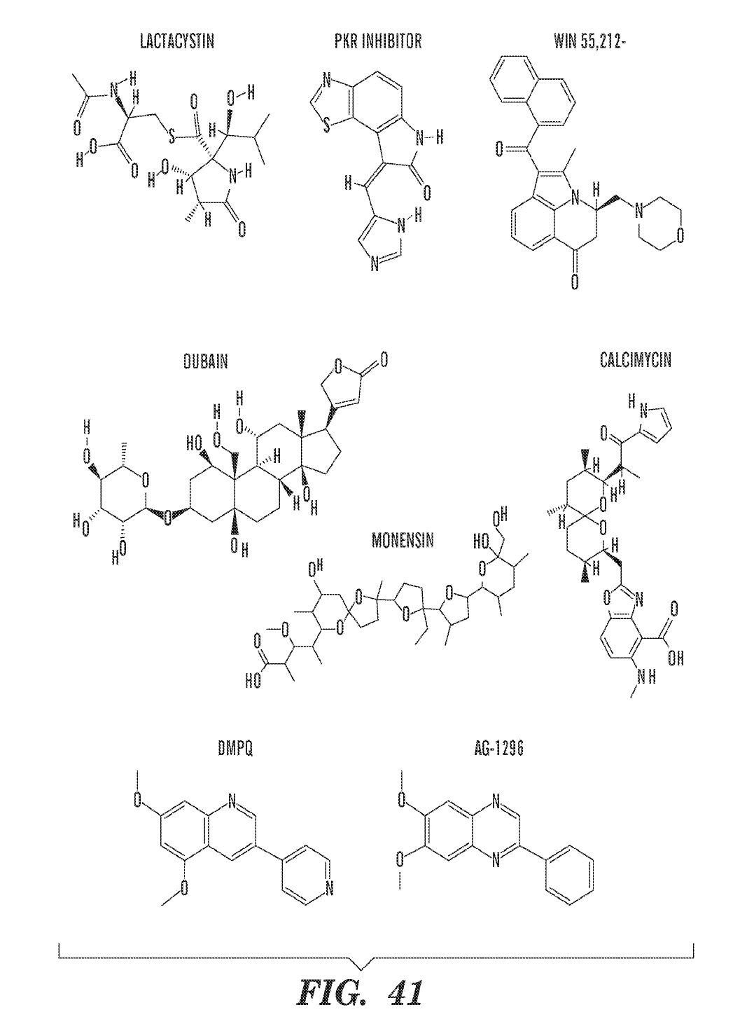

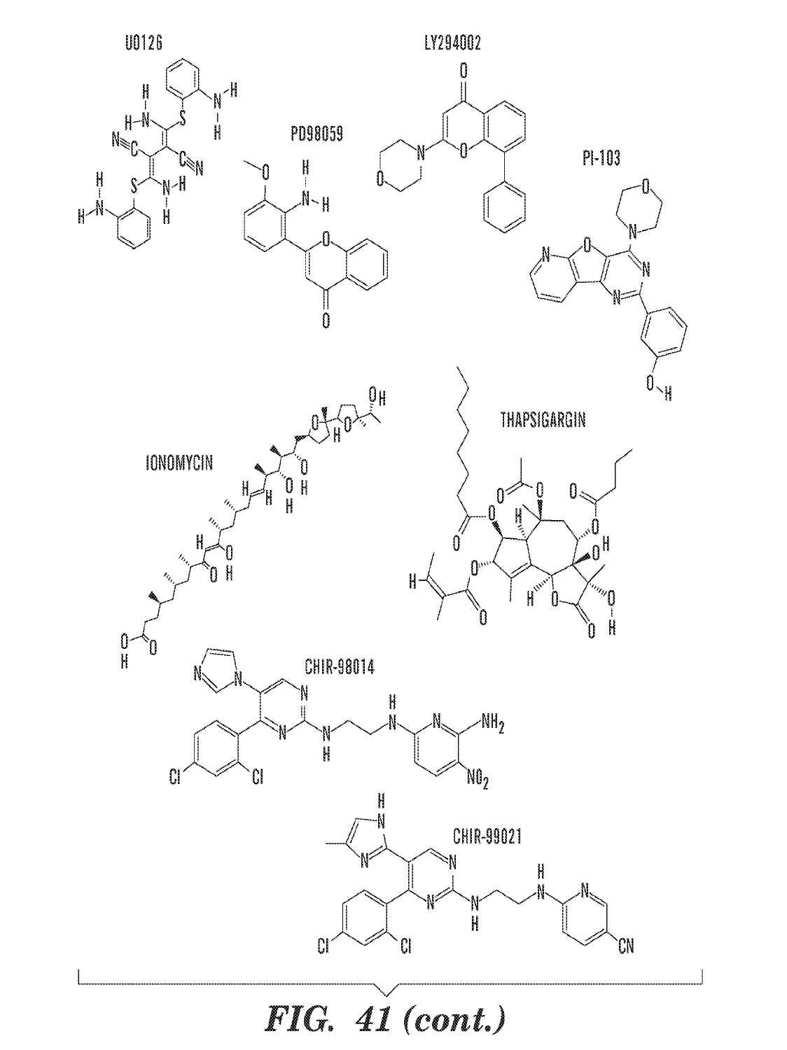

Interestingly, at low concentrations, alsterpaullone, a dual GSK-313, cyclin-dependent kinase inhibitor and its analog 2-Cyano-alsterpuallone (data not shown) were able to elevate SMN in patient fibroblasts (FIGS. 24e and 24g). When immunofluorescent staining assay in fibroblasts incubated with different doses of Alsterpaullone was performed, levels of p-GSK-Ser9 phosphorylations were also seen to increase in a dose dependent manner with this compound (FIG. 2h). We also tested an extensive list of other GSK kinase inhibitors, and several of them, most selective to date, were able to increase SMN (FIGS. 4a and 40b) and promoted p-GSK-Ser9 phosphorylation level increase (FIGS. 42c and 40d).

### Example 28

### Alsterpaullone Elevates Level of SMN in ES-Cell Derived Neurons and Corrects Survival Differences in ES-Cell Derived Motor Neurons with SMN Knockdown

As described herein, screening revealed many different classes of compounds that are capable of elevating SMN in different intracellular compartments. Because it is not entirely clear where the functional SMN resides (and whether the functional SMN has particular post-translational modification), potential of these compounds to promote survival of motor neurons with reduced Smn levels was investigated.

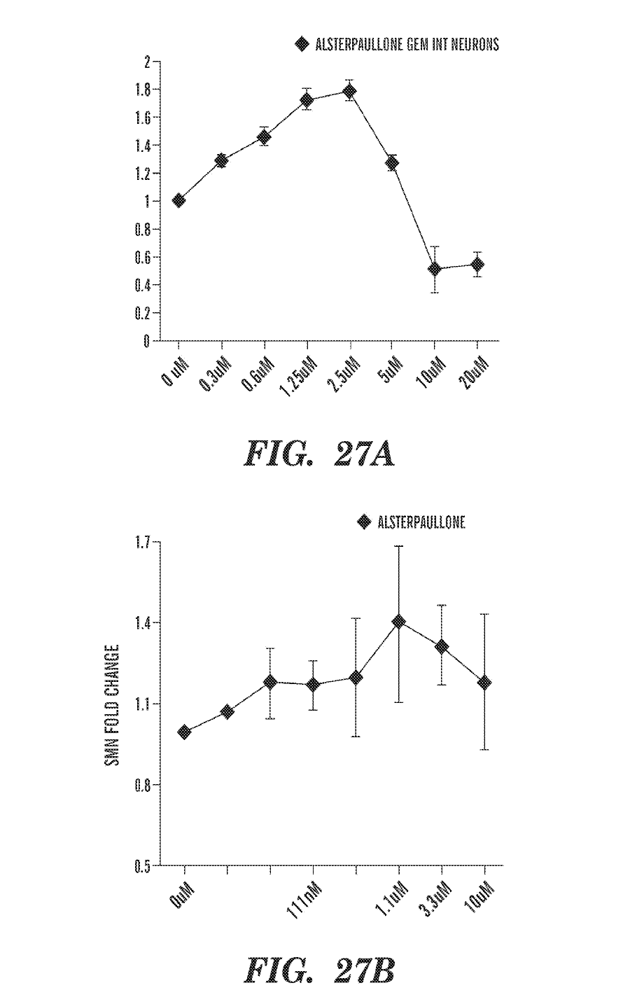

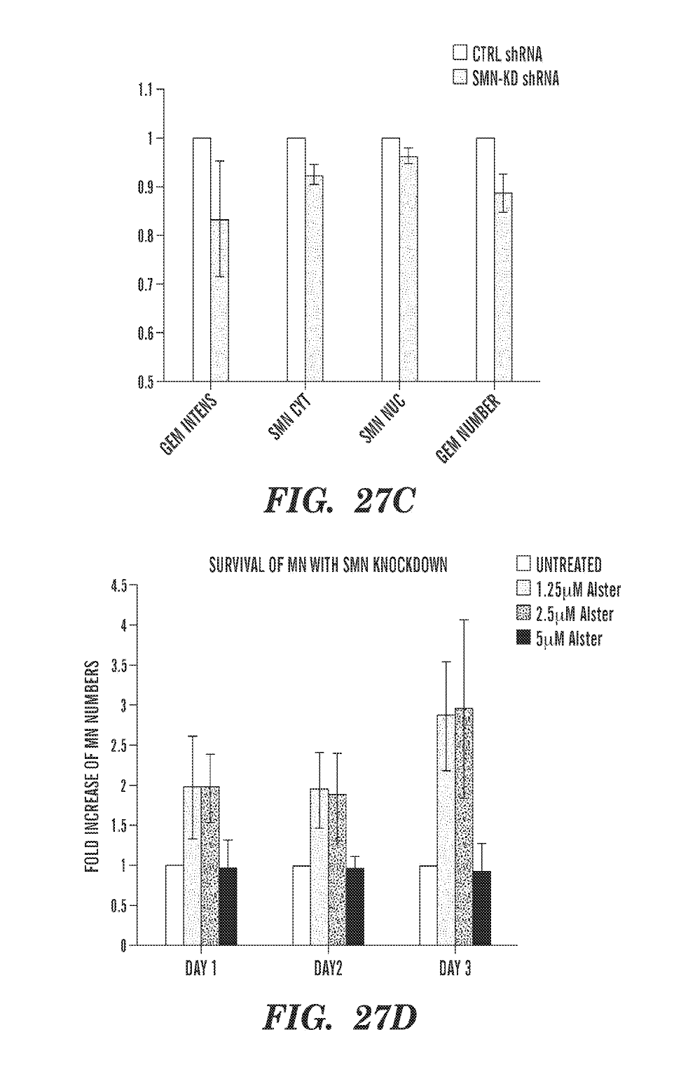

Motor neurons were produced from mouse ES-cells expressing cherry fluorescent protein (CFP) under the control of the motor neuron-selective Hb9 promoter, as previously described (Wichterle et al., 2002). Alsterpaullone (as well as several other GSK-3β inhibitors) were able to elevate Smn in the mouse motor neurons (data not shown). The main increase was observed in Gem Intensity parameter (FIGS. 27a and 27b).

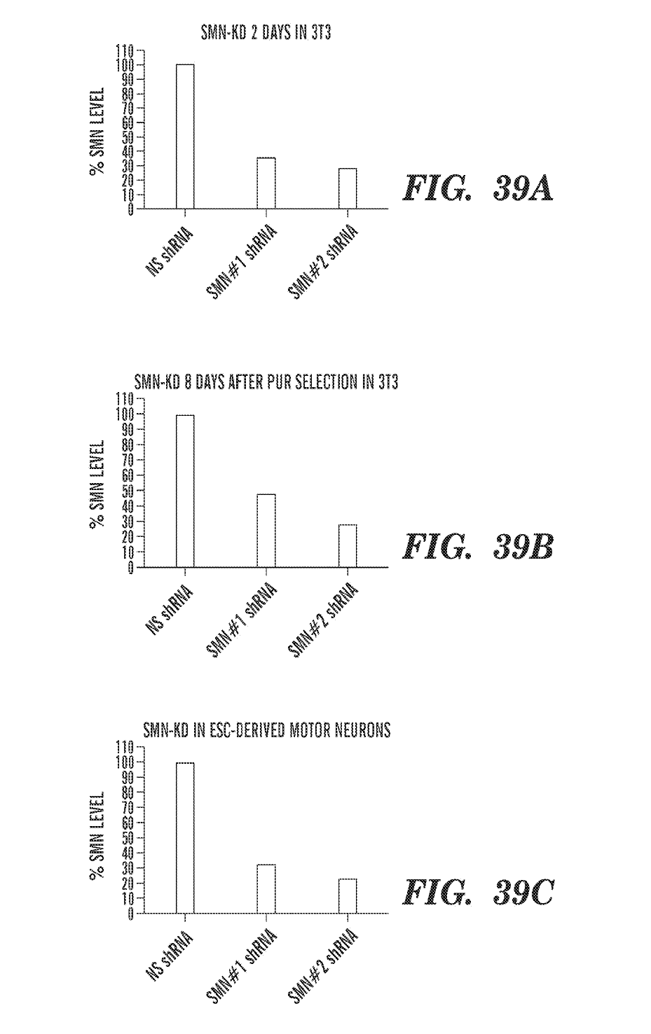

To test for survival, motor neurons were infected with three lentiviral constructs acquired from Open Biosystems (pGIPZ): a non-silencing (NS) shRNA and two with unique shRNA sequences directed against the Smn transcript. Virtually all of the motor neurons were infected under the conditions chosen for these experiments. Quantification of SMN knockdown were made in 3T3 cells (FIGS. 39a-39b) and in motor neurons by sorting ES cell derived motor neurons (cherry positive) infected with the pGIPZ lentiviruses (GFP positive cells) by FACS and quantifying the amount of SMN by Western blot. Uninfected and NS pGIPZ infected motor neurons had approximately the same level of SMN. In contrast, SMN#1 pGIPZ infected motor neurons had a greater than 65% knockdown in the level of SMN, with SMN#2 pGIPZ infected motor neurons having approximately a 75% knockdown of SMN protein (FIG. 39c).

Effect of reduced SMN levels on motor neuron survival was investigated next. Motor neurons were plated and infected with the various lentiviral constructs. After 4 days, reduced motor neuron survival was observed with SMN#2 pGIPZ virus, but not with SMN#1 (FIG. 26). Therefore motor neurons with an average level of SMN less than 30% of wildtype SMN experienced a significantly higher level of cell death. These results suggest that reducing SMN compromises motor neuron survival and that there is a very steep dose-response relationship between survival and SMN level. This may be reasonable since, according to available data, parents of children with SMA are phenotypically unaffected even though they have only 50% of normal SMN levels (citation based on clinical studies). To test the effects of SMN increasing compounds, we added them to motor neurons two days after lentiviral infection. Over the course of the subsequent three days, we counted live cherry expressing motor neurons on a daily basis. In the presence of 1.25 μM and 2.5 μM Alsterpaullone, the survival of motor neurons after SMN knockdown was similar compared to that of control cells with normal SMN level treated with Alsterpaullone (data not shown). If it was observed that by the second day 1.25 and 2.5 uM Alsterpaullone treatment produced twice the amount of surviving cells, and by the third day survival has reached three fold difference compared to MN treated with DMSO (FIG. 27d).

### Example 29

### Activators of RTK Signaling Elevate SMN Levels in Mouse Motor Neurons

GFP expressing motor neurons were derived from mouse ES cells as described below. After growing for approximately 1 week, motor neurons were treated with allsterpaullone. Two days after treatment with allsterpaullone, cells were fixed and stained with SMN antibody or GFP antibody. Increased SMN levels were seen in cells treated with allsterpaullone (data not shown).

### Example 30

### Activators of RTK Signaling Elevate SMN Levels in Human Motor Neurons (iPS Line)

GFP expressing motor neurons were derived from an ALS patient iPS cells as described below. After growing for approximately 1 week, motor neurons were treated with allsterpaullone. Two days after treatment with allsterpaullone, cells were fixed and stained with SMN antibody or GFP antibody. Increased SMN levels were seen in cells treated with allsterpaullone (data not shown).

### Example 31

### Generation of Motor Neurons

Derivation of ESC Lines from SOD1G93A ALS Mice

ESC lines were derived from embryos generated by crossing hemizygous mice carrying either pathogenic (mutant) SOD1^{G93A} transgene or the non-pathogenic (wild-type) SOD1^{WT} transgene with hemizygous mice carrying a transgenic reporter gene in which expression of the green fluorescent protein (GFP) is driven by promoter elements from Hb9 gene (Hb9::GFP). Briefly, transgenic Hb9::GFP females (Jackson lab, stock number 005029) are injected IP with 7.5 units of pregnant mares' serum (Calbiochem), followed 46-50 h later with 7.5 units of human chorionic gunadotropin (Calbiochem). After administration of human chorionic gunadotropin, females are mated with SOD1^{G93A} (Jackson lab, stock number 004435) or SOD1^{WT} (Jackson lab, stock number 002297) transgenic males. Females are killed 3 days later and blastocysts flushed from the uterine horn with mES cell medium (knockout-DMEM (GIBCO), 15% Hyclone fetal bovine serum (Hyclone), 100 units/ml penicillin and 0.1 mg/ml streptomycin (GIBCO), 2 mM glutamine (GIBCO), 100 mM nonessential amino acids (GIBCO), 55 mM 2-mercaptoethanol (GIBCO) and 100 o units/ml leukocyte inhibiting factor (Chemicon)). Blastocysts are plated individually into one 10-mm well of a tissue culture dish containing mitotically inactivated mouse embryonic fibroblasts and mouse ESC medium supplemented with MEK kinase inhibitor PD98059 (Cell Signaling). At 48 h after plating, one half volume of fresh medium is added to each culture well. Medium is changed daily after 3 d. At 4-5 d after plating, inner cell mass-derived outgrowths are dislodged with a Pasteur pipette, washed in a drop of PBS and incubated for 10 minutes in 0.25% trypsin at 37° C. ESC clumps are gently dissociated with a Pasteur pipette and transferred onto a fresh layer of fibroblasts. For routine culture, the mouse ESCs are generally split 1:6 with a solution of 0.25% trypsin (GIBCO) every 2-3 days.

Differentiation of mESC into Motor Neurons

mES cells were differentiated into motor neurons according to methods adapted from Wichterle et. al., Cell (2002), 110:385-397. Briefly, mES cells were grown to 70-80% confluence in 10-cm plates (Falcon) in ES cell medium. To form embryoid bodies, cells were washed once with PBS to eliminate mES medium and then incubated with 1 ml of 0.25% trypsin (GIBCO) for 5-10 min at 21-25° C. (RT). Cells were then resuspended in 10 ml of DM1 medium (DMEM-F12, GIBCO), 10% knockout serum (GIBCO), penicillin, streptomycin, glutamine (GIBCO) and 2-mercaptoethanol (GIBCO), counted and plated at a concentration of 200,000 cells per ml in Petri dishes (Falcon). Two days later, embryoid bodies were collected and resuspended into new Petri dishes containing DM1 medium supplemented with RAc (100 nM; stock 1 mM in DMSO, Sigma) and HhAg (100 nM). On day three day two treatment was repeated with the same concentration of RAc (100 nM) and high concentration of HhAg (1 μM). On day 6, the embryoid bodies were collected in a 15-ml Falcon tube, and incubated in Earle's balanced salt solution with 20 units of Papain and 1,000 units of DNase I (Worthington Biochemical) for 10 min at 37° C. The mixture was then triturated with a 10-ml pipette and centrifugated for 5 min at 800 rpm. The resulting cell pellet was washed with PBS and resuspended in F12 medium (F12 medium, GIBCO) with 2% fetal bovine serum (GIBCO), B-27 supplement (GIBCO), (GDNF and BDNF, 20 ng ml⁻¹, R&D Systems). The cells were counted and plated on poly-ornithine coated 96-well plates (VWR).

Derivation of Motor Neurons from Induced Pluripotent Stem (IPS) Cells Generated from SMA Patients.

Induced pluripotent stem cells were generated from an SMA patient fibroblast line from Coriell (GM09677) and differentiated into motor neurons according to methods adapted from Dimos, J. T., et. al. Science (2008) 321, 1218-122, Epub Jul. 31, 2008).

### Example 32

### Methods

### Compound Preparation and Addition

Compounds were stored at appropriate temperature and dissolved accordingly to the manufacturers (Tocris, Enzo, EMD and Sigma-Aldrich) in DMSO (Sigma-Aldrich), unless stated otherwise (Monensin Na in EtOH, DMPQ in water). Growth factors and PDGF neutralizing antibodies (R&D Systems) were reconstituted with buffers recommended by the supplier. For generation of the dose curves, 1×10³ fibroblasts were plated on to 96-well Greiner Senso plates (VWR) and treated with compounds on the second day then fixed and stained at the indicated time points. For GSK inhibitors and performing p-GSK3b(Ser9) detection, two days after plating growth media containing 5% FBS was replaced to the media with FBS reduced down to 0.5%, to starve cells for 72 hours. Addition of the GSK inhibitors was done in 5% FBS enriched media. As previously described, starvation increases levels of constitutively activate GSK3b kinase, allowing to achieve a more dramatic inhibitory effect when GSK inhibitors are added together with growth factors that are also known to inhibit GSK by Ser-9 phosphorylation. Full starvation was observed to be toxic for primary fibroblast cells, therefore media with reduced FBS levels was used. In addition certain compounds also worked better with starvation, for example Coumermycin A1. For all compounds and growth factors control wells received solvent in amount equivalent to the highest concentration of the compound in a dose curve. For PDGF inhibitory studies, cells were pre-treated with kinase inhibitors for 2 hours and PDGF was added with a fresh kinase inhibitor solution and incubated for an additional 3 days.

### Immunostaining and Imaging.

During the screen cells were fixed routinely by using MAPD robot that added equivalent volume of 8% PFA directly to the cells growing in the media, therefore giving a final concentration of 4% PFA. To find best SMN signal to noise ratio we tested several other fixation techniques, and fixation in ice cold methanol/acetone (1:1 volume) yielded the best results and was used for all the secondary compound assays using OperaPerkinElmer system. For detection of the SMN protein, after blocking with 5% goat serum for 30 min, we used mouse monoclonal a-SMN antibodies (BD Pharmingen) at the final concentration of 1.25 μg/ml also diluted in 5% goat serum/PBS, that we incubated with our cells for 2 hours. For p-GSK(Ser9) detection we incubated our plates with anti-p-GSK(Ser9) rabbit antibodies (SantaCruz) at 1:100 dilution for 2 hours. Following three washes, we incubated our plates with anti-mouse (for SMN) or anti-rabbit (for p-GSK(Ser9) secondary antibodies made in goat that were conjugated with 488 fluorophore for 1 hour. We used Hoechst nuclear die (Invitrogen) in 1:5000 dilution for 1 hour along with the secondary antibodies to detect nuclei.

qPCR

SRNA was purified using Tri-Reagent (Sigma-Aldrich) protocol and quantified on the Nanodrop (Thermo Fisher Scientific). From 500 ng of total RNA cDNA was made using Superscript®VILO™ cDNA synthesis kit (Invitrogen). Reverse transcription was performed on a thermocycler (Biorad) with the following temperature conditions: 25° C. for 10 minutes, 42° C. for 2 hours, 85° C. at 5 minutes. Synthesised cDNA was diluted 5 times and 1 μl of cDNA sample was PCR amplified using RT²SYBR Green/ROX PCR Master Mix and primer assays for SMN1, SMN2, GAPDH (SABiosciences) genes on 7900HT Fast Real-Time PCR System (ABI). qPCR results were analyzed with RQ Manager Software (ABI) by absolute quantification method.

### Immunoblot Analysis

Cells were lysed with RIPA buffer (Thermo Fisher Scientific) supplemented by addition of complete protease and phosphatase inhibitor cocktails (VWR). Protein in cellular lysates was quantified by using DC protein assay based on the protocol of the manufacturer (Biorad), and 10 ug of lysate was resolved by electrophoresis through 10% Tris-Glycin Novex gel and transferred to PVDF membranes (Invitrogen) by semi-dry transfer (apparatus). Membranes were incubated overnight with 1:10,000 monoclonal anti-SMN (MAB8, BD Biosciences, CA, USA), rabbit a-GSK3-13 antibodies overnight 1:1000 (Sigma) and 1 hour with monoclonal anti-human 13-tubulin diluted 1:10,000 (Abcam). Primary antibodies were detected by secondary antibodies conjugated with horseradish peroxidase-conjugated goat anti-mouse for SMN and goat anti-rabbit IgG for p-GSK(Ser9) (ThermoFisher) and enhanced chemiluminescence (West Pico Reagent, Pierce, Rockford, Ill., USA).

### Phospho-Kinase Array

Cellular lysates from fibroblasts GM09677 treated with 50 ng/ml of PDGF-BB for 30 minutes and untreated control fibroblasts were collected and hybridized with the phosphor-kinase array membranes accordingly to the manufacture protocol (R&D Systems). Nitrocellulose membrane contained pre-arrayed antibodies against 46 different kinase phosphorylation sites and control antibodies. The array is washed to remove the inbound proteins followed by the incubation with a cocktail of biotinylated detection antibodies. Streptavidin-HRP and chemiluminescent detection reagents are applied and signal is produced at each capture spot corresponding to the amount of phosphorylated protein bound. Levels of phosphorylated protein were detected by measurement on the X-ray film (Kodak).

### Lentivirus Preparation

All recombinant lentiviruses were produced by transient transfection of HEK 293T cell. Briefly subconfluent 293T cells were cotranfected with 3 μg PAX2, 3 μg pMD2G, and 3 μg pGIPZ construct (Openbiosystems) by lipofectimine (Invitrogen). Lentiviral particles were concentrated by collecting supernatant and harvesting sample after a centrifugation with an Amicon Ultra-15 filter with a 100 kDa filter. Vector titers were determined by titering virus on HEK 293T cells.

### Motor Neuron Survival Assay

Mouse ES-cells expressing cherry fluorescent protein (CFP) under control of a Hb9 promoter, a motor neuron specific transcription factor, were used to differentiate motor neurons as described in Example 31. Motor neurons were infected with the three constructs that were acquired from Open Biosystems (pGIPZ): a non-silencing (NS) shRNA and two constructs with unique shRNA sequences directed against the Smn transcript. Virtually all of the motor neurons were infected under the conditions chosen for these experiments. First we performed motor neuron survival assays to test whether reduced levels of SMN will affect viability of these motor neurons. We differentiated mES cells with Hb9-Chemy Fluorescent Protein (Hb9-CFP) in to motor neurons expressing CFP, that later we dissociated and plated on DIV0. Cells were infected with the NS pGIPZ virus, SMN#1 pGIPZ virus, SMN#2 pGIPZ virus or no virus on DIV 1. The percentage of surviving motor neuron was normalized to the uninfected control culture. For testing several of our hit compounds for their ability to rescue the survival of Motor Neurons with SMN knockdown, after motor neurons were plated (DIV0), and infected with the lentivirus (DIV2), we added compound two days later (DIV4). Over the course of three days after first compound addition (DIV4-6), time when usually the effects of SMN knockdown become the most prominent, we counted live cherry expressing motor neurons on a daily basis. The percentage of surviving motor neuron was normalized to the culture infected with control non-targeting shRNA. Results of such a motor neuron survival assay are shown in FIG. 27d.

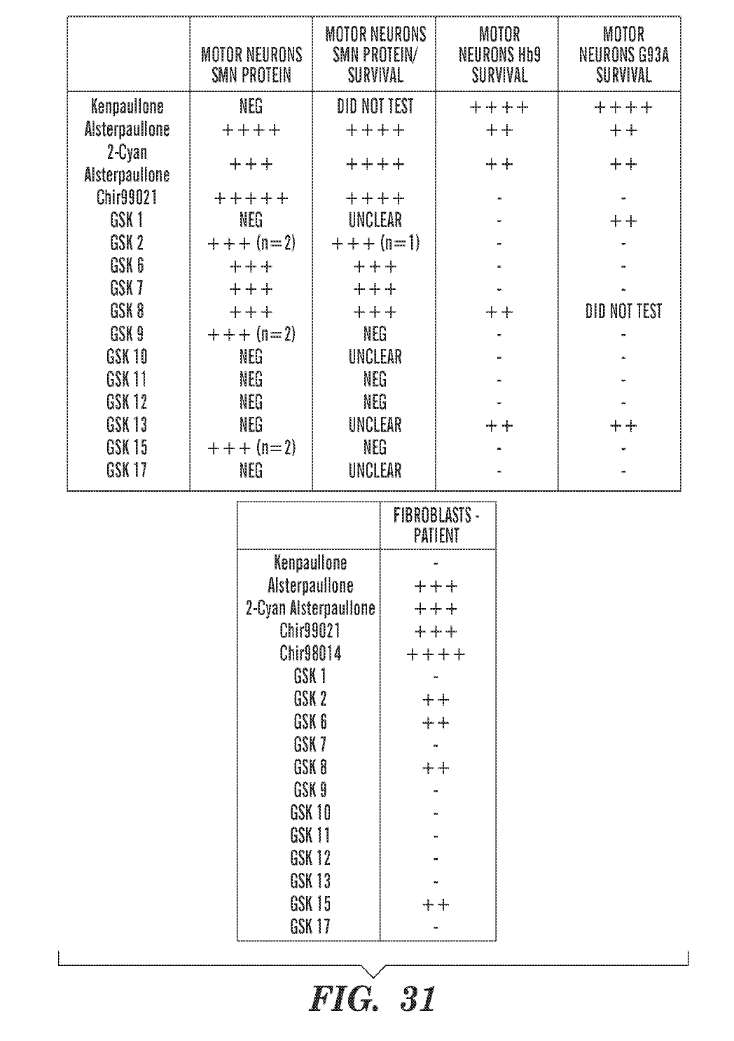

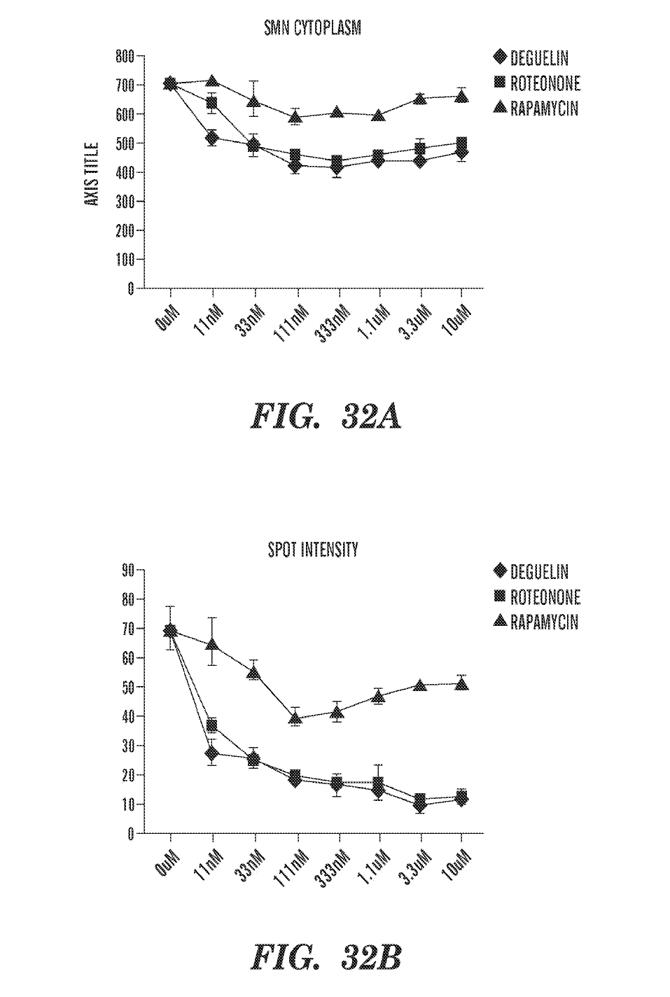

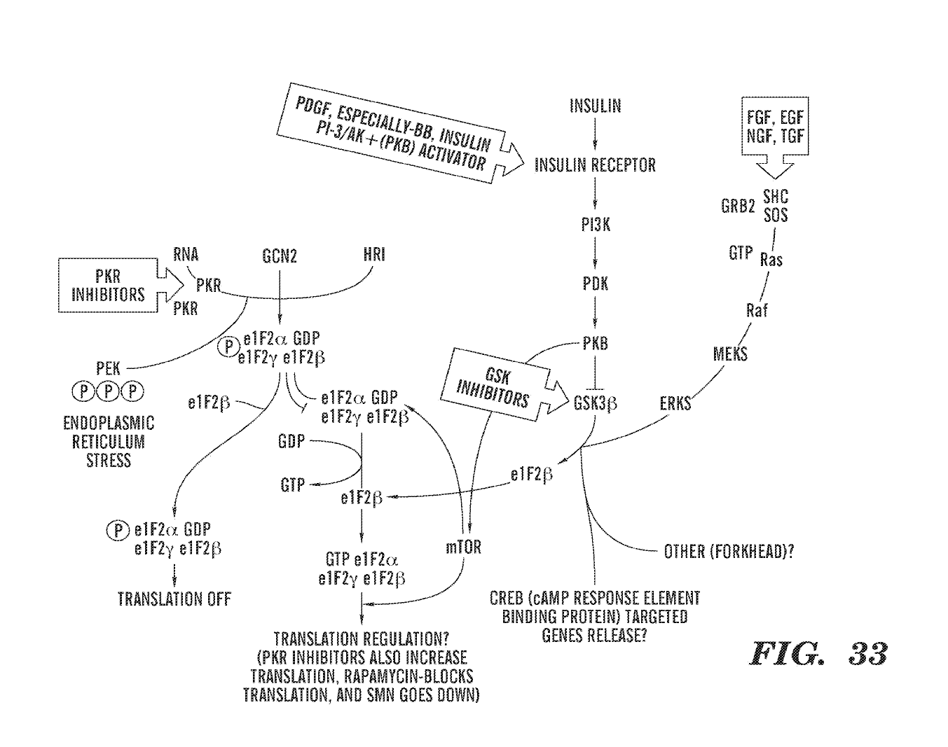

Another way of assessing motor neuron survival is by withdrawing the neurotrophic factors from the media. Briefly, motor neurons are grown in media containing neurotrophic factors. The neurotrophic factors are then withdrawn from the growth media, e.g., cells transferred to media having no neurotrophic factors or neurotrophic factor containing media exchanged with neurotrophic factors lacking media. The compound of interest is added and motor neurons are counted on a daily basis. Results of such a motor neuron survival assay are shown in FIG. 31a.

### Fluorescence-Activated Cell Sorting (FACS)

Hb9-CFP mouse ES cell derived motor neurons that had been plated on Day 0 and infected with pGIPZ lentivirus on Day 3 were sorted on Day 7. Cells were gently removed from the with 20 units of Papain and 1,000 units of DNase I (Worthington Biochemical). Cells were washed and resuspended in PBS. Motor neurons which were CFP positive and GFP positive, the marker contained in the pGIPZ constructs to mark lentiviral transduction, were isolated via FACS on a Mo-Flo cell sorter (Beckman Coulter).

### Example 33

### Na,K ATPase Inhibitors Increase SMN Levels

Na⁺/K⁺ ATPase inhibitors, such as the cardiac glycoside ouabain, consistently emerged as hits during screening. Multiple cardiac glycosides could increase SMN in the nucleus and cytoplasm at concentrations close to those at which they are known to inhibit the Na/K ATPase (FIG. 35a). Since these enzyme inhibitors produce a net gain in intracellular Na⁺ which may then lead to increased intracellular Ca²⁺ via activity of the Na, Ca exchanger, we also tested compounds that directly increase Na⁺ or Ca²⁺ levels. Monensin, a sodium ionophore, was able to increase SMN levels in the nucleus 2 fold (FIG. 35b). Next we tested both agonists and antagonists of intracellular Ca²⁺. We also found that Ca²⁺ ionophores, such as A23187 (calcimycin) (FIG. 35c) and ionomycin (FIG. 35d) were active. As an alternative way of increasing Ca²⁺, we tested Thapsigargin the inhibitor of intracellular calcium (SERCA) pumps that releases Ca²⁺ in to the cytoplasm from the storages, also caused SMN upregulation (FIG. 35e). At the same time Ca²⁺ antagonists were not active (data not shown). This demonstrates that one way in which ouabain and other Na⁺/K⁺ ATPase inhibitors increase SMN is by increasing intracellular calcium. Since the Cα agonists also affect cellular viability at the higher concentrations, additional work on these types of agents is currently being carried out. The objective is to find the way to increase intracellular Ca2+ in a more controlled way to promote SMN increase in SMA patient-derived fibroblasts without the toxicity, or to find Ca2+ downstream signaling targets that would be suitable for increasing SMN in ES-derived motor neurons.

### Example 34

### Growth Factors Increase SMN Levels

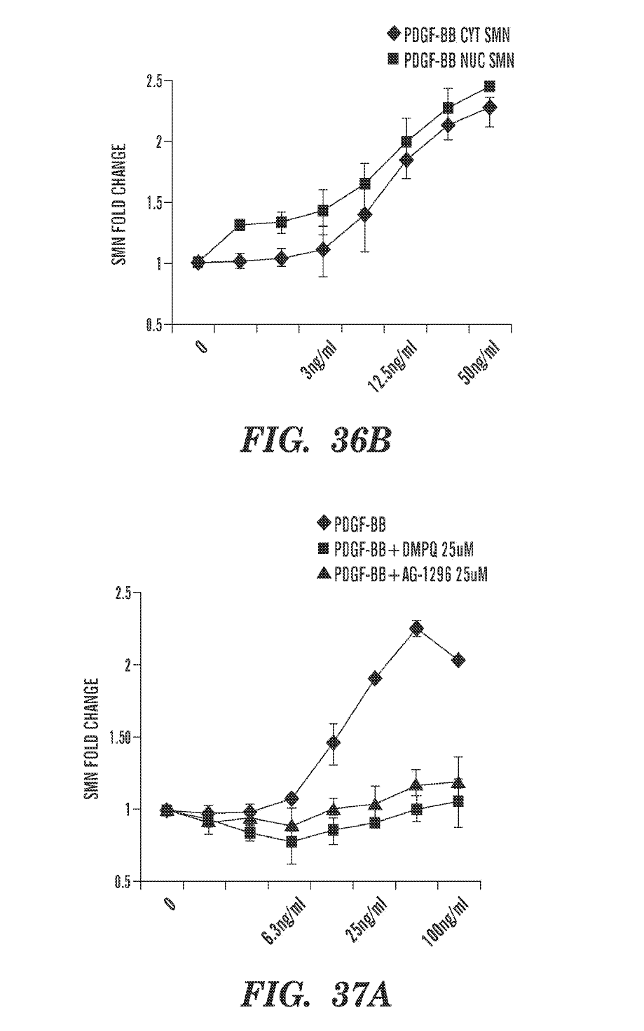

Another potential mode of action for ouabain is as an activator of receptor tyrosine kinase (RTK) signaling (citations). We carried out a set of experiments to see if RTK signaling might also regulate SMN levels. So we next wanted to know whether growth factors that specifically stimulate pathways downstream of RTK like PI3 and MAPK would be able to increase SMN level. Thus, fibroblasts were incubated with various RTK ligands for 2 days and their SMN levels were measured. We tested several different ligands for RTKs and several of the were active including bFGF (FIG. 36a) and identified that platelet-derived growth factor (PDGF) and in particular PDGF-BB was the most potent growth factor that elevated SMN protein levels (FIG. 36b).

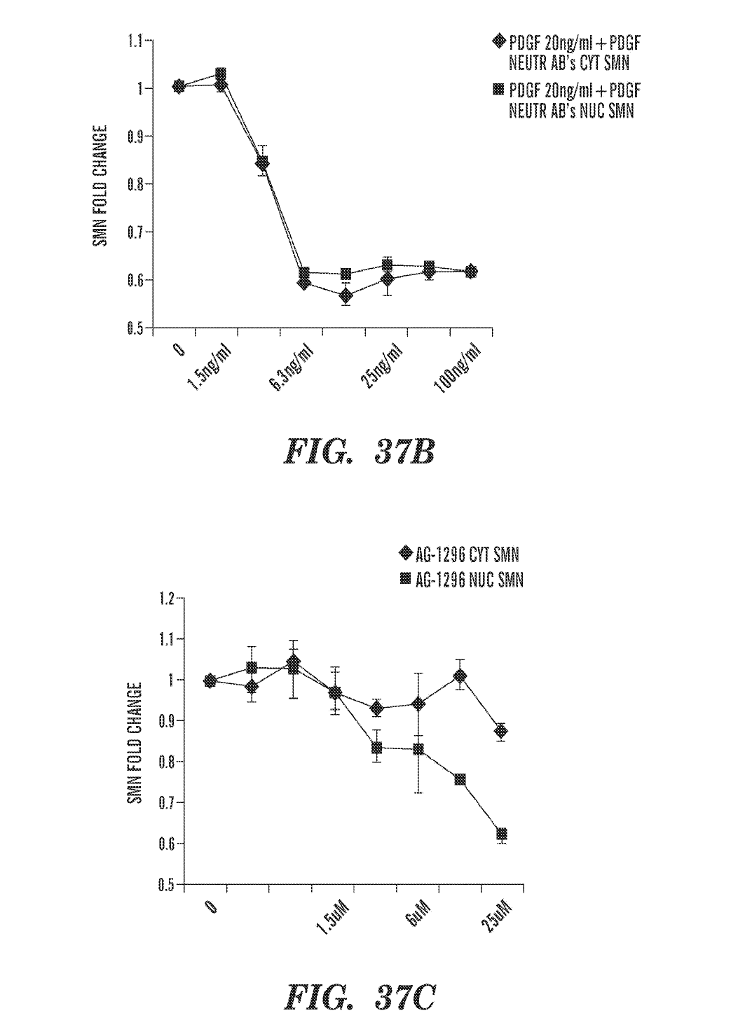

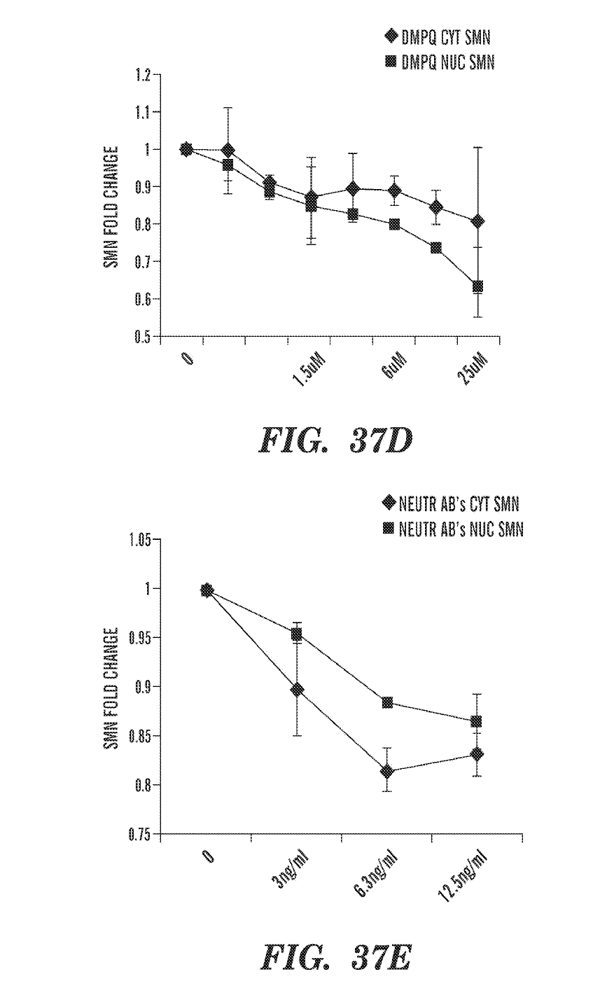

The PDGF effect was specific since it could be abolished by pre-incubating cells either of two small molecule PDGF receptor (PDGFR) inhibitors, DMPQ and AG-1236, (FIG. 37a) or with a PDGF neutralizing antibody (FIG. 37b). Since we normally maintain our cells in PDGF-containing serum medium, we were also interested in seeing if SMN amounts could be reduced below their normal “basal” levels. This also appeared to be the case since either reducing serum from 10% to 0.5% (data not shown) or adding the PDGF neutralizing antibody or PDGR inhibitors to serum could decrease SMN (FIGS. 37c-37e) This suggests that RTK ligands such as PDGF might serve as physiological regulators of SMN.

1. A method of promoting motor neuron cell survival, the method comprising:
contacting a motor neuron cell with a compound of formula (VI) 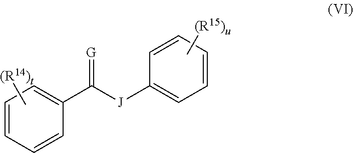
wherein:
t is 0, 1, 2, 3, 4 or 5; and
u is 0, 1, 2, 3, 4 or 5;
G is 0 or S;
J is O, S, NH or CH₂;
each R¹⁴ and R¹⁵ is independently alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R)₂, —C(═O)OR^{c}, —OR^{d}, —NR^{e}₂, or —SR^{f}, each of which is optionally substituted with 1-4 R¹⁶;
each R¹⁶ is independently halo, —NO₂, —SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl; and
each R^{f} is independently hydrogen, alkyl, alkenyl, alkynyl, aryl, heteroaryl, arylalkyl, alkylhydroxy or haloalkyl.

2-6. (canceled)

7. The method of claim 1, wherein the motor neuron comprises a mutation in gene encoding SMN1 or a mutation in gene encoding superoxide mutase 1 (SOD1).

8. (canceled)

9. The method of claim 1, wherein the motor neuron comprises a G->A mutation at position 93 of gene encoding SOD1.

10. The method of claim 1, wherein the compound modulates survival of motor neuron (SMN) protein levels.

11-21. (canceled)

22. The method of claim 1, wherein the contact is in vitro.

23. The method of claim 1, wherein the contact is in vivo.

24. (canceled)

25. The method of claim 23, wherein in vivo contact is in a subject, where the subject is selected for treatment of a neurodegenerative disorder characterized by degeneration of motor neurons.

26. (canceled)

27. The method of claim 25, wherein the neurodegenerative disorder is characterized by a mutation in the SMN gene or by diminished levels of SMN protein.

28. (canceled)

29. The method of claim 25, wherein the neurodegenerative disorder is spinal muscular atrophy (SMA) or amyotrophic lateral sclerosis (ALS).

30. (canceled)

31. A method of promoting motor neuron survival, the method comprising: contacting a motor neuron with a compound that modulates a biological pathway selected from the group consisting of PI-3K signaling pathway, Akt signaling pathway, MAPK signaling pathway, PDGF pathway, RAS pathway, eIF2 pathway, glycogen synthase kinase (GSK) pathway, PKR pathway, Insulin Receptor Pathway, mTOR pathway, EGF pathway, NGF pathway, FGF pathway, TGF pathway, BMP pathway, receptor tyrosine kinase (RTK) pathway, and any combinations thereof or a target selected from the group consisting of Na⁺/K⁺ channel, MAPK, cannobinoid receptor, GPCR, Ca²⁺ channel, K⁺ channel, PDE5, GSK/CDK, PKR, CDK2, CDK5, GSK-313, IKK-2, IKK-213, proteasome, BMP/TGFbeta receptor, dopamine receptor, and any combinations thereof.

32-36. (canceled)

37. The method of claim 31, wherein the compound is selected from the group consisting of formula (I): 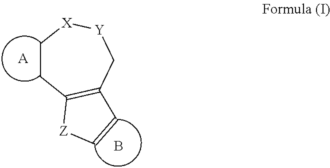
wherein:
A represents, with the adjacent ring, an optionally substituted aryl or an optionally substituted heteroaryl;
B represents, with the adjacent ring, an optionally substituted aryl or an optionally substituted heteroaryl;
X is NR^{N}, O, S, or CH₂;
Y is C(O), C(S), CH—SR^{N}CH—NHOH or S;
Z is NR^{N}, O, S or CHR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy; and physiologically acceptable salts thereof, formula (II): 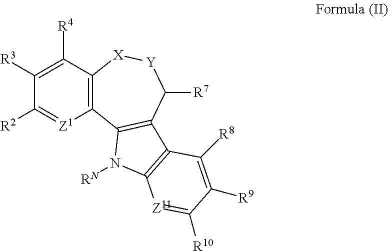
wherein:
Z¹ is N or CR¹;
Z¹¹ is N or CR¹¹;
R¹, R², R³, R⁴, R⁷, R⁸, R⁹, R¹⁰ and R¹¹ are each independently hydrogen, optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂—SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂. NR^{N}, C(O), NR^{N}C(O)O, or OC(O)NR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy;
R¹² is independently for each occurrence optionally substituted linear or branched alkyl, optionally substituted alkenyl, optionally substituted alkynyl, optionally substituted aryl, optionally substituted heteroaryl, optionally substituted arylalkyl, optionally substituted haloalkyl or optionally substituted alkylhydroxy;
X is NR^{N}, O, S, or CH₂;
Y is C(O), C(S), CH—SR^{N}CH—NHOH or S; and physiologically acceptable salts thereof, formula (III): 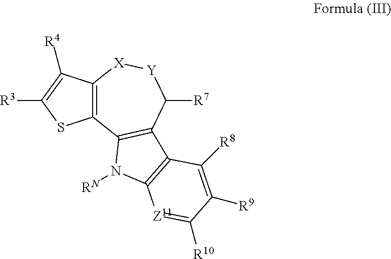
wherein:
Z¹¹ is N or CR¹¹;
R³, R⁴, R⁷, R⁸, R⁹, R¹⁰ and R¹¹ are each independently hydrogen, optionally substituted linear or branched alkyl, optionally substituted alkenyl, optionally substituted alkynyl, optionally substituted aryl, optionally substituted heteroaryl, optionally substituted cycloalkyl, optionally substituted heterocyclic, optionally substituted arylalkyl, optionally substituted haloalkyl, halo, —OH, —NO₂—SO₃⁻, —CN, —CF₃, C(O)-halo, —C(O)R¹², —C(O)N(R¹²)₂, —C(O)OR¹², —OR¹², —NH₂, —N(R¹²)₂, or —SR¹², wherein backbone of the alkyl, alkenyl or alkynyl can contain one or more of O, S, S(O), SO₂, NR^{N}, C(O), NR^{N}C(O)O, or OC(O)NR^{N};
R^{N} is hydrogen, optionally substituted linear or branched alkyl, optionally substituted alkenyl, optionally substituted alkynyl, optionally substituted aryl, optionally substituted heteroaryl, optionally substituted arylalkyl, optionally substituted haloalkyl, or optionally substituted alkylhydroxy;
R¹² is independently for each occurrence optionally substituted linear or branched alkyl, optionally substituted alkenyl, optionally substituted alkynyl, optionally substituted aryl, optionally substituted heteroaryl, optionally substituted arylalkyl, optionally substituted haloalkyl or optionally substituted alkylhydroxy;
X is NR^{N}, O, S, or CH₂;
Y is C(O), C(S), CH—SR^{N}CH—NHOH or S; and physiologically acceptable salts thereof, formula (IV): 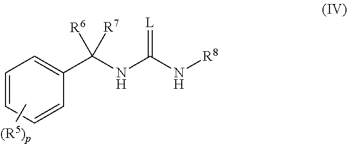
wherein:
L is O or S; and
p is 0, 1, 2, 3, 4 or 5;
each R⁵ is independently alkyl, alkenyl, alkynyl, aryl, heteroaryl, arylalkyl, haloalkyl, alkylhydroxy, halo, —NO₂—SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R⁹;
each R⁶ and R⁷ is independently hydrogen, alkyl, alkenyl, alkynyl, aryl, heteroaryl, arylalkyl, haloalkyl, or alkylhydroxy;
R⁸ is alkyl, alkenyl, alkynyl, aryl, heteroaryl, arylalkyl, haloalkyl, alkylhydroxy, halo, —NO₂, —SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹⁰;
each R⁹ and R¹⁰ is independently halo, —NO₂—SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, alkylhydroxy, haloalkyl or halo;
each R^{b} is independently hydrogen, alkyl, alkenyl, alkynyl, aryl, heteroaryl, arylalkyl, alkylhydroxy, or haloalkyl;
each R^{c} is independently hydrogen, alkyl, alkenyl, alkynyl, aryl, heteroaryl, arylalkyl, alkylhydroxy, or haloalkyl;
each R^{d} is independently hydrogen, alkyl, alkenyl, alkynyl, aryl, heteroaryl, arylalkyl, alkylhydroxy, or haloalkyl;
each R^{e} is independently hydrogen, alkyl, alkenyl, alkynyl, aryl, heteroaryl, arylalkyl, alkylhydroxy, or haloalkyl;
each R^{f} is independently hydrogen, alkyl, alkenyl, alkynyl, aryl, heteroaryl, arylalkyl, alkylhydroxy, or haloalkyl; and physiologically acceptable salts thereof,
formula (V): 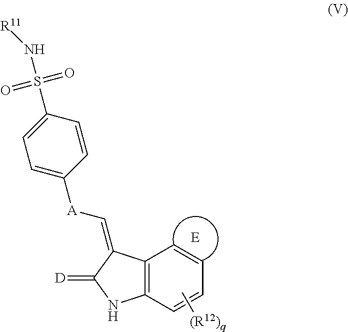
wherein:
A is NH, O, S or CH₂; and
D is O or S;
E is an aryl or heteroaryl moiety;
q is 0, 1 or 2;
each R¹¹ and R¹² is independently hydrogen, alkyl, alkenyl, alkynyl, aryl, heteroaryl, arylalkyl, haloalkyl, alkylhydroxy, halo, —NO₂—SO₃⁻, —CN, —C(═O)R^{a}, —C(═O)N(R^{b})₂, —C(═O)OR^{c}, —OR^{d}, —NR^{e}₂, or —SR^{f}, each of which is optionally substituted with 1-4 R¹³;
each R¹³ is independently halo, —NO₂—SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl;
each R^{f} is independently hydrogen, alkyl, alkenyl, alkynyl, aryl, heteroaryl, arylalkyl, alkylhydroxy or haloalkyl; and physiologically acceptable salts thereof,
formula (VI): 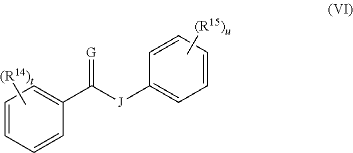
wherein:
t is 0, 1, 2, 3, 4 or 5; and
u is 0, 1, 2, 3, 4 or 5;
G is 0 or S;
J is O, S, NH or CH₂;
each R¹⁴ and R¹⁵ is independently alkyl, alkenyl, alkynyl, aryl, heteroaryl, arylalkyl, haloalkyl, alkylhydroxy, halo, —NO₂—SO₃⁻, —CN, —C(═O)R^{a}, —C(═O)N(R^{b})₂, C(═O)OR^{c}, —OR^{d}, —NR^{e}₂, or —SR^{f}, each of which is optionally substituted with 1-4 R¹⁶;
each R¹⁶ is independently halo, —NO₂—SO₃⁻, —CN, —C(═O)R^{a}, —C(═O)N(R^{b})₂, —C(═O)OR^{c}, —OR^{d}, —NR^{e}₂, or —SR^{f};
each R^{a} is independently hydrogen, alkyl, alkenyl, alkynyl, aryl, heteroaryl, arylalkyl, haloalkyl, alkylhydroxy or halo;
each R^{b} is independently hydrogen, alkyl, alkenyl, alkynyl, aryl, heteroaryl, arylalkyl, alkylhydroxy or haloalkyl;
each R^{c} is independently hydrogen, alkyl, alkenyl, alkynyl, aryl, heteroaryl, arylalkyl, alkylhydroxy or haloalkyl;
each R^{d} is independently hydrogen, alkyl, alkenyl, alkynyl, aryl, heteroaryl, arylalkyl, alkylhydroxy or haloalkyl;
each R^{e} is independently hydrogen, alkyl, alkenyl, alkynyl, aryl, heteroaryl, arylalkyl, alkylhydroxy or haloalkyl;
each R^{f} is independently hydrogen, alkyl, alkenyl, alkynyl, aryl, heteroaryl, arylalkyl, alkylhydroxy or haloalkyl; and physiologically acceptable salts thereof, and any combinations thereof.

38. The method of claim 31, wherein the motor neuron comprises a mutation in gene encoding SMN1 or a mutation in gene encoding superoxidase mutase (SOD1).

39. (canceled)

40. The method of 38, wherein the motor neuron comprises a G->A mutation at position 93 of gene encoding SOD1.

41. The method of claim 31, wherein the compound modulates survival of motor neuron (SMN) protein levels.

42-51. (canceled)

52. The method of claim 31, wherein the contact is in vivo.

53. (canceled)

54. The method of claim 52, wherein in vivo contact is in a subject, where the subject is selected for treatment of a neurodegenerative disorder characterized by degeneration of motor neurons.

55. (canceled)

56. The method of claim 54, wherein the neurodegenerative disorder is characterized by a mutation in the SMN gene or by diminished levels of SMN protein.

57. (canceled)

58. The method of claim 54, wherein the neurodegenerative disorder is spinal muscular atrophy (SMA) or amyotrophic lateral sclerosis (ALS).

59. The method of claim 31, wherein the compound is selected from the group consisting of Kenpaullone, Alsterpaullone, 2-cyanoethyl-alsterpaullone, CHIR9814, CHIR99021, GSK1, GSK2, GSK6, GSK7, GSK8, GSK13, and any combinations thereof.

60-119. (canceled)

120. A method of increasing SMN protein levels in a cell, the method comprising: contacting a cell with a compound, wherein the compound modulates a biological pathway selected from the group consisting of PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and any combinations thereof or a target selected from the group consisting Na⁺/K⁺ channel, MAPK, cannobinoid receptor, GPCR, Ca²⁺ channel, K⁺ channel, PDE5, GSK/CDK, PKR, CDK2, IKK-2, proteasome, BMP/TGFbeta receptor, dopamine receptor, and any combinations thereof.

121-133. (canceled)
